(19) United States
(12) Patent Application Publication
(10) Pub. No.: US 2026/0185071 A1
(43) Pub. Date: Jul. 2, 2026

(54) COMPOSITIONS AND METHODS FOR ALTERING A NUCLEOBASE IN A LIPOPROTEIN(A) POLYNUCLEOTIDE

(71) Applicant: Beam Therapeutics Inc., Cambridge, MA (US)

(72) Inventors: Tanggis Bohnuud, Cambridge, MA (US); Margaret Parker, Cambridge, MA (US); Tianyu Zou, Cambridge, MA (US); Chad Orrin Brown, Cambridge, MA (US)

(73) Assignee: Beam Therapeutics Inc., Cambridge, MA (US)

(21) Appl. No.: 19/563,909

(22) Filed: Mar. 11, 2026

Related U.S. Application Data
(63) Continuation of application No. PCT/US2024/047459, filed on Sep. 19, 2024.
(60) Provisional application No. 63/583,795, filed on Sep. 19, 2023.

(51) Int. Cl. C12N 9/22; A61P 9/10; C12N 9/78; C12N 15/11; C12N 15/88

(52) U.S. Cl. CPC C12N 9/226; A61P 9/10; C12N 9/78; C12N 15/11; C12N 15/88; C12Y 305/04004; C12Y 305/04005; C12N 2310/20

(57) ABSTRACT

The disclosure features compositions and methods for treating cardiovascular disease by introducing one or more alterations into a lipoprotein(a) (LPA) polynucleotide in a cell. In particular embodiments, the disclosure provides a base editor system (e.g., a fusion protein or complex comprising a programable DNA binding protein, a nucleobase editor, and gRNA) for modifying an LPA polynucleotide, where the modification is associated with reduced expression, and/or reduced activity of the LPA polypeptide encoded by the polynucleotide.

### CROSS REFERENCE TO RELATED APPLICATIONS

This application is a continuation under 35 U.S.C. § 111(a) of PCT International Patent Application No. PCT/US2024/047459, filed Sep. 19, 2024, designating the United States and published in English, which claims priority to and the benefit of U.S. Provisional Application No. 63/583,795, filed Sep. 19, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Sep. 12, 2024, is named 180802-056102PCT_SL.xml and is 2,559,791 bytes in size.

### BACKGROUND

Cardiovascular disease (CVD) is the leading cause of death globally. An estimated 17.9 million people died from CVDs in 2019, representing 32% of all global deaths. Of these deaths, 85% were due to heart attack and stroke. Over three quarters of CVD deaths take place in low- and middle-income countries. Out of the 17 million premature deaths (under the age of 70) due to noncommunicable diseases in 2019, 38% were caused by CVDs. Therefore, improved methods for treating cardiovascular disease are urgently required.

### SUMMARY

As described below, the disclosure features compositions and methods for treating cardiovascular disease by introducing one or more alterations into a lipoprotein(a) (LPA) polynucleotide in a cell. In particular embodiments, the disclosure provides a base editor system (e.g., a fusion protein or complex comprising a programable DNA binding protein, a nucleobase editor, and gRNA) for modifying an LPA polynucleotide, where the modification is associated with reduced expression, and/or reduced activity of the LPA polypeptide encoded by the polynucleotide. Non-limiting examples of alterations include base edits and/or double-strand cuts.

In one aspect, the disclosure provides a method of editing a nucleobase of a lipoprotein A (LPA) polynucleotide in a cell. The method involves contacting the LPA polynucleotide with a base editor system containing a guide RNA, or a polynucleotide encoding the guide RNA, and a base editor containing, or one or more polynucleotides encoding the base editor, where the base editor contains a fusion protein or a protein complex. The base editor contains a nucleic acid programmable DNA binding protein (napDNAbp) domain and a deaminase domain. The guide RNA targets the base editor to effect an alteration of the nucleobase of the LPA polynucleotide.

In another aspect, the disclosure provides a method of treating atherosclerosis and/or cardiovascular disease in a subject in need thereof The method involves contacting a cell of the subject with a base editor system containing a base editor, or one or more polynucleotide encoding the base editor, where the base editor contains a fusion protein or protein complex. The base editor contains a nucleic acid programmable DNA binding protein (napDNAbp) domain and a deaminase domain. The base editor system further contains a guide RNA, or a polynucleotide encoding the guide RNA, where the guide RNA targets the base editor to effect an alteration of a nucleobase of an LPA polynucleotide.

In another aspect, the disclosure provides a modified cell containing an alteration in a nucleobase of a LPA polynucleotide. The alteration is prepared by the method of any aspect of the disclosure, or embodiments thereof.

In another aspect, the disclosure provides a base editor system containing a fusion protein, or one or more polynucleotides encoding the fusion protein, where the fusion protein contains a nucleic acid programmable DNA binding protein domain (napDNAbp) and a deaminase domain. The base editor system further contains a guide RNA, or a guide RNA encoding the guide RNA. The guide RNA targets the base editor to effect an alteration of a nucleobase of an LPA polynucleotide.

In another aspect, the disclosure provides a polynucleotide or set of polynucleotides encoding the base editor system of any aspect of the disclosure, or embodiments thereof, or a component thereof.

In another aspect, the disclosure provides a lipid nanoparticle containing the base editor system, the polynucleotide, or the set of polynucleotides of any aspect of the disclosure, or embodiments thereof.

In another aspect, the disclosure provides a pharmaceutical composition containing the modified cell, the base editor system, the polynucleotide, the set of polynucleotides, or the lipid nanoparticle of any aspect of the disclosure, or embodiments thereof, and a pharmaceutically acceptable excipient.

In another aspect, the disclosure provides a kit containing the modified cell, the base editor system, the polynucleotide, the set of polynucleotides, the lipid nanoparticle, or the pharmaceutical composition of any aspect of the disclosure, or embodiments thereof, for use in the method of any aspect of the disclosure, or embodiments thereof. The kit further contains a container.

In another aspect, the disclosure provides a guide RNA containing a sequence listed in Table TA-1, 1A-2, 1B-1, 1B-2, 1C-1, or 1C-2.

In any aspect of the disclosure, or embodiments thereof, the cell is in vivo or in vitro. In any aspect of the disclosure, or embodiments thereof, the cell is a mammalian cell. In any aspect of the disclosure, or embodiments thereof, the cell is a liver cell in a rodent or human. In any aspect of the disclosure, or embodiments thereof, the cell is a hepatocyte.

In any aspect of the disclosure, or embodiments thereof, the cell is contacted with or the base editor system contains two or more guide RNAs, where each guide RNA binds a different location within the LPA polynucleotide.

In any aspect of the disclosure, or embodiments thereof, the guide RNA is selected from the guides listed in Tables TA-1, 1B-1, and 1C-1.

In any aspect of the disclosure, or embodiments thereof, the deaminase domain is an adenosine deaminase or a cytidine deaminase. In any aspect of the disclosure, or embodiments thereof, the adenosine deaminase contains an amino acid sequence with at least about 90% identity to the following amino acid sequence or a fragment thereof lacking the N-terminal methionine and contains one or more amino acid alterations selected from one or more of I76Y, V82S, Y123H, Y147R, and Q154R compared to the following amino acid sequence: TadA*7.10 MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1). In any aspect of the disclosure, or embodiments thereof, the adenosine deaminase contains the amino acid alterations I76Y, V82S, Y123H, Y147R, and Q154R. In any aspect of the disclosure, or embodiments thereof, the adenosine deaminase has at least about 95% identity to SEQ ID NO: 1. In any aspect of the disclosure, or embodiments thereof, the cytidine deaminase contains an amino acid sequence with at least about 90% identity to the following amino acid sequence or a fragment thereof lacking the N-terminal methionine:

ppAPOBEC1
(SEQ ID NO: 23)
MTSEKGPSTGDPTLRRRIESWEFDVFYDPRELRKETCLLYEIKWGMSR
KIWRSSGKNTTNHVEVNFIKKFTSERRFHSSISCSITWFLSWSPCWEC
SQAIREFLSQHPGVTLVIYVARLFWHMDQRNRQGLRDLVNSGVTIQIM
RASEYYHCWRNFVNYPPGDEAHWPQYPPLWMMLYALELHCIILSLPPC
LKISRRWQNHLAFFRLHLQNCHYQTIPPHILLATGLIHPSVTWR.

In any aspect of the disclosure, or embodiments thereof, alteration of the nucleobase is associated with a reduction in transcription of a polynucleotide sequence encoding the LPA protein. In any aspect of the disclosure, or embodiments thereof, alteration of the nucleobase in the LPA polynucleotide results in a missense mutation. In any aspect of the disclosure, or embodiments thereof, alteration of the nucleobase in the LPA polynucleotide disrupts a splice site. In any aspect of the disclosure, or embodiments thereof, the alteration introduces a single nucleotide polymorphism (SNP) into the LPA polynucleotide, where the SNP is associated with reduced serum concentrations of LPA in a subject and/or reduced incidence of atherosclerosis and/or cardiovascular disease in a subject. In various embodiments, the SNP is selected from one or more of, chr6:160531784:T>C, chr6:160532531:C>T, chr6:160548552:G>A, chr6:160532610:A>G, chr6:160591049:A>G, chr6:160635134:G>A, and chr6:160547886:A>G. In any aspect of the disclosure, or embodiments thereof, the alteration results in a premature STOP codon. In any aspect of the disclosure, or embodiments thereof, the alteration is associated with a reduction in incidence of cardiovascular disease in a subject.

In any aspect of the disclosure, or embodiments thereof, the napDNAbp domain is a Staphylococcus aureus Cas9 (saCas9) or a Streptococcus pyogenes Cas9 (spCas9). In any aspect of the disclosure, or embodiments thereof, the napDNAbp has specificity for a protospacer adjacent motif (PAM) selected from one or more of: NG, NGA, NGC, NGG, NNGRRT, NNNRRT, and NRCH, where “N” indicates A, C, G, or T, “R” indicates A or G, and “H” represents A, C, or T. In any aspect of the disclosure, or embodiments thereof, the Cas9 is a dead Cas9 (dCas9) or Cas9 nickase (nCas9). In any aspect of the disclosure, or embodiments thereof, the base editor contains an amino acid sequence with at least about 90% identity to a sequence listed in Table 2. In any aspect of the disclosure, or embodiments thereof, the guide RNA contains a nucleic acid sequence containing at least 10, 11, 12, 13, 14, 15, 16, 17, 18, 19, or 20 contiguous nucleotides of a spacer nucleic acid sequence listed in Table 1A-2, 1B-2, or 1C-2. In any aspect of the disclosure, or embodiments thereof, the guide RNA contains a nucleic acid sequence listed in Table 1A-I, 1B-1, or IC-1.

In any aspect of the disclosure, or embodiments thereof, the guide RNA contains a nucleic acid analog. In any aspect of the disclosure, or embodiments thereof, the guide RNA contains one or more of a 2′-OMe and a phosphorothioate.

In any aspect of the disclosure, or embodiments thereof, the base editor further contains one or more uracil glycosylase inhibitors (UGIs).

In any aspect of the disclosure, or embodiments thereof, the base editor further contains one or more nuclear localization sequences (NLS). In any aspect of the disclosure, or embodiments thereof, the base editor contains two NLS.

In any aspect of the disclosure, or embodiments thereof, the method involves altering the nucleobase of the LPA polynucleotide at a rate of greater than 50%. In any aspect of the disclosure, or embodiments thereof, the method is associated with at least a 50% reduction of serum concentrations of LPA in the subject. In any aspect of the disclosure, or embodiments thereof, the method is associated with a reduction of serum concentrations of LPA in the subject to less than about 50 mg/dL. In any aspect of the disclosure, or embodiments thereof, the method is associated with at least a 10% reduction of incidence of coronary heart disease in the subject.

In any aspect of the disclosure, or embodiments thereof, the deaminase domain is inserted within the napDNAbp domain.

In any aspect of the disclosure, or embodiments thereof, the base editor system is administered to the subject using a lipid nanoparticle containing the guide RNA and an mRNA molecule encoding the base editor.

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “atherosclerosis” is meant a pathology associated with the deposition of plaques of fatty material on the walls of blood vessels.

By “cardiovascular disease” is meant a pathology affecting the heart and/or blood vessels.

By “lipoprotein(a) (LPA or LP(a)) polypeptide” is meant an LPA protein or fragment thereof having at least about 85% amino acid sequence identity to a polypeptide sequence provided at Ensemble Transcript Accession No. ENST00000316300.10 and capable of stimulating secretion of plasminogen activator inhibitor-1. An exemplary LPA polypeptide amino acid sequence from Homo Sapiens is provided below (Ensemble Transcript Accession No. ENST00000316300.10):

(SEQ ID NO: 426)
MEHKEVVLLLLLFLKSAAPEQSHVVQDCYHGDGQSYRGTYSTTVTGRTCQ
AWSSMTPHQHNRTTENYPNAGLIMNYCRNPDAVAAPYCYTRDPGVRWEYC
NLTQCSDAEGTAVAPPTVTPVPSLEAPSEQAPTEQRPGVQECYHGNGQSY
RGTYSTTVTGRTCQAWSSMTPHSHSRTPEYYPNAGLIMNYCRNPDAVAAP
YCYTRDPGVRWEYCNLTQCSDAEGTAVAPPTVTPVPSLEAPSEQAPTEQR
PGVQECYHGNGQSYRGTYSTTVTGRTCQAWSSMTPHSHSRTPEYYPNAGL
IMNYCRNPDAVAAPYCYTRDPGVRWEYCNLTQCSDAEGTAVAPPTVTPVP
SLEAPSEQAPTEQRPGVQECYHGNGQSYRGTYSTTVTGRTCQAWSSMTPH
SHSRTPEYYPNAGLIMNYCRNPDAVAAPYCYTRDPGVRWEYCNLTQCSDA
EGTAVAPPTVTPVPSLEAPSEQAPTEQRPGVQECYHGNGQSYRGTYSTTV
TGRTCQAWSSMTPHSHSRTPEYYPNAGLIMNYCRNPDAVAAPYCYTRDPG
VRWEYCNLTQCSDAEGTAVAPPTVTPVPSLEAPSEQAPTEQRPGVQECYH
GNGQSYRGTYSTTVTGRTCQAWSSMTPHSHSRTPEYYPNAGLIMNYCRNP
DAVAAPYCYTRDPGVRWEYCNLTQCSDAEGTAVAPPTVTPVPSLEAPSEQ
APTEQRPGVQECYHGNGQSYRGTYSTTVTGRTCQAWSSMTPHSHSRTPEY
YPNAGLIMNYCRNPDAVAAPYCYTRDPGVRWEYCNLTQCSDAEGTAVAPP
TVTPVPSLEAPSEQAPTEQRPGVQECYHGNGQSYRGTYSTTVTGRTCQAW
SSMTPHSHSRTPEYYPNAGLIMNYCRNPDPVAAPYCYTRDPSVRWEYCNL
TQCSDAEGTAVAPPTITPIPSLEAPSEQAPTEQRPGVQECYHGNGQSYQG
TYFITVTGRTCQAWSSMTPHSHSRTPAYYPNAGLIKNYCRNPDPVAAPWC
YTTDPSVRWEYCNLTRCSDAEWTAFVPPNVILAPSLEAFFEQALTEETPG
VQDCYYHYGQSYRGTYSTTVTGRTCQAWSSMTPHQHSRTPENYPNAGLTR
NYCRNPDAEIRPWCYTMDPSVRWEYCNLTQCLVTESSVLATLTVVPDPST
EASSEEAPTEQSPGVQDCYHGDGQSYRGSFSTTVTGRTCQSWSSMTPHWH
QRTTEYYPNGGLTRNYCRNPDAEISPWCYTMDPNVRWEYCNLTQCPVTES
SVLATSTAVSEQAPTEQSPTVQDCYHGDGQSYRGSFSTTVTGRTCQSWSS
MTPHWHQRTTEYYPNGGLTRNYCRNPDAEIRPWCYTMDPSVRWEYCNLTQ
CPVMESTLLTTPTVVPVPSTELPSEEAPTENSTGVQDCYRGDGQSYRGTL
STTITGRTCQSWSSMTPHWHRRIPLYYPNAGLTRNYCRNPDAEIRPWCYT
MDPSVRWEYCNLTRCPVTESSVLTTPTVAPVPSTEAPSEQAPPEKSPVVQ
DCYHGDGRSYRGISSTTVTGRTCQSWSSMIPHWHQRTPENYPNAGLTENY
CRNPDSGKQPWCYTTDPCVRWEYCNLTQCSETESGVLETPTVVPVPSMEA
HSEAAPTEQTPVVRQCYHGNGQSYRGTFSTTVTGRTCQSWSSMTPHRHQR
TPENYPNDGLTMNYCRNPDADTGPWCFTMDPSIRWEYCNLTRCSDTEGTV
VAPPTVIQVPSLGPPSEQDCMFGNGKGYRGKKATTVTGTPCQEWAAQEPH
RHSTFIPGTNKWAGLEKNYCRNPDGDINGPWCYTMNPRKLFDYCDIPLCA
SSSFDCGKPQVEPKKCPGSIVGGCVAHPHSWPWQVSLRTRFGKHFCGGTL
ISPEWVLTAAHCLKKSSRPSSYKVILGAHQEVNLESHVQEIEVSRLFLEP
TQADIALLKLSRPAVITDKVMPACLPSPDYMVTARTECYITGWGETQGTF
GTGLLKEAQLLVIENEVCNHYKYICAEHLARGTDSCQGDSGGPLVCFEKD
KYILQGVTSWGLGCARPNKPGVYARVSRFVTWIEGMMRNN.

By “lipoprotein(a) (LPA or LP(a)) polynucleotide” is meant a nucleic acid molecule encoding an LPA polypeptide, as well as the introns, exons, and regulatory sequences associated with its expression, or fragments thereof. In embodiments, an LPA polynucleotide is the genomic sequence, mRNA, or gene associated with and/or required for LPA expression. An exemplary map of a LPA polynucleotide sequence is provided at FIG. 2. An exemplary LPA nucleotide sequence from Homo Sapiens is provided below, where underlined text represents untranslated regions, regular text represents introns, and bold-faced text represents exons (Ensemble Transcript Accession No. ENST00000316300.10):

(SEQ ID NO: 427)
GTAAGTCAACAATGTCCTGGGATTGGGACACACTTTCTGGGCACTGCTGGCCAGTCCCAAAATGGAACAT
AAGGAAGTGGTTCTTCTACTTCTTTTATTTCTGAAATCAGGTAAGACATAGTTTTTTTAAATTATAAGAA
TTATTTTTTCTCCCACAATGTAGTAAAAATACATATGCCATGGCTTTATGTGCAATTCATTTAATTTTTG
ATTCATGAAATTCCCAGTTCAAAATCTTGTATATGATTGAAAAATTCTTAAAAAAATAAGTTTAATTTCC
CCGTGAAGACTGTCACGGTGCTGGAATGAATGGGCAGAAAAAATAATGGTTGATTTTTCTAATCTAAAAG
AGTGTGCCTACATGATGGCCAGTCTGGCTGAAAAATAAATAGCCATTGTAGCTAACTATGCAAAGGATGG
CTAAGCTCTTCGCTTGGTTCTCAGTTTCATTAATTTATATCATCTCTGTTCAGGTGCCATGCTCCCCTCA
CTAGCAAGTTGAAACAATGAAATAACTCTTTGAATATGTTTGGTTCCTTGACCTGTTCATGGAGTGGGAC
TCAGCATTTCTCTCTTTGTTATGGCCTGAGTAAGGCTTTCCATCGGTATACATTTGCTTCTTATCCCTGG
AGAAATTATACACATCCATTTGCCAGATGATATACGCATATAATGATTCAACAAATACTCAGGGTATTTG
TTGAGTGGGTTAGGTCCCCACATTTTTATACATACATACACACATACACACCGTGTGTGATTGTGAATGT
AAGTGTGTGTCCTTTACAAATACTAGCTTATTTAGCTCATGGTATAGGTAGGGTAGCATAGTCATCCCCA
TTTTATAAACAAAGAAATCTAGACTTAGGAAAATCATGTTATTTGTCTCGTGACCAAATTCCCAAATCAA
GGAAATAAAGAAACCTGGATTTAAGCCAGATTTCCAAGAAAAAATCTAGGGCTCTTCTCACTTTTTCATC
TTTGTTCCAACATTTGAAAAAATAAATCTAAACACATTCCAATGTAACTGAAGAGCAGGTTAATTGTTTG
CCACTTGCAGAATCCAATTAAGAAGAGAGAAGTCTGGTATAAAGAAAGTGATTTGCTTCCAAAGCTAGCT
TAGGGGAAGAAATGCAGCAGTCCTGCCGTACTACTTCACTTTAGGAGCAGAAAGTGGCACTTTTAAAAGG
CAACAGAGGAGGCGAGCAAGGATTCAGGGGTCCATGCTAGCTTGGGCACCTTATCCACCAGGTAGTTGAG
CAGTTGCCTGCTGGTGCCTTTGTGAGCAGGGTGTTGTCCCTTGAGGCAAATCTCTGGAGGGTGAGAGTTT
TGTAGTGGGCATGCTTTGGTTTATAAATCACCTGTGAACTCAGGAGTTCCATCTTGAAGCACATACATAG
TTAGATGAACTTGCCCTGCAGGGAGAGTCTGATGAAAGGGAGGTAGATGCTTGCAATTTAATCTATAAAT
TACCAGATAAAATTTTACAAGTTGACTTTAAAGTCAAACACATTTGAATTTAGTGGAAGCCATTCAAGAA
AATATCAAAGAAAATACAGAGCAGGAGAAGATTAAGCAAAGAGTTTTTTGGGGAAATTGGTGTCTATGTC
TGTGTGTGTAGGGAGTGCAGGGGATATGAATATTCTATTTCAGCCCATGGAAACTAGGATGTAGATCACT
GTGAACTTATTCAGCAGGCTACACCCAAAGGCTAGAACAAACTTCTCTGCCACAGGATTAACATATGTTT
TAATCGACCTGGGGGGCACATTCTCTGATAAGCTCTTTTGGAAAGCCAGGCTTTCTGTGGACGTGTTATC
TTTCCAATGTGTGCTGGAATGCCCGGGGAGAGGAAAAAGTTTCTTTTACAGCCATGCTCAGTGAGAAGCG
GAGAAACATCTTCTATTCACAAATTGCTAAGTCTTTTACACATGCAAATATGCATACACATTCACACACC
ACAGTGAGGAAGAAATTCTCACACCATTAATAAAATACATTTACTTCAGTAGCAATATACATCTACATTT
TGCCTATAATATAAAAGTATTTTTCCTATTAAAAGATTTGTTTAATGTTTCTTCACCAACAAATAAACCC
TATTAAATCCCCATTGCCATATGAGCCCTGGAGGTGAATCAGAGAAACAAAAGGATTGTGGAAAAATCAT
CAGGTTAAAAAAAGAAAAATTGATTCTGTTTTGGGATATTTCCTAGCAACATGAGCTGGGGAGGGGATCT
CAGCAGTGATGCTCTATGAAGCATAATAAAATGACACAGTTACAGGTAACTTAGTTAAAGGGGGAAATAA
ATGGAAGTTTCCTCTTTTTGAATATCAATTGTAGCCTGCTCTGCTACATTTCAAAAACACTCTTCAAAAT
GTTTAACTGAACTCACTGTAGGAAGCACCTTATTAATTTATTGTGTGTTTTGAAGTCACACTGTGAGCTA
TAGAATTTACCCAAGCACAACTCTTCCTGGAAAAGAGAGTTCAAATGAGAAACAGTGCGGGGTGAAGACA
TGGATATGGGCCTAAAATATCTATTTCTCAATGATATTTTGATATATCTATCAAGTGCTTTTTAGTGGAT
TAGGTTCAGAATGCATCAGCCAATGCCTGTTCAATAATCCAGTTTTCCAGCATAGAGCATATTAAATTGA
GGAAGGACAAAGTCACAGAGGTGGGGAGCAGGTGGACTGTGGCCAAGGACTTTGCATGAAACAGTGAGCG
TGCATCCTCCTCCTTGCCCTGCCCTCATGGTCTGTGTACTCTCAGGAGGTCAGGACAGGCCTTTCTGAGA
ATGAGAATCTGTTCATCTGCCTTTCTACTGGATACTTGTCATCGGCATACAAACACATGTTCTCTGCAGT
GTGTCATCTTTCAGAACCTCCCCTGACCCTGTATTCCCTAGAAGTCTCGCTGCTTTCAGAGCCAGGCTTC
TCTCCTGCTGCCACCCCCACTGCTCTTCTAGTCACTCTTTAACCCACTCCATCTGCATGTGGCCCCCACC
ACACCCCTCAAAGTGGTCAAGGTTGTCCTGTTGCTTAATTCCATGGAAGCTTGGCTATCTTCATTTTATT
AGCCTCTTTTGGCCTCTCACCCTGTGAAAATCACTACATTTTGTGCCAGAGATGGAGCTGGCATCTCCAG
GCTTGGAAGAGGGCTGCTGAAGCTCAGCCAGGTGTCCTAAGGAGCCTCAGGACAGGGGATGCTCAGTAGC
CTTGCAATGGGAACACAGCTGAGCCCCACTTGGCCACCCTTTGCCACAACCAGGCAGAAAGCAGCTTTTG
AACAGATTTGTTGCCTCAGATTTGATCTCAAAGAAAAATCGTGGGCAGTATTGGTCCCAGGTTCTGCTTT
TTTACAATTTCCTCTGAAATCTGGATGCCTATCAACACCTTGGAAAAACTGAATTCTCCCCAACTAATAG
TGGTGTGTCACTGTAGTAAGCCTAGTACAAAAATGGCCTTCTTTGTGGAGGAGCTTCATATCCTCCATTT
TTTTTTTGCTTAATTTTTGCCCAAGATGAGAACATAATTTAGTTCACTTTTTATTTATTCCCAACATCAT
CCATGCACCAACATTTTTGTAACTAAAGGAGGGACCATTCAGAAGATGCTTATCAACTGTCAAAGTGACA
GTGTTACAACCAATGCACATATTGTAAGAAATCAAACAATGGCCTCCAAGGTTCATTTCTACACAGGGAT
TAGCAGATCAACATCAATCTTGGCAACACAGTTGCCACTGATGGTGTCTTATTTTTTTTATCATGACATG
GCAATCAAGAGCAAACATGATTTATTCTTATTTAAGATTTTATGGTTAGACTAGGCAGATAGCTAGATAT
GAGCAGGAGGTGGAAGCCCCTGAGAGAATGGAGGTCTGGAGAATCTGAAACCCCAGAGATTACCCAAGTC
CTGCATGCTAGACATGAGTGGAGGAGGGGGAATACCTAGGTAGAAAAGAATGCCCCTTAAGATGCCCAGC
AGTCGCTCACTGTGCAGTTAACTTTTCAGAATGCTGCTAGATACATGCTGATAGGGAGGGAAGAGGGCAA
AGGAGAAATTCCTAAGAGATACACGGTTGCAGTTAGTATACATCTGAGTGCTATACAACCTTCTTTGGGT
GGTGGCAAGAAGCAATGCAGCCATTACGTAGAATTCATATCAAACACCTGTATCACAGGTGTTAAAGAAA
CAAGAAACATTGTACTTCTTGTATTCTTAATAATGATTTGCAATATTGTCTTTAGTATCACTGCAAACCT
CTATAAATATGATTTTTAAAAAGTATTTCTTTAGGTTGGAATTACTTCTACGCATTGACTTATCTTCCTG
GGTTTCATTAGCCGTACCCGTTGTACTTTCTTCCTTACCACTGTTTATCTCAAACTCTTGAGATTAAAGT
ATGGGCTCAGGAGGGAGCGAGGAGCTTCAGGACTCTCACGGACCTCCAGCACAGTGTAGCTGCCTTATGG
AAAAGTGGCCACACTGTTTTCTGCACTGGTCCCTGCCCCTACTATTCCTCACTGGGCAGAGCACAGCCAC
CCTGGCCCTGCCTGAACATTTTAGTCAGTGTTGGCTCTGTGCTTCTCTGGGGAGGAAATCCAAGAGACAA
CCCACAGCCCCTCTGCCATTTCAGCTGCAGCAGTACCACCGTTAATGCCCTTGGGCTTGAGAAAGAAGGG
ACCTGGCCACTTCCCTGACACCTCCAGCACACAGCAGGGAAAGAATTCCAGTTTCTCTTTCTTGTGAGCT
TTCACCTGCTACTCTTCACCAGGCAAGGCTCCTGGCTTGGGCCCACAGTGCAGGCACCTCGAACTCAGTT
GAACATTTCCACTGGCTGCACTCTGTGTTTTTGTGGGGTGAAGCTCCCAGAGGTGACTGAAAGTCCTTCT
GCCACTAACACTGCAGTCATACTGCCCTTGCTGTACTTGGACTAGGGAAGGAAAAAAGATCCTGAGTGCT
TTACTCACACCCCAGTGTGCCCCAGCCACCCTATGGAAAAGAGGCCAGTGTGTCATCCCTGCAAGCACCC
TGAGGCCCCTGCCCCTGCTGCCCCCAAGCTGTAGAGCCAGAATATAAAGCTGGCAGAAAAATGTAAAAAG
GCTAGACTGGCTTAGCCTCCCAGCCTACATCTTTCTCCTGTGCTGGATCCTTCCTGCTCTTGAACATCGG
ACTCCAAGTTCTTCAGCTGTGGGACTTGGACTGTCTTCCTTGCTCCTCAGATTGCAGGTGGCCTATTATG
GGACCTTGTAATCTTGTGAGTTAATACCACTTAATAAGCTCCCCTTTGTGTGAGTATATCTATATCTATA
GATAGATATAGGTATACTCACTATATATACACATATATACATATACTCTCTCTCTCTCTCTCTCATATAT
ATATATATATAATCTCCTATTAGTTCTGTCCCTCTAGAGAACCCCGACTAATACAGATTTTCATACCAGA
AGTGGTTCTTGAGGAACAGAATATTAAGGATGGAATTCTTTCATTGGTTTTGGGACTTCTGGTGTTGGCT
GATTAATATGATTAGACCAAAAAATGCTAAGGACTCTACTTCTAATAGTATGGAGAACACTGATAGTACT
TGGCCTGAATTGTTTAGAGAGTTATGCAAAATAAATGCATTTGACACTACTGATTCATCACTTATGAGAG
GCAAGGAGTTTAGTGACTCTATACATAATACCTTTGACTATATGTGGAGAACCAAGGAACATAATGAAGT
TGGTTGATTGCTCCTAAGTTCTCTGGAGAAAGAGATGAAAGAAAATGATGATCTCAGGGGATCTGTCTCC
CACCTTCAGAAGCAGATACTGAGCCACAAATCTGCTAAGATTGCCCTGAATGAGAGTTTTAACTCCTGTA
GAGAAAGAGTTGAAATTGTGAAAAAACAGAGACAAGCTGTTATCATGCGAGTAGCTGATCTGCAACAAGA
GGTGCATGCACAGCCTTGCCAGGTGTTTACTGTTAAAGTGAGGGCATTGACTGGAAAAAAATGGGACCCT
GGAACTTGGAGTGGGGATGTGTGGGAGAACCCTGATGAAGCTGAGGACACTGAGTTTGTGAACTCTGATG
AAACTTTTTTGCCAGAAGAAACAGTTTCCCCATCCCCAGTAGTGGTAACATCCCCTCCCTGACCCGTGCT
GCCATTAGCCTTTCCACCTTTGTCTGAGGATGTAAACCCTGCACTGCTTGAGGCAACAGTGATGGCCTTC
CCTGAGGCAGCTGCCAGGCAAGATAATGTTGATTCTCCTCAAGAGGCACCCCTAATGCCCCTGAATGCTT
CTAGACCTATAACTAGGCTAAATTCCTTGCGGGCCCCAGAGGTGAGGTTCAGAGTGTGACCCATGAGGAG
GTGCATTATACTCTAAAAGAACTGCTTAAGCTTTCTAATTTATATTGGCAGAAATCTGGAGAACAGGCAT
GGGAATGGATATTAAGGGTAAGGGATAATGGTGGAAGGGACATAGAGTTGGATCAAGCTGAATTTATTGG
TTTGGCCCTACTAAGTAGGGATTCTGCATTTAATGTTGCAGCTCGGGGACTTAGAAAAGGTTCTGATAGG
GCCGGGAGCAGTGGCTCACGCCTGTAATCCCAGCACCTTGGGAGGGGGGGGGGGGCAGATCACGAGATCA
GGAGATTGAGACAATTCTGGCTAAAATGGTGAAACCCCATCTCTGCTAAAAATACAAAAATTAGCTGGGC
ATGGTGATGCGTAACTGTAATCTCATCTACTTGGGAGGCTGAGGCAAGAGAACTGCTTGAACCTGTGAGG
CAGAGATTGCAGTGAGCCAAGATCGCCCCACTGCATTCCAGCCTGGTAACAGAGCAAGACTCCATTTCCA
AAAAAAAAAAAAAAAAAGTTATAATAGTTTATTTGCTTGGTTAGCTGAAATATGGATTAAAAGATGGTCC
AATGTTAGTGAGCTGGAAATGCCTTGGTTTAATGTAGAGGAAGTGATCCAAAGGCTTAGGGAGATTAGGA
TGGTGGAGTGGATTAGTCACTTTAGACCTACTCATCCCAGCTGGGAGGGTCCAGAAGATACACCCTTGGC
CGAAGCTTTGTGAAATAGATTTGTGAGAGCAGCACCTGTATTTTTGAAGAGCCCGTAATTGCTCTTCTCT
GTATGTCAGATCTAACAGTAGGAACCACAGTCACTCAACTACAAAATTTAAATACAATGGGAATAATTGG
ATCCTGAGGTGGCAGGGGCCAAGTGTTGGCACTGAACCATCAAAGGCAAGGTGGGCATAACTACCATAAT
AGACAGCAGAGGCAAAGCAGCCATCAGAATAGTCTGACTCATGTAGAGCTCTGGCATTGGCTAATTAATC
ATGGTGTTCCTAGAAGTGAAATTGATGGGAAACCTACTGTATTCCTACTTGATTTATATAAACAAAAAAC
TGCCAGGTAGAATGGACTAAAGACTAATCTGAATTATAAAAACAGAGAATCATGGGCCCTCAATCAATTT
CCAGACTCGAACCTGTTACAGTTCCAGAACCCACTGAATGAAGGGGAGGCTGGATCCCCTTGAGGAAGGA
CACCACTAGGCTACTGACAACTTATGCTGTTACTCTTTCTCCCATCCTTCCCTAAGGAGACCTCTGGCCT
TTTACCAGGGTAACTGTGTGTACTGGAGAAAGGGAAGTAATGAGACATTTCAGAAAGTACTGGACACTGG
CTCTGAGCTGACGTTGATTCCAGGGTACCCAAAACGTTATTGTGGTTCCCCAGTTAAAGTAGGGGCTTAT
GGAGGTTAGGTAATTAATGGAGTTTTAGCTCATTTCTGACTTACAGTGGTTCCAGTGGGTCCCTGGACTT
ATCCTCTGGTCATTTTCCCAGTGCCAAAATGCATAATTTGTATAGACATACTTATTAGCTGGCAGAAATG
CCACATTGGCTCCCTGACTGGTAGGATGAGGGCTATTATGGTGGGAAAGGCCAAACAGAAGCCATTAGAG
CTGTCTCTACCTAGAAAAATAAAAAAATCAAAAACAATATCCCATCCCTGGAGGGACTGAAGTGATTAGT
GTCACCATCAAGGACTTGAAAGACGCAGGGGTGGTGATTCCCACCACATCCCTGTTCAACTCTCCCATTT
GACCTGTGCAGAGGACAGATGGATCTTGGAAAATGATGGTGGATTATTTTAAGCTTAACCAAGTGGTGAC
TCCAATTGCAGCTGCTCTACCAGTTGTGGTTTTGTTGCTTGAGCAAATTAACACATCTCCTGGTGCCTGG
TATGCAGCCATTGGCTTGGCAAGTGGCTTTTTCTCCATTCCTGTCCATAAGACCCACCAGAAGCAATTTG
CCTTCAGCTGACAAGGCCAGCATTATACCTTTACCACCCTACCTCAGGGGTGTATCAACTCTCCAGCTTT
GTGTCATAATCTTATTTGGAGAGACCTTGCTCGCTTTTCACTTCCACGAGATATAACACTGGTCCATTAC
ATTCATGACATTATGATGATTGGATACAGTGAGCAAGAAGTAGCAAACACACTGAACTTATTGGTGAGAC
ATTTGTATGCCAGAGGATGGGAAATAAATCCAGCTAAAATTTAGGGACTTTCTACCTCGGTAAAATTTCT
AGGGTTCCAGTGGCATGAGACCTATGGAGATATTCCTTCTAAGGTGAAGCATAACTTGCTGCGTTTGGCC
CCTCTTACAACCAAGAAAGAGGCACAATGCCTGGTGGGCCTATTTGGATTTTGGAGGCAACACATTCCTC
GTTTGGGTGTGTTACTCTGGCCCATTTATCGAGTGACCTGAAAGGCTGCCAGATTTAAGTGCAGTCTAGA
ACAAAAGAAGGCTCTGAAACAGGTCCAGGCTGCTGTGAAAGCTGCTCTGCCATTTGGGCCACATGACCCC
GCAGATCCAATGGTGCTTGAGGTGTCAGTGGCAGATAGGGATGCTGTTTGGAGCCTTTGGCAGGCCCCCA
TAGGTGAATCACAGTGGAGACCTCTAGGATTTTGGAGCAAGGCCCTGCCACTTCTGCAGATAACTACTCT
CCTTTTGAGAGACAGCTATTGGTCTGTTATTGGGCTTTGGTGGTAACTGAACGTTTGACTGTGGGTCATA
AAGTCACCATGCTACCTGAACCTGCCTATCATGAACTGGTTGCTTTCTGACCCATCTAGCCATGAAGTGG
GTCAGCACAGCGGCATTTCATCATCAAATTGAAGTGGTGTGTATGTGATCGGGCTTGAGCAGGTCCTGAA
GGCACAAGTAAGTTACATAAGGAAGTGGCTCAAATGCCCATGTTCTCCACTCATGCCACCCTGCCTTCCC
TCCCCCAGCCTGCACCAATGGCCTCATGGGGAGTTCCCTATGATCAGTTGACAGAGGAAGGGAAGACTAA
GGACTGGTTCATAGATGGTTCTGCACGATATGCAGGCACCACCCGAAAGTGGACAGCTGCAGCACTATAT
CCACTTTCTAAATGCATGTGTACACTTGTGCTAAGAAAATATCTTTATTTTATTTCCTTTATTTTTCCTT
TATCATGTGACCTTAGATTTATGGACTTCACATCAGCATTTAAGCATTTAAGTGTTGTTCATATCAGCAT
TTAAATATTGTTAACCTTATGTAATAACTTTTGGTTTGGGGATTGGTGCGTTTCTGGTTGTATGAGGATA
GTTGTATTATATTAGGCATAATTATGACCTTATTATTGTCTTTATTTGAAGATTATGTATGATTTCAGGA
TGTGTGTATGGGTTCAAGTTGACAAGGAGTTGGACTTGTGATGGTTAATACTGTCAACTTGATTGGATTG
AAAGATGCAAAGTATTAATCTCGGTTATGTCTGTGAGGGTGTGGCAAAAGGAGATTAACATTTGAGTCAG
TGGGCTGGGAAGGCAGACCCACCCTTAATCTGGGTACACACCATCTAATCAAGTTCCAGTGTGGCCAGAT
TGTAAAGCAGGGAGAAAAATGTGAAAAGACTAGACTGAATTAGCTTCCCAGCCTACATCTTTCTCCTGTG
CCAAATGCTTCCTGCTCTTGAACATCGGACTCCAAGTTCTTCAGCGTTGGGAGTTGGACTGGCTTTCTTG
CTCCTCAGCTTGCAGAGGGCCTGTTGTGGAACCTTGTGATCCGCTGAGTTAATACTACTTAATAAGATCC
CCTTTATATACATATAATATATTATATTATATATAATATATATAATATATATTATATATAATATATATAA
TATATTATATATTATATATAATATATATTATATATTATATATAATATATATTATATATAATATATATTAT
ATATTATATATTATATATAATATATATTATATATAATATATATAAAATATATATATATCCTATTAGTTCT
GTCCCTCTAGAGAACCCTGACTAATACAATTTATGTCATTAATCTCATTTATTGATTTGTATACATTGAA
CCAACCTTATATCCCAGGAATAAAACCTACTTGATTGTGGTGGATTAGCTTTTTGATGTACTCTTGGATT
CAATTGCTGGTATTTTATTGAGAATTTTTGCATCTGTGTTCATCAAGGATATTGGCTTGAAGTTTTCTTT
TTTTGTTGTTCCATATCAGAATGATGACGACCTCATAGAATGAGTTAGTCTGTCCTCTTTTATCTTTTGG
AATTGTTTCAGGAGGCTTGATATCAGCTCTTCTTTATATGACTGGTATACTTTGGCTAGGAATCTCTCTG
GTCCAGGGGTTTTTCTGGTGTAGGTTTTTAATTACTGATTCAACTTCAGAACTCATTACTCATTATTGAG
TTCTAAAACTCACTTTCATGTACTCTTCAAAAGACTGTCTTCTTCTGTTGTTGAGCGGGGTGTTCTCTCA
AGGTCGTTTAGGTGAAGGTGGTTGCTGGTGTTCTTCTGTATCCTTACTGCTTGTCTTTCTCTTTTTTTAT
TGACTACTGAGGATTAATGGTGATGTGTCCAACTTTAACTCTAGATTAGTCTATTTCTCTTTTAGATTGT
AACTCTGTTTTATATATTTTGAAGCTCTGTTGTTAGGCATGTGTATTTGGATTGTTAGGTCTTCTTGATG
ATGACCTTTATCATTATGTAATGTTTCTTCTTATCTCTGGAAGTATTCGTTGTTCTGAAGTCTATTTGTG
CTGATATGAATACAGCCTTCACAGCTCTATTTTCACTAGTATTTGTATATCTTTTTCTCAGCTTTTAAAT
TGAGATGTTCAGACCATTTGCATTAAAGTAGTTGTTAATAGGATTAAATTTAAATCTACCATTAAGTTGG
TTATTTCTCTTTGTCCCATTTAAACTTTGTTCCTTTTTTCATATTTTTCTGCCTTCATTTATATTGAGTT
TATCTCCACGACTTACTTATTAAATTAATTTTTAATGGTTTTAGTATTTTCCACAATGTTTATAATATAT
ACTTTGATTTTTTCACATTCCACCTTCAAATGACAGAATTATACTGGATATATAGAAATCTTACATCATT
GCACTTCTCCTTCCTCCCTCTCAAAATGTTGTGCTATTGCTCTTTGTAATAGAGGCTTACTTCTATTATG
TTATAGCTCTCATAATACATTGACACTATTTTTACCCTGAATAATCAGTTGTTTTTTAAAGTGATTATGA
CTACAAATATTTTGAATAATTTCTTTATTTTACCATTTCTGGTGCTCCTTATCTTTTACAGTAGATCCCA
ATTTCCATCTGGAGTCACATTCTTTCTGTGAAAAACAACCTTTAGCATTTCTTATAGCACGGGACTGCTG
TTGCTGTTGTCTTTCAGCTTTTCTTTGTCTGAAGAAGTCTTTATTTTGCCTTCAGTTTTTAAAAGTGATT
TTGCTGAGTATAGATACTGGGTTGAGAGTTTCATTCCTTGTATCATTTTAACAATGATGTTCCATTATAT
TCCGTTTTGAATAGTTTCTGACTAGAAATCTGATCTTTGTTTCTTTGTATTCAATAGTTCCTTTTTCTCT
GACTGCCTTTAAGATATTCTCATCTTTGTTTTTCAACAGTTTGACTATAATTTGTTTATTATTAACTTTT
TGTATTTATTCTGCTTGAGGTTTCCTGAGCTCCTTGGATTTGCAGATTGTTGATTTTTATTGTTTTTGTA
AAATTCATAGCCATTATCTATTCTACTGTTTTGTTTTTTTTTTCACTTCTCTCTCTCTGTATTCTTCTTT
TTGGACTGTAAGTATTCAAATGTTAGATCATTCATATTGCTTCATAAACCTTATATGCTTCTTCTGCTTT
TTTTTTTTTGTCAGGAACTCTTTTTTTGTATCTGTGTTGGTTTGGATAAGTTCTAGTAGACTATGTTCAA
GTTTATGGATTATTTTGTTAGTTGTGTCTAATTGACTCCTCAGTGCATTCAGAGAATTCTTCATCTCTGA
TATTATAAATCTCTTCCTAGCATTTTCATGTTACTCTTTTCTATAGTTTCCATCTCTTTGCTGAAATTCT
CCCCCTATCCATGGATATTGTCCACCTTTACCACAAGATTCTTTAACATATTAACATAGGTATCATACAA
ACCCAAACTGATAGTTTCCAGATGGTGTCTTTTCTGAGTCTGTCTGTCTTGATTGCTTTATTATTTAACA
GTGACTTATCTTCCCTCTTCAGCTTTTGGTGTGTCTTGTAATTGTTTAATCAAACACTGGGTATCATAAA
TGGAGGAACAGTAGAGATTGCAGTAAATATTATTTATGCTTTGAAATGGGCACCCATCTTCTGTTGAAAA
TATGTTTTGTGGTCAATTGAGTCAACCTAGTAACTGGTTGAACTGAATTTGGCATTTGTGCTTGTTGCTT
TTATCTTAAATGCACCACAGGTTTAAATTCCTCCAGTGATGGGTTGCTGCTATCTTTTGCTTAGAGTGGG
GCCTGGGGTGTGGAAGAATTTTCTCAGTGTTCCTATCTATTATTAGATTTTAGCAGTCACTGCATGCCTG
CACTACAGAGGGGATATCTTCATACACATAATCTAACCCCATTGAAACTGCTGTTTCTTCTTAATGAATG
CTCAATCTTTGGTGGAAATAAACAAATGCTGTATCTCCTGGAGCCACTTCAGTCTTAGTCAGGTTCTGCA
GGGCTTTGAAGGGAATGCATTCTCAGTATTCTTGTGCCTTATTTGGATGGAACTTGAACCTGTGGTGGGT
TTGGAGAGAAAGAGTAGCAGACGTCTGCTATGTTGCAATGCAGGATGCTGGGCACAAGAAAATTTCCAGT
CTCTCCTCCAAGGAAATAAGATTTGATCATCTACCTATCCCTGAGAAGTGAAGGGCTTTGCCTGCGGTGC
TAGATGCAAAACCATTTTTCTCCCCCCATTGCCCAGAAACTTAAGGCTTTGGCTTTTCTGAGCAGTGGTC
TAGGGAATTGTGCAAGGTTTTCATATTTGACCCTGACAGCCCATCACCACCTACAGCTTGCAGTGCCAAA
TGTATCTCCCTCTGATCTCTCCTGTCCTGTGGTCCTCATGAACATTAAGAAGAGATTTCTAAAAAAGAGC
TTGCACATGAGCATAGTTTCTGGTGAGAAGAATTCTGATATGTTAACTTCCTCTAAACTTTTAAATAAAA
TATTTCTAAGAATTAAATAAAGTTCTAGAATGATATGAATCTATTCCTTTGGTTTTTTGCACGTCTGTCT
GCCTGCTAATCAAGAGAAGAGAATGGTCGTAATTCTCAGAGACTTTTTCCTGTTTGTGTCATAAATGACT
TCACATTTTTTTCTGTTCTAAGAACTATTCAGCTTGATTTCTTCTGTTTTAATTTTAGCAGCACCTGAGC
AAAGCCATGTGGTCCAGGATTGCTACCATGGTGATGGACAGAGTTATCGAGGCACGTACTCCACCACTGT
CACAGGAAGGACCTGCCAAGCTTGGTCATCTATGACACCACATCAACATAATAGGACCACAGAAAACTAC
CCAAATGCGTATGTCATTAATCTTACAGTAAGCAAAACAAGGTCCAAGTAAAATTTGTCTTAGAAAAGGT
GTGCGTCAAGCTAACTTCTTATGATTAAATTTTTCTCACACATAGAATGCATGGCAAAATGTCTGAGAAA
CATTACTTTGAGCAAAGAGTATGATAGAAGAGAAATGTTAAGCTGGCTCTCTTTCCTGAGAGTTTGATAA
AATCAGGAGAATATCTGGCGGTGGTGAGGCCACAATAATGGAAAATCAGAATGTTTAGACAGAGTCAGCT
TCAACAACACTCACTAAAGGTCAATGTGATCTTTACCCCTTGAAATTCTATAATTCTAATCTCCAATTCC
TGAAGTGAAGGTTGTGTTGGCCTTTTCTGTCTTGGCTCACAAGTAAATGATATGTGCATATCTATGGAAA
GGCGAATCTATCTTTTTCTATATCTATGTCTATTCCAACGGGTAGAAACACCCTGGGTCCTGAGCACCAG
TGGTCTGAAGGAATACGGGTTGCCAGGAAGAGAGAAGCAAAGGCAGGAAGGCAGATGAAAGTAAGAAATG
AGACAGATGCTAAACAATAAAAAGTGCGGGAAGATAGACAGAAGCTGGGGTCTGACCACACCATGGCCAG
TCTTTCACACATAAGTGACTACCAAAGACAAGAAAAAATGATTTCCGCTTGTTGGACAATAGATGGTAGA
GGACCAAGGGAATTGCGAGAGAGAGAACAATGAGATCAACTCAACAGATGCACTGGTTTTCTTCCTGGAG
ACCCTTCCTGCACTGAAGGGCAGGAGATGGAGCCCAAAAAAAACTGTAGCCATCTTGCTGAACAGAGGAG
GGACATTGGAGTTTGGGATTATTCAGGTGGCTAGGATTTTCTAGGCCTGCTAACAATGAGAACAGATTTG
TGGAGGAAAGGAGTTCTAGAAATATGCATAGAAATCTCCTCGAGTCATTGGCTAAACATGAAGCTGCATG
TACACAGAAAATAGATCCACAAGAAAGTAGGGCAAAGAACATCTACGGAAGAGCAGCAACTACAATGGAA
CAGTGAGCTCAATAAACATGACAGAGCTCAAATAGCACTAAGGGATATTGGAGTTTGGACCACACAGAGG
AGAGAGACTTCACTGAACATCTTGGGCATTCAGTAGAGACCCAGGAAAAGCCATACTTTAGGAGTAGAAT
TAGTATATTCTTAGAATAAAGGCAGCTCCACACAAACAATAGCAAAACTGAAAAGGAAGTCTCCAAGCAT
CAGAATGATGTCCAAGTCAATGAACTGCCTCTGAGAGGAAAACTCAACCATCTTTAGAGGTAAACATCAA
AGTCAAGTGGCTCAGCTATGCAGTATCCACAGTGTGAGGCCTAAATATAAAACTTGACTACACATAGAAA
CCTTTTAGTGTGACCCACAAGCAGGAGGAAAATCAGCCAATACAAACAGACCCAGAAGAGACAGAAATGA
TTAGAATGGCATAAAAATTTGACATATCACTATATAATAATTGAGTTCTAGGATTTAAGAAAACATGAAT
ATAGAATGCAACAGACACCTTATCCAGAGACAGTAAGAGTATAAAGAGCCAAATCGAAGAACTACTAAGA
GATATGTCTTAAATGAAAAAATTACTAGATGGCCTCCCCATCTAGTTAGACATTTCAGAAGAAAATACCA
AATGAAAAATAATTGCATAGAACCTACAGAACCAGATACACACATACAAAACACACGCATGCATACACAC
ACACTCAAACATGTATAAGCTTACAAACACACACACACATCCACAAATGCTGAAAAATGAAATCAACCGA
GCCACACAGACATAAAGGAAAACATAAAAAGATTTCCTACATGTGGGAAGCAAGTCACAGAAAGGGGGAA
GGAGATTGGAACAGAAATATATACTGAAAGCAAGGATGGCTGAAAATTTTCCAAATATAAAGAAGATTAA
AAAATCACGGACTCAAGAAGCTCAATGGATCAGAAAAATAATTTCTAAAATGACAATTATAGGATGCCAC
TGGGTACATAGCAGTTCAACTGTCAGAGGGCAAAGACATAATACACAGAAAAATCTCGTAAGGAACGGGA
AAAACAAAAAGCTGTGTCTTGCTAGAGGAACAGTGATACAAGTGACTAATGTGTTCCCATCAGAAACACT
GCAACCTGGACACAAAAGAATAACATTAAAGTAATAAACGTAAGAAAGAAGAGCTCAACTGAGAAGGCTA
CATCCAGCAATAAAATGCCTTGAAGTTCATCCATGTTGGAGGAATGCACATTGTGCACTCCCCTAAACAA
AGAAACCGGAAACTGTAAGACTTTGGAATCAGCAGGCTTATGTAACAAAAGAGGTGACCCTAAGGAATTA
AGGAGAAGAAGAATAGAACAAGAAGGGAACTTTCTGCAGCCTATATAATGAAGAACCTAGCAATTGGCAA
ATGTAGATGAAAATGCTACATGTTTTCTTGATCAAACGTTTATATCTTTTTAAATGAGAGTTGACGAGTT
GAAGCAAAATGATACCAATATATTTAACTTTACCATATGTAGAAGTAAAAATTTGAACATGTAGCATAAA
TCATGTAGGGATTAATTGGAAGTGTACCACTGTAAGTTTCTTACCTCATGCACGATAGTATGTAATACTA
ATAAAAGGTTAATGTGTGGGTTCAAAGGGATATTGCAAATCCTAGAGCAATCACAAAGTTTTTAACTCTG
AGGTTTGTTGTATAATAACAATATTTTATGTATTCAAAAGAGGGAAGCCAAGGAAGAAAAAAAAGTCTTT
AAAGAGCTCTGGCTCTTAGTACATCCAGTTGCTCATTGAATGAGCTTCCTGGAATGGAGGGTCTGGGACT
GAGACTAGGCCACATGTGTAGAGCCACTAGAGACACAATGTTGGATCCCCATGGCCCATAATACATTTCC
CATTTTCTCAGGCAGCCACAGGTCATGAATGTGAGGATACTGAGAGGTTGGAGCAACGTTCTTGGGAGGC
ATAAGGAAGAGCGAATGCTTCAAGATCCCCGCAGCCCAAACTCCTCAGCTGCTTTGCCTCCTAATTCATT
GTTTTTTGCTCCTCCATAGCTGTCCGACCTCTTCAGATCTCTTAGTCTTCCTGCCATCTTCCTTTATGCC
ATGGGACCCACTGTTCTTTCAACTCATCCCCCAGTTCTGGAGTGGCTGTGGACAGCAGAGGATAGACTGA
GAGCAGGAGAGAAGGTCCTGCCCAGGAACCCATTCTAGAGATACTGCATTCTGCCTGGGAGCAAGTTTTC
CAGGGCAGCTTTGAGAAGTCTTGCAGAAACAAACCTACTTGACCGACATGATATGGGAATGACAGACAGT
AATACTATTTGCACAATGCTTTTCCATGGGAAAGGTAGAGCCTTTTCACTAGGTTTTGAGTACATGGAGT
GTGAGAGTTGACCTGGAAAGGTTATCCTCCTTGATGCCATGTTTTCTCTGAAGAACTACATGTTCGTTGC
AACTCCCACATTAGAATATGAAGTCCTACCGAGAGAGATACGGAGACTAGACAGATACAGATGCATTTGC
ATGTGAATACACAATCCCACAATACAGACGTCAAAACCCATACCAGTTATTCCAGAGAGATGGATTGGGC
AGAAGGCAGAAGGAGAATACTCTGATCGTTTTTCGGCCACGTGTGTGTGTTATCTCAGTGTTTCTAAGAA
GCGTTTGCTACTTTAGATTTTTTATTTAAAAAAAATAGTAATAATCTATTAAGTATGAGAGATGTGCAGA
GAGGATTAGTGATCGAGAGCCATTTTTGCTGGTGGCAATCATATGGTACTTTTAATGGGAATATTAGAAA
GGCACCGGTAATGACCTTGTTGCAGCACAAAGGAGAGAGTGTGGGGTGCCCCTGCATGTTGTCCCACCTC
TTGTGACGTGTATCGTTTTGGAATTTCCAGTGGCTTGATCATGAACTACTGCAGGAATCCAGATGCTGTG
GCAGCTCCTTATTGTTATACGAGGGATCCCGGTGTCAGGTGGGAGTACTGCAACCTGACGCAATGCTCAG
ACGCAGAAGGGACTGCCGTCGCGCCTCCGACTGTTACCCCGGTTCCAAGCCTAGAGGCTCCTTCCGAACA
AGGTAAGGAGTCTGTGGCCAGACATCTACACGCTTCGATGCTGGGATGAAAAGCCATGGAAATTCCCACT
GATGCAGCCGCCTTCAATGGTAAACGGATGCTCGAGTGTTGCCTGAGTTCTACCATGTAGGAGGAAGCCT
CCGTGCACTCTCTGGGGGAGCCAGCGGAGTGATTTCTGGTGCAACGTGGTTGGGCTTTGTCTTTAGGATG
GGCACAAACCCTCCAGGGGGATCGACTTCAAAATTCACCTTGTTGTAAAACGGGCTACCTCAGTGTCCCA
GCCAAAATTTTTATTGTAACATGCTGTCAGGTGTGTCACTCTTTCCAAGCCAGTAAGCTTTTCCGGGGAT
TTCTTCAAGTAGCCAGCATTCAGAGCAATCTTCAGCATTGCAGATTCTGAGAAATGTGGCTCTGGAGCCT
GTCACCCTCGAGAAACCTAAGAGGGCTGCATTGATTCCATGTGGCCCTGGGTCTATGGAGCAGTACATGA
GCTCCCAGTGCTCTAAGGCTCTTCAGCCCTAGGCTTTGAAGGGAGTGATTTCTCAGTATTCTTAAACCTC
TTTCTGATGACACTTGTACCTGTGAGGGGTCTAGAGAGAAAGAGTAGTAGACTCCTACTTTACTACAATT
CAGGATGCAGGGCATGAGAGGATTCCCTCTCTCCTCCAAGGGAAGAAGCTTTTGGCGTGCACACATCCCT
GAGAAGCAAAGTGTCTTTGTCTTCAGTCAGATACATAGGACCGTTTTCTGCCCCATGGCCCGGAAGCCAA
AGGCCTTGGCTTTCATGATCAACGGTCTAGGGAAACATGCAAAATTTCCATGTCTGTCCCAAACTCTGCC
CCCGACAGCCAATTACCACCTGCAGCCCGCATTGCCAAATGCGGTGCCGTTTGCATGAAGATTCAGTAGA
GTTTCCTAGAAAGGTGCTACCTCGTGAGCTCACTTTCCAATGAGGAATCTGATCTGTTGTGTTTCTCTAA
GGTGTCAGGTGAAATATTTCCAAGAACTTACTACAGTTCTAGAATGGGAGGAATCTGTTGCTTTGGTGTT
TGTTTGTTGGTCGGTTTTCTCACATCCATCTGCCTATGGATAAGGAAAAGAGAACGGTCGTAATTCTCAT
AGACTCCTTTCTGGTTGTGTCACAAATGGCTTCACATGTTTCTCTATGCTCAGAGATACTCAGCTTGATT
TCCCGTGTTTTCATTTCAGCACCGACTGAGCAAAGGCCTGGGGTGCAGGAGTGCTACCATGGTAATGGAC
AGAGTTATCGAGGCACATACTCCACCACTGTCACAGGAAGAACCTGCCAAGCTTGGTCATCTATGACACC
ACACTCGCATAGTCGGACCCCAGAATACTACCCAAATGCGTATGTCTTTGTTCTTTACCATAAGAGAAGA
AAGGGCCAAGTGAAGTTTCTGTTACAAGAGATGTGTCTCAAGCTGAGTTCTCCGAACTCAACTTGTGACA
GATGCAGATGGCGTAGCAAAATGTCTCAGGATGATTGCCTTGGAGCTAAGGGTCTGAGAGAAGGGAAATG
TTAAGCTCCCTCTCCTTCCTCCTAGTTCTATTGAGCAGAAGGGAAATCTGGAGGTGAGGAGATCACATTA
TGAAGAAAGTCAGAATGACAAAGGACCAGACACTTAGATTACCCTTCCACAACACCAACTAAACGTCAAT
GGAGACTTTCCAGTTGGAATTCCGTTATTCTGGCTTCCACTTCCTGAAGGGAAGGTTGCGTTTGCCTTTT
CTCTCTGGGTTCAAGAGGAAAGAATAGGTGCTTATTTATGGACAGGTGAATTGATCTGTTTCTATATCTA
CGTATATTCCGATTGTCAGAAAAACACTCGTTCCTAAGTACCAGTGGCCTGAAGGGATACAGGTTCCCAG
CAAGAGAAGATCCAAGGAAGGAAGGCAGATGAGAGTCAGCACAGAGAGGGATGCTGAAAAGTAAAAGGGA
TGGGTGGATGGAGAGAAGCCCGGGTCTGACCACCCAATGGCCAATATTTTGGCCACAAGCGACTACCAGA
GACATGGAAAAATGGTTTCTACATGTGGGACAACAGATGGTAGAGGACCTAGAGAATTGAGAGAGGGGCA
ATGATGGGCTCCACTCCGCAGATGCCTTGGCTTTCTTCCTGGATACCCTTCCTGCACTGAATAGCAAGGA
GATGGAGCCCAAGCAGACTGTAGCCATCTTGCTGAATGGAGGAGAGGGATTGGAGTTTGGGATGACTGTG
GTAGCTGAAATTTTTCTAGGTCTGCTAGAAATAAGAACTGGTTTGTGTGGAGGAAAAGAGCTCTACAAAT
ACGCATAGAAGTCTCCTCCAGTCGTTGGCCTGACATGACGCTGCCTGTGCACAGGAAATGGTTCCACGAG
AAAGTGTGGCAAAGAACATTTACTGAGAAACAGCAAGTACAAGAGCACAGGAAGCTCAATAAAGAAGAGA
GAGATCACATAGCACTCTGGGATACTGGAGTTCTTCCCAGCTAGACCAGAGAGTCCTCACGGAGCACATT
GCCAATTCAGTGGAGACCCCAGAACAGCCGTAATTTAAAGGTACACTTAGTATATTACTAGAATAAAGTC
AGCTGCAGACAACCCCTTGCACAGCTGGAAAGCAAGTGTCCAAGCATCAAATCGGTTTCCAATCAATGAA
GTGCCTGTGAGAGGAAATCTCAACTCTCTTTAGAAGTAAACAACAAAGTCGATTGCCTCAGCTATGCGGT
ATCCGCAGAGTGAGTCCTAAATTTAAAATCTGACTACATGTAGAAAAGCGTTTCGTGTGACCCATGACCA
GGAAATAAATCGGGTAATACAAACAGGCTCAGGAATGAGAGAAATGATTAGAATTGCGTGAAAATTTGAC
ATATCAGTATGATAACTGATTTCAAATATTTAAAAAAACAACATGCAAGAAAGCAGATATCATATCAAGA
GAAATTAACAGTACAGAATAGCCAAATTAAATTAAAGAGGTAGTATAAAAAAAGTATGTCTTAATTGAAA
AAAATTACTGTATGGCCGGCTGATCAATTTAGACGTTTCAGAGGAAAACATTACCCAACACACAATTCTA
GAGAACCTACAGAATGAGCTACACACACACACACACACACACACACACACACTGAAAACACACCCATACT
CACACACACGCAGAAACTCACAAGTTCTAACACACACAGACACGCGCACCCCTGAAGAAACAGTGAAATA
TAAAATTAAGCGAGCCTCACAGACATGTAGGAAAATATGAAAAGATTTCCTGCATGTGGGAAGCAAGTCA
CAGTAAAGAGCAAGGGAGTTTATAATAGAAACAAATACCAGAATCAAGGATGGCTGATAACTTTTCAATT
ACGAAGAACATTAAAAAAAATCACAGAATCGTGAAACTCAAGGGATCATATAGGGAATTTCGGAAAAAAA
ACCCAACCTGTATGATGTACTTTTGTACATCACAGTTCGAAGGTAACAAGGCAAAGATGTAATAAGAAGA
AACCTGTCACGAGAAACTGGAGGAAAAAGAGCTGTGTCTTCCTACAAGTACACTGATACAAATTGCCAAT
GTGTTCACCTCAGAAACACTGGAAGCCAGATACCAGGGAATATTGTTAAAATGATAATCAGGAACAAAAA
GAGATCAACCGGGAATGCTGAATCCAGCAATAAAATGCCTTGAAGGTCATCCATGTCGGATAAATGCATA
TTGTGCACTGCCCCAAAGAAAGAAACCGGAAACTGTAAGAATTGGAAATCAGCAGGCTTATGTAACAAGA
GAGGTGACCCGAAGGAATTAGGTAGAAGAAGAATTGAACAAGAAAGGAACTTTCTGCAGCCCACGTAATG
AAGAATCCAGCAATTGGCAAATGTAGATAGATGTAAATGCAAAATATTTTCTTGATCAAATTTCTATATC
TTTGTAAATGAGAGTTGACTACTTGAAACAAAATGATAGCAAGATATTTAACTTCAGCATATGTAGAGGT
AAGAATTTGAAATGGTAGCATAAATCACGAAGGGATTAATTCGAAGTGTACCGTTGTAAGTTTCTTTACC
TCATGCACGATGGTGTGTCATATTAATAAAAGGGTACTGTGCGGGTTCGAAGGGATATTGCAAATCCTAG
AGCAATCACAAAGGTTTGAACTCTGAGGTTTTTGGTATAATAAGAATAGTCCATGCATTCAAAAGAGGGA
AGCCAAGGAAGAACTAGAAGTCTTTCAAGAGCTCAGGCTCTTATACATCCAGTTGCTCATTGAACCAGCT
TCCTGGAATGGAGGGTCTGGGGTTGAGACTAGGCCACAAGTCTAGAGTCTCTAGAGAGACAGTGTTGGAA
CCCCATGGCCCATAATACATTTCCCATTTTCTCAGGCAGCCAGAGGTCATGAATGTGAGGATACTGGGAG
GTTGGAGCAACGTTCTTGGGAGGCATAAGGAAGAGCGAATGCTTCAAGATCCCCGCAGCCCAAACTACTC
GCCTGCTTTGCCCCCTAATGCATTTTTCTCTGCTGCTCCGTAGCTGTCCGACCTCTTCAGATCTCTTAGT
CCACCCTGCCGTCTTCCTTTATGCCATGGGTCCCACTGTTCTTTCAACTCATCCCCCTTTCCCTCAGTCC
CGGAGTAGCTGCGGCCAGCAGAGGGTAGACTGAGAGCAGGAGAGAAGGACCTGCCTAGGAACCCCTTCTA
GAGATACTGCATCCTGCCTGGGAGCAAGTTTTCCAGGGCAGCTTTGAGAAGTCTTGGAGAAACAAACCTA
CTAAACCTGACAGACAGTAATACTATTTGCACAATGCTTTTCTGTGGGAAAGGTAGAGCCTTTTCACTAC
GTATTGAGTACATAGAGTGTGAGGGTTGACCTGGAACGGCTATCCTCCTGGATGACGTGTGTTTTCTGAA
GAACTACATGTTCGTTGCAACTCCCACATTAGAATATGAAGTCCTACCGAGAGAGATACGGAGACTAGAC
AGATACAGATGCATTTGCATGTGAATACACAATCCCACAATACAGACGTCAAAACCCATACCAGTTATTC
CAGAGAGATGGATTGGGCAGAAGGCAGAAGGAGAATACTCTGATCGTTTTTCGGCCACGTGTGTGTGTTA
TCTCAGTGTTTCTAAGAAGCGTTTGCTACTTTAGATTTTTTATTTAAAAAAATAGTAATAATCTATTAAG
TATGAGAGATGTGCAGAGAGGATTAGTGATCGAGAGCCATTTTTGCTGGTGGCAATCATATGGTACTTTT
AATGGGAATATTAGAAAGGCACCGGTAATGACCTTGTTGCAGCACAAAGGAGAGAGTGTGGGGTGCCCCT
GCATGTTGTCCCACCTCTTGTGACGTGTATCGTTTTGGAATTTCCAGTGGCTTGATCATGAACTACTGCA
GGAATCCAGATGCTGTGGCAGCTCCTTATTGTTATACGAGGGATCCCGGTGTCAGGTGGGAGTACTGCAA
CCTGACGCAATGCTCAGACGCAGAAGGGACTGCCGTCGCGCCTCCGACTGTTACCCCGGTTCCAAGCCTA
GAGGCTCCTTCCGAACAAGGTAAGGAGTCTGTGGCCAGACATCTACACGCTTCGATGCTGGGATGAAAAG
CCATGGAAATTCCCACTGATGCAGCCGCCTTCAATGGTAAACGGATGCTCGAGTGTTGCCTGAGTTCTAC
CATGTAGGAGGAAGCCTCCGTGCACTCTCTGGGGGAGCCAGCGGAGTGATTTCTGGTGCAACGTGGTTGG
GCTTTGTCTTTAGGATGGGCACAAACCCTCCAGGGGGATCGACTTCAAAATTCACCTTGTTGTAAAACGG
GCTACCTCAGTGTCCCAGCCAAAATTTTTATTGTAACATGCTGTCAGGTGTGTCACTCTTTCCAAGCCAG
TAAGCTTTTCCGGGGATTTCTTCAAGTAGCCAGCATTCAGAGCAATCTTCAGCATTGCAGATTCTGAGAA
ATGTGGCTCTGGAGCCTGTCACCCTCGAGAAACCTAAGAGGGCTGCATTGATTCCATGTGGCCCTGGGTC
TATGGAGCAGTACATGAGCTCCCAGTGCTCTAAGGCTCTTCAGCCCTAGGCTTTGAAGGGAGTGATTTCT
CAGTATTCTTAAACCTCTTTCTGATGACACTTGTACCTGTGAGGGGTCTAGAGAGAAAGAGTAGTAGACT
CCTACTTTACTACAATTCAGGATGCAGGGCATGAGAGGATTCCCTCTCTCCTCCAAGGGAAGAAGCTTTT
GGCGTGCACACATCCCTGAGAAGCAAAGTGTCTTTGTCTTCAGTCAGATACATAGGACCGTTTTCTGCCC
CATGGCCCGGAAGCCAAAGGCCTTGGCTTTCATGATCAACGGTCTAGGGAAACATGCAAAATTTCCATGT
CTGTCCCAAACTCTGCCCCCGACAGCCAATTACCACCTGCAGCCCGCATTGCCAAATGCGGTGCCGTTTG
CATGAAGATTCAGTAGAGTTTCCTAGAAAGGTGCTACCTCGTGAGCTCACTTTCCAATGAGGAATCTGAT
CTGTTGTGTTTCTCTAAGGTGTCAGGTGAAATATTTCCAAGAACTTACTACAGTTCTAGAATGGGAGGAA
TCTGTTGCTTTGGTGTTTGTTTGTTGGTCGGTTTTCTCACATCCATCTGCCTATGGATAAGGAAAAGAGA
ACGGTCGTAATTCTCATAGACTCCTTTCTGGTTGTGTCACAAATGGCTTCACATGTTTCTCTATGCTCAG
AGATACTCAGCTTGATTTCCCGTGTTTTCATTTCAGCACCGACTGAGCAAAGGCCTGGGGTGCAGGAGTG
CTACCATGGTAATGGACAGAGTTATCGAGGCACATACTCCACCACTGTCACAGGAAGAACCTGCCAAGCT
TGGTCATCTATGACACCACACTCGCATAGTCGGACCCCAGAATACTACCCAAATGCGTATGTCTTTGTTC
TTTACCATAAGAGAAGAAAGGGCCAAGTGAAGTTTCTGTTACAAGAGATGTGTCTCAAGCTGAGTTCTCC
GAACTCAACTTGTGACAGATGCAGATGGCGTAGCAAAATGTCTCAGGATGATTGCCTTGGAGCTAAGGGT
CTGAGAGAAGGGAAATGTTAAGCTCCCTCTCCTTCCTCCTAGTTCTATTGAGCAGAAGGGAAATCTGGAG
GTGAGGAGATCACATTATGAAGAAAGTCAGAATGACAAAGGACCAGACACTTAGATTACCCTTCCACAAC
ACCAACTAAACGTCAATGGAGACTTTCCAGTTGGAATTCCGTTATTCTGGCTTCCACTTCCTGAAGGGAA
GGTTGCGTTTGCCTTTTCTCTCTGGGTTCAAGAGGAAAGAATAGGTGCTTATTTATGGACAGGTGAATTG
ATCTGTTTCTATATCTACGTATATTCCGATTGTCAGAAAAACACTCGTTCCTAAGTACCAGTGGCCTGAA
GGGATACAGGTTCCCAGCAAGAGAAGATCCAAGGAAGGAAGGCAGATGAGAGTCAGCACAGAGAGGGATG
CTGAAAAGTAAAAGGGATGGGTGGATGGAGAGAAGCCCGGGTCTGACCACCCAATGGCCAATATTTTGGC
CACAAGCGACTACCAGAGACATGGAAAAATGGTTTCTACATGTGGGACAACAGATGGTAGAGGACCTAGA
GAATTGAGAGAGGGGCAATGATGGGCTCCACTCCGCAGATGCCTTGGCTTTCTTCCTGGATACCCTTCCT
GCACTGAATAGCAAGGAGATGGAGCCCAAGCAGACTGTAGCCATCTTGCTGAATGGAGGAGAGGGATTGG
AGTTTGGGATGACTGTGGTAGCTGAAATTTTTCTAGGTCTGCTAGAAATAAGAACTGGTTTGTGGAGGAA
AAGAGCTCTACAAATACGCATAGAAGTCTCCTCCAGTCGTTGGCCTGACATGACGCTGCCTGTGCACAGG
AAATGGTTCCACGAGAAAGTGTGGCAAAGAACATTTACTGAGAAACAGCAAGTACAAGAGCACAGGAAGC
TCAATAAAGAAGAGAGAGATCACATAGCACTCTGGGATACTGGAGTTCTTCCCAGCTAGACCAGAGAGTC
CTCACGGAGCACATTGCCAATTCAGTGGAGACCCCAGAACAGCCGTAATTTAAAGGTACACTTAGTATAT
TACTAGAATAAAGTCAGCTGCAGACAACCCCTTGCACAGCTGGAAAGCAAGTGTCCAAGCATCAAATCGG
TTTCCAATCAATGAAGTGCCTGTGAGAGGAAATCTCAACTCTCTTTAGAAGTAAACAACAAAGTCGATTG
CCTCAGCTATGCGGTATCCGCAGAGTGAGTCCTAAATTTAAAATCTGACTACATGTAGAAAAGCGTTTCG
TGTGACCCATGACCAGGAAATAAATCGGGTAATACAAACAGGCTCAGGAATGAGAGAAATGATTAGAATT
GCGTGAAAATTTGACATATCAGTATGATAACTGATTTCAAATATTTAAAAAAACAACATGCAAGAAAGCA
GATATCATATCAAGAGAAATTAACAGTACAGAATAGCCAAATTAAATTAAAGAGCTAGTATAAAAAAAGT
ATGTCTTAATTGAAAAAAATTACTGTATGGCCGGCTGATCAATTTAGACGTTTCAGAGGAAAACATTACC
CAACACACAATTCTAGAGAACCTACAGAATGAGCTACACACACACACACACACACACACACAAACTGAAA
ACACACCCATACTCACACACACGCAGAAACTCACAAGTTCTAACACACACAGACACGCGCACCCCTGAAG
AAACAGTGAAATATAAAATTAAGCGAGCCTCACAGACATGTAGGAAAATATGAAAAGATTTCCTGCATGT
GGGAAGCAAGTCACAGTAAAGAGCAAGGGAGTTTGGAATAGAAACAAATACCGGAATCAAGGATGGCTGA
TAACTTTTCAATTACGAAGAACATTAAAAAAAATCACAGAATCGTGAAACTCAAGGGATCACATAGGGAA
TTTCGGAAAAAAAACCCAACCTGTATGATGTACTTTTGTACATCACAGTTCGAAGGTAACAAGGCAAAGA
TATAATAAGAAGAAACCTGTCACGAGAAACTGGAGGAAAAAGAGCTGTGTCTTCCTACAAGTACACTGAT
ACAAATTGCCAATGTGTTCACCTCAGAAACACTGGAAGCCAGATACCAGGGAATATTGTTAAAATGATAA
TCAGGAACAAAAAGAGATCAACCGGGAATGCTGAATCCAGCAATAAAATGCCTTGAAGATCATCCATGTC
GGATAAATGCATATTGTGCACTGCCCCAAAGAAAGAAACCGGAAACTGTAAGAATTGGAAATCAGCAGGC
TTATGTAACAAGAGAGGTGACCCGAAGGAATTAGGTAGAAGAAGAATTGAACAAGAAAGGAACTTTCTGC
AGCCCACGTAATGAAGAATCCAGCAATTGGCAAATGTAGATAGATGTAAATGCAAAATATTTTCTTGATC
AAATTTCTATATCTTTGTAAATGAGAGTTGACTACTTGAAACAAAATGATAGCAAGATATTTAACTTCAG
CATATGTAGAGGTAAGAATTTGAAATGGTAGCATAAATCACGAAGGGATTAATTCGAAGTGTACCGTTGT
AAGTTTCTTTACCTCATGCACGATGGTGTGTCATATTAATAAAAGGGTACTGTGCGGGTTCGAAGGGATA
TTGCAAATCCTAGAGCAATCACAAAGGTTTGAACTCTGAGGTTTTTGGTATAATAAGAATAGTCCATGCA
TTCAAAAGAGGGAAGCCAAGGAAGAACTAGAAGTCTTTCAAGAGCTCAGGCTCTTATACATCCAGTTGCT
CATTGAACCAGCTTCCTGGAATGGAGGGTCTGGGGTTGAGACTAGGCCACAAGTCTAGAGTCTCTAGAGA
GACAGTGTTGGAACCCCATGGCCCATAATACATTTCCCATTTTCTCAGGCAGCCAGAGGTCATGAATGTG
AGGATACTGGGAGGTTGGAGCAACGTTCTTGGGAGGCATAAGGAAGAGCGAATGCTTCAAGATCCCCGCA
GCCCAAACTACTCGCCTGCTTTGCCCCCTAATGCATTTTTCTCTGCTGCTCCGTAGCTGTCCGACCTCTT
CAGATCTCTTAGTCCACCCTGCCGTCTTCCTTTATGCCATGGGTCCCACTGTTCTTTCAACTCATCCCCC
TTTCCCTCAGTCCCGGAGTAGCTGCGGCCAGCAGAGGGTAGACTGAGAGCAGGAGAGAAGGACCTGCCTA
GGAACCCCTTCTAGAGATACTGCATCCTGCCTGGGAGCAAGTTTTCCAGGGCAGCTTTGAGAAGTCTTGG
AGAAACAAACCTACTAAACCTGACAGACAGTAATACTATTTGCACAATGCTTTTCTGTGGGAAAGGTAGA
GCCTTTTCACTACGTATTGAGTACATAGAGTGTGAGGGTTGACCTGGAACGGCTATCCTCCTGGATGACG
TGTGTTTTCTGAAGAACTACATGTTCGTTGCAACTCCCACATTAGAATATGAAGTCCTACCGAGAGAGAT
ACGGAGACTAGACAGATACAGATGCATTTGCATGTGAATACACAATCCCACAATACAGACGTCAAAACCC
ATACCAGTTATTCCAGAGAGATGGATTGGGTAGGAGGCAGAAGGAGAATACTCTGATCGTTTTTCGGCCA
CGTGTGTGTGTTATCTCAGTGTTTCTAAGAAGCGTTTGCTACTTTAGATTTTTTATTTAAAAAAAATAGT
AATAATCTATTAAGTATGAGAGATGTGCAGAGAGGATTAGTGATCGAGAGCCATTTTTGCTGGTGGCAAT
CATATGGTACTTTTAATGGGAATATTAGAAAGGCACCGGTAATGACCTTGTTGCAGCACAAAGGAGAGAG
TGTGGGGTGCCCCTGCATGTTGTCCCACCTCTTGTGACGTGTATCGTTTTGGAATTTCCAGTGGCTTGAT
CATGAACTACTGCAGGAATCCAGATGCTGTGGCAGCTCCTTATTGTTATACGAGGGATCCCGGTGTCAGG
TGGGAGTACTGCAACCTGACGCAATGCTCAGACGCAGAAGGGACTGCCGTCGCGCCTCCGACTGTTACCC
CGGTTCCAAGCCTAGAGGCTCCTTCCGAACAAGGTAAGGAGTCTGTGGCCAGACATCTACACGCTTCGAT
GCTGGGATGAAAAGCCATGGAAATTCCCACTGATGCAGCCGCCTTCAATGGTAAACGGATGCTCGAGTGT
TGCCGGAGTTCTGCCATGTTGGGGGAAGCCTCCGTGTACTCTCTGGGGGAGCCAGCGGAGTGATTTCTGG
TGCAACTTGGGTGGGCTTTGTCTTTAGAATGGGCACAAACCTTCCAGGGTGATGGGCTTCACAACTCACC
TCCTTCTAAAATGGGCTATCTCAGTGTCTTAGCCAAAATTTTTATTGTAACGTGCTGTCAGGTGTGTGAT
TCTTTCTGTCGCAGTAAGCTTTTCTGGGGATTTCTTCAAGTAGCCAGCAGTCAGTGCAATCTTCAGCATT
GCAGATTTCAAAAAATGTGGCTCTGGAGCCTGTCATCCTCGAGAAACCTAACAGGGCTGCATTAATTCCA
TATGGTCCTGGGTCTATGGAGCAGTATATGAGCTCCCAATGCTCTAAGGCTCTTCAGTCCTAGGCTTTGA
AGGGAGTGATTTCTCAGTGTTCTTAAACCTCTTTCTGATGGCACTTGTACCTGTGAGGGGTCTAGAGAGA
AAGGTTAGTAGACTTCTCCTTTACTGCAATTCAGGATGCAGGGCATGAGAAGATTCCCTCCCTCCTCCAA
GGGAAGAAGGTTTTGGCGTGCACACATCCTTGAGAAGCAAAGTGTCTTTGCCTTCAGTCAGATATATAGG
ATCGTTTTCTGCCCCATGGCCTGGAAGCCAGAGGCCTTGGCTTTCATGATCAACGATCTAGGGAAACATG
CAAAATTTCCATGTCTTTCCCCTCCTCTGCCCTCGACAGCCAATTACCACCTGCATCCTGCATTGCCAAA
TGCAGTGCCCTTTGTATGAACATTCAGTAGAGTTTCATAGAAAGGTGCTACTTCGTGAGCGCACTTTGCA
GTGAGAAGGAGTCTGTTCTGTTCTGTTTTTCTAAGGATTTCAGGTGAAATATTTCCTAGAACTTACTACA
GTTCTAGATTGGTAGGAATCTGTAGGTTTGCTGTATGTTTTTTGGTTGGTTTTCTCCCATCCATCTGCCT
ACAGGTAAGGGAAAGATAACGTTCGTAATTCTCATAGACTCCTTTCTGGTTGTGTCATAAATGGCTTCAC
ATATTTCGTTATTCTCAGAGATACTCAGTTTATTTCTTGTGTTTTCATTTCAGCACCGACTGAGCAGAGG
CCTGGGGTGCAGGAGTGCTACCACGGTAATGGACAGAGTTATCGAGGCACATACTCCACCACTGTCACTG
GAAGAACCTGCCAAGCTTGGTCATCTATGACACCACACTCGCATAGTCGGACCCCAGAATACTACCCAAA
TGCGTATGTCTTTGTTCTTTACCATAAGAGAAGAAAGGGCCAAGTGAAGTTTCTGTTACAAGAGATGTGT
CTCAAGCTGAGTTCTCCGAACTCAACTTGTGACAGATGCAGATGGCGTAGCAAAATGTCTCAGGATGATT
GCCTTGGAGCTAAGGGTCTGAGAGAAGGGAAATGTTAAGCTCCCTCTCCTTCCTCCTAGTTCTATTGAGC
AGAAGGGAAATCTGGAGGTGAGGAGATCACATTATGAAGAAAGTCAGAATGACAAAGGACCAGACACTTA
GATTACCCTTCCACAACACCAACTAAACGTCAATGGAGACTTTCCAGTTGGAATTCCGTTATTCTGGCTT
CCACTTCCTGAAGGGAAGGTTGCGTTTGCCTTTTCTCTCTGGGTTCAAGAGGAAAGAATAGGTGCTTATT
TATGGACAGGTGAATTGATCTGTTTCTATATCTACGTATATTCCGATTGTCAGAAAAACACTCGTTCCTA
AGTACCAGTGGCCTGAAGGGATACAGGTTCCCAGCAAGAGAAGATCCAAGGAAGGAAGGCAGATGAGAGC
CAGCACAGAGAGGGATGCTGAAAAGTAAAAGGGATGGGTGGATGGAGAGAAGCCCGGGTCTGACCACCCA
ATGGCCAATATTTTGGCCACAAGCGACTACCAGAGACATGGAAAAATGGTTTCTACATGTGGGACAACAG
ATGGTAGAGGACCTAGAGAATTGAGAGAGGGGCAATGATGGGCTCCACTCCGCAGATGCCTTGGCTTTCT
TCCTGGATACCCTTCCTGCACTGAATAGCAAGGAGATGGAGCCCAAGCAGACTGTAGCCATCTTGCTGAA
TGGAGGAGAGGGATTGGAGTTTGGGATGACTGTGGTAGCTGAAATTTTTCTAGGTCTGCTAGAAATAAGA
ACTGGTTTGTGGAGGAAAAGAGCTCTACAAATACGCATAGAAGTCTCCTCCAGTCGTTGGCCTGACATGA
CGCTGCCTGTGCACAGGAAATGGTTCCACGAGAAAGTGTGGCAAAGAACATTTACTGAGAAACAGCAAGT
ACAAGAGCACAGGAAGCTCAATAAAGAAGAGAGAGATCACATAGCACTCTGGGATACTGGAGTTCTTCCC
AGCTAGACCAGAGAGTCCTCACGGAGCACATTGCCAATTCAGTGGAGACCCCAGAACAGCCGTAATTTAA
AGGTACACTTAGTATATTACTAGAATAAAGTCAGCTGCAGACAACCCCTTGCACAGCTGGAAAGCAAGTG
TCCAAGCATCAAATCGGTTTCCAATCAATGAAGTGCCTGTGGGAGGAAATCTCAACTCTCTTTAGAAGTA
AACAACAAAGTCGATTGCCTCAGCTATGCGGTATCCGCAGAGTGAGTCCTAAATTTAAAATCTGACTACA
TGTAGAAAAGCGTTTCGTGTGACCCATGACCAGGAAATAAATCGGGTAATACAAACAGGCTCAGGAATGA
GAGAAATGATTAGAATTGCGTGAAAATTTGACATATCAGTATGATAACTGATTTCAAATATTTAAAAAAA
CAACATGCAAGAAAGCAGATATCATATCAAGAGAAATTAACAGTACAGAATAGCCAAATTAAATTAAAGA
GCTAGTATAAAAAAAGTATGTCTTAATTGAAAAAAATTACTGTATGGCCGGCTGATCAAATTAGACGTTT
CAGAGGAAAACATTACCCAACACACAATTCTAGAGAACCTACAGAATGAGCTACACACACACACACACAC
ACACACACACACACTGAAAACACACCCATACTCACACACACGCAGAAACTCACAAGTTCTAACACACACA
GACACGCGCACCCCTGAAGAAACAGTGAAATATAAAATTAAGCGAGCCTCACAGACATGTAGGAAAATAT
GAAAAGATTTCCTGCATGTGGGAAGCAAGTCACAGTAAAGAGCAAGGGAGTTTGGAATAGAAACAAATAC
CGGAATCAAGGATGGCTGATAACTTTTCAATTACGAAGAACATTAAAAAAAATCACAGAATCGTGAAACT
CAAGGGATCATATAGGGAATTTCGGAAAAAAAACCCAACCTGTATGATGTACTTTTGTACATCACAGTTC
GAAGGTAACAAGGCAAAGATATAATAAGAAGAAACCTGTCACGAGAAACTGGAGGAAAAAGAGCTGTGTC
TTCCTACAAGTACACTGATACAAATTGCCAATGTGTTCACCTCAGAAACACTGGAAGCCAGATACCAGGG
AATATTGTTAAAATGATAATCAGGAACAAAAAGAGATCAACCGGGAATGCTGAATCCAGCAATAAAATGC
CTTGAAGATCATCCATGTCGGATAAATGCATATTGTGCACTGCCCCAAAGAAAGAAACCGGAAACTGTCA
GAATTGGAAATCAGCAGGCTTATGTAACAAGAGAGGTGACCCGAAGGAATTAGGTAGAAGAAGAATTGAA
CAAGAAAGGAACTTTCTGCAGCCCACGTAATGAAGAATCCAGCAATTGGCAAATGTAGATAGATGTAAAT
GCAAAATATTTTCTTGATCAAATTTCTATATCTTTGTAAATGAGAGTTGACTACTTGAAACAAAATGATA
GCAAGATATTTAACTTCAGCATATGTAGAGGTAAGAATTTGAAATGGTAGCATAAATCACGAAGGGATTA
ATTCGAAGTGTACCGTTGTAAGTTTCTTTACCTCATGCACGATGGTGTGTCATATTAATAAAAGGGTACT
GTGCGGGTTCGAAGGGATATTGCAAATCCTAGAGCAATCACAAAGGTTTGAACTCTGAGGTTTTTGGTAT
AATAAGAATAGTCCATGCATTCAAAAGAGGGAAGCCAAGGAAGAACTAGAAGTCTTTCAAGAGCTCAGGC
TCTTATACATCCAGTTGCTCATTGAACCAGCTTCCTGGAATGGAGGGTCTGGGGTTGAGACTAGGCCACA
AGTCTAGAGTCTCTAGAGAGACAGTGTTGGAACCCCATGGCCCATAATACATTTCCCATTTTCTCAGGCA
GCCAGAGGTCATGAATGTGAGGATACTGGGAGGTTGGAGCAACGTTCTTGGGAGGCATAAGGAAGAGCGA
ATGCTTCAAGATCCCCGCAGCCCAAACTACTCGCCTGCTTTGCCCCCTAATGCATTTTTCTCTGCTGCTC
CGTAGCTGTCCGACCTCTTCAGATCTCTTAGTCCACCCTGCCGTCTTCCTTTATGCCATGGGTCCCATTG
TTCTTTCAACTCATCCCCCTTTCCCTCAGTCCCGGAGTAGCTGCGGCCAGCAGAGGGTAGACTGAGAGCA
GGAGAGAAGGACCTGCCTAGGAACCCCTTCTAGAGATACTGCATCCTGCCTGGGAGCAAGTTTTCCAGGG
CAGCTTTGAGAAGTCTTGGAGAAACAAACCTACTAAACCTGACAGACAGTAATACTATTTGCACAATGCT
TTTCTGTGGGAAAGGTAGAGCCTTTTCACTACGTATTGAGTACATAGAGTGTGAGGGTTGACCTGGAACG
GCTATCCTCCTGGATGACGTGCGTTTTCTGAAGAACTACATGTTCGTTGCAACTCCCACATTAGAATATG
AAGTCCTACCGAGAGAGATACGGAGACTAGACAGATACAGATGCATTTGCATGTGAATACACAATCCCAC
AATACAGACGTCAAAACCCATACCAGTTATTCCAGAGAGATGGATTGGGCAGAAGGCAGAAGGAGAATAC
TCTGATCGTTTTTCGGCCACGTGTGTGTGTTATCTCAGTGTTTCTAAGAAGCGTTTGCTACTTTAGATTT
TTTATTTAAAAAAAATAGTAATAATCTATTAAGTATGAGAGATGTGCAGAGAGGATTAGTGATCGAGAGC
CATTTTTGCTGGTGGCAATCATATGGTACTTTTAATGGGAATATTAGAAAGGCACCGGTAATGACCTTGT
TGCAGCACAAAGGAGAGAGTGTGGGGTGCCCCTGCATGTTGTCCCACCTCTTGTGACGTGTATCGTTTTG
GAATTTCCAGTGGCTTGATCATGAACTACTGCAGGAATCCAGATGCTGTGGCAGCTCCTTATTGTTATAC
GAGGGATCCCGGTGTCAGGTGGGAGTACTGCAACCTGACGCAATGCTCAGACGCAGAAGGGACTGCCGTC
GCGCCTCCGACTGTTACCCCGGTTCCAAGCCTAGAGGCTCCTTCCGAACAAGGTAAGGAGTCTGTGGCCA
GACATCTACACGCTTCGATGCTGGGATGAAAAGCCATGGAAATTCCCACTGATGCAGCCGCCTTCAATGG
TAAACGGATGCTCGAGTGTTGCCTGAGTTCTACCATGTAGGAGGAAGCCTCCGTGCACTCTCTGGGGGAG
CCAGCGGAGTGATTTCTGGTGCAACGTGGTTGGGCTTTGTCTTTAGGATGGGCACAAACCCTCCAGGGGG
ATCGACTTCAAAATTCACCTTGTTGTAAAACGGGCTACCTCAGTGTCCCAGCCAAAATTTTTATTGTAAC
ATGCTGTCAGGTGTGTCACTCTTTCCAAGCCAGTAAGCTTTTCCGGGGATTTCTTCAAGTAGCCAGCATT
CAGAGCAATCTTCAGCATTGCAGATTCTGAGAAATGTGGCTCTGGAGCCTGTCACCCTCGAGAAACCTAA
GAGGGCTGCATTGATTCCATGTGGCCCTGGGTCTATGGAGCAGTACATGAGCTCCCAGTGCTCTAAGGCT
CTTCAGCCCTAGGCTTTGAAGGGAGTGATTTCTCAGTATTCTTAAACCTCTTTCTGATGACACTTGTACC
TGTGAGGGGTCTAGAGAGAAAGAGTAGTAGACTCCTACTTTACTACAATTCAGGATGCAGGGCATGAGAG
GATTCCCTCTCTCCTCCAAGGGAAGAAGCTTTTGGCGTGCACACATCCCTGAGAAGCAAAGTGTCTTTGT
CTTCAGTCAGATACATAGGACCGTTTTCTGCCCCATGGCCCGGAAGCCAAAGGCCTTGGCTTTCATGATC
AACGGTCTAGGGAAACATGCAAAATTTCCATGTCTGTCCCAAACTCTGCCCCCGACAGCCAATTACCACC
TGCAGCCCGCATTGCCAAATGCGGTGCCGTTTGCATGAAGATTCAGTAGAGTTTCCTAGAAAGGTGCTAC
CTCGTGAGCTCACTTTCCAATGAGGAATCTGATCTGTTGTGTTTCTCTAAGGTGTCAGGTGAAATATTTC
CAAGAACTTACTACAGTTCTAGAATGGGAGGAATCTGTTGCTTTGGTGTTTGTTTGTTGGTCGGTTTTCT
CACATCCATCTGCCTATGGATAAGGAAAAGAGAACGGTCGTAATTCTCATAGACTCCTTTCTGGTTGTGT
CACAAATGGCTTCACATGTTTCTCTATGCTCAGAGATACTCAGCTTGATTTCCCGTGTTTTCATTTCAGC
ACCGACTGAGCAAAGGCCTGGGGTGCAGGAGTGCTACCATGGTAATGGACAGAGTTATCGAGGCACATAC
TCCACCACTGTCACAGGAAGAACCTGCCAAGCTTGGTCATCTATGACACCACACTCGCATAGTCGGACCC
CAGAATACTACCCAAATGCGTATGTCTTTGTTCTTTACCATAAGAGAAGAAAGGGCCAAGTGAAGTTTCT
GTTACAAGAGATGTGTCTCAAGCTGAGTTCTCCGAACTCAACTTGTGACAGATGCAGATGGCGTAGCAAA
ATGTCTCAGGATGATTGCCTTGGAGCTAAGGGTCTGAGAGAAGGGAAATGTTAAGCTCCCTCTCCTTCCT
CCTAGTTCTATTGAGCAGAAGGGAAATCTGGAGGTGAGGAGATCACATTATGAAGAAAGTCAGAATGACA
AAGGACCAGACACTTAGATTACCCTTCCACAACACCAACTAAACGTCAATGGAGACTTTCCAGTTGGAAT
TCCGTTATTCTGGCTTCCACTTCCTGAAGGGAAGGTTGCGTTTGCCTTTTCTCTCTGGGTTCAAGAGGAA
AGAATAGGTGCTTATTTATGGACAGGTGAATTGATCTGTTTCTATATCTACGTATATTCCGATTGTCAGA
AAAACACTCGTTCCTAAGTACCAGTGGCCTGAAGGGATACAGGTTCCCAGCAAGAGAAGATCCAAGGAAG
GAAGGCAGATGAGAGTCAGCACAGAGAGGGATGCTGAAAAGTAAAAGGGATGGGTGGATGGAGAGAAGCC
CGGGTCTGACCACCCAATGGCCAATATTTTGGCCACAAGCGACTACCAGAGACATGGAAAAATGGTTTCT
ACATGTGGGACAACAGATGGTAGAGGACCTAGAGAATTGAGAGAGGGGCAATGATGGGCTCCACTCCGCA
GATGCCTTGGCTTTCTTCCTGGATACCCTTCCTGCACTGAATAGCAAGGAGATGGAGCCCAAGCAGACTG
TAGCCATCTTGCTGAATGGAGGAGAGGGATTGGAGTTTGGGATGACTGTGGTAGCTGAAATTTTTCTAGG
TCTGCTAGAAATAAGAACTGGTTTGTGGAGGAAAAGAGCTCTACAAATACGCATAGAAGTCTCCTCCAGT
CGTTGGCCTGACATGACGCTGCCTGTGCACAGGAAATGGTTCCACGAGAAAGTGTGGCAAAGAACATTTA
CTGAGAAACAGCAAGTACAAGAGCACAGGAAGCTCAATAAAGAAGAGAGAGATCACATAGCACTCTGGGA
TACTGGAGTTCTTCCCAGCTAGACCAGAGAGTCCTCACGGAGCACATTGCCAATTCAGTGGAGACCCCAG
AACAGCCGTAATTTAAAGGTACACTTAGTATATTACTAGAATAAAGTCAGCTGCAGACAACCCCTTGCAC
AGCTGGAAAGCAAGTGTCCAAGCATCAAATCGGTTTCCAATCAATGAAGTGCCTGTGAGAGGAAATCTCA
ACTCTCTTTAGAAGTAAACAACAAAGTCGATTGCCTCAGCTATGCGGTATCCGCAGAGTGAGTCCTAAAT
TTAAAATCTGACTACATGTAGAAAAGCGTTTCGTGTGACCCATGACCAGGAAATAAATCGGGTAATACAA
ACAGGCTCAGGAATGAGAGAAATGATTAGAATTGCGTGAAAATTTGAAATATCAGTATGATAACTGATTT
CAAATATTTAAAAAAACAACATGCAAGAAAGCAGATATCATATCAAGAGAAATTAACAGTACAGAATAGC
CAAATTAAATTAAAGAGCTAGTATAAAAAAAGTATGTCTTAATTGAAAAAAATTACTGTATGGCCGGCTG
ATCAATTTAGACGTTTCAGAGGAAAACATTACCCAACACACAATTCTAGAGAACCTACAGAATGAGCTAC
ACACACACACACACACACACACACAAACTGAAAACACACCCATACTCACACACACGCAGAAACTCACAAG
TTCTAACACACACAGACACGCGCACCCCTGAAGAAACAGTGAAATATAAAATTAAGCGAGCCTCACAGAC
ATGTAGGAAAATATGAAAAGATTTCCTGCATGTGGGAAGCAAGTCACAGTAAAGAGCAAGGGAGTTTGGA
ATAGAAACAAATACCAGAATCAAGGATGGCTGATAACTTTTCAATTACGAAGAACATTAAAAAAAATCAC
AGAATCGTGAAACTCAAGGGATCACATAGGGAATTTCGGAAAAAAAACCCAACCTGTATGATGTACTTTT
GTACATCACAGTTCGAAGGTAACAAGGCAAAGATATAATAAGAAGAAACCTGTCACGAGAAACTGGAGGA
AAAAGAGCTGTGTCTTCCTACAAGTACACTGATACAAATTGCCAATGTGTTCACCTCAGAAACACTGGAA
GCCAGATACCAGGGAATATTGTTAAAATGATAATCAGGAACAAAAAGAGATCAACCGGGAATGCTGAATC
CAGCAATAAAATGCCTTGAAGATCATCCATGTCGGATAAATGCATATTGTGCACTGCCCCAAAGAAAGAA
ACCGGAAACTGTAAGAATTGGAAATCAGCAGGCTTATGTAACAAGAGAGGTGACCCGAAGGAATTAGGTA
GAAGAAGAATTGAACAAGAAAGGAACTTTCTGCAGCCCACGTAATGAAGAATCCAGCAATTGGCAAATGT
AGATAGATGTAAATGCAAAATATTTTCTTGATCAAATTTCTATATCTTTGTAAATGAGAGTTGACTACTT
GAAACAAAATGATAGCAAGATATTTAACTTCAGCATATGTAGAGGTAAGAATTTGAAATGGTAGCATAAA
TCACGAAGGGATTAATTCGAAGTGTACCGTTGTAAGTTTCTTTACCTCATGCACGATGGTGTGTCATATT
AATAAAAGGGTACTGTGCGGGTTCGAAGGGATATTGCAAATCCTAGAGCAATCACAAAGGTTTGAACTCT
GAGGTTTTTGGTATAATAAGAATAGTCCATGCATTCAAAAGAGGGAAGCCAAGGAAGAACTAGAAGTCTT
TCAAGAGCTCAGGCTCTTATACATCCAGTTGCTCATTGAACCAGCTTCCTGGAATGGAGGGTCTGGGGTT
GAGACTAGGCCACAAGTCTAGAGTCTCTAGAGAGACAGTGTTGGAACCCCATGGCCCATAATACATTTCC
CATTTTCTCAGGCAGCCAGAGGTCATGAATGTGAGGATACTGGGAGGTTGGAGCAACGTTCTTGGGAGGC
ATAAGGAAGAGCGAATGCTTCAAGATCCCCGCAGCCCAAACTACTCGCCTGCTTTGCCCCCTAATGCATT
TTTCTCTGCTGCTCCGTAGCTGTCCGACCTCTTCAGATCTCTTAGTCCACCCTGCCGTCTTCCTTTATGC
CATGGGTCCCACTGTTCTTTCAACTCATCCCCCTTTCCCTCAGTCCCGGAGTAGCTGCGGCCAGCAGAGG
GTAGACTGAGAGCAGGAGAGAAGGACCTGCCTAGGAACCCCTTCTAGAGATACTGCATCCTGCCTGGGAG
CAAGTTTTCCAGGGCAGCTTTGAGAAGTCTTGGAGAAACAAACCTACTAAACCTGACAGACAGTAATACT
ATTTGCACAATGCTTTTCTGTGGGAAAGGTAGAGCCTTTTCACTACGTATTGAGTACATAGAGTGTGAGG
GTTGACCTGGAACGGCTATCCTCCTGGATGACGTGTGTTTTCTGAAGAACTACATGTTCGTTGCAACTCC
CACATTAGAATATGAAGTCCTACCGAGAGAGATACGGAGACTAGACAGATACAGATGCATTTGCATGTGA
ATACACAATCCCACAATACAGACGTCAAAACCCATACCAGTTATTCCAGAGAGATGGATTGGGCAGAAGG
CAGAAGGAGAATACTCTGATCGTTTTTCGGCCACGTGTGTGTGTTATCTCAGTGTTTCTAAGAAGCGTTT
GCTACTTTAGATTTTTTATTTAAAAAAAATAGTAATAATCTATTAAGTATGAGAGATGTGCAGAGAGGAT
TAGTGATCGAGAGCCATTTTTGCTGGTGGCAATCATATGGTACTTTTAATGGGAATATTAGAAAGGCACC
GGTAATGACCTTGTTGCAGCACAAAGGAGAGAGTGTGGGGTGCCCCTGCATGTTGTCCCACCTCTTGTGA
CGTGTATCGTTTTGGAATTTCCAGTGGCTTGATCATGAACTACTGCAGGAATCCAGATGCTGTGGCAGCT
CCTTATTGTTATACGAGGGATCCCGGTGTCAGGTGGGAGTACTGCAACCTGACGCAATGCTCAGACGCAG
AAGGGACTGCCGTCGCGCCTCCGACTGTTACCCCGGTTCCAAGCCTAGAGGCTCCTTCCGAACAAGGTAA
GGAGTCTGTGGCCAGACATCTACACGCTTCGATGCTGGGATGAAAAGCCATGGAAATTCCCACTGATGCA
GCCGCCTTCAATGGTAAACGGATGCTCGAGTGTTGCCTGAGTTCTACCATGTAGGAGGAAGCCTCCGTGC
ACTCTCTGGGGGAGCCAGCGGAGTGATTTCTGGTGCAACGTGGTTGGGCTTTGTCTTTAGGATGGGCACA
AACCCTCCAGGGGGATCGACTTCAAAATTCACCTTGTTGTAAAACGGGCTACCTCAGTGTCCCAGCCAAA
ATTTTTATTGTAACATGCTGTCAGGTGTGTCACTCTTTCCAAGCCAGTAAGCTTTTCCGGGGATTTCTTC
AAGTAGCCAGCATTCAGAGCAATCTTCAGCATTGCAGATTCTGAGAAATGTGGCTCTGGAGCCTGTCACC
CTCGAGAAACCTAAGAGGGCTGCATTGATTCCATGTGGCCCTGGGTCTATGGAGCAGTACATGAGCTCCC
AGTGCTCTAAGGCTCTTCAGCCCTAGGCTTTGAAGGGAGTGATTTCTCAGTATTCTTAAACCTCTTTCTG
ATGACACTTGTACCTGTGAGGGGTCTAGAGAGAAAGAGTAGTAGACTCCTACTTTACTACAATTCAGGAT
GCAGGGCATGAGAGGATTCCCTCTCTCCTCCAAGGGAAGAAGCTTTTGGCGTGCACACATCCCTGAGAAG
CAAAGTGTCTTTGTCTTCAGTCAGATACATAGGACCGTTTTCTGCCCCATGGCCCGGAAGCCAAAGGCCT
TGGCTTTCATGATCAACGGTCTAGGGAAACATGCAAAATTTCCATGTCTGTCCCAAACTCTTCCCCCGAC
AGCCAATTACCACCTGCAGCCCGCATTGCCAAATGCGGTGCCGTTTGCATGAAGATTCAGTAGAGTTTCC
TAGAAAGGTGCTACCTCGTGAGCTCACTTTCCAATGAGGAATCTGATCTGTTGTGTTTCTCTAAGGTGTC
AGGTGAAATATTTCCAAGAACTTACTACAGTTCTAGAATGGGAGGAATCTGTTGCTTTGGTGTTTGTTTG
TTGGTCGGTTTTCTCACATCCATCTGCCTATGGATAAGGAAAAGAGAACGGTCGTAATTCTCATAGACTC
CTTTCTGGTTGTGTCACAAATGGCTTCACATGTTTCTCTATGCTCAGAGATACTCAGCTTGATTTCCCGT
GTTTTCATTTCAGCACCGACTGAGCAAAGGCCTGGGGTGCAGGAGTGCTACCATGGTAATGGACAGAGTT
ATCGAGGCACATACTCCACCACTGTCACAGGAAGAACCTGCCAAGCTTGGTCATCTATGACACCACACTC
GCATAGTCGGACCCCAGAATACTACCCAAATGCGTATGTCTTTGTTCTTTACCATAAGAGAAGAAAGGGC
CAAGTGAAGTTTCTGTTACAAGAGATGTGTCTCAAGCTGAGTTCTCCGAACTCAACTTGTGACAGATGCA
GATGGCGTAGCAAAATGTCTCAGGATGATTGCCTTGGAGCTAAGGGTCTGAGAGAAGGGAAATGTTAAGC
TCCCTCTCCTTCCTCCTAGTTCTATTGAGCAGAAGGGAAATCTGGAGGTGAGAAGATCACATTATGAAGA
AAGTCAGAATGACAAAGGACCAGACACTTAGATTACCCTTCCACAACACCAACTAAACGTCAATGGAGAC
TTTCCAGTTGGAATTCCGTTATTCTGGCTTCCACTTCCTGAAGGGAAGGTTGCGTTTGCCTTTTCTCTCT
GGGTTCAAGAGGAAAGAATAGGTGCTTATTTATGGACAGGTGAATTGATCTGTTTCTATATCTACGTATA
TTCCGATTGTCAGAAAAACACTCGTTCCTAAGTACCAGTGGCCTGAAGGGATACAGGTTCCCAGCAAGAG
AAGATCCAAGGAAGGAAGGCAGATGAGAGTCAGCACAGAGAGGGATGCTGAAAAGTAAAAGGGATGGGTG
GATGGAGAGAAGCCCGGGTCTGACCACCCAATGGCCAATATTTTGGCCACAAGCGACTACCAGAGACATG
GAAAAATGGTTTCTACATGTGGGACAACAGATGGTAGAGGACCTAGAGAATTGAGAGAGGGGCAATGATG
GGCTCCACTCCGCAGATGCCTTGGCTTTCTTCCTGGATACCCTTCCTGCACTGAATAGCAAGGAGATGGA
GCCCAAGCAGACTGTAGCCATCTTGCTGAATGGAGGAGAGGGATTGGAGTTTGGGATGACTGTGGTAGCT
GAAATTTTTCTAGGTCTGCTAGAAATAAGAACTGGTTTGTGTGGAGGAAAAGAGCTCTACAAATACGCAT
AGAAGTCTCCTCCAGTCGTTGGCCTGACATGACGCTGCCTGTGCACAGGAAATGGTTCCACGAGAAAGTG
TGGCAAAGAACATTTACTGAGAAACAGCAAGTACAAGAGCACAGGAAGCTCAATAAAGAAGAGAGAGATC
ACATAGCACTCTGGGATACTGGAGTTCTTCCCAGCTAGACCAGAGAGTCCTCACGGAGCACATTGCCAAT
TCAGTGGAGACCCCAGAACAGCCGTAATTTAAAGGTACACTTAGTATATTACTAGAATAAAGTCAGCTGC
AGACAACCCCTTGCACAGCTGGAAAGCAAGTGTCCAAGCATCAAATCGGTTTCCAATCAATGAAGTGCCT
GTGAGAGGAAATCTCAACTCTCTTTAGAAGTAAACAACAAAGTCGATTGCCTCAGCTATGCGGTATCCGC
AGAGTGAGTCCTAAATTTAAAATCTGACTACATGTAGAAAAGCGTTTCGTGTGACCCATGACCAGGAAAT
AAATCGGGTAATACAAACAGGCTCAGGAATGAGAGAAATGATTAGAATTGCGTGAAAATTTGACATATCA
GTATGATAACTGATTTCAAATATTTAAAAAAACAACATGCAAGAAAGCAGATATCATATCAAGAGAAATT
AACAGTACAGAATAGCCAAATTAAATTAAAGAGGTAGTATAAAAAAAGTATGTCTTAATTGAAAAAAATT
ACTGTATGGCCGGCTGATCAATTTAGACGTTTCAGAGGAAAACATTACCCAACACACAATTCTAGAGAAC
CTACAGAATGAGCTACACACACACACACACACACACACACAAACTGAAAACACACCCATACTCACACACA
CGCAGAAACTCACAAGTTCTAACACACACAGACACGCGCACCCCTGAAGAAACAGTGAAATATAAAATTA
AGCGAGCCTCACAGACATGTAGGAAAATATGAAAAGATTTCCTGCATGTGGGAAGCAAGTCACAGTAAAG
AGCAAGGGAGTTTGGAATAGAAACAAATACCGGAATCAAGGATGGCTGATAACTTTTCAATTACGAAGAA
CATTAAAAAAAATCACAGAATCGTGAAACTCAAGGGATCACATAGGGAATTTCGGAAAAAAAACCCAACC
TGTATGATGTACTTTTGTACATCACAGTTCGAAGGTAACAAGGCAAAGATATAATAAGAAGAAACCTGTC
ACGAGAAACTGGAGGAAAAAGAGCTGTGTCTTCCTACAAGTACACTGATACAAATTGCCAATGTGTTCAC
CTCAGAAACACTGGAAGCCAGATACCAGGGAATATTGTTAAAATGATAATCAGGAACAAAAAGAGATCAA
CCGGGAATGCTGAATCCAGCAATAAAATGCCTTGAAGGTCATCCATGTCGGATAAATGCATATTGTGCAC
TGCCCCAAAGAAAGAAACCGGAAACTGTAAGAATTGGAAATCAGCAGGCTTATGTAACAAGAGAGGTGAC
CCGAAGGAATTAGGTAGAAGAAGAATTGAACAAGAAAGGAACTTTCTGCAGCCCACGTAATGAAGAATCC
AGCAATTGGCAAATGTAGATAGATGTAAATGCAAAATATTTTCTTGATCAAATTTCTATATCTTTGTAAA
TGAGAGTTGACTACTTGAAACAAAATGATAGCAAGATATTTAACTTCAGCATATGTAGAGGTAAGAATTT
GAAATGGTAGCATAAATCACGAAGGGATTAATTCGAAGTGTACCGTTGTAAGTTTCTTTACCTCATGCAC
GATGGTGTGTCATATTAATAAAAGGGTACTGTGCGGGTTCGAAGGGATATTGCAAATCCTAGAGCAATCA
CAAAGGTTTGAACTCTGAGGTTTTTGGTATAATAAGAATAGTCCATGCATTCAAAAGAGGGAAGCCAAGG
AAGAACTAGAAGTCTTTCAAGAGCTCAGGCTCTTATACATCCAGTTGCTCATTGAACCAGCTTCCTGGAA
TGGAGGGTCTGGGGTTGAGACTAGGCCACAAGTCTAGAGTCTCTAGAGAGACAGTGTTGGAACCCCATGG
CCCATAATACATTTCCCATTTTCTCAGGCAGCCAGAGGTCATGAATGTGAGGATACTGGGAGGTTGGAGC
AACGTTCTTGGGAGGCATAAGGAAGAGCGAATGCTTCAAGATCCCCGCAGCCCAAACTACTCGCCTGCTT
TGCCCCCTAATGCATTTTTCTCTGCTGCTCCGTAGCTGTCCGACCTCTTCAGATCTCTTAGTCCACCCTG
CCGTCTTCCTTTATGCCATGGGTCCCACTGTTCTTTCAACTCATCCCCCTTTCCCTCAGTCCCGGAGTAG
CTGCGGCCAGCAGAGGGTAGACTGAGAGCAGGAGAGAAGGACCTGCCTAGGAACCCCTTCTAGAGATACT
GCATCCTGCCTGGGAGCAAGTTTTCCAGGGCAGCTTTGAGAAGTCTTGGAGAAACAAACCTACTAAACCT
GACAGACAGTAATACTATTTGCACAATGCTTTTCTGTGGGAAAGGTAGAGCCTTTTCACTACGTATTGAG
TACATAGAGTGTGAGGGTTGACCTGGAACGGCTATCCTCCTGGATGACGTGCGTTTTCTGAAGAACTACA
TGTTCGTTGCAACTCCCACATTAGAATATGAAGTCCTACCGAGAGAGATACGGAGACTAGACAGATACAG
ATGCATTTGCATGTGAATACACAATCCCACAATACAGACGTCAAAACCCATACCAGTTATTCCAGAGAGA
TGGATTGGGCAGAAGGCAGAAGGAGAATACTCTGATCGTTTTTCGGCCACGTGTGTGTGTTATCTCAGTG
TTTCTAAGAAGCGTTTGCTACTTTAGATTTTTTATTTAAAAAAAATAGTAATAATCTATTAAGTATGAGA
GATGTGCAGAGAGGATTAGTGATCGAGAGCCATTTTTGCTGGTGGCAATCATATGGTACTTTTAATGGGA
ATATTAGAAAGGCACCGGTAATGACCTTGTTGCAGCACAAAGGAGAGAGTGTGGGGTGCCCCTGCATGTT
GTCCCACCTCTTGTGACGTGTATCGTTTTGGAATTTCCAGTGGCTTGATCATGAACTACTGCAGGAATCC
AGATGCTGTGGCAGCTCCTTATTGTTATACGAGGGATCCCGGTGTCAGGTGGGAGTACTGCAACCTGACG
CAATGCTCAGACGCAGAAGGGACTGCCGTCGCGCCTCCGACTGTTACCCCGGTTCCAAGCCTAGAGGCTC
CTTCCGAACAAGGTAAGGAGTCTGTGGCCAGACATCTACACGCTTCGATGCTGGGATGAAAAGCCATGGA
AATTCCCACTGATGCAGCCGCCTTCAATGGTAAACGGATGCTCGAGTGTTGCCTGAGTTCTACCATGTAG
GAGGAAGCCTCCGTGCACTCTCTGGGGGAGCCAGCGGAGTGATTTCTGGTGCAACGTGGTTGGGCTTTGT
CTTTAGGATGGGCACAAACCCTCCAGGGGGATCGACTTCAAAATTCACCTTGTTGTAAAACGGGCTACCT
CAGTGTCCCAGCCAAAATTTTTATTGTAACATGCTGTCAGGTGTGTCACTCTTTCCAAGCCAGTAAGCTT
TTCCGGGGATTTCTTCAAGTAGCCAGCATTCAGAGCAATCTTCAGCATTGCAGATTCTGAGAAATGTGGC
TCTGGAGCCTGTCACCCTCGAGAAACCTAAGAGGGCTGCATTGATTCCATGTGGCCCTGGGTCTATGGAG
CAGTACATGAGCTCCCAGTGCTCTAAGGCTCTTCAGCCCTAGGCTTTGAAGGGAGTGATTTCTCAGTATT
CTTAAACCTCTTTCTGATGACACTTGTACCTGTGAGGGGTCTAGAGAGAAAGAGTAGTAGACTCCTACTT
TACTACAATTCAGGATGCAGGGCATGAGAGGATTCCCTCTCTCCTCCAAGGGAAGAAGCTTTTGGCGTGC
ACACATCCCTGAGAAGCAAAGTGTCTTTGTCTTCAGTCAGATACATAGGACCGTTTTCTGCCCCATGGCC
CGGAAGCCAAAGGCCTTGGCTTTCATGATCAACGGTCTAGGGAAACATGCAAAATTTCCATGTCTGTCCC
AAACTCTGCCCCCGACAGCCAATTACCACCTGCAGCCCGCATTGCCAAATGCGGTGCCGTTTGCATGAAG
ATTCAGTAGAGTTTCCTAGAAAGGTGCTACCTCGTGAGCTCACTTTCCAATGAGGAATCTGATCTGTTGT
GTTTCTCTAAGGTGTCAGGTGAAATATTTCCAAGAACTTACTACAGTTCTAGAATGGGAGGAATCTGTTG
CTTTGGTGTTTGTTTGTTGGTCGGTTTTCTCACATCCATCTGCCTATGGATAAGGAAAAGAGAACGGTCG
TAATTCTCATAGACTCCTTTCTGGTTGTGTCACAAATGGCTTCACATGTTTCTCTATGCTCAGAGATACT
CAGCTTGATTTCCCGTGTTTTCATTTCAGCACCGACTGAGCAAAGGCCTGGGGTGCAGGAGTGCTACCAT
GGTAATGGACAGAGTTATCGAGGCACATACTCCACCACTGTCACAGGAAGAACCTGCCAAGCTTGGTCAT
CTATGACACCACACTCGCATAGTCGGACCCCAGAATACTACCCAAATGCGTATGTCTTTGTTCTTTACCA
TAAGAGAAGAAAGGGCCAAGTGAAGTTTCTGTTACAAGAGATGTGTCTCAAGCTGAGTTCTCCGAACTCA
ACTTGTGACAGATGCAGATGGCGTAGCAAAATGTCTCAGGATGATTGCCTTGGAGCTAAGGGTCTGAGAG
AAGGGAAATGTTAAGCTCCCTCTCCTTCCTCCTAGTTCTATTGAGCAGAAGGGAAATCTGGAGGTGAGGA
GATCACATTATGAAGAAAGTCAGAATGACAAAGGACCAGACACTTAGATTACCCTTCCACAACACCAACT
AAACGTCAATGGAGACTTTCCAGTTGGAATTCCGTTATTCTGGCTTCCACTTCCTGAAGGGAAGGTTGCG
TTTGCCTTTTCTCTCTGGGTTCAAGAGGAAAGAATAGGTGCTTATTTATGGACAGGTGAATTGATCTGTT
TCTATATCTACGTATATTCCGATTGTCAGAAAAACACTCGTTCCTAAGTACCAGTGGCCTGAAGGGATAC
AGGTTCCCAGCAAGAGAAGATCCAAGGAAGGAAGGCAGATGAGAGTCAGCACAGAGAGGGATGCTGAAAA
GTAAAAGGGATGGGTGGATGGAGAGAAGCCCGGGTCTGACCACCCAATGGCCAATATTTTGGCCACAAGC
GACTACCAGAGACATGGAAAAATGGTTTCTACATGTGGGACAACAGATGGTAGAGGACCTAGAGAATTGA
GAGAGGGGCAATGATGGGCTCCACTCCGCAGATGCCTTGGCTTTCTTCCTGGATACCCTTCCTGCACTGA
ATAGCAAGGAGATGGAGCCCAAGCAGACTGTAGCCATCTTGCTGAATGGAGGAGAGGGATTGGAGTTTGG
GATGACTGTGGTAGCTGAAATTTTTCTAGGTCTGCTAGAAATAAGAACTGGTTTGTGTGGAGGAAAAGAG
CTCTACAAATACGCATAGAAGTCTCCTCCAGTCGTTGGCCTGACATGACGCTGCCTGTGCACAGGAAATG
GTTCCACGAGAAAGTGTGGCAAAGAACATTTACTGAGAAACAGCAAGTACAAGAGCACAGGAAGCTCAAT
AAAGAAGAGAGAGATCACATAGCACTCTGGGATACTGGAGTTCTTCCCAGCTAGACCAGAGAGTCCTCAC
GGAGCACATTGCCAATTCAGTGGAGACCCCAGAACAGCCGTAATTTAAAGGTACACTTAGAATATTACTA
GAATAAAGTCAGCTGCAGACAACCCCTTGCACAGCTGGAAAGCAAGTGTCCAAGCATCAAATCGGTTTCC
AATCAATGAAGTGCCTGTGAGAGGAAATCTCAACTCTCTTTAGAAGTAAACAACAAAGTCGATTGCCTCA
GCTATGCGGTATCCGCAGAGTGAGTCCTAAATTTAAAATCTGACTACATGTAGAAAAGCGTTTCGTGTGA
CCCATGACCAGGAAATAAATCGGGTAATACAAACAGGCTCAGGAATGAGAGAAATGATTAGAATTGCGTG
AAAATTTGACATATCAGTATGATAACTGATTTCAAATATTTAAAAAAACAACATGCAAGAAAGCAGATAT
CATATCAAGAGAAATTAACAGTACAGAATAGCCAAATTAAATTAAAGAGCTAGTATAAAAAAAGTATGTC
TTAATTGAAAAAAATTACTGTATGGCCGGCTGATCAAATTAGACGTTTCAGAGGAAAACATTACCCAACA
CACAATTTTAGAGAACCTACAGAATGAGCTACACACACACACACACACACACACACACACACAAACTGAA
AACACACCCATACTCACACACACGCAGAAACTCACAAGTTCTAACACACACAGACACGCGCACCCCTGAA
GAAACAGTGAAATATAAAATTAAGCGAGCCTCACAGACATGTAGGAAAATATGAAAAGATTTCCTGCATG
TGGGAAGCAAGTCACAGTAAAGAGCAAGGGAGTTTATAATAGAAACAAATACCAGAATCAAGGATGGCTG
ATAACTTTTCAATTACGAAGAACATTAAAAAAAATCACAGAATCGTGAAACTCAAGGGATCATATAGGGA
ATTTCGGAAAAAAAACCCAACCTGTATGATGTACTTTTGTACATCACAGTTCGAAGGTAACAAGGCAAAG
ATGTAATAAGAAGAAACCTGTCACGAGAAACTGGAGGAAAAAGAGCTGTGTCTTCCTACAAGTACACTGA
TACAAATTGCCAATGTGTTCACCTCAGAAACACTGGAAGCCAGATACCAGGGAATATTGTTAAAATGATA
ATCAGGAACAAAAAGAGATCAACCGGGAATGCTGAATCCAGCAATAAAATGCCTTGAAGGTCATCCATGT
CGGATAAATGCATATTGTGCACTGCCCCAAAGAAAGAAACCGGAAACTGTAAGAATTGGAAATCAGCAGG
CTTATGTAACAAGAGAGGTGACCCGAAGGAATTAGGTAGAAGAAGAATTGAACAAGAAAGGAACTTTCTG
CAGCCCACGTAATGAAGAATCCAGCAATTGGCAAATGTAGATAGATGTAAATGCAAAATATTTTCTTGAT
CAAATTTCTATATCTTTGTAAATGAGAGTTGACTACTTGAAACAAAATGATAGCAAGATATTTAACTTCA
GCATATGTAGAGGTAAGAATTTGAAATGGTAGCATAAATCACGAAGGGATTAATTCGAAGTGTACCGTTG
TAAGTTTCTTTACCTCATGCACGATGGTGTGTCATATTAATAAAAGGGTACTGTGCGGGTTCGAAGGGAT
ATTGCAAATCCTAGAGCAATCACAAAGGTTTGAACTCTGAGGTTTTTGGTATAATAAGAATAGTCCATGC
ATTCAAAAGAGGGAAGCCAAGGAAGAACTAGAAGTCTTTCAAGAGCTCAGGCTCTTATACATCCAGTTGC
TCATTGAACCAGCTTCCTGGAATGGAGGGTCTGGGGTTGAGACTAGGCCACAAGTCTAGAGTCTCTAGAG
AGACAGTGTTGGAACCCCATGGCCCATAATACATTTCCCATTTTCTCAGGCAGCCAGAGGTCATGAATGT
GAGGATACTGGGAGGTTGGAGCAACGTTCTTGGGAGGCATAAGGAAGAGCGAATGCTTCAAGATCCCCGC
AGCCCAAACTACTCGCCTGCTTTGCCCCCTAATGCATTTTTCTCTGCTGCTCCGTAGCTGTCCGACCTCT
TCAGATCTCTTAGTCCACCCTGCCGTCTTCCTTTATGCCATGGGTCCCACTGTTCTTTCAACTCATCCCC
CTTTCCCTCAGTCCCGGAGTAGCTGCGGCCAGCAGAGGGTAGACTGAGAGCAGGAGAGAAGGACCTGCCT
AGGAACCCCTTCTAGAGATACTGCATCCTGCCTGGGAGCAAGTTTTCCAGGGCAGCTTTGAGAAGTCTTG
GAGAAACAAACCTACTAAACCTGACAGACAGTAATACTATTTGCACAATGCTTTTCTGTGGGAAAGGTAG
AGCCTTTTCACTACGTATTGAGTACATAGAGTGTGAGGGTTGACCTGGAACGGCTATCCTCCTGGATGAC
GTGCGTTTTCTGAAGAACTACATGTTCGTTGCAACTCCCACATTAGAATATGAAGTCCTACCGAGAGAGA
TACGGAGACTAGACAGATACAGATGCATTTGCATGTGAATACACAATCCCACAATACAGACGTCAAAACC
CATACCAGTTATTCCAGAGAGATGGATTGGGCAGAAGGCAGAAGGAGAATACTCTGATCGTTTTTCGGCC
ACGTGTGTGTGTTATCTCAGTGTTTCTAAGAAGCGTTTGCTACTTTAGATTTTTTATTTAAAAAAAATAG
TAATAATCTATTAAGTATGAGAGATGTGCAGAGACGATTAGTGATCGAGAGCCATTTTTGCTGGTGGCAA
TCATATGGTACTTTTAATGGGAATATTAGAAAGGCACCGGTAATGACCTTGTTGCAGCACAAAGGAGAGA
GTGTGGGGTGCCCCTGCATGTTGTCCCACCTCTTGTGACGTGTATCGTTTTGGAATTTCCAGTGGCTTGA
TCATGAACTACTGCAGGAATCCAGATGCTGTGGCAGCTCCTTATTGTTATACGAGGGATCCCGGTGTCAG
GTGGGAGTACTGCAACCTGACGCAATGCTCAGACGCAGAAGGGACTGCCGTCGCGCCTCCGACTGTTACC
CCGGTTCCAAGCCTAGAGGCTCCTTCCGAACAAGGTAAGGAGTCTGTGGCCAGACATCTACACGCTTCGA
TGCTGGGATGAAAAGCCATGGAAATTCCCACTGATGCAGCCGCCTTCAATGGTAAACGGATGCTCGAGTG
TTGCCTGAGTTCTACCATGTAGGAGGAAGCCTCCGTGCACTCTCTGGGGGAGCCAGCGGAGTGATTTCTG
GTGCAACGTGGTTGGGCTTTGTCTTTAGGATGGGCACAAACCCTCCAGGGGGATCGACTTCAAAATTCAC
CTTGTTGTAAAACGGGCTACCTCAGTGTCCCAGCCAAAATTTTTATTGTAACATGCTGTCAGGTGTGTCA
CTCTTTCCAAGCCAGTAAGCTTTTCCGGGGATTTCTTCAAGTAGCCAGCATTCAGAGCAATCTTCAGCAT
TGCAGATTCTGAGAAATGTGGCTCTGGAGCCTGTCATCCTCGAGAAACCTAACAGGGCTGCATTAATTCC
ATATGGTCCTGGGTCTATGGAGCAGTATATGAGCTCCCAATGCTCTAAGGCTCTTCAGTCCTAGGCTTTG
AAGGGAGTGATTTCTCAGTGTTCTTAAACCTCTTTCTGATGGCACTTGTACCTGTGAGGGGTCTAGAGAG
AAAGGTTAGTAGACTTCTCCTTTACTGCAATTCAGGATGCAGGGCATGAGAAGATTCCCTCCCTCCTCCA
AGGGAAGAAGGTTTTGGCGTGCACACATCCTTGAGAAGCAAAGTGTCTTTGCCTTCAGTCAGATATATAG
GATCGTTTTCTGCCCCATGGCCTGGAAGCCAGAGGCCTTGGCTTTCATGATCAACGATCTAGGGAAACAT
GCAAAATTTCCATGTCTTTCCCCTCCTCTGCCCTCGACAGCCAATTACCACCTGCATCCTGCATTGCCAA
ATGCAGTGCCCTTTGTATGAACATTCAGTAGAGTTTCATAGAAAGGTGCTACTTCGTGAGCGCACTTTGC
AGTGAGAAGGAGTCTGTTCTGTTCTGTTTTTCTAAGGATTTCAGGTGAAATATTTCCTAGAACTTACTAC
AGTTCTAGATTGGTAGGAATCTGTAGGTTTGCTGTATGTTTTTTGGTTGGTTTTCTCCCATCCATCTGCC
TACAGGTAAGGGAAAGATAACGTTCATAATTCTCATAGACTCCTTTCTGGTTGTGTCATAAATGGCTTCA
CATATTTCGTTATTCTCAGAGATACTCAGTTTATTTCTTGTGTTTTCATTTCAGCACCGACTGAGCAGAG
GCCTGGGGTGCAGGAGTGCTACCACGGTAATGGACAGAGTTATCGAGGCACATACTCCACCACTGTCACT
GGAAGAACCTGCCAAGCTTGGTCATCTATGACACCACACTCGCATAGTCGGACCCCAGAATACTACCCAA
ATGCGTATGTCTTTGTTCTTTACCATAAGAGAATAAAGGGCCAACTGAAGTTTCTGTGACAAGAGACATG
CTTCAAGCTGAGTTCTCCGAACTCAACTTGTGTCAGATTCAGATGGTGTAGCAAAATGTCTCAGGATGAT
TTCCTTGGAGCTAAGGGTCTGAGAGAAGAGAAATGTTAAGCTGCCTCACCTTCCTCCTAGTTTTGTGGAG
CAGAAGGGAAATGAGGAGGCGAGGAGATCACCTTATGAAGAAAGTCAGAATGACGAACCACCAAACACTT
AGATTACCCTTGCCCAACACCCACTAAGCGTCAATGAAGACTTTCCAGTTGGAATTCCGTTATTCTGACT
TCCAATTCCTGAAGGGAAGATTGTGTTTGCCTTTTCTGTCTGGGCTCATGAGGAAAGTTTATGTGCTTAC
TTATGGACAGGTGAATTGATCTGTTTCTATTTCTACCTGTATTCCAATAGGGAGAAAATCTCTTGGTCCT
AAGTACCAGTGGCCTGAAAGGATAGAGGTTCCCAGCAAGAGAAGATCCAAGGAAGGAAGGCAGATGAGAG
TCAGCACAGAGAGGGATGCTGAAAAGTAAAAGGGATGGGTAGATGGATAGAAGCCCTGGTCTGACCACCC
CATGGCCAATCATTTGGCCATAATCAACAACCAAAGACATGGAAAAATGGTTTCTACATGTGGGACAACA
GATGGTAGAGGACCTAGAGAATTGAGAGAGGGCCAATGATGAGCTCAACTCCATAGATGCCTTGGCTTTC
TTCCTGGATACCCTTCCTGCACTGAATAGCAAGGAGATGGAGCTCAAGCAGCCTGTAGCCATCTAGCTGA
GCAGAGGAGAGGGATTGGAGTTTGGGATGACTCTGGTATTTTCTAGGTCCGCTACAAATAAGAACTGGTT
TGTGGAGGAAAGGAGCTCTACAAATACGCATAGAAGTCTCCTCCAGTAGTTGGCCTCACATGACACTGCA
TGTGCACAGAAAATGGTTCTACAGAAAGTGTGGCAAAGAACATTTACTGAGAAACAGCAACTACAAGAGA
ACAGCAAGCTCAATTAAGAAGATAGAGATCACATAGCACTCTGTGTTATTGGAGTTCTTACCAGCTAGAT
GAGAGAGTGCTCACGGAACACATTGCCAATTCAGTGGAGACCCCAGAACAGCCATAATTTCAAAGTACAA
TTAGTATATTACTAGAATAAAGGCAGCTGCAGACAACCCCTTGCACAGCTGAAAAGCAAGTGTCCAAGCA
TCAAATGGGTTTCCAATCAATGAAGTGCCTGTGAGAGGAAATCTCAACTCTCTTCAGAAGTAAACAACAA
AGTCAATTGCCTCAGCTATGCGGTATCCCCAGAGTGAGTCCTAAATTAAAAATTTGACTACGTGTAGAAA
AGAATTTCGTGTGATCCATGACCAGAAAATAAATCAGGCAATACAAACAGGCTCAGAAATGACATCGATA
ATTAGAATTGCATGAAAATTTGACATATCAGTATGATAACTGATTTCAGATATTTAAAAAAAGTGCAACA
AAGCAGGTATCATATCAAGACAAATTAATAGTATAGAATAGCCAAATCAAATTAAAGAACTATTATACAA
AAAGTATGTCTTAAATGAAGAAATTACTGTATGTCCGCCTGAAAAATTTAGATGTTTCAGAAGAAAAAAT
TAACCAAAAACAATTCTGCAGAACCTACAGAATGAGCCACACACACACACATTCAAAACACACCCATACA
CACACACATGCAAAAACTCACAAGTTCTAACACACACACAAACACACACACACATGCACATCCCTAAAGA
AATAGGGAAATATAAAATTAACCGACCCTCAGAGACATGCAGGAAAATATAAGAAGATTTCCTGCATGTG
GGAAGCAAGTCACAGTAAAGAGCAAGGGAGTTTGGAGTAGATACAAATACCGGAATCACGGATGGCTGAT
AACTTTTCAATTATGAAGAACGTTAGAAAAATCACAGATTCATGAAACTAAAGGGATCAAATAGGAAATT
TCGAGAAAAAAAACTACATGATGCACTTCTCTACATCACAGTTCAAAGGTAACAAGGCAAGGATATAAGA
AGAAGAAACATCTCACGAGAAACTGGAGAAAAAAGAGCTGTGTCTTCCTAGAGTACAGTGATACAAATTG
CTAATGCGTTCACCTCAGAAACACTGGAAGCCAGATACCAGGGAATATTATTAAAATGATAATGAGGAAC
AAGAAGAGATCAACCGAGAATGCTGAATCCAGCAATAAAATGCCTTGAAGATCATCCATGTTGGATAAAT
GCATATTGTGCACTGCCCAAAACAAAGAAACTGGAAAGTGTAAGACTTTGGAATCAGCAGGCTTATGTAG
CAACAGAGGTGACCCGAAAGAATTAGGTATAAGAAGAATAGAAGAATTGCATGAAAATTTGACATATGAC
TAAGATAACTATTTCAAATATTTAAAAAAAGATGAATATGTAATAAAACAGATAAAATATCAAAAGAAAG
TAACAGTATTGACTAGCCAAATCAAATTAAAGACTTAGTGTAAAAAGCTATGTCTTAAAAGAAAAAATTA
CTGGATGGCTGCCTGATCAATTTAGACATTTCTGAATAGGAAACTAACCAAAAATCAATTCTACAGAACC
AACTACACACATATATACACATACAACACACCCATACACACCCACGCAAAAACTCACAAGTTCACACACA
CACACACACACACACAACCCTCAAGAAATAGTGAAATAGAAAACCAACCGAACCTCACAGACATGTTGCA
AAATAGGAAAAGATTTCCTGCATATGGGAAGCAAGTCACAGAAAAGAGAACGGGAGATTGGAAACAGAAA
CAAATACCGGAATCAAGGATGGCCGAAAACTTTTCATTGATCAAGAATATTAACAAAATCGCAAAAACAC
GAAATTCAATGCATCAAATAGGCGTTTCGAAAAAAAGAAAAAATCTGGTATGATGCACTTTTGTACTTCA
CATTTTCACGGTAAGAAGACAAAGATATAATAACAAGAAACTTCTTATGAGAAACTGGGGAAAAACAAGC
TGTTTCTTGCTAGAAGAACAGTGATACAAATTGCTAATGCATTCTCGTCAAAAACACTGGAAGCCAGATA
CCGGGAATGTTATTAATGTGGTAAACAGGAACAAGAAGAGATCAACCAAGAATGCTAAATCCAGCAATAA
AATGCCTTGAAGATCATCCATGCTGCATAAATGTATGTTGTGCACTGCCCCAAACAAAGAAACCGGAAAC
TGTAAGAATTTGGAATCAGCAGGCTGATGTAACAAGAGAGGTGACCCAAAGGAATTAGGTAGAAGAAGAA
TAGTACAAGAAGGGAACTTTCTGCAGCCCATGTAATGAAGAACCCAGCAATTGGCAAATGTAGATGTAAA
TGCAAAATATTTTCTTGACCAAATTTCTATATATTTTTAAATGAGCGTTGACTACTGGAAACAAAATGAT
AGCAATATATTTAATTTTAGCATATGTAGAGGTAAGAATTTGAACAAGTAGCGTAAATCATGTAGGGAAT
AATTAGAAGTGTACCATTGTAAGTTTCTTACCTCATGCACAATGGTATGTAATATTAATAAAATGTTACT
GTGTGGGTTCAAGGAGATATTGCAAATCCTAGAGCAATCACAAAGTTTTGAACTCTGAGGTATATTGTAT
AATAAGAATATTCCATGTATTCAAAAGAGAGAAGCCAAGGAAGAAAGAAATTTGTCACGAGTTTGGGCTC
TTAGTACATCCTGTAGCTCATTGAACCAGCTTCCTGGAATGGAGGGTCTGGGATTGACACTAGGCCACAT
GTATAGAGTCTCTAGAGAGACAGTGTTTCATCCCCATGGCCCGTAATACATTTCCCATTTTCTCAGGCAG
CCACAGGTCATGAATGTGAGGATAGAGAGAGGTTGGAGCAACGTTCTTGGGAGGCATAAGGAAGAGCAAA
TGCTTCAAGATCCCCGCAGCCCAAACTCCTACCTGCTTTGCCCCCTAATGCAGTGTTCCTCCGTAGCTGT
CCGACCTCTTCAGATCTCTTAGTCTACCCTGCCATCTTCCTTTATGCCATGGGTCCCACTGTTCTTTCAA
CTCATCCCCCTTTCCCTCAGTGCAGAGTAGCTGCGGCCAGCAGAGGGTAGACTGAGAGCAGGAGAGAAGG
TCCTGCCCAGGAACCCATTCTAGAGATGCTGCATTCTGCCTGGGAGCAAGTTTTCCAGGGCAGCTTTGAG
AAGTCTTGCAGAAACAAACCTATTTGACCCACATGATATGGGAATGACAGAAAGTAATACAATTTGCACA
GTGCTTTTCCATGGGAAAAGTAGAGCCTTTTCGCGAGGTTTTGAGTACATAGAGAGTGAAGGTTGACCTG
GAAAGGTTATCCTCCTGGATCCCATGTTTTTTCTGAAGAACTACCTGTTAGTTGCAACTTGCACATTAGA
ATATGAAGTCCTACCGAGAGAGATACGGAGAACTAGATAAATACAGATACTTTTGTATGTGAATAAACGA
TTCCACAATACACACATCAAAATCCATACCAGTTATTCCAGAGAGATGGATTGGGCAGAAGGCAGAAGGA
GAATACTCTGATCGTTTTTTGCCCACGTGTATGTATTATCTCAGTGTTTCTAAGAAGCGTTTGCTACTTT
AGATTTTTTTTTATAATAATAATCTTTTAAGTATGAGAAATGTGCAGACAGGATTAGTGATTGAGAGCCA
TTTGTGCTTGTGGCAATCATATGGTACTTTTATGGGAATATTAGAAAGGCACTGGTAATGACCTTGTTGC
AGCACAAAGGAGAGGGTGTGGGGTGCCCCTGCATATTGTCCCACCTCTTGTGACGTGTATCGTTTTGGAA
TTTCCAGTGGCTTGATCATGAACTACTGCAGGAATCCAGATCCTGTGGCAGCCCCTTATTGTTATACGAG
GGATCCCAGTGTCAGGTGGGAGTACTGCAACCTGACACAATGCTCAGACGCAGAAGGGACTGCCGTCGCG
CCTCCAACTATTACCCCGATTCCAAGCCTAGAGGCTCCTTCTGAACAAGGTAAGGAGCCTGTGGCCAGAA
ACCTACACGTTTCGATGCTGGGATGAAAAGCCATGGAAATTCCCACTGATGCAGCAGCCTCCAATGGTAA
ACGGATGCTCGAGTGTTGACTGAGTTCTGTCATGTAGGAGGAAGCCTCCGTGCACTCTCTGGGGGAGCCA
GCGGATTGATTTCTGGTACAACGTTGGGTGGGCTGTGTCTTTAGAATTGGCACAAACCCTCCAGGGTGAT
CGACTTCACAACTCACCTCGTTGAAAAATGGGCTATCTCAGTGTCTTAGCCAAAATTTTTATTGTAACAT
GCTGTCAGATGTGTGACTCTTTCCAAGCCAGTAAGCTTTTCCTGGGACTTCTTCAATTAGCCAGCATTCA
GTGCAATCTTCAGCATTGCAGATTCAGAGAAATGTGGCTCTGGAGCCTGTCACCCTTGAGAAACAGGGCT
AACAGGGTTGCATTAATTCCAAATCACCCTGGTTCTATGGAGCAGTACATGAACTCCCAATGATCTATGT
TTCAGGACTTCCTCAGTCATAGGTGGGCTCTGCAGCCCTAGGTTTTTAAGTGAGTGACTGCCCCGTGTTC
TGGTGGCAGTTGTACCTGTGAGCGGTCTGGATAGAAAGAGTCGGAGACTTCTGTATTATTGCAACTCAGG
ATGTGGGTCATGAGAGGATTTCATCTCTCCTGCAGGGGAGTAAGCTGTTCGCCTCCACCCATCCCTGATA
ACTGAAGTGTCTTTGTCTGCAGTCCTAGACGAAGGACTGTTGTCTCTCCCATGGCCCAGAAGCTGAAGAC
CTTGCCTTTTGTTATGAAACGTTCATTGTTTTCATGTCTGTCCGTTTCTCTGCCCCTAACACCCAATCAC
CATGTATGGCCTGTACCCCCAAATGCATCGTGCTTTGCTGTTTGCTGCCCCATAGTCCTCATGAACATTC
AGTAGAAATTCCCATAAATGTGCTTGCACGTGAGCACAGTTTCCATTGAGAAGCCCTCTCATTTGTCCTT
TTTTTCTAAGCTTTTATGTGAAATATTTCTAAGAACTTACTACAGTTCTAAAGTGTTAGGAATTTGTTTC
TTTGGTGTTTTTGTTTGTTGGTTGGTTGTTGCTTTTCTCAAGTCCATCTGCCTACAAATAAAGAAACAAG
AATGTTACTTGTCATATTCTCCTGAGGTCATAATTCTCAGAGACTTTTTTCTGGTTTGTGCCATAAGTGG
CTTCACATGTTTGTCTCTTCTTGGAAACACTCAGTTTGATTTCTTTTCTTTTCATTTCAGCACCAACTGA
GCAAAGGCCTGGGGTGCAGGAGTGCTACCACGGAAATGGACAGAGTTATCAAGGCACATACTTCATTACT
GTCACAGGAAGAACCTGCCAAGCTTGGTCATCTATGACACCACACTCGCATAGTCGGACCCCAGCATACT
ACCCAAATGCGTATGTCTATTTTCTTTACCATAAGTGAAGGAAGGGTCAGTGGAAATTTCTGTTAGTAGA
GTCATGCTTCAAGCTGAGTGTTCAGGACTCAAGTTGTCTCAGATGAACAGTGCATAGCAAAATGTCTCAG
GAACATTGTCTTTGAGCAAAGAGTCTAAGAGAAGACAAATGTTAATCTGGCTCTCCTTCCTCCTAGTTTA
ATGGAGCAGAAAGGTATCTGGAGGCAAGGATATCACATTAAGAAACAAGTCAAGATGACAAATGATGAAA
CTCTTAGAGTACCCTTCCACAACACCCACTAAGGTTCAATGCAGCCTTTTCTCCTTGGAATTCTATTAAA
CTAAACTCCAATTCCTGAAGTGAAGGTTCTGTTGGGGTTTTCTGTTTTGGCTTACAAGGAAAGTATATAT
GTATATCTATGGAGAGGCAAATCTATCTCTTTCTATATCTACGTCTATTCCAATATGTAGAAACACAGTC
GGTTCTGACCACCAGTGGTCTGAAGGGATACTGGTTGTTAGAGAATAAAAATGGCAGGAAGGCAGATGAG
AGTCAGCAAAGAGAGAGATCCTGTAAAGTAAAAGGGTGGATAGATGGACAGAAGCCCAGGTCTGACCAGC
CCATGGCCAGGCTTTAGGCCATAAGTGACACCAAAGACATGGAAAAATGGTTTCTACATGTTGGACAACA
GACAGTAGTGGACCAAAAGAATAGTGACAGGGGGAACAATGAGATCAACTCCATAGATACCTTGGCTTTC
TTCCTGGAGGCCCTTCTTGCACTGAAGAGCAAGGTGATGGAGCCCAGATGGACTGTAGCCATCTTCCTGA
ATGCAGGAGAGAGATTGGAATTTGGGACTACTGTGGTAGCTAGGATTTTATAGGCCTGCTGAGAATGAGA
ATGGATTTGTGGATGAAAGGAGCTCCAGGGGCACGCATAGTAGTCTCCTCGAATCTTTGGCTAAACATGA
CGTTGCATGTGCCCAGAAAAAGGTTCCACAAGAAAGTAGAGAAAAGAATATATCCTGAGGAATAGCAACT
GCGATTGAACAGTGAGCTCAATAAAGAGGACAGAGCCCTCATAGCATTCTGGGATACTGGAGTTCTGACC
AGCTGGAGGAGAGACCTCACTGAACCTCTTGGGAATACAGTAGAGACTCCAGAAAAGTCATACTTTAGGA
GTAGAATTAGTAAATTTCTAGAAAAAAAGGCAGCTCTAGACAAACCCTGGCAAAACTGAAAAGCAAGTCT
CCAAGCATTAAAATCATTTCCAAGTCAATTAACTGCCTGGGAGAGGAAAACCCTCTTTAGAGGTAAACAA
CAAAGTCAAGTGGCTCAGCTATGTGGTGTTCACAGTGTGAGTTCTAAATTTAAAACTTGACTACACATAG
AGAAGCTTTTAGTATGAACCATGACCAGGTGAAAAATCAGTCAATACAAATAGACCTAGAAATGACAGAA
ATGATTAGAATGGCAAAAAATTTGACATATCAATATGTCAACTGAGTTTTAGGTTTTAAGAAAACATGAA
TACGGAATGAAGCAGATACCATATCAAGAGACAGTAACAGTATAGAAGAGCCAAATTAAATTAAAGAACT
AGTATAAGAAGGTATGTCTTAAATGAAAAAATTACTGGATGTATTCCCAATGGAGTGAGATGTTTCAGAA
GTAAAAACTAACTGAAAAACAATTTTATACCACCTACAGAACCAGCTACACATACACAAATGACACACAC
ATATACACACATACTCACACATGCACAGGCTTAGAAACATGCACGCACACACACACACACACACACACAC
ACCTCCACAAATACTAAAAAATGAAATCCACTGATCCTCACAGACAGGGGGGAAAATATAAAAAGATTTC
CTGCATGTGGGTAGGAAGTCACAGAAGGAGAGGAAGGAGAGATTGCTACAGGAACAAATACTGGAAGCAA
GGATAGCTAAAAACTTTTCAAATAAGAAGAATATTAAAAACCACAGATTCAAGAAGCTGAATGAATCAGA
CAGGGAATTTCCAAAAAAAAAAAAAAAAAAACTGTATGATTCACTTTTGTACATCACCGTTCAACAGTCA
GAAGGCAAAGATATAATAACAAGAAACATCTCATGAGAAACTGGAGGAAAAAGAGCTGTGTCTTGCTAGA
AGAACAGTGATACAAATTGCTAATGCATTCTCATCAGAAACACTGGAACCCAGTTAACAGGGGATATCAT
TAAAATGATAAACTAGAAAAAAAAGAGATCAAATGAGAATGCTACATCCAGCAATAAAATGCCTTGAAGA
TCATCCATGTTGGATAAATGCATATTGTGCACTGCCCCAAATAAATAAACCAAAAACTAATAATTTGGAA
TCAGCAGGCTTGTGTAACAAGAGATGTTGCCCAAAGAAAATTAGCTAGAAGAAGAATAGTTCAAGAGGAG
AACTTTCTGCAGCCCACGTAATGAAGAACCCAGCAAATGGCAAATGTAGATGTAAATGCAAAATATTTTC
TTGATCAAATTTCTATATCTTTTTAAATGAGAGTTGACTACTTGAAGCAAAATGATAGCAATATATTTAA
CTTTAGCATATGTAGAGGTAAAAATTTGAACATATAGACTAAATCATGTGGGGAATAATTGGAAGTGTAC
CATTGTAAGTTTCTTACCTTATCCACGATGGTATGTAATATTAATGAAAGGTTGAATTTGTGGGTCCAAA
GGGATATTGTAAATCCTAAAGCAATCATAAAATTTTGAATTCTGAGGGATATTATATAATAAGAATTTTC
CATGTATCCAAAAGAGGGAAGCCAAGGAAGAAAAAGAAGTCTTTCAAGTACTCAAGCTCTGAGCACATCC
AGTTGCTCATTGAACCAGCTTCCTGGAATGGAGGGTCTGGGCTTGAGACTAGGTCACATGTGTAGAGTCT
CTAGAGAGACAGTGTTGGATCCCCATGGCCCATAATACATTTCCCGTTTTCCCAGGCAGCCACAGGTCAC
GAATGGGAGGATTCTGAGAGGTTGGAGCAATGTTCTTAGGAGGCATAAGGAGGAGTGAATGCTCTGAGAT
TTCCCCAGCCTGAGGTCCTCCATAGCTGCCCGACCTCTTCAGACCTCATAGTCTGCCCAGCTGTCTCCCT
TTATGCCATGAGTGCCACTGTTCTTTCAACTCATCCCCCATTCCCTCAGTCCCGGAATTGCTGTGGCCAG
CAGAGGATGGACTGAGAGCAGGAGAGGAAGTCCTGACCAGGAACCCATCCTAGAGATACTGCATCCTGCC
TGAAAGCTAGGTTTCCAGGGCAGCTTTGAGAAGTCTTGCAGAAAGAAACCCACTTGACCCACCTGATACG
GTATCGACAGACAGGAATACTTTTTGTGCAATGGTTTTACATGCTGAACATAGAGCCTTTTGGCTACATT
TTGAGTACATTGAATGAGACTGCTGGCCTGGGAAGGATATCATGCTGGATGCCATTTTTTTCTCTGGAGA
ACTATGTGTTAGTTCCAACTCGCACATTACTATATGAAGTCCTACACAGAGAGATACGGAGAGCTAGACA
GATAGAGATACTTTTGTATGTGCATAACCAATTCCACAATACACACGTCAAAATCCATACCAGTTATTCC
AGAGAGATGGATTGGGCAGAAGGCAGAAGGAGGATATTCTGATCCCTTTTTGGCCACATGTATGTATAAT
CTCAGTGTTTCTAGGAAGTGTGTGCTGCATTAGATTTTTTTTCTTTAAAAAAAGTGATAATATATTAAGT
ATGAGAAATGTGCAGAGAGGATTAGAGATTGAGAGCCATTTGTCATTGTGGCAATTGTATGGTATCTCTT
TTGGGAATATTTCAAAGGCACCAGTAATGACCTTGTTGTAGCAAAATATACAGTGTTCCTGCATATGTAC
CCATTTTTTGTGATGTGTATTCTTTTGGAATTTCCAGTGGCTTGATCAAGAACTACTGCCGAAATCCAGA
TCCTGTGGCAGCCCCTTGGTGTTATACAACAGATCCCAGTGTCAGGTGGGAGTACTGCAACCTGACACGA
TGCTCAGATGCAGAATGGACTGCCTTCGTCCCTCCGAATGTTATTCTGGCTCCAAGCCTAGAGGCTTTTT
TTGAACAAGGTAAGAAGTTGTGCCAGACATTTACCTGCTTGGATGCTGGGATGAAAAGCCATGGATACCC
CCACTGACGCACAACCCTTCAGTGCTACACTGGTTCTCGTGTGTTGGTTCTGGGTCTGCCATGTGGGAGG
AAGCCTTAGCGCACTCTCTGGGGGAGCCAGAGGTGTGATTTTTGGTGCAACCTGTGCGAGCTGTGTCTTT
AGGATGGGCGGAAACCATTCTGGGTGCTCGACTTCACCACTCCCCTCATTGTAAAAGGGGCTATCTCATT
GTCCTAGACAAAATTCTTATTGTAATATGCTGTCAGATGTGTGTGTCTTTCCAAGCCAGTAAACTTTTCC
AGGGATTTCTTCAAGTAGACAGCATTCAGTGCAATCTTCAGCATTGCAGATTCCGAGAAATGTGGCTCTA
GATCCTGTTATCCTTGAGAAACCTAACTGGGTTGCATTAATTCCATATCTCCCTGGGTCTGTGGAGTAGT
ACATGAGCTCCCGAAGCTCTATCTCTCAGGTCTTTTTCAGTCCGAGGCAGGTTGTGCAGTTCTTAGCTTT
GAAGGGAGTGATTTTTTCGTGTGCTTTTGCCTCTTTCTGATGGAACTTGTACCTGCGGGGGGTCTGGAGA
AAAAGAGTAGTAGACTTTTGCTTTATTGCAATGCATTATGCTGGGCACGAGAGGATTCCCTATCTTATTG
TAGGTGATAAGCTTTTGGCCTCCACTCATCCCTGAGAAGTGAAGTGTTGTTGCCTACAGTTTTAGCTGCA
GGACTGTTGTCTGCCCCATCACCAGGAGTTTAATGCTTTCTTTTTTGAGCAATCATCTAGGGACACATGC
AAGGTTTTTATATGTCCTTGCCTCCTCCCCAAAAAACCATTTTAATGCTTGGAGACTTGCTTTTCAGCTT
TGCCAAATGCATCACCCTTTCTTCTATGCTGTTCCATGTCGTCATGAACACTCTGTAGAGATTCCTAGAA
ATGAGCTTCCATGTTAGTGGAGTTTCCGATGAGAAGCAATCTGATATTTCTTTTCCACTAAGTTTTACAT
GAAATATTTCTAAGAACTTACTACAGTTCTAGAATGGTAGGCATCTCTTACTTTCGTGTTTGTTTGTGTG
TTTTCTCATGTCCATTTGCCTATTAATAAAGAATAGAGAATGGTTGTAAATCTCAGTGACTCTTTTTTGG
TTTATGTCATAAATGGCTTCCTGTATTTTTCTGTTCTAGGAAATAATAAGCTTGATGTCTTCTGTTTTAA
TTTCAGCACTGACTGAGGAAACCCCCGGGGTACAGGACTGCTACTACCATTATGGACAGAGTTACCGAGG
CACATACTCCACCACTGTCACAGGAAGAACTTGCCAAGCTTGGTCATCTATGACACCACACCAGCATAGT
CGGACCCCAGAAAACTACCCAAATGCGTACGTCTTTGTTCTTTACCATAAGCGAAGGAAGGGCCAATGGA
AGTTTCTGTTAGAAGAGTCATGCTTCAAGGTGACTGCTCAGGACTCAACTTGGCTCAGATGCAGAGGAAC
ATTTCCTGTGAGCAAAAGTTCTTAGAGAAGACTTTGTTTTTTTGAGACAGAGTCTTGCTTTGTTGCCCAG
GCTGGAGTGCAGTGGCATGATCTCGGCTCACTGCAAGCTCCGCCTCCCGGGTTCACACCATTCTCCTGCT
TCAGCCTCTCTAGCAGCTGGGACTACAGGCACCCACCACCACACCCGGCTAATTTTTTGTATTTTTAGTA
GAGACAGGGTTTCACTGTTCTAGCCAGGATGGTCTTGGTCTCCTGACCTCGTGATCCGCCTGCCTCAGCC
TCCCAAAGTGCTGGGATTACAGGCGTGAGCCACCGTGCCTGGCTGAGAAGACATTTTTTAAGCTGGCTCT
CCTTCCTCCTAGTTTTATGGAAGCAGAAGGATATATGGAGTTGAGAAGATCTTATTAATAAAACAGCCGG
GATGACAAATGACCAAAGAGTTAGAGTATCCTTCTACAACATCGGCTGAGGGTTAATACAACCTTTTCAC
CTTGGAATTCTATCATTCTAAGCTCTAGTCCCTGAAGTGAATGTTGTGTTGGCCTTTTGCATCTTGGGTC
ACAGGGAATTGATACTTGCACATCTATGGAGAGGCAAATCTTTTTCTATCTACTTCTTTTTCAATGGGTA
CAAACACACTTGGTCCTGAGCACCAGTGGTCTGAAGAGATACGGTCTGCCCAGAGGAGAAGAACAAAGGC
AGGAAAGCAGATGAGAGTCAGCAAAGGGGCGATGCTGAAAAGTAAAAGGGGGGGGTAGATGGACAGAAGC
CATGATCTGGCCATTCTATGGCCAGTCTTTCGGCCATAAGTGACTACCAAAGACACGGCAAAACGGTTTC
CACATGTTGAACAACAGATGCTAGAGGACCAAGAGTATTGCAAGAGGGAGAAAATGAGATCAACCCATCA
ATGCCTTGGCTTTCTTCAAGGAGACCCTTCCTGCACTGAAGAGCAAGGAGATGGAGCCCAAGCTGACTGT
AGCCATGTTGCTGAACAGAGGAGAGTGATTGGACTTTGGGATTACTCAGGTAGTTAGGATTTTCTAGCCA
TGCTAAGAGTAAGAATGGACTTGTGGAGGATAGGAGCTCCAGGCATAGAAGTCTCCTCAAGTGTTAGTCT
AAACATAAAGCAGCACTTGCATAGAAGATTTTCCACAAGAAAATATGGCAAAAAAACACCATATATTGAG
GAACAACAACTACAAGGGAACAGTGAGCTTAATAAAGGTGACAGAGCTCACATAGTGCTCTGGAATATTG
GAGTTTTGACCAGCTAGAGAGAAGAGACCTCATTGAAAATCTTGGGCATTCAGTAGAGACCTCAGAAAAG
TCAGACTTTATGAGTAGACTTTGTATATTCCTAGAATAAAGGCAGCTCCAGAAAAAACCTAGCAAAGCTG
AAAAGCAAATCTCCAAGCATTAAAATGGTGTCCTAGTCAATTAACTGCCTTCTAGAAGAAAACTCAACAC
TCTTTACAGGTGAACAACAAAGTTAAGTTGCTGAGCTATGCAATATCCACAGTGTGAGTCCTAAATTTAT
AACTTTACTACACATAAAAAAGCATTTAGTGTGAACCATAACCAGGAAAATAATCAGTCAATAAAAATAG
AACCAGGAATGATAGAAATGATTTAAATGGCATGAGAATTTGACATATTAGTATCATAACTGCATTGCTG
GATTTAAGAAAACATAAACATGGAACGTAACAGATATCATATCAAGGGAAAGTAAAAGGATAAAAGAGTC
AAATCAAATTAAAGGACTATTAAAAGGTATATCTTAAATGAAAAATTCACTGGATGGTCTCCCAATCAGG
TTAGTTGTTTCCAGGGAAAAAATTAACTGAAAAATAATTCAATAGAATCTACAGAAATAGCTGCACATAT
ATACACACAATGGCACACGTGCACACACCCACACCCACACAGGTGTGAATCCTAGAGCCACACGAGCATT
GAAACATAGAGAAGTAAAAATTGTTCATTGAGGAATATGTAGCAATGCTCAATGTGTTTTACCCTAATAA
GAGCTTTTGTGATGTATGATTGAAAAACTGACACAACTGAAGAGAGAAATAGATAAGCCCACACTCTGAG
TTAGAGATTTCCTTGATTCTCTCACTATGGTTATAAATCTTTCCCAAACACAACAGGCTAGAACAAATAT
GCAGAAAATTAGACATAGTATCTTTGTTCTCAATAAAAACGTCGACCTATTTAACATTATACCGAACTAC
CGAGTACACATTAAAGTGTGCATGGAGCATTCACTGAGGTGTACTCTACACATGACCTTCCAGCAAGTCT
CCATAGATTTAAAAGAATTAAAGTCATACAGAGTGTGTCACTTTATTCTCCCAGAATAAAGTGAGATATG
AATAATGAGAAGTTTGCCAGCTTCTCAAATATTTGGGAGTCATACGGTGCATTTCAAAATACTCTTTGGG
ACAAAGAAAACATCACTAAGGAATTTAGAAAAGTTTTGAACTGAGTAAGAATATAACACAATTTATCCAA
ACTTAGGAGATGCAGTGAATGTCTTTAGGCTTTTACATAATTTTAGATGCTCTTAGGGAAAAACAGAAGC
ATGTAATAATCAAGATTTCAAACTGCAATTCTCAAAGTGTAGTCTAGAGAAACCTGAGGACCTTTGAGTA
CCTTCAGAGACAGTCCATGAGGTTAAAGGACTTTGCTACGTGAAAAGTAAGATGCTATTGGCCCTTTTTA
CTTTCATTTTCCAACAAGAGAAGAGGGGAGTTTTCCAGCAGTTACATAATATGTAATGGCATCATGTCTC
TGATGGCTAAGAAAATGGGCAATTGTTGACTTTGTGTGTTAAAAAAATTCTCAGTGTTGGTTTCTTATAC
TATAAATATTCATCTTGTGTTTTGAAAAAGAAAAGCTCTTTGGAATCCCCTATGAACAAAGACTTTGACA
GTTGTTGATCTAAGACCACAGCTTAAATATCTACACAAGAAAAAAAAAAAAAGCAAATAAGAGCCAAGGA
AAGCAGATGGAAGGAAGTAGTCCAAACCAGTGACATTCAGTGAACAAGAAAAGAGACCAACAAGGGAGTA
AACTCTTGAAACAGAAAGTTGATTCTTTGAAAAGATCCATATGATTGAACACAGTCTGGCTAAACAAATG
ACAGACCAATGAGGGTGCACAACCATCACCATCTGGAGTAACAGAGGAGAGGTGCCATTACTATAGCATC
TTCCAGTTCTGAAAGCTGAAAAGAAGATTTTGAGAACAATTGTATGTGAATAAATTCAGGAATGTTAATC
ATGTGGGCCAATTCCTGAGGAAGACAACAAATCAGCAAACCAGATGCTGAATAGTTAGTGTAGTCCTGTA
GAGAGACATACAGAGAGGCTGACAGAGAAATATTTGTATGTGCATAAAACAATCTACAAGACACACTTCA
AAATCAATCTCAGTTAATCTGGAGGAACATATTTCACAGAAGGTGGAAGGAGGGTATTCTGATCCTCTTG
TACATTGTACAACATTGTACAATGTACAGAGTATAATTGTACAAGTACAATTGAAGTTGTACAAGTACAA
GTGCAACTTGCACAATGTACAGAGTAAACATTGATGTTTACTCTCAATTTTCTTATGGAGCACAGATGAC
TTTGGATGTGTTACAATATGAATGATAATTTGTCTTTGAGATGTTCGCAGTTGTTTAGAAGTTGAGGACC
ATTTGTGCATATTATGGGACCTTTAGTGAAAATATTTCAAAGTCTCTTTTTACACTTTGTTACAGCAAAA
TGTAGAGGGCGCTAAGTGCCCTTGAATCTTCTCCCATCTCTGGTGACCTGTGTTGTTTTGAAATTTGCAG
TGGCCTGACCAGGAACTACTGCAGGAATCCAGATGCTGAGATTCGCCCTTGGTGTTACACCATGGATCCC
AGTGTCAGGTGGGAGTACTGCAACCTGACACAATGCCTGGTGACAGAATCAAGTGTCCTTGCAACTCTCA
CGGTGGTCCCAGATCCAAGCACAGAGGCTTCTTCTGAAGAAGGTAGGAAGTCTATGGCCAGACAACCACA
CCCTAGGACGTTGGGATGAAAAGAGTTGCAAAATCTTAGTGATATAGAAGCCTTCCATGCTCACACAATT
CCAAGTAGAATGTGGACTCAGGGTCAGCCACTGGGAAGGAACACTCAGCGCCTTCTCTGGGAGAACCAGA
GCTGTGATGTTTGGTACCCTGTGAAAGGGTGGTATCTATAGGAAGGGTGCAGACCCTCTAGGGCACTGGA
CTTACCACTCCCCTGGTTATTCAAAGGATCATTTTAGTGTCTTAGCCAGAAGAATATTCTAACATTTTGC
CAAATTTGTGAAGATTTACCAAGCTCATGATAAGCCTTTCATGGTATTTCTTCAAGTAGTCAGTGTTCAT
TGCATCTTTGGCTTTGCGGTTTCGGAGGAATGCGGTTTTTGAGTCTGTCATCCTTGAGAAACCTAATATG
ACTTTTCTTAGTTCCATATACTTCTGGGTCCAGGTAGCAGTACATAGCCAACAAATGCTCCATCGTTCTG
GCCTATCTCCATCTTAAGCCAGTCCTGCACAACTAGGCTTTGATGGGAGGGATCTCTCAGTGTTCTTGCC
CCTCCTTCTCATGGAACATATATCTGTGTTGGTCTCTGAGAAGAAGAGTAGTGGATATCTACTTTGTTGC
AATGCAGAATCCTGGGCCAAAGATACCAGCCATCCCTCCAAGGGAATAAAATTTTGGCCAGTAGCCCTCT
CTGAGAGACAATTTGTCTTTGCCTACGAGTCCTAGATGCAGGACCGCTTCCTGCCCCATCTTCAAGAAGC
TGAAGGCTTTGGCTTTGGAGGATCAGCAGTCTAGGGAAATGTGTGACGGTTTCATGTCTGTCCCCACTGA
CAGTCAATCACCACCTACAACCTGCACAGCCTGATGCATAGCAGTCTAGTTTCCTGCCTTATTCTCAGGA
ACACCCAGAAGATGTCTATATTAAAGAGCATGCACATGAGTGCAATTTTGACTGATAGGCACTCTGATCT
TTCCTTTGGTGCCTGTGTTTTAAAGGAAATCTTTCTAAGAACTCGTTAAAGTTCTAGAATGCTATGAATC
TTTGGGTTTTATTATTGGTATGTCCATCTGCCTGCTAGTACAGAACAGAGCATGGTAGTCTTTCTCAGAG
ACAATGATCCTGTTTCAGTCACAGATTTCTTCTGATGCTTCTGTGTTCTAGAAATTACTCAGCTTGATTT
CTCCTCTTTGAATTTCAGCACCAACGGAGCAAAGCCCCGGGGTCCAGGATTGCTACCATGGTGATGGACA
GAGTTATCGAGGCTCATTCTCTACCACTGTCACAGGAAGGACATGTCAGTCTTGGTCCTCTATGACACCA
CACTGGCATCAGAGGACAACAGAATATTATCCAAATGGGTACAACCTTGAGTTTTCTTCAAAGACAGACA
GCAGCCCCCTTACATTTCTCTTGGAAGGGCCATGCTTCCAACTAACTTCTTATGACAAATTTATCTCAGA
TCTGGAATGTTGGGTAGAATGTCTCAGGCTTCTTTCTTCAGGCACAGTGTCTGAAAGGAGAGAAATGTCA
GGCCAGCTCTCTTTTCTCATAGTTGACAGAAGCAGGAGGATATTTGAAGGTGGTGAGTTCTCATGAATAG
AAAGCTCAGGACACATGGCCACGTGCTTAGAAATAGCACCATTCCACAATGCCCACTAAAGACCAATGCA
ATAGTTCAACCAGGGATTTCTGTCATTCTAATCTCCAAGTCCTGAAGTGAAGGTTGTATTAGCCATGTTC
ATCTTGGGCAACAAATAAAGGATATCTATGTTGACATCCAGATCTTCCAATCACTTTCTCCTCTAACCTG
TACCTGGGTTCTGAGAACAAGGTATCTGAAGAGCTATGTGTTGCCAGCACATGAGGGGCAAAAGTAGGAA
GGCAGCTGAGAGTCAGGAAGTATAAAGATTCTGAAGAGTTACACATGCAGGAAGATGGACAGAAACCCAG
TTCAGACCACGTCAGCGTTTCTGCCATGAAGGACTATCAAATACATAGGAAAAGTGTTTTCATAGGTTGG
ACAACAGACATGACAGGCCTGAGAAAATTCAGAAAGGGAATCAAAGGAGATCAACCTTATCATGTCCCTG
GCATCCTTCCTTGAGACCCTTGAAGGGCAAGCAGATGGAGCCCAGCTGACCACAGCAGTCTTGCTTAACT
GAGGAGAGAGACTGGAGTTTGTGATGCCTCAGGCATCTGACGTATTCTAGGCTGGCTAAGAATGAGAGGG
GATTTGTGGAGGAAAGGAGCTCCAAGAATACACACCGAAGTCTTCTCAAGGCTTTGGCTAAATACAAAGC
TGCGTATGCACAAGGAGAGTTTTCACAAAGAAAGAACAATAAAGAAAAGCTACTGGGGAAAGAACAACTG
CAAGGGAACAGTGAGCTCAATGGAGATGCTAGAGCTCACATAGCACTGGGGGATATTTGAGTTCTGACCA
CTCAGAGGAGAGACACCTCACTGAACATCTTGGGCATTCAGTAGAGGTCAAAGAAAGCCATAATTTGGGA
GTAGGATCTTCGGATTCCTAGAAATAAGGTGACTCCAGAAACACTCCAGCAACCCTTCTTCCAAGCCAGT
CTAAAAGGATCCAAATGATTTCCAAGTAAATTAACTGCCTTCCAGAAAAAAGTAAACTCAACCCTCCTTA
GAGGTAAGGAACGAATACAAGTTTCTCAGTTATATGACATCCCCAGAGTGCAACTTGCATTTAAAAATTT
ACTAGACACAAAAGAAGTTTTCACTGTGATCCATAACTGGGAGAAAAATCACTCAACACAAATAGGCCCA
GAAATAATAGAAATTATGGCATTGGCAAGAACATTTAAAATGCACCTCTGAGAACTGTGTTTCAGGAAAA
TGTCAGCAAAAGCTGACCATGAGAGAAATGAATGCATAATATCAGAAAAGAAAAGAATTGAAGAGCCAAA
TGGAAATTTAAAAACTGAGAAAAGTTATATCTGTAATGAGGAATTCACTGGATGGCCTTATAACCAGTTT
AGATATTATGGTAGGAAAAGGTGAACGAGAAAATGATTCAATTAAAGCTAGACAAACCACAAGACAGACA
GACAGACACAAATACACATACACACAATGACTGAACCAATTAATCAACAGAGCCTCAAGGACATCTAGGA
AAACATCCACACATTTAATATATGTGTTAGGCAAGTCACAGAAAGAGAGGAAAAAGATAATGTGACAGAA
GTTATACTTGAAGCCATGACGGCTGACAAATTTCCAAACATACAGAAAATGAGAAATTCATAGTCATGAA
GCTCAATGACTCAGGTATAGATTTTTAAAGAGCAAAACTCTGATTTACTGGGGTACATCATAGTTAAATT
GTCTGATTTCAAAGCTAAGAAGAAAAAAAGGGGGTTCCTATGAACAAACATTTTGACAGTTGATCTAAGA
CCACAGCTTAAATATCTAGGCAAGGAAAAGCAAATAAGACACAAGGAAAGGGGATGGATGGAAATAGTCC
AAACCAATGACATTCAGTGAACAAGAAAATAGACCAACAAAGGAGTAAATCCATGAAACAGAAAGTTGGT
TCTTTGAAAAGATTCATGTGATTGACCACAGTCTGGCTGAACAGATGACAGACCAAGGAGGGAGTACAAC
CATCACCATTTGAAGTAACAGGGGAGAGGAGCCATTGCTATACCATACTCCAGGTCTGAAAGCTGACAAG
AAGATATCAAGAAAAACTGTATGTGAATAAATTCATGAATGTAGATCATGTGGATCAATTCCTTAGGTAA
ACAACAAATCAGCAAACCAGATACTGAATAGATTGGGTACTCCTATAGAAAGACATACAGATAGCCAGAC
AGAGAAACATTTGTACGTGCATAAAACAATCTACAAGACTCACTTCAAAATCTCTCAGTTAATCCAAAGT
AACATATTTGGCAGAAGGTGGAAGGAGGGTATTCTGATCCTTTCTTGTACACATTGATGTTTTCTCTCGG
TTTTCTTATGGAGTATAGACGAGTTTGGATGTGTTACAATAAGAATGATAATCTGTCTTTGAAATGTTCA
CAGTTGTTTAGAAGTTGAGGACGATTTGTGATTGTTACAGGACCTTTAGTGAGAATATTTCAAAGTCACT
TTTTACCACTTTGTTACAACAAAATGTAGAGGATGTCTGGTGCCCTTGTATCTTCTCCCATCTCTGGTGA
ACTGTATTGTTTTGTAATTTGCAGTGGCCTGACCAGGAACTACTGCAGGAATCCAGATGCTGAGATTAGT
CCTTGGTGTTATACCATGGATCCCAATGTCAGATGGGAGTACTGCAACCTGACACAATGTCCAGTGACAG
AATCAAGTGTCCTTGCGACGTCCACGGCTGTTTCTGAACAAGGTAAGAAGTCTCTGGCCAGACAACCACA
CCCTTGGACGTTGGGATAAAAAGAGTTGCAAAATCTTAGTGATACAGAAGCCTTCCATGCTGCACGGGAA
TCTGAATGTGGACTCAGGGTCAGCCAATGGGAAGGAAGCCTCAGCGCCTTCTCTGGGGGAACCAGGGCTG
AGATTTTTGGCACCCCGTGACAGGGTGGTGTCTTTAGGAAGCGTGCAGACCTTCTAGGGCACTGGATTTA
CCACTCCCCTGGTTATTCAATAGATTATTTCAGTGTCCTAGTGAAAATGGATATTCTAACATCCTGCCAA
ATTTGTGATGATTTACCAAGCTCATCATGAGCCTTTCCTGGTATTTCTTCAAGTAGACAGTACTCATTGC
AAACTTCAGCTTTACAGTTTCAGAGGAATGTGGTTTTTGAGTCTGTCATCCTTGAGAAACCTGATATGAC
TTTACTTAGTTCCATATCCTCCTGGGTCTAGGTAACAGTACATAGCCAGCAAATGCTCTATCTCCCTGTC
TACCTTAATCTTAGGCAGGTGCTGCACACCTAGGCTTTGATGGAAGGGATTTCTTAGTGTTCTTGCCCCT
CCTTCTCATGGAACACGTATCTGTGTTGCTGTTTGTGAAGAAGAGTAGTGGATGTCTACTTTGTTGCAAT
GCAGGATCCTGGGCCCAAGATTTCCCGCCGTCCCTCCAAGGGAATAAAATTTTGGCCAGTACCCCTCTCT
GAGAGACAATGTGTCTTTGCCTGGAAGTCCTAGATGGAGGACCACTTCCTGCCCCATCTTCCAGAAACTT
AAGGCTTTGGCTTTGGAGGATCAGTGCTCTGGAGAAATGTGTGACGGTTTCATGTCTGCCCCCACTGACA
ACCACCACCTACAGCCTGCACCGCCTGATGCATGGCACTCTGGTCTCCTGCCTTGTTCTCAGGAACACCC
AAAAGAGATCTTTGCCAAAGAACAGGCACATGAGTGCAATTTTGACTGATAGGCACTCTGATCTGTCCTT
TGGTGCCCAGGTTTTAAAGAAAATCTTTCTAAAAACTCATTGAAGTTCCAGAATGCTATGAATCTTTGAG
CTTTGTTATTGGCATGTCCATCTGCCTACTAATGTAGAACAGAGCATGGTCGTCATTTTCAGAGATGATG
TCCTGTTTCTATCATGGATTTTTTTTCTCATGCTTCTGTGTTCTGGAAATTACTCAGTTTGTTTTCTCCT
CTTTGAATTTCAGCACCAACGGAGCAAAGCCCCACAGTCCAGGACTGCTACCATGGTGATGGACAGAGTT
ATCGAGGCTCATTCTCCACCACTGTTACAGGAAGGACATGTCAGTCTTGGTCCTCTATGACACCACACTG
GCATCAGAGAACCACAGAATACTACCCAAATGGGTATGTCTTTGAGTTTTCTCCCAAGAGAAACAGCCAC
CCACTTAAATTTCTCCTGGAAGAGCCATGCTTCCAGCTAACTTCTTATGACCCAATTTCTCTCAGACCCA
GAATGTTGGACAGAATGTCTCAGGCTTCTTGCTTTGGGCACAGGGTCTGAGAGGAGAGAAATGTCAGGCC
AGCTCTCTTTTCTCATAGTTGATAGAAGTAGGAGGATACTTGGAGGTGGTGAGGTCTCATGAATAGAAAG
CTCAGAAGAACATATGACCATGTGCTTAGAAATAGCACCATTCCACAATGCCCACTAAAGACCAGTGAAA
TAGTTCAACCAGGGAATTCTGTCATTCTAATCTCCAAGCCCTGGAGTGAAGGTTGTGTTTGCCATGTTTG
TCTTGGGTAACAAGTGAAGGATATCTATATTGACTTCGAGATCTTCCGATCACTTTCTCCTCTAACCTGT
ATAAACACATTGGGTTCTGAGAACAAGGTGTCTGAAAAGCTATGTGTTGCCAGCCCATGAGGGGCAAAAG
GAGGAAGGCAGCTGAGAGTCAGGAAGTATAGAGATGCTGAAGAGTTACACATTCAGGAAGATGGACAGAA
ACCCATGTCTGGCTATGCCAGCCTTTCTGCCATGAAGGACTATCAAATACATGAGAAAACAGTTTTCACA
GGTTGGACAACAGATATGGTAGGCTTGAGAGAACTGAGAAAGGGAATCAAAGGAGATCAACTTCATCATT
AACCTGTCTTCCTTCCTGGACACAGTGTTGGATTGAAGGACAAGCAGATGGAGCCCAGCTGACCACAGCA
GTCTTGCTTAACTGAGGAGAGAGACTGGAGTCTGCGATGCCTCAGGCAGCTGATGTGTTCTAGGCTGGCT
AAGAATGAGAAGGGATTTGTGGAAGAAAGGAGCTCCAGGAATACACACAGAAGTCTCCTCAAGGCTTTGG
CTAAATACAAAGCTGCGTATGCACAGGGAGAGTTTTCATAAAGAAAGAACAACAAAGAAAAGCTACTTGG
GAAAGAACAACTGCAGGGGAACAGTAAGCTCAATGGAGATGCCAGAGCTCACATAGCACTGGGGGATATT
TGAATTCTGACCACTCAGAGGAGAAACACCTCACTACATTTTGGGCATTCAGTAGAGACCAAAGAAAGCT
GTATTTTGGGATTGGGATCATCTTATTCCTAGAATCAAGGTGACTCCAGAAAAACTCCAACAACCCTTCT
TCCAAGCCAGTCTAAAAGGATCCAAATGATCTCCAAGTAAATTAACTGCATTCCACAAGAAAAAAAAAAC
TCAACCCCCCTTAGAGGCAAGGGACAAATACAAGTTGCTCAGTTATATGGCATTCCTATTGCGTTACTTC
TATTTAAAAATTTAATAGAGACACAAGAAGCTTTCACTGTGATACATAACTGGGAGAAAAAATCACTCAA
CACAAACAGGCCCAGAAATTATAGAATTGATGACATTGGTGAGAACATTTAAAATGCACCTCTGAGAACT
GTGTTTCAGGAAAATGTCAGCAAAAGCTGACCATGAGAGAAACAAAAGCAGAATAGCAAGAGAAAAGAAA
AGAACCGGAGAGCCAAATGAAAATTAAAGAACTGAGAAAAGGTACATCTCTAATGAAGAACTCACTGGAT
GGCCTTATCATCACTTTAGACATTACGGTAGGAAAGGTGACCTAGAAAATAATTCAATAGGAGCTACACA
AATCACAGGACAGACAGACAGACCAACAGACAGAAACACACACACACACACACACACACACACACACACA
CACACACACACACAAAGACTGAACCTATTAATCAACAGAGCCTCAAGGGCATCTAGGAAAAATCCACACA
TTTAATATATGTGTTAGGCAAGTCACAGAAGGAGAAGAAAAAGATATCATGACAGACATTATACTTGAAG
CGATGATGGCTCGCAACACGCCAAATATACAGAAAACAAGAAACTCATAGTCAAGAAGCTAAATGACTCA
GGTATAGAATTTTAAAGAGCAAAACTCTATGATTTACTGGGATATATCATAGTTAAGTTGCCTCAATTCA
AAGCTAAAAAGAAAAAAAGGGGGTTCCTATGAACAACAGCTTTGACAGCTGTTGATCTAAGACCACAGCT
TAAATATCTAGGCAAGGAAAAGCAAATAAGGCACAAGGAAAGAGGATGGAAGGAAATAGTCCAAACCAAT
GACATTCAGTGGAAAAGAAAATAGACCAACAAAGGAGTAAATCCATGAAACAGAAAGTTAGGTTCTTTGA
AAAGTCTATATGATTGGCCAAAGTCTGGCTAAACAGATGACAGACCAAGGAGGGAGCATATCCATCACCA
TCATGAGTAACAGGAGAGAGATGCCATTGCTATAGCATCCTCCAGGTGTGAAAGCTGAGAAGTAGATATT
GAGATCAACTGTATGTAAATAAATTCATGAATGTAGATCATGTGGATGGATTGCTTAGGTAAATAACAAA
TCAGCAAATCAAACACTGAATAGATCATGCAGTTTTATAGAGACTTACAGACAGCCTGACAGATAAACAT
TTGTATGTACGTGAAACAATCTCCAAGACACACTTCAAAATCCCTCTCGGTTAATCCAAAGGAATGTATT
TGGCAGAAGGTAGAAGGAGGGTATTCTGATCCTTTCTGGTACACATTGATGTTTTCTCTCAGTTTTCTTA
TAAAGCATAGATTACTTTGAATGTGTTACAATAAGAATCATAAGCTGTCTTTGAAATGTTGACAGTTGTT
TAGAAGTTGAGGACCATTTGTGAGTGTTATGGGACTTTAGTGAGAATATTTCAAATTTGCTTGTTTACAC
TTTGTTACAAGAAAACATAGAGGGTGCCAGGTGGTGCTGTATCTTCTCCAATCTCTGGTGACCTGTATTG
TTTTGGAATTTGCAGTGGCCTGACCAGGAACTACTGCAGGAATCCAGATGCTGAGATTCGCCCTTGGTGT
TATACCATGGATCCCAGTGTCAGATGGGAGTACTGCAACCTGACGCAATGTCCAGTGATGGAATCAACTC
TCCTCACAACTCCCACGGTGGTCCCAGTTCCAAGCACAGAGCTTCCTTCTGAAGAAGGTAAGAAGCCTGC
AGTCAGACAACCATACCCTCGGACATTGGGATAAAAAGATTTGCAAAATCTTTGTGATGCAGAAAACTTC
CATGCTGCACAGGAAGTCGAAGGTGAAGTCATGGACAGCCAATGGGAAGGAAGCTTCAGTGCCTTCTCTG
GGGGGACCAGAGCTGGGATGTTGAGTGCCTTGTGAGGGATGGTGTCTTTAAAAGGGGCACAGACCCTCTA
GGACACTGGATTTATCACTTCCCTGTTATCAAACGAATCATATTAGTGTCCTAGCCAAGATGGATATTCT
AACATCCTGCCAAACTTGTGAAGATATACCAAGCTCCTAAGCCTGTCCAGCCCTTTCTTCAAGTAGGCAG
TGTTTATTGCAGTCTTCAGCTTTACCATTTTGAAGGAATGCCATTTTTGAGGCTGTTGTTCTTGAGAAAC
CTAACATGTCTTCATTAGATCCGTATTGTCCTGAGACTTTGAAGCAGTACATAGCCACCAAATTGTTTAT
CTCCCCAGCCTACCTTCATCTTGGGCATGCCTTCCACACCTAGGATTTGAGGGAAGGGATTTCTCAGTGT
TCTCATCCCTGCTTCTCATGGAACATTTATCTCCGTTGTTTTTTGAGAAGAAGAGTAGTGGATGTCAGCT
TTCTTGTAATGAGGGATCCTGGGCCCAAGATTCCCTGTCTCCCCTCCTAGGCTATAAAATTTTGGCCTGT
ACTCCTTCTCCCTGAGAGGCAATGTGTCTTTACCTACAAGTCCTAGATGCAAGATCCTTTTCTGCCCCAC
ACCCCAGAATCTGAAGGCTTTTGCTTTGGAGGAGCAGTGGTCTAGTGTGCAAGGGTTTCATGTATACCCC
CCACTAACAGCCAATCACCACCTATAGCCTGAACAGCTTGATGCATGGCACCCTGGTCTCCTGCCTTGTT
CTCATGAACACCCAGAAGAGGTGTAAGCAAAAGACCATTCACATGAGTGTAATTTTGAAGTATAGGCACT
CTGATCTGTTTTTTGTTTGTTTCTTTGTTTGTTTGTTTTCCAGGGTTGAATTAAAATATTTATGACTACT
TATTAAATTTCTAGAATCCTATAAGTCTATTTGTATTTTTATTCTACATTTCAATTTGCATGCTAATATA
GAAGAGTGTAAATTGTTAATCCTCAGATTATTCCACTTTGTGTGTCATAATTTTTTTCACATTTCCCTTT
TCTAGGCAATACTGAGCTTGATTTTCTCTTTTAATTTCAGCACCAACTGAAAACAGCACTGGGGTCCAGG
ACTGCTACCGAGGTGATGGACAGAGTTATCGAGGCACACTCTCCACCACTATCACAGGAAGAACATGTCA
GTCTTGGTCGTCTATGACACCACATTGGCATCGGAGGATCCCATTATACTATCCAAATGCGTATGTCTAT
CATGTTAGCCATAAAAGGAACAATAGTCAACTAAAATTTCTCTTAGCTGGCCCATGCTACAAGCTCACTT
CCTAGGTCCAAATTTCTCATAGACTCAGAGTTTGTAGCAAAATGTCTCAGGAAACTTACTTTTGAGCAAA
AGGTCTGAATGAAGAGAAGTTTTAGGATTGCTATCTTTCATAACAATTTGATGGAAGCAGCAGGATATAT
GGAGGTGGTGAAGTCTCATTAATGTAAAGCTAAGGAGATCAAATGACCAAATGCTGAGACAAAGTATCAT
TCCACAATGCCCACTAAAGGTCCATGCAGTCTTTCAACCATGCAATTCTATCATTCTATCCTCCATTCCC
TGAAGTGAAATTTGTGTTTGCCATTTTTGACACGAATCAGAAGTAACAAATTCAGGCTGGGTGCAGTGGC
TCAGGCCTGTGATCCCAACACTTTGGGAGGACAAGACGGGCAGATCACCAGAGGTCAGGAGTTCAAGACC
AGCCTGGCTAACATGGCAAAACCCCATCTCTACGAAAAATTAAAAAATTAGCCGGTCATGGTGGTGGGTA
CCTGTAATTCCAACTACTTGGGAGGCTGAGGCAGGAGAAACACTTGAGCCTGGGATTCAGAGTTTGCTGT
GAGCCGAGAACATGCCACTGCACTCCAGCCTGGGTGACAGAGCAAGACTCAATCTCAAAAAAAAAAAAAA
AGAAGAAGAAGAAGAAAAGAAGAAGAGGAAGAAGAAGAAGAGGAAGAAGAAGAAGAAGAAGAAGAGGAAG
AGGAAGAGGAGGAGGAGGAGGAGGAGGAAGAAGAAGAAGAAGAAGAAGAAGAAGAAGAAGAAGAAGAAGA
AGAAGAAGAAGAAGAAGAAGAAGAAAATAGAAATGAGTGCATATATTTATATATGAGTACTAGCCTGTAT
GAACACACTGGGTTCTAAGCACCAGTTTTCTGAAGGGATATGGGTTGTCAGGCAGAGTAAAAGCAGGAAT
GCAGATGAGAGTCAGGAAGTAAACAGATGTGGTGATTAAAATGGGCAGGTACATGGACAAAAAAATGCAT
GTCTGACAAAAACTGGCCTCTTGCCATAAGTGAGTATGAATAATATGGAAAAACTGTTTGCACATGTTGA
ACAGCAGACAGTACAACCTGAGATAGTTTAGAAAGGGAAACAAATAAGATCAACCCCATAATTACCCTTC
CTAGACTTAAGGGCAAAGAGTTTTAACCAAAGCATTCCACAGCAGTCTTGCTAAACTGGGGAGAGAGACT
GGAGTTTTGTTTACTAATAAAACCGAGATTTTCTAGGTTAGGTAATAATGAGAAAGTATTTGTGGAGAAA
AGGAGCTCCAGGAATACACACAGAAGTCTCTTCAAGTCTCTGGCTGAACAGAAAGCTGTGTATGCACAGA
AAGAGTTTCCAGAGAGAAAGGAGAACAAAGAACAGCTACTGGGGAAAGAACAACTGCTGGGGAACAGTGA
GCTCAATGAAGATGCCAGAGCTCACATAGCACTGGGAGGTATTTGAGCTCTGACCAGCCTGAGGAGAGAC
ACTTCATTGAACATCTTGGGCATTCAGCAAAGACCCCAAAAAACCATACTTCAGGAGTAGAATTAATGCA
TTCCTAGAATAAAGTCTACTCCAGAAACACCCTAGAAAAGCTTAGAAACCAAGTCTAAAAAGATCCAAAT
GATCTCCAAGTAAATTAATTGCCTGTCAGAAGAAAACAACCTCTTCAGAGGTAAACAACAAAATTAAATT
GCTCAATTATATAGTATGCACAATGTGTGGCATACATTTAAAAATTTGCTAAACATACAAAAAGCATTTA
GTGTGACCCATAACCAGGAGAAAAATCAGTCAATACAAATAGACCCAAAAATGATAAAAATAACAGAATT
GGCAAGGAGATTTAAAATGTATGTATCATAATTGTGTTCAAGGATTTAAAGAAAGCGTGGACAAGAAATA
AATAAATGGATAATATCAACAGAAAGAAAAATTGTAAAAGGACCAAATGGAGAGTCAAGAACTGAAAAAA
AAGACATCTCTTTAATGAGAAAATCACTACATGGCCTTATAATCATATTAGATAGTACAGATGATAAAGC
TAACTAGAAAATATTAGGGTGGTGCAAACCATAGCACGCTTATACAAAGCCTGAGAAGATAAACAGAGCC
TCAAGGACATCTATGAAAATATCAAAATATTTAATATTTGTTTAAAGCAAGTCACAGAGGAAGGGAAAGA
GATATTGGAACAGAAAAAATACTTGAAGCAGTGATGGCTGATGACTTTCTAAATATGGAAAAAATGATAA
ACTCACATAGTCAAGAAGCTCAATGGATCAGATATAGGATTTTAAAAAGTAAAGCTGTATGATTTATTTG
GACACATCATAATTAAATTGTCCATAATCAAAGATAGAAAGTAAAATCTTATTTGAAGCCCAAGGGAAAA
AACATACCTTTACATAGAGTAACAGTGACACAAATGACTGATGCCTTCTCATCAGAAACAACACAAATCA
GAAACAATAGAATAACACCTTTAGAGTGGTAAGAAGAAAAAAAGATCAAATCAGAAACAACAAAATAACA
CGTTTAGAGTGGTAAGGAGGAAAACAAGATCAAATCAGAAACAATGGAATAACACCTTTAGAGTGTAAGA
AAGAAAAAAAGATCAAATCAGGAACAACAGAATAACGCCTTCAGAGTGGTAAGAAGGAAAACAAGATAAA
ATCAGAAACAATGAAATAACACCTTTAGAGTAGTAAGAAGAAGAAAAGATCAGGTCAGAAAAAATGGAAT
AATATGCTAAGAAGAAAAAAAAAGATCAAGTCAGAAACAATGGAATAACACCTTTAGAGTGAAAAGAAGG
AAAAAAACCCAGCAAGCTTAAACGCTATGCACAGCAAACAATTCCACTGAAAATGAATGTTACGTAAGTA
CATATTCTGTCCTCCTAAAAACAAAGAACAAATAAAAGAATGTTTCATCAGCAGGATTATGTAATAAAAG
ATGTGAAAGAATGCTATGTAAGTAGAAGAAAAATAATACCATATGGGAATTGGCATCAAAACCACAAAAT
ACTATCAAAACAAAAAAACTTTATTGATAAATTTAACACAATATGCAAAAGAACTATACCATGTATACTA
CATAACATTGGTGAGAAGAAAATTAGAAGATCTAAATAAAGACACATCATGCTTATAGATTAAAAAATCC
AATGTCACTTTTCACAAAACTGATCTTTAGTTTCAACCCACACCCAAGCAGAATTCCTGCAGTCTTTTCT
TGAAAACCTAACAGAATGTATATGCTAGAATCACCAAGACAATCTTTAAAAAGAATAAAAAACTTGGAAT
AAAATCACAAGTTTGTGGGATAGATGCATATGGTAATATGGAAATTCTCATAAAGACACAGTAATCAAGA
CATGTGGTATTGGCTGGGACGCTTGGCTGTAATCCTAACACTTTGGGAGGCCAAGATGAGAGGATTGCCT
GAGATGAGGAGTTGCAGACAAGCCTGGGCAACATAGCAAGACCCTCATCTCTACAAATATTTAAAAAAAT
TAGCCAGGTTTGGTGCCATGTGCCTGTAGTCCCAGCTATTCAGGAAGCTGAGGTGGGAGGATCACTGGAG
CCCATGAGGTGGAGGCTGAAATGAGCCATGATTGTGCTACTGAACTTTAGCCTGGGAGACAGATTAAAAC
CTTCCCTCTCTCTCTCAAACAAACAAACAAAAAATACATAGTATTGGGCAAAACATATGCAAACAAAAAC
AGAAAAGGGTCAGCATAAATTTACATATATGGTCAATTTATTTTCAATACAGGTAGCAAAGCAATTTAAT
GAGGAAATTTTTTTCCAAAATTGGTCTGAAACAACTGGATAGCCATAGAAAAAAACTATAACAAATGTGA
CGCTTGAATCCTACTGTATGACTCAAATTAAATTAATTTGAGATAGCTCTTAGACCTCAATGTAACAGCT
AATTCTGAGGCTGAAATATAAGACTGCTATGAAAAAGTATAGTATCTTATAACCTTGGAGAAGGAAAAAT
TTTTTGAGGGAAGAACCAGAAAACACTAACTGTAAAAGAAAACAAATGATAATGTGGACATTCATTGAAT
AAAAACTTATGCTCACCAAATATGACTGTTAAGAAAATAAATAAGTAAGTAACACACTGGAAGAAAAACA
CTCTCATCCATATATCTGACAAATGGCCTGTATCCAGAGTATAGAAACATTTCTCCCACTCACTAATCAG
AGGACAAACAACCTAATCAAAATGGGCAACAGGCTTGAATAGTCATTTCTTAGGAGAAGATGCACACAGA
GCCAACAATCACCTGAAAAAGTGCACAACATCTTAGCCATCAAAAATCAAGAGTTATAACCCTCATAAGA
TGACACTGAACATCCAGTGTACATGGATATCATTAAGAAGACACAATAATAAGTGGTGTCACCGATTTGG
AGCTAGAATGTGCCACTCTCTCATATGCTGGTGGAAGTTCAAAATCATACAACAAATTAAAAAATCAGTC
TGATGCTTTCTTATAAAGTTCGATAAATATGCATCTATCCTACAAACCTGTAATTCTATTCTTGAATATT
TACCCCCCAAAATGAAAACATAAGTCCACAAAAATCTATATAAATATTCATAGCAGCTTTATGTTTTATA
AACTCAAAATAAAAACTATTTCAATGTTTTCATCAAAAGAAAATGAAAACTATTTAAATGGTTTCATCAA
AAGAAAATGAAAAAAGAATTTCCAGTATATTTATACAAAGGAATACTATTCATCAACAAGGAACAAGTTA
CTGATAGTCTCAGAAGCATGAACAAACCTCAAAAATATATTAAGGAAAGAAGCCAGACGTCAAAGTGTAT
AGTCTGTATGAGTCCATTCATGTGAGTTTATAGAAAACACAATTTATGGTGAAAGAAACCAATAGCATTT
GACACTGGCCGTGGGAAGAGGGTAGCAGAGATTGATTGAGCAGCCACACAAGGGAGTTTCTGGGGTGGTG
AAAATGTTCTGCATTGTGAGGGCAGTGTGGGCTACACAAGTATATGTATTTATCAAATCTCATCCAGCTA
CATTTAAGATCTGTGCATCTCACTCTATGTGAAAATATACTCAACTGAAAAACAGAGCAGGTATCTGTTT
CAGGTGCTACATCACTTGATACGTCCAGTTGTGTTAAAAACCACTGCCTAACATCCTCAAATGGGGGATC
TGGGCTTGAGACTAGGTCACATGTGTAGAGTCTCTACAGAGACCGTGTTGGATTCCCATGCTCCATAATA
CGTTCCAAGTTTTCTCAGACAGCCACAGGTCATGAATGTGAGGATTCTGAGAGGTTGGAGCAACGTTCTT
GGGAGGCATAATGGGGAAGGCATTCTCCAAGATTCCTCCAGCCTGGGGTCTTCACCTGCTGTGCCTCTTA
CTGCATTGTTTTCTGACTCATCCATAGCCACTTGACCCCTTCAGATCCCATAGTCTACCTAGCCGTCTCC
CTTTATGCCTTGGGTCCCGCTGTTCTTTCAACTCATCACCCATTCCTTCAGTCCCAGAGTGGCTGCAGCC
AGCAGAGGATGGACTGAGAGCAGGAGAGGAGGTCGTGCCCATGAACCCATCCTAGAGAAGCAGCATCCTG
CCTGGGAGCTAGTTTTCCAGGGAAGCTTTTATAAGTCCTGTAGACCCAAACCCACTTGCTCTACCAGATA
CAGTATTTATAGTAATACTATTTTCATGATTATTTTATATTGCAAATGTAGAGCATTTATGCTACACTAT
GAGTAAATAGAGTAAGGGGGCTGGCATGGGAATTATATAATCTTGGATGCCACTTCTTCCTTGGGGAAAT
GTATTTGAGTTCCAACTTACATATTACTATATAGTCTTATAGAGAGAGAGACAAAGAGCTAGACAGACAG
AGATATCTTTGTATGTGCATTAAAAAATCTAAGATACATATTTCAAAATCTGTGTCATTTATTCTGGAGG
AAAGTATTTGGCAGAAGGTGAAAGGAAGATATTCTGATCCTTTCTTGTACAGACATGTATTATCTCAGTT
TTCATAGAGAGCATATACTACTTTTGATGTTTTAAAACAAAAATTATAATCTGTGATGTGTCCACAGTTG
TTTAAAAGTTGAAGCTGAAGACCATTTGTGCTTGTGGCAATATTATTGTGGTATAATGGGAATATTTCAA
AGGCACTTGTTAACACTTTGTTACAGCAAAATGTAGAGGGCGCTAAGTGCCCTTGAATATTCTCCCATCT
CTGGTGACCTGTGTTGTTTTGAAATTTGCAGTGGCCTGACCAGGAACTACTGCAGGAATCCAGATGCTGA
GATTCGCCCTTGGTGTTACACCATGGATCCCAGTGTCAGGTGGGAGTACTGCAACCTGACACGATGTCCA
GTGACAGAATCGAGTGTCCTCACAACTCCCACAGTGGCCCCGGTTCCAAGCACAGAGGCTCCTTCTGAAC
AAGGTAAGAAATTTGTGGTTAGACATCTATATACTGGGATGAAAAACCATGGAAAATCTTACTGATGCAG
AAGCCTTCAGTGGTACACTGGAGGGTTGGTTGAGGGTCTGCAATGTGGAGGAAAGCCTCAGCGCCCTCTC
TGGGGGATCCAGAACTGTGATTTTTGGCACGCTGTGAGGAGGCAGTGTCTTTAGGAAGGGCACGGTGTCT
TTAGGAAGGGCACAGACCCGCCAGGGCACTGGACTTACCACTCCCCTGGTTATTAAATGGGTCATTTCAG
TGTCCTAGCCAAAATGGATATTCTAACAGCCTGCCAAATATGTGAAGATTTCCAAGCCAATAAGCCTTTC
CAGTGATTTAAAGTAGACTTTTTTCATTGCAATCTACAGTTTGCAGTTTCTTAAGAACATGGCCTTTGAG
TATGATATCCTAGAGAAACCTAAGGAGACTGCATTATTTTTCTATTGTCCTGGGGCTGCATAGCAGGAGG
TAACCAACGAATGCTGTCTCTCCCTGGCCTATCTCAGTCTTTCACAGGCTCTGTTCACCTCAGCTTTGAA
GTTAGAAATTTCTAGGTGTTCTTGCCTCTTCTTCTCATGAAACCTGCATTGGCAGTGAGTCTACAGAAGA
AGAGGAAGAGAATTCTGCTTTGTTACAATTCAGGACTCTGGGCACTAGAAGATTCCCTATCTCTCCTCCA
AGGGAATAAGTTGTTTGTCTCTAACCCTCCTTGAGAAACAATGAGTCTTTGCCTGCACTCCTAAATGTAG
GATGATTTCCTGCCCAAATTTTCAAAAGATTAAGCCTTTTGCCTTGGTATGAGCAATGGTCTAGGGAAAT
GCGCAAGGGTCTTGTGTCGGCCCCTGACTGACCACCAGTCACCTCCTACAGCCTGCACCAAGGAATGCAT
TGCATTCTGGTCTTCTGCCCTGTGGTTCTCATGAAAACCAGCAGAGATTCATATGATGGAGCTGCACATG
AATGTAATTTCCAATGTCCAGCATTCTCCTCTGTTCTTTATCTTTAGATTTAAAAATAATGTTTCTATGA
ACTTATTAAAATTCTAGAATACTATGAATCTACTGGGTCTTTTCACATCCTTTTGCTACTAGTAGAAAAA
AGAATAGTAATAATTTTCAGAGGCTACTGTCCAGTATGTGACATAAATTGTCTCCCATGTTTCTCTGCTC
ATGCAATTACTGAGTATGATTTATTTTATTTTAATTTCAGCACCACCTGAGAAAAGCCCTGTGGTCCAGG
ATTGCTACCATGGTGATGGACGGAGTTATCGAGGCATATCCTCCACCACTGTCACAGGAAGGACCTGTCA
ATCTTGGTCATCTATGATACCACACTGGCATCAGAGGACCCCAGAAAACTACCCAAATGCGTATGTATTT
GATTAAAACCATAAGAGGAGCAACAGCCAACTCAAATATTGGTTAGAAGACCCATGCTTTAAGCTCACTT
CCTAGGGACAAATTTCTCTTAGACTCACATTTTGGCAAAATGTCTCAGGACCTTTGCTTTTGAGCAAAGA
GTCTAAGAGAAGAGAAATTTTAGGCCTGCTATTTTTCCTAATAGTTTTATGGAAGGAGTAGAATATACGG
AAGTGGCGAAGTCATATTAATGTAAAGCTCAGAAGATAAATGACCAAAGCTTAAACACAGCACCATTCCA
CAATGCCCACTAAAAATCAATGTCATCTTTCACTCGTGCAATTCTGTCATTCTAAATTTCAATTCCCGAA
GGTTTGTTTGCCATTTTTGTCATGGGTAATAAGTAAAAAAAAAAAAATTAAGATGTGTATATATATATAT
ATATATATATATATACACACACACACACACACACAAACATCTGAATATTTATATATATGTCTGAATATTT
ATATACTTGTGTATAAAACTTATATTTAAATTTTTGCATAAATTTATATATTTTTAATATTTCATTAAAA
ATTATATTGTTTCACTATGTATGTCTGAGTATTTTTATATATTTTAATATAACATTTTAAATATTTATAT
ATAAATATTCAGGTATGTAACTGAATATTCATTTACACACACAAATATATGTGTGCATGTGTGTATATAT
ATATATACCCATATATATATATATATATATATATACATATATATATATATATATATATGTATATATATAT
ATATATATATATATACACACACACACACACACACATACATACAGGTATAAACACACTGGGCCTGAAGCAC
CAGTGGTCTGAAAGGACATGTGTTGCCAGGACTTGAAGAGCAAAAGCAGGAAGGCGGATGAGAGTCAGGA
GGTACACAAACGCTGAAAAGTAAAATGGACAAGTACATGGACAAAAAGCAGGTATAAGCATAACAGCCTT
TTGGAAGTAAATGACTATAAAATATATGAAAATACTGTTTTCACAAGTTGCACAACAGATAGTAGTGTAT
TGAGATAATTTAGAACAGAAAACAAATGTGATCAACCCCATAAGTGTGCTGTATTTCATCATGGATTGAA
GGAAAAAGAGATGGAGCCCAAGAAGACCACAGCAGTCTTGATGAACTGAGAGACACCAGAGTTTGGGATT
ACAAAGGCAGCTGGGATTTTCTACACTTGGTAATAATGAGAAAGAATTTGTGGAGATAAAGAGCTACAGT
CATGTACCTAGAAGTCACCTCAGTGTAATATAAATCTGCATATGCACAGGGAGTGATTCCACAATGAAAG
TAGGACAAAGAACAGCTACTGGGGAAAGAATAACTACAAGGGAACAATGAGTTCAATGGAGATGGCAGAG
CTCACAAAGCACTGGGGGATATTTGAGTTCTTACCAGCTAGAAAAGAGACCTCATTGCAAATCTTGGGCA
TTCAGTAGAGACCCCAGAAAAGCCACTCTTTGGAAACAGAGTTGATGTATTTTAAGAGCAAAATCTACTC
CACAAAAATCCTAGCAAAATTGAAAAGCAAGTCAGAAAGACCAAAATCCTCTCAACATAAATTAGTTGCC
CATCAGAAGAAAGCTTAACCTCTTCATAGGTAAACAATAAAATCAAATTGCTCAGTTATCTGGCATCCAC
AATATGTGACATAAATTTAAAAATTTACTAGACATACAAGAAGCATTTAGTGTGATCCATAACCAGGAGA
AAAATCATTCAATACAAATAGACCCAGAAATGACAGAAATGATAGAATTAGCAAAAACATTTAAAATATA
CATATGATCATTTGATCTTGTGATCAGATATCACAAGAGAAGAAAGAGATACTTGAACAGAAAAAATGCC
TGAAGCAATGATGGCTGAAAACTTTCCAAATATGAAGAAAAAAAAGCTCACAGATTCAAGAAAACTAATC
AATCAGAAATATGATTTTGAAAAGTAAAAATGTATGATTTACTTTGGCAAATCTTCTTGGTTAAATTGTC
TAAAATCAAAGAAAGCTAGGAAAATTTTATAAGCCAGAGGAAAAAAGATTGTTTATATAAAGGAACAGTT
ACACAAATGACTGATGCCTTCTCATCAGAAACAATGAAAGTCAGAAACAATAAAGTAACATCTTTAAAGT
AATAGAAGAAAAACCCAAGAGGTGAGGGATCGTGGCAGACAGGAGGCAGGACTAGATTGCAGCTCTGGAC
AGAGCAGCATGCAGAGGCTCATATTGTGAATTTTAGCCCCATATTGACTGCAAGAACAGACCAGCAATCC
TGAGAGGACCCACAGACCGTGTGAAGGAAGCAGACTGCTCCTGCAGGATAAGGGAGACACCCCAAATACT
GTGAGTTCCCCAACTGCAGAAGTGGAAAAGGGAGGCCTTACTCCCTCAAACACACCCCACAACTGGAGAA
GCTGAAAGTCTGTTTGCAGGAGAAGTTCCCAACTTTACCTGGGCCTCAGTAAATTTAGAGAGCTGAGCCA
AGCAAAATATAGGGGTAGAGGAAGCAGCAGAGAAGACCTCAGAGCTTGCTGGATCCCCAAGCAGCTCATT
CCTGCCTGGCACCACAGAGATCCATCAGAAGTGTGGCCAAAGGAACAGAGGGTAAAACTCCACATGGAGG
ACTGCTCTACCTGAACTTTCTAACAATTTGAACAGGGGGAGAAGCCTCCTGGCCAGAACTTGGGGGAGGG
CATGAATCTGGTTTGCAGACTTCACAGGTGGGGGAAGGACTAAAGCCCTTTTCTTTCACAGCTGGGAGGT
GGAAAGCCTCAGGCAAGTTTTCAAGCCTGACTTTCCCCCCACCTGGAAACAGACTTGGAGCTGTTGCGGG
GTTGGGGGCATGGTGGGAGTAAGACCAGCCCTTCAGTTTGCATGGGTGCTGGGTGAGGCCTGTGACTGAC
AGCTTCCCTCCACTTCCCCGACAACTCAGATGACTCAGCAGAGGCAGCCATAATCCTCCTAGGTACACAA
CTCCAGTGACCTGGGAACTTCACCCCCACACCATACAGAAGCTTCAGTAAGACGTGCCCAAGGAAAGTCT
GAGCTCAGACACGCCTAGTCCCACCCCCAACTGATGGTCCTTCCCTACCCACCCTGGTAGCAGAAGACAA
AGAGCATATAATCTTTGGAGTTCTAGGGCCCACCCACCTCTAGTCCCTCTCCACACTAGTATAGCTGATG
CAGGAGGCCAACCAGCACAAAAATAGAGCATTAAACCACCAAAGCTAGGAACCCCTATGGAGTCCATTGC
ACCCTCCTCCACCTCCACCAGAACAGGCACTGGTATCCACAGCTGAGAGACCCATAGATGGTTCACATCA
CAGGACTCTGTACAGACAGTCCCCAGTACCAGCCCAGAGCTGGGTAGACTTGCTAGGTGGCAAGACCCAG
AAGACAGGCAATAATCACTGCAGTTCAGCTCACAGGAAGCCACATCCATAGGAAAAGAGGGAGAGTACTA
CATCAAGGGAACACCCCATGGGATAAAAACATCTGAACAACAGCCTTCAGCCCTACCTTCCCTCTGACAC
AGTCTACCCAAATGAGAAGGAACCAGAAAACCAACCCTGGTAATATGACAAAACAAGGCTCATCACACTC
CCAGTTCACCAGCAATGGATCCAAACCAAGAAGAAATCCCTGATTTACCTGAAAGAGAATTCAGGAGGTT
AGTTATTAAGCTAATCAGGGAGGGACCAGAGAAAGGCAAAGCCCAATGCAAGGAAATCCAAAAAAAAAAA
GGTATAAGAAGTAAAAGGTGAAATATTCAACAAAATAGATAGCTTAATAAAAAAACAATAAAAAATTCAG
TAGACTTTGGACACACCTTTGGAAATGTGACATGCTCTGGAAAGTCTCAGCAATAGAACTGAACAAGTAG
AAAAAATAAATTCAGAGCTCAAAGACAAGGACTTCAAATTAACCCAATCCAACAAAGACAAAGAATAAAG
GATAAGAAAATATGAACAAAGCCTTCAAGATGTCTGGGATTATGTTAAATGACCAAATATAAGAATAATC
GTGGCTCCTGAGGAAAAAGACAATACTAAAAGCTTGGAAAACATATTTGGGGGAATAACTGGGGAAAACT
TACCTGGCCTTGCTGGACACCTAGACATGCAAATACAAGAAACACAAAGAACATGTAAATACAAGCAGCA
CAAAGAACACCTGGGAAATTCATCACAAAAAGATCTTAGCCTAGGCACATTCTCATCAGGTTATGCAAAG
TTAAGACGAAGGCAAGAATCTTAAGAGCTGTGAGACAGAAGCACCAGGTAATGTATAAAGGAAACCCTAT
CAGATTAACAGCCAGTTTTTCAGCAGGAACTGTACAAGCTATAAAGGATTGGAGCCCTATCATAGCCTCC
TCAAACAAAACAATTATCAGTCAAGAATTTTGTATCCAGCGAAAGTAAGCATCATATATGAAGGAAAGAT
ACAGTCGTTTTTGGACAAACAAATGCTAAGAGAATTCACCATTACCAAGTCACCACTAGAAGAACTGCTA
AAAGGAGCTCTAAATCTTGAAACAAATCCTAGAAACACATGAAAACAGAATCTCTTTAAAGCATAAATCA
CACAGGACCTATAAAACAAAAGTACAAGTTAAAAAACAAAAACAAAAAACAAAACCAAAGTACGGAGGCA
ATAAAGAATATGATGAATGCAGTGGCACCTCACATTTCAATGCTAAAATTGAATCTAAATGGCCTAAATG
CTCCACTTAAAGGATACAAAAAGAGTTGGTGGCTGGCAAGATGGCTGAATAGGAACAGCTCCAGTCTGCC
GCTCCCCGTGAGATCAACACATAGGGTGGGTCATTTCTGCATTTCCAACCAAGGTACCCGGCTCATCTCA
TTGGGACTGGTTAGACAGTGGGTGCAGCCCACAGAGGGTGACCTGAAGCAGGGTGGGGTGTCACCTCACC
TGGGAAGTGGAAGGGGTCAGGGAACTCCCTCCCCTAGCCAAAGGAAGCCGTGAGGGACTGTGCCGTGAAG
ACCAGTGCATTCTGGCACAAATACTATGCTTTTCCCACGGTCTTTGCAACCTGAAGACCAGGAGATTCCC
TTGGGTGCCTACACCACCAGGGCCCTGGATTTCAAGCCCAAAACTGGGCTGGCATTTGGGCAGACACTAA
GCTAGCTGCAGGAGTTTTTTTTCATACCCCAGTGGTCCCTGGAATGCCAGCAAGACAGAACCATTCACCC
CCGTGAAGAAAGGGCTGAAGCCAGGGAGCTAAGTGGTCTTTCTCAGTGGATCCCACCCCCATGGAGCCCA
GCAAGCTAAGCTCCACTGGCTTGAAATTCTTGCTGCCAGCACAGCAGTCTGAAGTTGACCTGGGACGCTC
AAGCTTGGTGGGAGGAGGGGTATCCACAAATACTGGGGCTTGAGTAGGAGGTTTTCCCCTCACAGTGTAA
GCAAAACCGCTAGGAAGTTTGAACTGGGCAGGGTGCACTGCAGCTTGGCAAAGCCATTGTAGCAAGAGTG
CCTCTCTAGATTCCTCCTCTCTGGGCAGGGCATCTCTGAAAGAAAGGCAGCAGCCCCAGTCAGAAGCTTA
TAGATAAAACTCCCATCTCCCTGGGACAGAGCAACTGGAGGAAGGGGTGGCTGTGAGTGCAGCTCCAGCA
GACTTAGTTTCCTGCCTGCCAGCTCTGAAAAGAGCACCAGATCCCCCAACACAGCACTAGAGCTCTGATA
AGGGACAGACTGCCTCCTCAAGTGGGTCCTGGTTTCAGAAGATAATAAGAAACTCCTCTGAGCTAAAGGA
GCATGTTCTAACACAATGCAAGGAAGCTAAGAACCTTGAAAAAGGTCAGAGGAATTGCTAACTACAGTAA
GCAGTTTAGAGAAGAACATAAATGACCTTAGGGAGCTGAAAAACACAGCACGAGAACTTCATGACACATA
CACAAGTATCAATAGCAAAATCGATCAAGTGGAAGAAAGGATATCAGAGATTGAAAATCAACTTAATGAA
GTAAAGCGTGAAAACAAGATTAAGGAATAAAGAATGAAAAGGAATGAACAAATCCTCCAAGTATGGGACT
ATGTGAAAAGATTGAACCTACGTTTGATTGGTGTACCTGAAAGTGATGGGAGAATGGAACCAAGTTGGAA
AACACTCTTCAGGATATTATCCAGGAGAACTTCCCCAACCTAGCAAGACAGGCCAACATTCAAATTAAGG
AAATACAGAGAATACCACATTCAAATTCAGGAAATACAGAGAACACCACAAAGATACTCCTCAAGAAGAG
CAACCTGAAGACACATAATCGTCAGATTCACCAAGGTTGAAATGAAGGAAAAAAATGTTGAGGGCAGCCA
GAGAGAAAGTTTGGGTTACCCACAAAGGGAACCCCATCAGACTAACAGTGGATCTTCCTGCAGAAACTCT
ACAAGCCAGAAGAGAGTGGGAGGCCAATATTCAACATTCTTTTTTACTATTATTATACTTTAAGTTCTAG
GGTACATGTGCACAAGGTGCAGGTTTGTTACATATGTATACATGTGCCATGTTGGTGTGCTGCACCCATT
AACTCTTCATTTACATTAGGTATATCTCCTAATACTATCCCTCCCCACTCCCCCCATCCCATGACAGGCC
CCGGTGTGTGATGTTCCCCACTCTGTGTCCATGTACTCTCATTGTTCAATTCCCACCTATGAGTGAGAAC
ATTCGGTGTTTGGATTTCTGTCCTTGTGATAGTTTGCTGAGAATGATGGTTTCCAGCTTCATCCACATCC
CTACAAAGGACATGAAGTCATCCTTCTTTATGGCTGCATAGTATTCCATGGTGTATATGTGCCACATTTT
CTTAATCCAGTCTACCATTGATGGACGTTTGTGTTGGTTCCAAGTCTTTGCTATTGTGAATAGTGCCGCA
ATAAACATATGTGTGCATGTGTCTTTATAGCAGCATGATTTATAATCCTTTAGATATATATCCAGTAATT
GTATGGCTGTGTCAAATGGTATTTCTAGTTCTAAATCCTTGAGGAATCACCGCACTGTCTTCCACAATGG
TTGAACTAGTTTACAGTCCCACCACCAGTGTAAAAATGTTCCTATTTCTCCACATCCTCTCTAGCATCTG
TTGTTTCCTGACTTTTTAATGATCACCATTCTAACTGGTATGAGATGGTATCTCATTGTGGTTTTGATTT
GCATTTCTCTGATGGCCAGTGATGGTGAGCACTTTTTCATGTGTCTCTTGACTGCATAAAAGTTTTCTTT
TGAGAATTGTCTGTTAATATCCTTTGCCAACTTTTTGATGGGGTTGTTTGATTTTTTTTCTTGTAAATTT
GTTTATGTTCTTTGTAGATTCTGGATATTAGCCCTTTGTCAGATGGGTAGATTGTAAAAATTTTCTCCCA
TTCTGTAGCTTGCCTGTTCATTCTGAGGGTAGTTTCTTTTGCTGTGCAGAAGCTCTTTAGTTTAATTAGA
TCCCATTGGTCAATTTTGGCTTTTGTTGCTATTGCTTTTGGTGATTTAGTCATGAAGTCCTTGCCCATGC
CTATGTCCTGAATGGTATTGCTTAGGTTTTCTTCTAGGGTTTATATGGTTTTAGGTCTAACATTTAAGTC
TTTAATCCATCTTGAATTAATTTTTATATAAGGTGTAAGGAAGGGATCCAGTTTCAGCTTTCTACATATG
GCTAGGCAGTTTTCCCAGCACCATGTATTAAATAGGGAAACCTTTCCCTATTTCTTGTTTTTGTCAGGTT
TGTCATAGATCAGATGGTTGTAGATGTGTGGTATTATTTCTGAGGGCTCTGTTCTGTTCCATTGGTCTAT
ATCTCTGTTTTGGTACCAGTACCATGCTGTTTTGGTTACTGTAGCCTTGTAATGTAGTTTGAAGTCAGGC
AGAGTGATGCCTCCAGCTTTGCTTTTTTGGCTTAGGATTGTCTTGGCAATGCATGCTCTTTTTTGTTCCA
TATGAACTTTAAAGTAGTTTTTTCCAATTCTGTGAAGAAAGTCATTGGTAGCTTGATGGGGATGGCATTG
AATCTATAAATTACCTTAGGCAGTATGGCCATTTTCACAATATTGATTCTTCCTATCCATGAGCATGGAA
TGTTCTTCCATTTGTTTGTGTCCTCTTTTATTTCATTAAGCAGTGGTTTGTAGTTCTCCTTGAAGAGGTC
CTTCCCATCCCTTGTAAGTTGGATTCCTAGGTATTTTATTCTCTTTGAAGCAATTGTGAATGGGAGTTCA
TCCATGTCCCTACAAAGGACATGAAGTCATGTATGGGAATGCTTGTGATTTTTGCACATTGATTTTGTAT
CTTGAGACTTTGCTGAAGTTGCTTATCAGCTTAAGGAGATTTTGGTCTGAGAAGATGGGGTTTTCTAAAT
ATACAATCATGTCATCTGCAAACAGGGACAATTTAACTTCCTCTTTTCCTAACTGAATACCCTTTATTTC
CTTCTCCTGCCTAATTGCCCTGGCCAGAACTTCCAACACTATGTTGAATAGGAGTGGTGAGAGAGGGCAT
CCCTGTCTTGTGCCAGTTTTCAAAGGGAATGCTTCCAGTTTTTGCCCATTCAGTATGATATTGGCTATGG
GTTTGTCATAAATAGCTCTTATTATTTTGAGATATGTCCCATCAATACATAGTTTATTGAGAGTTCAGCA
TGGAGAGCTGTTGAATTTTGTCAAAGGCCTTTTCTGCATCTATTGAGATAATCATGTGGTTTTTGTCTTT
GGTTCTGTTTATATGATGGATTACATTTATTGATTTGCATATGTTGAACCAGCCTTGCATCCCAGGGATA
AAGCCAACTTGATCATGGTGGATAAGCTTTTTGATGTGCTGCTGGATTCGGTTTGCCAGTATTTTATTGA
GGATTTTTGCATCAATGTTCATCATGGATGTTGGTCTAAAATTCTCATTTTTGTTGTGTCTCTGCCAGGA
TTTGGTATCAGGATGATGCTGGCCTCATAAAATGAGTTAGGGAGGATTCCCTCTTTTTCTATGATTGGAA
TAGTTTCAGAAGAATTGGTACCAGCTCCTCTTTGTATCTGTGGTAGAATTCGGCTATGAATCTCTCCTGG
ACTTTTTTTGGTTGGTAGGCTCTTAATTATTGCCTCAATTTCAGAGCCTGTTATTGGTCTATTCAAGGAT
TCAATTTCTTTCTGGTTTAGTCTTGGTAGGGTGTATGTGTCCAGGAATTTTTCCATTTCTTCTAGATTTT
CTAGTTTATTTGCACAGAGGTGTTTATAATATTCTCTGATGGTAGTTTGTATTTCTGTGGGATTGGTAGT
GATATCCCCTTTATCATTTTTTATTGCATCTATTTGATTCTTCTCTCTTTTCTTCTTTATTAGTCTTGCT
AGTGGTCTATCAATTTTGTTGATCTTTTCAAAAAACCAGCTCCTGGATTCATTGATGTTTTGAAGGTTTT
TTTGTGTCTCTATCTCCTTCAGTTCTGCTCTGGTCTTAGTTATTTCTTGCCTTCTGCTAGCTTTTTAATG
TGTTTGCTCTTGCTTCTCTAGTTCTTTTAATGGTGATGTTAGGGTGTCAATTTTAGATCTTTCCTGCTTT
CTCTTGTGGGCATTTAGTGCTGTAAATCTCCCCCTACACACTGCTTTAAATGTGTCCCAGAGATTCTGGT
ATGTTGTGTCTTTGTTGTCATTGGTTTCAAAGAATATCTTTATTTCTGCCTTCATTTCGTTACATACCCA
GTAGTCACTCAGGTGCAGGTTGTTCAGTTTCCATATAGTTGAGCAGTTTTTAATGAGTTTCTTAATCCTG
AGTCCTAGTTTGATTGCACTGTGGTCTGAGAGACAGTTTGTTATAATTTCTGTTCTTTTACATTTGCTGA
GGAATGCCTCACTTCCAACTATCTGGTCAATTTCAGAATAAGTGCGATGTGGTGCTGAGAAGAATGTATA
TTCTGTTGATTTGGGGTGGAGAGTTCTGTAGATGTCTATTAGGTCTGCTTGGTGCAGAGCTGAGTTCAAT
TCCTGGATATCCATGTTAACTTTCTGTCTCATTGATCTGTCTAATGTTGACAGTGGGGTGTTAAAGTCTC
CCATTATTATTGTGTGGGAGTCTAAGTCTCTTTGTAGGTCTCTAAGGACTTGCTTTATGAATCTAGGTGC
TCCTGTATTGGGTGCATATATATTTAGGATAGTTAGCTCTTCTTGTTAAATTGGTCCCTTTACCATTATG
TAATGGCCTTCTTTGTCTCTTTTGATCTTTGTTAGTTTAAAGTCTGTTTTATCAGAGACTAGGATTGCAA
CCCCTGCTTTTTTTGTTGTTTTCCATTTGCTTGGTAGATCTTCCTCCATCCCTTTATTTTGAGCCTATGT
GTGTCTCTGCACGTGAGATGTGTCTTCAGAATACAGCACACTGATGGATCTTGACTCTTTATCCAATTTT
CCAGTCTGTGTCTTTTAATTGGAGCATTTAGCCCATTTACATTTAAGGTTAATATTTTTATGTGTGAATT
TGATCCTGTCATCATGATGTTCGCTGGTTATTTTGCTCATTAGTTGATGCAGTTTCTTCCTAGCATCGAT
GGTTTTTACAATTTGGCATGTTTGTGCAGTGGCTGATACCGATTGTTTCTTTCCATGTTTAGTGCTTCCT
TCAGGAGCTCTTGTAAGGCAGGCCTGGTGGTGACAAAATCTCTCAGCATTTGCTTGTCTGTAAAGGATTT
TATTTCTCCTTCACTTATGAAGCTTAGTTTGGCTGGATATGATATTCTCAGTTGAAAATTCTTTTCTTTA
AGAATGTTGAATATTGGCTGCCACTCTCTTCTGGCTTGTAGAGTTTCTGCTGAGAGATCTGCTGTTAGTC
TGATGGGCTTCCCTTTGTGGGTAACCCGACCTTTCTGGTGAATCTGACAATTATGTGTCTTGGAGTTACT
CTTCTCGAGGAGTATTTTTGTGGCATTCTCTGTATTTCCTGAATTTGAATGTTGGCCTGCCTTTGTAGGT
TGGGGAAGTTCTCCTGGATAATATCCTGAAGAGTGTTTTCCAACTTGGTTCCATTCTCCTCGTCACTTTC
AGGTACACCAAGCAGATGTAGATTTGGTCTTTTCACATAGTCCCATATTTATTGGAGGCTTTGTTCATTT
CTTTTTACTCCTTTTTTTCTCTAAACTTCTCTTCTCGCTTCATTTCATTCATTTGATCTTTAATCACTGA
TACCCTTTCTTCCACTTGATTGAATCAACTACTGAAACTTGTTCATGTGTCACGTAGTTCTCGTGCCATG
GTTTTCAGCTCCATTAGATCATTTAAGGTCTTCTCTATGCTGTTTATTTTAGTCTGCCATTCATCTAAAC
TTTTTCAAGGTTTTTAGCTTCTTTGCAATGGGTTCGAACATCCTTCTTTAGCTCGGAGAAATTTGTTATT
ACAGATCGTCTGAAGCCTTCTTCTCTCAACTCATCAAAGTCATTCTCTGTCCAGCTTTGTTCTGTTGCTC
GTGAGGAGCTGCGTTCCTTCGGAGGAGAAGAGGCACCCTGATTTTTAGAATTTTCAGCTGTTCTGCTCTG
GTTTCTCCCCATCTTTGTGGTTTATCTACCTTTGGTTCTTGATGATGGTGATGTACAGATGGGGTTTTGG
TGTGGATGTCTTTTCTGTTTGTTAGTTTTCCTTCTAACAGTCAGGACCCTCAGCTGCAGGTCTGTTGGAG
TTTGCTGGAGGTCCACTCCAGTCCCTGTTTGCCTGGGTATTACCAGTGGAGGCTGCAGAACAGCAAATAT
TACAGAACAGCAAATGTTGCTGCCTGATTCTTCCTCTGGAAGCTTCATCTCAGAGGGGCACCCAGCTGTA
TGAGGTGTCAGTTGGCCCCTACTGGGAGGTGTCCCCCAGTTAGGCTACTCGGGGGTCACGGACCCACTTG
AGGAGGCAGTCTGTCCATTCTCAGATCTCAAACTCTCTGCTGGGAGAACCACTACTCTCTTCAAAGCTGT
CAGACAGGGATGTTTAAGTCTGCAGAAGTTTCTGCTGCCTTTTGTTCAGCTATGCCCTGCCCCCAGAGGT
GGAGTCTACAGAGGCAGGCAGGTCTCCTTGAGCTGTGGTGGGCTCCACCCAGTTTGAGCTTCCTGGTCGC
TTTGTTTACCTACTCAAGTCTCAGCAATGGCAGACGCCCCTCCCCCAGCTTTGCTGCCGCCTTGCAGTTC
GGTCTCAGACTACTGTGCTAGCAGTTCAATCTCAGACTGCTGTACTAGCAGTGAGCAAGGCTCTGTGGGC
ATGGGACCCTCTGAGCCATGTGCAGGATATAATCTCCTGGTGTGCCGTTTGCTAAGACCATTGGAAAAGT
GCAATATTAGGGTGGGAGTGTCCCGATTTTCCGGGTACATCTGTCATGGCTTCCCTTGGCTAGGAAAGGG
AATTCCCTGACCCCTTACACTTCCCGGGTGAGGCAATATCCCGCCTTGCTTCGGCTCACTCTCCGTGGGC
TGCACCCACTGTCTGACAAGCCCCGGTGAGATGAACCCAGTACCTCAGCTGGAAATGCAGAAACCACCCA
TCTTCTGCTTTGCTCATGCTGGGAACTGTGGACTGGAGCTGTTCCTATTCGGCCATCTTGAAACCTCCCC
TCTCTCACGATCACAAGGTCCCACAATAGGCCGTCTGCAGGCTGAGGAGCAAGAAAAGCCAGTCTGAATT
CCAAAACTGAAGAAATTGGAGTCTGATGTTCAAGGGCAGGAAACATCCAGTGCCAAAGAAAGATGTAGAA
TATTCAACATTCTTAAAGAAAATAATTTTCAACCTAGAATTTCATATCCAGCCAAACTAAGCTTTATAAC
AAAGGAGAAGTAAAATCCTTTACAAACAAGCAAATGCTGAGGAATTTTGTCAACACCAGGCCTGCCTTAC
AAGAGGTCCTGAAGAAAACACTAAATATGGAAAGGAAAAACCAGTAACAGCTACTGCAAAAACATACCAA
ATTGTAAACACCATCAACACTATAAAGAAACTGCATCAACTAATGGGCAAAATAGCCAGCTAGCATCATA
ATGACAGGATCAAATTCACACATAACAATATTAACCTTAAATGTAAATGGGCTAAATGCCCCAATTAAAA
GACACAGACTGGGAAATTGAATAAAGAGTCAAGACCCATTGGTTTGCTGTGTTCAGAAGACCCATCTCAG
GGTGAAAAGACATACATGGGCTCAAAATAAAGAAATGAAGGAATATTTACCAAGCAAATGGAAAGAAAAA
AAAAGCAGCGGTTGCAATCTTAGTCTTTGATGAAACAGACTTTAAACCATCAAAGATCAAAAGAGACAAA
GGAGGGCATTACCTAATGGTAAAAGTATCAATGCAACAAGAAGATCTGACTGTCCTACTTATATATGCAC
CCAATACAGGAGCACCCAGATTAATAAAGCAAGTTCTTAGAGACCTACAAAGAGACTTAGACTTCCACAC
AAAAATAGTGGGAGACTTTAACACCCCACAGCCAATATTAGATCGACGTGACAGAAAATTAACAAGGATA
TTCAGGACGTGAATTCAGCTCTGGACCAAGCTGACCTAATAGACATCTACAGAACTCGACACCACAAATC
AACAGAATATACATTCTTCTCAGCACCACATTGCACTTATTCTAAAATTGACCACATAATTGGAAGTAAA
ACACTTCTCAGCAAATGCCGTAGAATGGAAATCATAACAAACAGTCTCTCAGACCAAAGTGCAATCAAAC
TAGAACTCAGGATTAATAAACTCACTCAAAACCACACAACTATATGGAAACTGAACAACCTGCTCCTGAA
TTACTACTGGGTAAATAACAAAATTAAGGCAGAAGTAGATAAGTTCTTAGAAACCAAAGAGAACAAAGAC
ACAATGTGCCAGAATCTCTGGTACACAGCTAAAGCCATGTTTAGAGGGAAATTTATAGCACTAAATGCCC
ACAGGAGAAAGCGGGAAAGATCTAAAATCAACACCCTAACATCACAATTCAAAGAACCAGAGAAGCAAGA
GCAAACAAATACAAAAGCTAGCAGAAGACAAGAAATAACTAAGATCAGAGCAGAACTGAAGGGGATAAAG
ACACGAAAACCCTTTAAAAAATTAATAAATCCAAGAGCTGGTTTTTTGAAAAGATTAACAAAATACATAG
AAGCCTAGCCAGACTAATAAAGAAGAAAATAGAGAAGAATCAAATAGACACAATAAAGAATAATAAAGGG
GATATCACCAATGATGCCACAGAAATACAAACTACCATCAGAGAATACTTTAAACACCTCTATGCAAATA
AAATAGAAAATCTAAAAGAAATGGATAAATTCCTGGACACATACACCCTCCCAAGACTAAACCAGGAAGA
AGTCAAATCCCTGAATAGACCAATAACAAGTTCTGAAATCGAGGCAGTAATTAATAGCTTACCAACCAAA
AAAAGCCCAGACCAGAGGGATTAACAGTCAAATCCTAACAGAGGTACAAAGAAGAGCTAGTACTATTCCT
TCTGAAACTATTCCACACAATAGAAAAAGAGGGACTCCTGCCTAACTCATTTTATGAGGCCAGCATCATT
CTGATACCAAAACCTGGCAGAGACACAACAAGAAAAGAAAATTTCAGGCCAACATCCCTGATGAACATCA
ATGTGAAAATCCTCAATAAAATACTGGCAAACTGAATCCAGCAGCACATCAAAAAGCTTATCCACCATGA
TCAAGTTGGCTTCATCCCTGGGATGCAAGGCTGGTTCAACATATTCAAATCAATAAACATAATCCATCAC
ATAAACAGAACCAATGACAAAAACCGTATGATTATCGCAATAGACGCAGAAAAGGCCTTTGATAAAATTC
AATACCCAATCATGCTAAAAACTCTTAATAAACTAGGTATTGATGGAGCATGTCTCAAAATAATAAGAGC
TACTTATGACAAATGCATAGCCAATATCATACTGAATGAGCAGAAGCTGGAAGCATTCCCTTTGAAAACC
AGCACAAGACAAGGATGCCCTCTCTCACCACTCCTATTCAACATAGTATTGGAAATTCTGTCCAGGGCAA
TCAGGCAAGAGAAAGAAATAAAGGTATTCAAGTGGGAAGAGAGGGAGTCAAATTATTTCTCTTTGCAGAT
GACATGATTGTATATTTAGAAAACTCTATCATCTCAGCCCAAAATCTCCTTAAGCTGATAAGCAACTTCA
GCAAAGTCTCAGGATACAAAATCAATGTGCAAAAATCACAAGCATTCCTATACACCAATAAGAGACACAG
AGCCAAATCCTGAGTGAATTCCCATTCACAATTGCTACAAAGAGAATAAAATATACCTAGGAATCCAACT
TACAAGGGATGTGAAGGACCTCTTCAAGGAGAACTACAAACCACTGCTCAAGGAAATAAGATAGGACACA
AACAAATGGAAAAACATTCCATGCTAATGGATTGGAAGAATCAATATTGTGAAAATTGCCATACTGCCCA
AAGTGATTTATAGATTCAATGTTATCCCCATCAAGCTACCATTGATTTCTTCACATAATTAGAAAAAACT
ACTTTCAATTTCATATGGAATAGAAAAAGGGCCTGTATATCCAAGACAACCTAAGCAAAAAGAACAAAGC
TGGAGGCATCATGCTATCTGACTTCAAAATATACTACAAGGCTACAGTAACAAAAACAGCATGGTATGGT
ACTGGTACCAAAACAGATATATAGACCAATAGAACAGAACAGAGGCCTCAGAAATAACACCACACATCTA
CAACTATTGGATCTTTGACAAACTGGACAAAAATAAGCAATGGGGAAAGGATTCCCTATTTAATAAATGG
TGTTGGGAAAACTGGCTAGCCATATGCAGAAAACTGAAACTGGATCCCTTCCTTACACCTTATACACAAA
TTAACTCAAGATAGATTAAAGAATTAAATGTAAGACCTAAAACCATAAAAACCCTAGAAGACACTTTGGG
AGGCCGAGGTGGATGGATCACGAGGTCAGGAGATCGAGACCATCTTGGCTAACACAGTGAAAGCCCATCT
CTACTAAAAATACAAAAAATTAGCTGGGTGTGGTCGTGGGCACCTGTAGTCCCAGCTACTTGGGAGGCTG
AGGCAGGAGAATGGCATGAGCTGAGGAGGTTGAGCTTGCAGCAAGCCAAGATTGTGCCACTGCACTCCAG
CCTGGGCAACAGAGTGAGACTCCATCAAAAAAACAAAAACAAAAACAAAAAATCAAACCCTAGAAGAAAA
CATAGGCAATACCATTCAGGACATAGGCATGGGAGAAGACTTCATGACTAAAACAGCAAAACCAATGGCA
ACAAAAGCCAAAATTTACAAATCAGATCTAATTAAAATAAAGAGCTTCTGCACAGCAAAAAACTCTCATC
AGAGTGAAAAAGCAACCTATGGAGAAAAATTCTGTGGTCTAGCCATCTGACAAAGGGCTAATGTTTAGAA
TGTACAAGCAACTTAAACAAATGTACAAGAAAAAAAAAACAACCCCATCAAAAAGTGGGCAAAGGATATG
AACAGACACTTCTGACAGGAAGACCTTTATGTGGCTGACAAACATGAAAAAAGCTCATCATCACTGTTAA
TTAGAGAAATGCAAATCGAAACCACAATGAGATACCATCTCATGCCCGTTAGAATGGCGATCATTAAAAA
GTCAGGAAACAACAGATGCTGAAGAGGATGTGTGGAGAAAGAGGAACACATTTACACTGTTGGTGGGAGT
GTAAATTAGTTCAACCATTGTGGAAGACAGTGCGGTGATTCCTCAAGGATCTAGAACCAGAAGTACCATT
TGACCCAGCAATCCCATTACTGGGTATATACCCAAAGGATTATAAATCATTCTACAATAAAGACACATGC
ACACGTATGTTTATTGTAGCACTATTCACAATAGCAAAGACTTGGAACCAACTGAAATGCCCATCAATGA
TAGACTGGATAAAGAAAATGTGGCACATATACACTGTGGAATACTATGCAGCCATAAAACAGGATGAGTT
CATGTCTTTTGCAGGGACATGGATGAAGCTGGAAACCATCATTCTCAGCAAACTAACACAAGAACAGAAA
ACCAAACACCATATGTTCTCACTCATAAGTGTGAGTTGAACAATGAGAACACATGGACACAGGAAGGGGA
ACATCACACACAGGGGCCTGTTGGGGAGTTGAGGCTAGGGGAGGGATTGGATTAGGAGAAATACCTAATG
TAGATGATGGGTTGCTGGGTGCAGCAAACCACCATGACACGTGTATACCTATGTAACAAACCCACACATT
CTACACATGTATCTCAGAACTTAAAGTATAATAATAATAAGATACAGAACTGCAGAATGAATAAGAACTC
ACCAACCATCTGCTGCCTTCAGGAGACTCATTTAAGACATAAGGACTCACATAAACTTAAAGTAAATGGG
TGGAAATAATAATAAGTGGTGTCACTGATGTGGAGGTAGATTATAAAACTCTTATCATATGCTGGTGGAA
GATCAAAATGATAAAACGAATTAAAAAATCAGTCAGATGGTTTCTTAAAAAGTTCCATCAATATGCCTCT
ATCTTACAAACCTGCAATTCTATTCCTGAATCTTTATCCCAAGGAAATGAAAAAGTAAGTCCACAAAGAG
TTCTATATGAATATTTATAGGAGCTTTATTTATTATAATTCAAACTGTAAAAATAATTTCAATGTTCATC
AATAACAAAATGAAAAAATAATTTGCAACCTACTGGTACACTTGAATACTATTCAGCACTGAGTATCTTA
AATAGCATGGATGGAGCTCAAAAATATACTCAGGAAAGAAGCCATGTATATTCTGTATGAGTTCATTTAC
ATGAGATCATTTACATTTCCTCCAAAAGAGGAAAAACTAATTTCTGTTGAAAGAAACCAATGTATTTGCC
TCTGGCAGTGGTAAGGGGGTAGCACAGATTAATTGGGTAGGGACTCAAGAGAGTTTCTGGGGTCACAGAA
ATGTTCCGTGTGGTGATGGGAGTTTGGGCTCCACAGGTATAGGTGTTGATCCAAAATCATCAAAAAAACA
ACATTGCAGATCTGTGCATCTCACTCTGTGGGAAAGTATATCTCAACTGTAAAAAGGGCAGAAATTGCTT
TTAAACGCTCAGCCTTTTAGCACATCCAGTTGCTTGGAGAACCAGCTTACTCAAATGGGGGTCTAGGCTG
GAGACTAGGTCACAGGCATAGAGTCTCTAAACTTTCCCATGGCACATAATACGTTTCAGGTTTTCTCAGA
GAGCTGCAGGTTAGTAATCTGAGGATTCTGACAAGTTGGGTCAACGTTCCTAGGAGGCATGAATGGGAGT
GCATTCTCTAAGATCCCTCCACCCCAGGGTCCTTGCTTTCTGTGCCTCTTACTCCATTGTTTTCTGACTC
CTCTGTAGCCACTCGACCTCTTCAGATCCCATTGTCTACCCAGCCATCGCCCTTTATGACTTGGGTCCCA
CTGTTCTTTCATCTCATCCTCCATTCCCTCAGTTTCGGAGTGGCTGCCGCTAGCAGAGGATGGACTGAGA
GCAGGAGAGGTGGTCCTGCCCAGGAACCCATCCTAGAGAAATGGCATCCTGTCTGGGAGCTAGTTTTTTA
GGGCAGGTTTTATAAGTCTTGTAAAGCCAGACACACTTGATCTACCTGGTATGTTATTTACAGTAATACT
ATTTTCATAATTGCTTTTCACTCTAAAAGTAGAGCCTTTTAGCTACACTGTGAGTAAATAAAGGGGCTGG
CCTGGGAATGGTATCATGTTGGATGTTGTTTCTTCCCTGAAGTAATATATATCAGTTACAATTTACATGT
TACTGCAGAGTCCTAGAGAGAGACACAGAGAATGAGACAGATACCAATACATTTTTATGTGCATTAAAAA
AATCTAAGGCCAGGCGCAGTGGCTCACACCTGTAATCCCAGCACTTTGGGAGGCCGAGGTGGGTGGATCA
CGAGGTCAGGAGATTGAGACCATCCTGGCTAACACGGTGAAACCCTGTCTCTACTAAAAATACAAAAAAT
TAGCCAGGCGTGGTGGCGGGCGCCTGTAGTCCCAGCTACTCAGGAGACTGAGGCAGGAGAATGGCTTGAA
CCCAGGAGGCAGACCTTGCAGTGAGCCGAGATTGCGCCACTGCACTCCAGTCTGGGCGACAGAGCGAGAC
TCCGTCACAAAAAAAAAAAAAAATCTAAAATGCACTCTTCAAAATCTATGTCATTTATTCTGGAGGAATG
CAGTTGGCAGAAGGAGGAAGATATTCCGAATTTTTCTTGTATACATTTATGTATGATCTCAGTTTTTTTA
TGGATCATAGACCAATTTTGATATTTTAAAATAAAAATTATAATCTATCTTGGAAATTTACATGGTTCTT
TAGAACTTGAGGACCGTTTTTGCTTTTCGGAATATTATTGTACCTAAAATGGGAATATTACAACGTCACT
TTTTAACACTTTGTTATAACAAAGTTTAGACAGCGCTGGGTGCCCCTGAATTTTTTCCCGCCTCTTGTGA
CCTGTGTTGTTTTGGAATTTGCAGTGGCCTGACCGAGAACTACTGCAGGAATCCAGATTCTGGGAAACAA
CCCTGGTGTTACACAACCGATCCGTGTGTGAGGTGGGAGTACTGCAATCTGACACAATGCTCAGAAACAG
AATCAGGTGTCCTAGAGACTCCCACTGTTGTTCCAGTTCCAAGCATGGAGGCTCATTCTGAAGCAGGTAA
GAAGTCTGTGGCCAGATATCTACACATTTGAACATTGGGATGAAAAGAGATGGAAAATCTGACTGATGCA
GAAGCCTTCCATGCTACACAGAAACTTGAGGGTATGGCAGGTGGAAAGAAGCCTCAGCACTCTCTCTGGT
GGAGCAATTTTTGGCGCAACGTGCGTGGGCGGTGACTTCAGGAATGGTGCAAACCCACCTGGGCACTTGA
CTTACCACTCACTTTGTTATGAAAGGGGTTATCTCGGTGTTCCAGACAAAATTCCAATTCTAACATCAGG
CCAAATTTGTGCCAAATTTCACACTAGTGAGTGTTTCCAGGCATTTATTAAAATGGACAGTGTTCATTGC
AATCTTCAGCATTGCAGTTGCTGAGGTATGTGGCCGCTGAGTTTGTCATCCTGGGGAAACCTAATATGAT
GATATTTATTCCATCTAATCCTGGGGCTATTTGGCAGTAAATACCACAGAATACACTATTTCTCTGGCTT
ATTTCAGTCTTAGGTAGGCTCTGCACACCTATGCTTGGAAGGCAGGAATTTCTTGGTGTTCTTGTGCCTT
CTTCTCATGGAACGTGCATCTTTGGTGTGTGTTGAGAGGAAGGGTAGTAGACTTCTGCTTTGTTGCAATG
CAGGATGCTGGAACAAGAGGATTCCCTGTCTCTACTGTAAGGGAATAAGATTTTAGCCTCCATCCTTCTC
TAAGAAGCAATGTGTCTTTGCCTCCAAGTACTAGATGCAGGACCATGAACTGCCCCGTCCACCAGAAGCT
TAAGGCTTTGGCTTTTCAGGAGCAATCATCTAGGGAACTGTGCAGGGTTTTCATGTCTGTCCCCTACTGA
CAGCCAATCACCATACAGCCTGCATAACCTAATCCATCATCGTCTGGTTTCCTGCCTCATTGTTTTCATG
AACAACCAGTAGAGAGCCATACGAAAGAGCTTGCACATGAGTCTTTGTTCCAATTGTAAGAGCACTGATA
GGTCCTTTTCCCACCAGGTTTTGAATATAAAATTTCTAAGAACTTATTAAAATATTAGAATGTTATTAAT
CTATTGTTTTTGCTTCAGCATGTCCTTCTGCTTGTGAGTATACTAAAGAGAACAGTCATAATTCTGAAAC
TACTGTCCTGTTTGTGTCATAAATTGCTTCACATGTTTCTGCATACTAGTAGTTACTCAGCTTGATTTTG
TCTATTTTCAGCACCAACTGAGCAAACCCCTGTGGTCCGGCAGTGCTACCATGGTAATGGCCAGAGTTAT
CGAGGCACATTCTCCACCACTGTCACAGGAAGGACATGTCAATCTTGGTCATCCATGACACCACACCGGC
ATCAGAGGACCCCAGAAAACTACCCAAATGAGTATGTCTTTGATGTTACTTGTAAGAGGAGCAACAGCCA
ACTTAAGTTCCTCCTAGAAGAGCCTTGCTTCAAGCTAACTTGTTAGGACAAATTTCCCTTAGACCCAGAA
GGTGTGTCAAAATGTCCAGACAACTTTGCTTTTGATCAAAGAGTCTGAGAGAATAGGTATTTTAGGCTTG
CTATCTTTTCTAATAGTCTGATGGAAGCAGAAGGCTACATGGAGCTGATGAGGTCTTTTTAATATAAAGC
TCAAGAGATCAAATGATCAAATACTTAGAGTGCCATTCTACAAGGCTCATAAAAGATCAATGCACTCTTT
CACCCATGCAATTCTATCATTCTAACCTCCCTTCTCTGAAATGAAGGCTTTTTGCCATTTTTGTCATGGG
TCACAAGTAAATAATTCACATGTATATGAGTATATATATAACCAGGTGTGTTTATTCAGACTAGTATGTA
TATATATACATATATATGTTCATATAAGTTAGTATTCATATATATGTTCATATATATATGTTCATACAGA
CTAGTATTCATATATATATACATATATATATACACACACATATATATATATATATATATGTTCTAGGGAA
ACATGCAAGGTTTTTATGTCTGTCCCTGACTGATGACCAAATACCCTATAGCCTGCACAGCTGCAAGCTG
TATAGCCATACAATTTGCAGGACACACACACATACACACACACACACACACACACACACACTAACATATA
ATATAATATAATATAATATAATATAATATAATATAATATAATATAATTAATATATATAAACCTGTGTGAA
CACACTGGGTTCTAAGCTCCAGTTTTCTGAAGGGATATGGGTTGCCAGGAGAGGAAGAGCAAAAGCAAGA
ATGTAGATGAGAATTAGGAAGTAAACAGATATGGAGATTAAAATGGGCAGGTACATGGACAAAAAACCAG
GTCTGACAAAAACTGGCTTTCTGCCATAAATGACTATAAAAGATATTAAAAAACACTTTCCACATGTTGG
ACAAGAGACAGTACAGGACTGAGATAATTTAGAAAAGGAAATGAATGAGCGCAACTCCGTAACTATTATG
ACTTTCTTCCTGGAGAACCTTCCTGGACTGAAGGGCAAGGAATTGGAGCCAAAGCCAACCACAGCAGTCT
TGCTGAACTGAGGAAAGAGACTGGAGTTTGGGATAGCTAAGAAAATGTGTATTTTCTATGCTAGGTAATA
ATGAGAAAGAATTTGTGGTGAAAAGGAGCTGAAGGAATATGCATGGAAGTCTAATATAAACTGCATATGC
ACAGGGAGAAATTCTACAAAGTGGGACAGAGAACCACTACTGGGGAAAGGACAAATTCAGGGAAACAGTG
AGCTCAATGGTGACGCCAGAGCTCACGTAGCACTGGGGGATACCGGGGTTCTGATCAGCCCGAGGAGAGA
CACCTCATTGAACATCTCGGGCATTCAGTAGAGACCCCAGAAAAGTCATACTTTAGGAGTAGGATTTATG
CCTTCTTAGAATAAAGACTACCCCAGAAACACCCTAGTAAAGCTTAAAAACCAAGTCTAAAAGGACCCAA
ATGATCTCCAAGTAAATTAACTGCCTGACAGAAGAAAACTCAACCATCACTGGAGGTAAATAACATGATT
ACAGTGCTCTGTAATGTTGCATTCACAAGGAGTGACATCATTTAAAAATTTATGAGGCAGGAAAAAGCAA
TTAGTGTGATCCATAACTAGGAGAAAAACCAGTCAATACAAATAGACCAAGAAATAGTAGAAACGATGGA
ATTGACAAAGAAATTAAAACTGTATATATGATAATTGTGTTCAAAGATTTAAAGAAAACATGAACATGAG
GGAAACAAATGCAGAATATAAAAAAAAGCAAATGCGTAAAACAACCAAATGGAAATTAAAGAACTACAAA
AAAGTATAACCTTAATAAAATACTCACTGGATGGCCTTAATATTAGTTTATACATTACAGAAGAAAAAGT
GAACCAGAAGATAACTCAATGAAAGCCATACAATCTGTAAGACACACACACACGCACACGCGCGCGCGCG
CACACACACACACACACAGAGAGAGAGAGAGAGAGAAAGAGAGAGAGAGAAAGGCTGAAAAAAATAAATA
GAACCTTAAGGATATCAGTGAAAATAGCAAAAGATTTAATATATGGGTAAAGCAAGTCACAGAAGGACGG
GAAGGAGATATTGGGACAGAAAAAAATACTCAAAGCAATGATGGCTGAAGACTTTACACGTATGAAGAAA
ATGATAAACTCACAGTCAAGAAGCTCAATGAATCAGAAATAGTATTTTTAAAAGCAAAACTCTATGATTT
ACTTGGGTACATTATAGATAAATCGTCCAACATCAAAGATAACAAGGATAATCTTATAAGCCAGAGGAAA
ACAATATCATTTACATAGAGGGACAGTAATGAAAGTGACCGATGCCTTCTCCTTGGAAACAATGGCATAA
CATCTTTAAAGTGATAAAGAGAAATAAAAACAGATCAACCTAGGACGACATGTCCAGCCAAAACAAACAA
ATAAACAAAAAAACCCTTTAAAATAAACGTGATGTAAATACGTATTCTGCCACCTCCAGAGGAAACAAGC
AAAAAAACAAAAGAATGTTTCCAAGGCAGGCTTCTGTATTAAAAGATTTTAAGGAAAGTTATTCAGGTAG
AAGAAAAATAATACCAGATGGGAACTTTAATCCATACTAAGTAATGAAGAGCCCTGGAAATGGCAAATGG
CAATGTCAATATAAAATACTCTTATTTATCTAATTTTTAAATGTATTTAAAGGACAATTTGTGATATTAA
TTAAAATAATAGGAATATATTGTTGTTTCAACGTATGTAGTAGTAAAATTCATAAAAACAGTAGCACAAA
TAATGCAGATGATAACTGGAAGTATACTGTTAATGAGTTTTTTGCATTATCCATGAAGTTATATAATATT
AATAGATGGTTGAATGTGATAGTTTAAGGTGGGATATTATAAATCCTAGGACAACCAAAAAAATTTAAAC
TGAGAGGAATGGATAGTAAGAGGAATAGTCCTTTTATGCAAAAGAAGGAAGAAAAAGAGGAATAAAGAAT
ATAAAAGATATGGTGTAAACAGAAAATACATAGCATTATTGTAGACACAAACTGAACTACCTTATGAGTA
TATTAAATATAAAAGGATTAAGCATTACAAATAAAAGGCAGAGATTGTAAATTGAATAAAAACCACAGCT
AAGTGTGTTCTTTTTAGAATAAATACTCTTTAAGTGTAAAGATCTACTTTAAACACCAAAATATGAAAAA
GGATATATACCATGAAAACCTGAATCATAAATAAGCTGGAGTGGTGATTAATGGATGCAGGCACTCCTAA
AGACTAATAAGTGAATGTGGTCAAATTGAAGAAACAAAAGTATATACGTGCTCAATGTGCAAAAACTTTT
TCTGTATACATGCTATGATCCTTTGGAAAATTAAAGTTTTAAAGCAATATCACTGACAATAGTATCAAAA
CCAAAAAATATTTAGTGATAAATTTCACACACTATGCTCAAGGACTATACACCTTGCACTAGAAAACAAT
GTTGAGGAAAGAATTAAAAGATCTAAATATACACCATGCTTATAGATTAAAAGACTCCATATCAGTTCTC
GTGAAATTGATCTTTGGATGAAACCCACACCCAAGCACTATTGCAACAGTCCTTTTTTGGAAAAAAAAAT
TGGAGGACTTATATACCTTAATATAAAGACTTATAAAAGTACAGGAATCAAGACATGTGGTATTGGCCTG
GCCCCTTGGCTCATGCCTGTTACCCCAACATTTTGGGAGGCTGAGTCTGGAGGATGGCTTGAGCCCAGAT
GTTCAAGACCAGCCTTAGCAACAGAGTGAGACCCTCTCTCTACAAAAAATAAACAATTAGATCGATGTGA
TGACTTGCACATGTAGTTTCAGCTACTCGGAATGCTGAGGTGAGAGGATTGCTTGACTCAGGAGGTCTAG
CCATGAGTGAGCATTGATCATGCCTCTGCATTCCAGCCTGGATGATGGAATGAGACACTGTCTCAAAAAA
AAAAAAAAAAAAGGATATGTGTTATTGGCCAAAAAAGTATGCAAACCTAAAAAGGGATGGCCCACCACCA
GACCCACATACATATATGGTAAATGGATTTTCCGTATAGATGGCAAAGCAATTCAATGGAGACAAAAATG
TTTTACAAAATCATTCTGAACCATTTGGATATCCATGATACAAAACAAAAGCAGAACTTGACTTTTGCTT
TTCATCTCAAATTATTTTGATATCTCTTCCACCTAAGTGTCAGAGCTAAAACTGAACCTGAAATATGAAA
GTTCCATGAAAAAATATAAAATCTTCACAACCTTGGAGAAGGCAAACTTTTTTGAGGCAGGAGTCTGTAA
ACACTCACTATAAAATAAAACAAATTATAATGTGGGCTTTCATGAAAACTCATGCTTACCAAAAGTCATT
GTTAAGAAAATAAATAGGCAAGTAACACATGAGAAGAAAAATGCTCTCTGTCCATATATCTGACAAATGG
CTTGTGTCCAGAATATAGGAACATTTCTCCCACTCACTAAACAGAGGACAAACAACTAATGGGCAACAGA
TTGAATAGGCATTTCTTGGGGATAGATAGATGTACACATAGCCAATAAGCACCTGAAAAAATGTCCAGTA
TCTCAGCCATGAAAAATAAAGAGTTATAATCATCATGAGATGTCACCAAACACCCAATGGACATGGATAT
TATTAAGAAGACACCACAGTAACTGATGTCACTGATGTAGAGCAAGGATGTGAAACTCTCTCATATGCTG
GTGAAAGTGCAAAATGATACAACCACTTTTGAAATCAGTCTGATAGTTTCTCCAAAAGTTCAATAAATGC
ACTTTTACCCTACAAACCTGCAATCCTGTTTGTGAATATTTACCCCACAGAAATGGAAACATAAGTCCAC
GAAGACATCTCCAAGAATATTCATAGCAGCTTTATTTTTTATAACCCCAAACTGTAGACAATTTCAATGT
CAATCAATAAGAAAATGAATAAATAATTTGTGAACTAGTCATACAATGGCATACTGTTCAGCAATAAAAG
GGAGCATGTTTTTGATACTCTCAAATAGTATGGAAGATGCTCAAAAATATTACATTAAAGAAAGATGCCA
GATAACAAAAATGAACATTATGTATGAGTCTATTGATGTAAGGTTCCAGAAAGGTAAAACTAATTTCTGG
TGAAAGAAACCAATATCATTTGCCTCTGGCCATGGGAAGAGAGTAGCAGAGATTGATTGAGCAGTAAAAC
GAAGTTTTTTTCTGGGGTGATGTAAATGTCCTGTATTGTGATTGAAGTGTGAGTTACACAAGTGTACATG
TTCATCAGAAGTCATCAAACTACATCTAAGATCTGTGCATTTGACTATACATGAAAATATACCTCAGTTG
AAAATAGATCAATAACCTCCCTCATATACTATACTTGCTAACACAGCCAGCTGCTTGGAGAACCAGCTTG
CTGGAATGGAGAATCTGGGCTTGAGACTGGGTCACATGTATAGAGTCTCTACAGAGACAATGTTGCATTC
CCACGGTACATAATACATTTCAAGGTTTCTCAGACAGCCACATGTCATGAATGTGAGGATTCTGAGAGGT
TGGAGCAACATTCCTGGGAGGAACGAAGGGGAGCACATTCTCCAAGATCCCCCACCACCGGGGTCCTCAC
CGGCTGTGCTTTTTTTTTTTTTTTTCTTGACAGAGTCTCGCTCTGTCGCCAGGCAGGAGTGTAATGGCCC
AATCTCGGCTGATTGCAGCCTCCAACTCCAGGGTTCAAGAGATTCTCCTGCCTCAGCTTCATGAGTAGCT
GGGACTACAGATGTGCGCCACTGCGCCCAGCTAATTTTTGTATTTTTAGTAGAGACGGGGTTTTGCCATG
TTGGCCAAGATGGTCTCGCTCTGTTGACCTCGTGATCCACCCGCCTTGGCTTCCCAAAGTGCTGGGATTA
CAGGCGTGAGCCAAAGCACCCAGCCTGTGCCTCTCACTTACTCAATTGTTTTTCTGAACCCTCCATAGCT
GGTGGACCTTTTCAGATCCCATAGTCTAGCCAGCCCTCTCACTTTATGCCTTGGGTCCCACTGTTCCTTC
ATCTCATCCCCCTTCTGTCAGTCCCGCAGTGGCTGTGGCCAGTAGAGGATGGACTGAGAGTAGGAGAGGA
GGTTCTGCCCAGGAACCCATCCTAGAGAAACAGCATCCTGCCTGGGACCTAGTCTTCCAGGTCAGCTTTT
ATAAGTCTTTTAGACTCAAACTCACTTGACCCACCTGAAGTGGTATTGACAATAATGCTATTTTCATGGT
TGTTTTTCACTGTAAATGCAGAGCCTTTTAGCTACACGACTAGTACAGAGAGTAAGGGAGGCTGGCCTGG
GAATGATATCATCTTGGATGGCATTTCCTCCTTGGAGAAATATATGTTAGTTCCAACTCACATGTTACTA
TACAGTCCTGTAGAAAGAGATACAGAGAGTTAGACAGGTATAGACGCATTTGTATATGCATAACAATCTA
TAAGACACACATCAAAATCCGTATACCGGTTCCTCTAGGGGTATGTGCTTGGCAGAAGGTAGAAGGAGGG
TATTCTGGTTCCTTTCTTTTGCACATTTATGTATGATCTCAGTTTTTATATGGAGCATTGATAGGGTTTG
GCTATGTCCCCACCCAAAATCTCATCTTGACTTGTAATCTCTATAATCCTGATAATCCCCATGTGTCAAG
GGCAGGACCAGGTGGAGGTAACTGGATCATGGGGGCAGTTTCTCCCAGGCTGTTCTCATGACAGTGAGAG
AGTCTCCTGAGATCTGATGGTTTTGTAAGTGTCTGGCATTTCCCCTACTTGCACTTACTCTGTCCTGCCG
CCTGTGAAGAAGGTGCCTGTTTCTCCCTTGCCTTCTGCCATGACTGTAAATTTCCAGAGGCCTCCCCAGC
AATGTGGAACTGTGAGTCAATTAAAACTCTTTTCTTTGTAACTTACCCAGTCTGTCTCGGGTATTTCCTC
ATAGCAATGTGAGAACGGGCTAATACAAGCATATACTACTTTTGATATTTTAAAATAAAAATTATCATCT
ATCTTTGAAAGGCATGCACAAATGGGAAGTTGAGGAACATTTGTGTTGTGGCAATTGTATGATACCTTTA
ATGGGAATATTTCAAAGACACTTGTTAAGACTTTGTTAGAACAAAATGTAGAGGGTGCTGGATGTCCCTG
AATATTCTTCCGCCTCCTGTAACTTGTATTGCTTTGGAATTTCCAGTGGCCTGACAATGAACTACTGCAG
GAATCCAGATGCCGATACAGGCCCTTGGTGTTTTACCATGGACCCCAGCATCAGGTGGGAGTACTGCAAC
CTGACGCGATGCTCAGACACAGAAGGGACTGTGGTCGCTCCTCCGACTGTCATCCAGGTTCCAAGCCTAG
GGCCTCCTTCTGAACAAGGTAAGAAGTCTGTGTCTTACCTTGTCTAGCACATACCTCTCTATGTGCTTGG
ACAACGGGATGAAAAGACATGAAAAACCACACTGATGCAGAAGCCTTTAGTGCTACACGGGAGCTCGAGT
GTTGGTTGAGGTTCTGCCATGACCAAGGAAGTCTCAGTGCCGTCCCTGGGAAAGCCAGAGCTGTGATTTT
TGGCACAACTTGTGGGAGTAGTGACTTTAGGACTGGCGCAAAACCTCCAGGGTGCTCAACTTAACCACTC
ACCTTATTCTAAAATGGGTTATTTCAGTGTCCCAGTCAAATTCCTATTCTAACATGCTGTCAACTGTGTG
ATTATTTCCAAGCCAATAAGCATTTCCAGTAATTTCTTAAAATAGTGTTCATTGCAGTCTTCAGCGTTGT
GGCTCCTGAGGGATGTGGCCCCTGATTCTGTCGTCCTAGAGAAGCCTGACATGACTGCATTGATTCTGTA
TCGTCCTGGGTCTATGTGGCTGCCTGGCTGTCTGTAATCATCTGTTTTATTTTTATTTTTTTCTACAGAC
TGTATGTTTGGGAATGGGAAAGGATACCGGGGCAAGAAGGCAACCACTGTTACTGGGACGCCATGCCAGG
AATGGGCTGCCCAGGAGCCCCATAGACACAGCACGTTCATTCCAGGGACAAATAAATGGGCAGGTCTGGA
AAAAAATGTAAGCCACTTTGATTTGGACTCTTTTTCCCTTTGCTGACAAATCTTTTCAAACAGAAGAGGG
GCAGAGGAAAATACTGGAAAGACTTCAGGAGGCTAAGCGTAATTAGCCTTAGCATGGAAAGTGCAAGCAG
CACAGGCCAGCAAAGCCCCACGCGTGTGGGGGTTCTCAGGCCTCTTCTCTTTTGACATTTCTTTACTGTT
TCCATTGTTGGGTGCTGTTTCTCGTTTCTAGTGCTTGTCCTCTAAGCCAGGGGTCCCCACTCCAGTACTG
GTACTGGTACTGGTACTGGAACTGGTAATTATCTGTGGCCTGTTAGGAACTGGGCTGCACAGCAGGAGGT
GAGCTTCGGGGGAGCAAACAAAGCTTCATCTGTATTTTCTGCTGCTTCCCATCACTCTCATAGCTGCCTG
AGCTCTGCCAGCTGTCAGATCAGAGGCAGCATTAGATTATCATAGCACAAACCCTATTGTGAACTGCACA
TGTGAGGAATCTAGATTGCATGCTCCTTATGAGAATCTAATGCCTGATGATCTGTCATGCTTCCATCACC
CCCAGATGGGACCACCTACTTGCAGGAAAATTAGCTCAGGGCTCCCACTGATTTTACCTTATGGTGAGAT
GCACATTTATTTCATTATATATTACAATGTAATAATAATTGAAATAAAGTGCACGATAAATGGAAGGTAC
TTGAGTCATCCTTTAACCATCGCCCCCTCACCCCAGGTGCACAGAAAAATTGCCTTTTATGAAACTGGTC
TCTGGTGCCAAAAAAGTTGGGGAACCACACTGCTCTGGGTTCTAGTAGTCAGAGATGCCCTCTATGAGGC
TTAAGTCAGATTTTTCTAGAAAAGATTTGGATGGGCCATCAGGTCACCATGAGACTTCCCTTAGCCTCAT
GCATTCTCTGTGATGGTTTACTTTGGGGCCTATGAATAGGGAAGACTGAGATATAGGAAAAACCAAAGTG
TCTGTGTTCCCCCACTCTCACACCCATGTAACATAACACTTCTCACACCAGATATGGGGGGATTTCTCCT
CACACCCCAAGCGAGTCTCCAGCAGATACCAGCTGGGTGTCCTACAATGTAACTCGGTCCTGACACTCTA
TCTGGAGACAGTGTCAGATCCCACAAGTTAAGGCTCAGTCCTACAAGACTGCCCCACTGCAGATGCCAAT
CCCAAGTTGCAGGCTGTGACCTGTACTTCTGCCCAGCTGGATAAAGATCTGTTTTTCTATATGACCCTCC
ATGGGTTTGATTACTTTGCTAGAGTGGCTCACAGAACTCAGGGAAACACGTTACTTTTATTTACCCATTT
ATTATAAAAGATATTAAAAAGGATCCTGGTGAACAGCCAGGTGGAAGAGATGCACAGGGCAAGGCACGTG
GGAAGGGGCTCAGAGCCTCTATGCCCTCTCCAGTGCACCAGTCCCCAGTACCCTAAGTGTTCAGCAACCC
AGAAGCTCTCCAAGTGCAGTCTTGTTGGGTTTTTATGGAGGCTTCATTACAGAGGCACAGTTGATTACAT
CATTGGCCATCGGTGATCGGCTCACCTTCGGCCCCTCTTCCCTCCCTGGAGGTTGGAGGGTGGGGCTGAA
CAGTTCCAACCCTCAAGTCACATGGTTGGTTCCCTTGGCAACCAGCCCCTGGGGCTATCCAGGAACCCAC
CAAGAGTTGCTTCATTGCAGCTCCCTTCACCCAGGAAACTCCAAGGGATTTAGGAGCTCTGTGTTAAGAA
CTGGGGGGCAGAGACCCAATATACATTTCTTATTCTATCACAATATCACAGGAAGCTAAGGATGATACTG
CCTTTGTGTGTCTTGGCTGTGGATGGTGCATAATGCATGGAAGTAAGCATTTCTGAATCAACAGCAAACA
GGCTTTATCAGGTAGAAGACCCCTCAGCGCCCCAGGGACAAAGCTCATCAATGATGTCCCACTGTCCTCT
GAGGCTCTAGCTCTAAGACCTCCAGTGGGTCAAGCTCCTGGAGAAGTGGCACATTCTCCAAAGACCCTTC
AGGGTCACCACACCCTGGTTAAGGGTGTGGCCTCATAACTCCTTTTGACTATGACTGATGGCTTACAGCA
TAGAAAGAAATAACTTTGTCAAAAAATATAATAATGATAGAAAGGAAGAAGGAACGCTCCCTTTTGTCTT
CTAAGAATAGATGTGAAATGTGTGTGCCTTAGAATATCTTCTCCCTCTCCTGCTCCACGTGAGCTGGAGC
TTACATGCCTGCTTGTTTTCAGTACTGCCGTAACCCTGATGGTGACATCAATGGTCCCTGGTGCTACACA
ATGAATCCAAGAAAACTTTTTGACTACTGTGATATCCCTCTCTGTGGTAAGTTGCCTTCTGTTTTGGTAA
GGAAACTGCTTCCTTAATATGGATTTGGAAAAAAAAAAGCAAAAAAAACAGAAAATGGCTTTTGAGCTGA
GTGCTTCTGGGGAGGAGATGGCTGCCCTCTCCACCAGAGCCTGCTTTTCATCATGGCCACCTTGAACCTG
CCCTACTATTGGCCCCATTTGTTAGGAAAACACCCGCCCCTCCCACCACACACACATAAATAAAATAAAT
GTCAAATTCCCAAAGGGCAAACTTAGAGGTGATCTAATCAGCCCGGGATAGTCCCACCGAACCCTTCTTT
GTCTAGCGTGGGATGCATGAAAAACAAATTTAGAGTCATTATGATGAAAAACTGTCCTCTTCTGCAGCTG
AGAAGAAAAAAAAAATACGAGCAGCAGGAAACAGCTAAGCATGTAATGCACATTGTAAACCTCAGATGGC
CATCCTAGGAAATCAATGAAGGGTAGTGCAGCTCTTTAGCCCCAGATGGCCTTTCTCGTAAGATTACTAC
TCATGAGTCCCATTAGCGACATTGCTTAGAGACTGCTTGTTAGGTTCCTTCCTCATTGCTCTGAGACTCT
TATTGGGAGTATGAGGCTTGGATCAGGGGAAGGGGAATTGACATTAGATCTTAAATGATTGGGGTAACAA
ATCCATGGGGGAAAAAAAGCCACTTGTACTTGTTCCCTATTTTCTTCCTGCTGACCAATCAACTTGTCTG
TCCGAGTTACAGAACACCACCCTGGACTTTTCTTTTGTGTAATTTGGTTGCTTGTGGTTGGGTCTGCCAT
GTGAAGGGACCTTGAGCTGGGGGAAGAAGGTTGGCCTCCAAGTCCACTGAAGACCAGCATCCTGAGATTG
CCTGGGGAGGTGGTACAGGGCAGTGATGAAGATCATGGGAGCCACACTGCCCATCGTCACATTTGGGCCA
CTCCTGGGGAGAGCAAGAGGGAAGAAGGAGAGGTTAGGGTGATAGGAAAGATTCTACTTGGCCAATATTA
TTATAATGTGGCATTGTGGTCTCTGGATTTAGTGTGAGTTGATAGCTGACTTTTTTCTCGAGTGGGTGCT
TTTGTTCTATTTTGTCGGTGCTATTGCAGAAGCATCTTGGTGGTTCCTCTACCTCAAAGTCTCTTGATGG
GGTCAGTTCCAGTTCTCCGCTTCTGGCCCCATCTAGTACACGCCACTGCCTCTCACTGCCTGGGCTCTCT
ATCCTTGACAGGCTGCCTTGAATTTAAGCCCAGTCTGACTTACCTGCCTCAAACACCCACAGTAGTGCCT
GGGACTCATGCACCTTTGACTCCCATGGAAGGGAAGTGCAGTAGCTTCCCAGGTGCAATTCTGCTGTCCT
CACCCACATTGAGGATGTATGAGAATCAGGTTCTTAGAGATTGGAGAAAGAAGGAAGAATGGGAACAAGA
TTTCTTCCAATGGACTGTGAGGTTCCCCACCTTACTTTGATGTAAGACAAGTGAGGTTAACCCCAAGCCT
GGTGAGGAGGGTTCCCATCAGACACTTGGAAATCCTGAGGACTGTTTCCTGCAGAAGGATGTGGTTGGTG
GGATATTCAGGTTTGACTCATGATTGAGAAAGTTAGAGCCTCTGGTTGGAGAAAGAGTTTAATAACTATT
TCATTTCCACCAACACATTCAGTACGAATAATAAATAAGTAAAAATAAATAGAAACATTCAGTTTTATTT
TGAATAGTAGGAGTAGGGTATAATTTCTGTAGTTACTCTTTTAGTACAATGATGCATGTTTACTGTATGT
AAGGCATACTAGCAGAAATTGAGCTCAGCACTAGAAAAGATGATTGCATTCCATGCCATGCTTCTTTTTT
ACAAAAGACTTCTATAGATAGATTCTCAAAACAACCCACAGCAAATGAAAAGTTATTTGGAAAACTCAGG
TTCCAGATTCACTGGAGTGTAGAATCTCTGGTTGGTTGGGGAGGAATTTCCTCTTGCAGTTGTTATTAAT
AATTATATGAATAATTATTAACTATATTAATATTTATAGTTTTGAAGACCTTGAAGGGCTGGAGACAACA
GAGAAGCATTTTTGAACACCCTCTGTAGCCCCTGCACTGTTGTAGGCATTGATGGGTGGTACCAAAGATG
GGACACTTTCCCTACCTCCAGAGACCTTGTGGGCTTGCTGCAGAGAGAAGGCAGGGAGGAGGAAAAGAAG
AATAGAGGCACATGTGTGTAAATTACCCCCACAGCAGTCAGTTAGTCATGGGAGGCTCCCCAGAAGAACT
GTCCTGAAGCTGGCTGAGAGAAGGCAACATTTCAACATAGGACAGTTATCCTTGCTACATAAAATCACAT
ACACACATGCACATATGTCCACACACAGAGACTCACATGCAAAAGAATCCTTTGTGCCTTTCAGTAAACT
TTACATGGTTTAGAAAGAACTTATATTTCCTTGAAAGGAGAGTGTCCTTTGTTGTTTACTACCACTTTTT
AAACTTAGAAAGAAAAATCTAAAGAGTGTTTATGATTTTACCATTTAATTTCACCTTTGAGATGTGAAAA
ACTAGTGCTTGGAATTCGTCCTGAATTAAACGACACAATTGCTAACTTGGACTCAAATGCGACTTCTTTT
CCCACCTTGTGCCACAGCATCCTCTTCATTTGATTGTGGGAAGCCTCAAGTGGAGCCGAAGAAATGTCCT
GGAAGCATTGTAGGGGGGTGTGTGGCCCACCCACATTCCTGGCCCTGGCAAGTCAGTCTCAGAACAAGGT
AAGAACAGGCCCAGAAACCATCTATACTGTCCTTCCATGTAAGCCCCACAAAACCCTTCTACATTTACAC
AGAACCCACACAGCTGATGCATCAATACCTGCCTCTCTGTTTTCTGAAGGAGGAAAAAATATAGAAAAAT
TAAAAAAAGTTATATTATTATAGGTTCTCTACTTGGAAAATAGCCAAAATACAAATCTTTTTCTTGATCT
GGGCAGTTCCATCAAAATCTGTAGGCACAGTGATTTGCACCAAGTTCCAATACTTTTGGAAAATATTGAA
GATGCTCTGAGGGTTTCTATGGATATCCATTGTCTCACTGTCAGATGAAAAGAAAGGGAAGTTTTTAGAA
ATGTGACACTTTGCAGTGAGGGAGGACAAGAGCAAACTTACCTACAGTCTATCACAGGCACAGATTTTTT
TTTACACTTTTGTGAATCATTGAATTCAATGCCGAGGCTATTCATCTATTCACAAACACATGAACAAATT
ATGGGTTGTGATCCCCATAAATGAAGAGTAATCAGTCCGAACCCACAGAACCTGGACATTTTGGGTATCG
TTTCAGTGGAACATGCAATTCGTAAGTTCAGTTTGCTTGGGTGTCTCTTAGGAAGAACACATAGGACACA
GACCCATCTGCCTGCATGTTTTGCTTCCTCATCTCCTTTCTACACCAGGGCACCTGTGCTCAATTGCTGT
TCTCCTCTAAAGAGACTTCCTTCTGTAAGTTTGTGAAATGCCATCGACAAACCTGATCGCATCGCATTTC
ACTCTGCTGTTGAGTTGATTTTTCTTTACTTTATCGTTTGTAACTTCTTGCTCTACAGAGCTTTCACCTT
CCACATATTTCAGATTCATTCTTTCCTAAACTGTGTGGTGGTCTATGTCCTCACTGACTATCAACATACT
GCCATCATGCACTTCCTATCTCTATTCCTCTTCGTTGCAATCTGGCTCCAAGTGGCTCACACCATTATTC
TGATCTATCAACTGCCTACACAGTCCTAGAAAGTAAGTGAGTCAAGAAACATCCCCCAAAAGTAAACTTT
TCAGGTAAGATCAGAAGACCCTCATGAGTCACTGCTGCTCAGGATCGTATCTGGCTCCTTGAAGAGTGAC
CTTGCATAGATCTTGTCATAAAAAATGAAAGAGACCTTGGGAAGGTCTTGGGCTGGTCACTTTTGTCAGA
GTCCAGGGCTGTGGGGTGAAAGCCACAGCTATAGAGCTTCATTCTGGAGTCACTTAGCTTTGCTCTCCTG
GGGACAGGCTGTGCCTATTCTTGCCTCAGGCATCAAAAAAAGTGGCACAGATGGGCCCTTCTGAAAAATC
TCACTACTGGAGCACAGCTCGAAGTTTCTACTATCCTGACGTTGGGCGGTAGTCCTTTGCTTTGGGAATA
TGAACATGATCAAAACTGAGTGAACTTGTCTTCCTGGCTTTCTGTACAATGAAGTAGAACAAACCATCCA
ATTTGACCAAAGCCTTGGCATGTTTTCTTTCTAGGTTTGGAAAGCACTTCTGTGGAGGCACCTTAATATC
CCCAGAGTGGGTGCTGACTGCTGCTCACTGCTTGAAGAAGTACGTTTAAGGGAAAACTGACATGGGGTCT
TATCTTCAAGACTTTTTTCCTCCCTCTCTTCCTCCATCCCTTCTTTCTTCCCACCCTCCCCTTCCTTCCT
CCCCACCTCTCTTCCTTTTCTGGAAGGAACACTAGGAACCAGGGAATGCATGCAGAATCCTGAGGCAGAA
TTTCCAGGGCAATTGGATGAGAGAGGAGGGAAGTGTTTCTAGAGGGAATCTGCAGAGGGAAGACCCAGTG
CAAGTGATTTTTTGGACCTGTATAAACCGCAGGACAGAGCTGTTCACTACCAGAGGCATCAATCTGTATT
GCATTGCTCTAGAGCAATATCTGAGGCTGAATAATTTATAAAGAAAAGAGTTTAATTGGCACATGTTTCT
GCAGGCTTTACAGGAAGCAGGATGCTGTCATCTCCTCTGCTTCTGTGTGGGCCTAAGGAAGATTACAATC
ATGGTGGAGGGCAAAGTGGGAGCAGGCATGTCACATGGCCAGAGCAGGAGCAAGAGACAGAGAGAGATGG
GGTGGGGGTGCTGCACAATACCAAATGACCAGACTTTGCAAGAACTAAGAGTGAGAGCTCACTGATCACC
ATGAAGATGTGGCCCAAGCCATTCAAGAGGGATGCACCTCTATGATCCAAACCCCTTTCACAGGCCATAG
CTCCATCACTGGGGACTACAGTTGAACACGAGATTTAGGTGGGGACAAATATACAAACTATATCACAGTC
TCTGATGAAACAGATTGAGAACAGACCTTAACTGTCAGTTTCCAGCAAATTGTGAATTTTGTTTCTTGCC
ACTCATAAGTCACTGATTCTGGGTGGCCGAGGGTGTCAGAGGGACAGCGCCAAGTTCATGGCACAGAGGA
TACCTGAAGGGGCTGGACCATATTTTTCTCTTGACATCCTCATCTTTTCTAGGTCCTCAAGGCCTTCATC
CTACAAGGTCATCCTGGGTGCACACCAAGAAGTGAACCTCGAATCTCATGTTCAGGAAATAGAAGTGTCT
AGGCTGTTCTTGGAGCCCACACAAGCAGATATTGCCTTGCTAAAGCTAAGCAGGTACTCGCTCACCTGTG
GTCTTCACCCCACGCTGGTGAAGATATTTGCTTTATGTCTGGGTTTTATGGGCCATGGCCACTGCATGGC
AGTGGGGAGGAACTGTCTATCACATGAAAGGCTCAAGGGCTTTGGGGACAGCATCAATCTTCAACCCCAG
CCCTGCCACATGTTAGTTGTGCTCTTTAAAAAGGCAGAAGGATTCGTTTCCTCACGTGGAAAAAGAGATA
CCCTGTTACCCGTAAAACTTACTTAATGTTCACCAGTTCATCCACATTCATGATCAGGGAAAGGTTGTTA
TTCCAGGCTAACTATTCTCCTTTCATAATAATATGCTGGAGAGAATCAAATGAGATTGCATTTCAAAGCG
CTTGAAAAACCACCATATCGAGCCATGCTTAGTGTGGGCGCCTCTAATCACTGCTATTCAGGAGGCTGAC
GAGGAAGAATTGCTTGAGCCCAGGACTTCAAGGCTGTAGGCAGCTATGATTGTGCCACTGCACTCCAGGC
TGGGTGACAGATCAAGACCCTGTCTCAACAAAAGAAAAGAAAACAAAACAAATGAACAGAAATATTCCAC
AATGTCAAAAAAAAAAAAAACCCACACAACATACAATTTACAAATGCAAATAATAATATTATTGTTGTCT
TCTTTGATTTTCTCTTTCCTGGTGAAATTTTGTTTTATTAAGCCTGACAAAGTGATACCTTTGCTTACAT
CACTTAAAGTTAGTCTATTTGGACCTAGGTGACAGTACAATCAGCTAAGAAACAGTATTTGTAGGAGAGG
CAGGTTTGGGACAGGTGACAAGGCATGTGGGGTGCTCGCTGTGCTGGTGGCTCTGGAAGGCAGGGTGTCA
ATGCAGACAGGGATGAGCATGGCCTGGTTGGGAAGGCATGGGGCAGGCAGGAGCCTGAGCTGCTCTCCTG
GGCCTGGTCACAAGCCCATGGCAGCTTCTCTGGGTCTGTGAACTGAGGGGTGATGTCCTGGAATCCTCTG
ACACTCTAGGAAGGAGAGAAGGGCCTTTCTGGCTCAGCCTTTATAAACAGTAGCTGATCTCCCTCTTGCT
CCCCAGGGTCCTCCCCACCATCCCAGCAAATGTGCAAATACAAGATCTCTGCTCCTCATGGTCCTCAGAG
AGCTGGGGTGTTCTGATGGCTTGAACAAGTCACTTAGGAAATGTGGGGTTTTGGAGGCATTCTCTGATAG
GCTGATACGTTTTGAGTTTAGAGTTCCCACCGCACATCCCCACACCCCTAGAGTCTAGGGCATTTAGTGC
TCCATGAGGGAACCTGTAGAGTGAGGACATCTGCATCACAGGCTGGGCCTTCTAGTGTCCAGAAGCAGAA
AGTGTGTCTGCTTCAAAGTTGGTGCTAATGATGATTTTTGGTCAGAATACGGCATTTCTCATTTCCATTC
CTTTATCCCCTTGAACTTACTAAAGTAGAATCAGGTCTAAAAACCAGAGTTCTAATCTTTAAGAGTCCCT
GGGATTCTAAGGTATATGAATGTCCTTGGAAAACAATACCATTTAGTTCATGCAAGGTGCTTATTTCCCA
TCCTCTTTCATTTGATGTCTAGCATTTTACTGCATTCTTACCACCACGGTTTAGTAACATTCACGAGGAG
GAAGTGGAGGATCCAGATGGAGCAACTTGCTCTGGGCACACAAGGCATTTGCAATTTTATACCCTCTTGA
TGATGTCTCAGCCAGACATTCTGCCCAGTCATCAATGCCCTCTTCAATTAATATGAAAGGACACACTTGG
CATGAGATTCCAATCGTGCACAGAATATACATGAGAAGTGTGCCTTTGTCATCCCTACTTTCAAAGGCTA
AGGCCACCCTCAGTTTCTTGCATGCAACTGATGCCTTTCAAATGAAACCTTACATCTGTGTAGTCCATAG
GCAACCACAGGCAAATGTGAGGGTGAAACGCTGTGTTCTACATTGTTCTGTGTCAGTGAAGCAAGGCAGT
GCCAGCTCAGAGGGCTCTGGGGCTTCAAGGCAGGGATGCCTGGTTGTAGGTACTGCCACTTCCAGCTGGG
CAGTGAAACATAACTGCTAATACTTTCCTTACAGGCCTGCCGTCATCACTGACAAAGTAATGCCAGCTTG
TCTGCCATCCCCAGACTACATGGTCACCGCCAGGACTGAATGTTACATCACTGGCTGGGGAGAAACCCAA
GGTGAGATCAATTCCATTGCCCACGTAACAAATTGTTTTTGACCTTCAGTGCATGTTACAAAATGAGCAT
TTTGGAGATAGTTGTACAAATTCCTACCCATGAATGTGGTCTACCCACTCCTGACTTTGCCTGGACACCT
GTCTATGTCTCCATAATCAGTCTTCAAGGGACTTGGGCAAGGGGAGCGGTGCCATTTCCTTGAGTCTCTC
TCTTTTTTGTTTTCAGAATCTTTTAATTTTTTTTGTAATGATTGTATGTTTCCCTTACAACAAAAACAAA
CACCAGTAGAGGTCTTTGAGTCTCTTAATCATAATTTCAGCATTCATATTGCTTCCCCAGGTAAGTGGGG
TTTTGACCCAGCCCTCAAGTTAAGGGTGTTAGATTATTTTTCATGTGAAATTAGACAGACTGCGTTTCTA
AACATGGTGCAAAACAGTAACGACAAAAGTTGTAATTAAACTATTCTTCTTCCCAAATACCCACATGTCT
AATGTGTGTGTGAGGGTGTTAGGCAGGGGACCTGAAGCTGGGGGAGAGGCAGACAGTTCCCATGGCCCCA
AGTCTAGGATGGCATTTGGTATTGGTTGATGGGTGAGAGCAAGAGAGGGAATATTTTTGTGCATGATGTG
GTATCAGCACCTGTACTACATTTTATGGATTCCTTCTTCTCTTTGCGGTATGCCCTGACAATAATTATAT
CCGTCAGCCTTACCCCCTTGGCAGTAGGAAAACTGAAACTGTCTTAAAGTCTCAGCTCTACTTTCTCAGA
GGTGCAGGCAAGGGCACTGGGAGTCTGGGGCCCTGGAAAACTGTTCTGACTCTGCCACTTGCCAGATAGA
CCTGAACTAGACACGTTACCTCTTTGTACCACTTGGCTCTAATCCCTTATCTGTAAAACCAGCATTTTCA
AATGGTGCTTTGCACATCAGCCTTTTGCATAAGCTTTGATTTGATAAAATGTTTTTTGTGTTTTTAAAAA
GATTAAAAACCACAGGTTTAGATAATTTCAAAGTAGGCTTCCCTTTTTCTGTCATTTTCCTATTATTTTT
AAAACCTCACCTCCTTGACTCCTTGTTCCCTTTTTCTGCACTGCTGAGTCTGGGAGCACTGAGGCCAGGT
AAAAGGAAACTTGGCAAATGAGGGGCACCTATGGGTGTGGGAGGCTGCTCCTGGTGTTTGCATATTTTAA
AATTTAAATGCTACAAACCACTGTGAGTTAGGTATTATTGTTCCTATTTTACCATTGAGGAAGCTGGGGC
TCAGAGAAGGTGGAGGGTGGTACAGACAAACCTGAATTGGAACCCTGGCTCCTGCCTATGGGCTGTCAGG
ACTTAGAAAAGTCGTGAGCTCTCGCTGATTGTTTCCTCAGCTGATGTGGGCTGCAGGGCTGTTATGGGGG
AAATAATAAGAAAGTGCATCAAGTGCTGAGCACATCCTAAGCACTCCATCATGGCAGCTCCTACTACTAA
TAAAGAATAGAATTATATCTAACATGATTCTTTCTTGCAAGTGACAGAAAATCCAACTCAAATTGGATTA
AGCAAAACAAGGGAAATTCTTAGTGAGCTGCAAAGTTTTCAGGCTCACATGATGGCCCCAAATCCCAGGT
CCTCCCAATCATGGAGTAGGCACTATTTGGGGGCACAAAGGTGACATTCCCATGGCTGCAGATGCTGTGG
TGCTGTGGCTGTACCGGGAAAGAATAAGAAAGGCCACTCTCCCAATTATGTGAACAATAGTCTGCCCACT
CTGAGAAGTCAAACTTGGGTCACAGTCCTGCCCCTGAACCCATCACTGACTGGCTCTGACCTGCACCAAT
TGTTCCATGTTGGAGGTGAAGGCAAGACCCCACTAATACCCATAAGGGGCAAAAGTTAGATAGATCCTTC
AAGAGGATTATGGGAGGTAGGGCAAAAAGCTGCTGGGCAGCCAGAAAGCAAACAGAGCCTCTATGATACC
TCAACTGATGAAAGCATGAAGCTAAAATCATAAGGATCTGGGTGTGAGTTCTGGCTCTCCCATCTTCCAT
GTGACATTGGGCAGTTATTTAATCTCTTTTAGCCTCCGCTTTCTCATCTTACATATGAGATAATTGTGAG
GATTAAGATTACACATAATCATCATCATCACCGTCCACCACTACCACCATCATCCCCATCAACATCATCG
CCACCACTATCATCATTCTTACTGGCACTACCATCACCATCACCACCATTCCACCACCATCACCAATATC
ATCACTGTCAACATCATTACCACCATCACCATCACCACCACCATCATCATTACTACCACTACCACTACTA
CCACCATCACCATCACCACCATTCCACCACCATCACCAATATCATCACTCTCAACATCATCACCATCACC
ATCACCACCACCATCATCATCATTACTACCACTACCACTACTACCACCATCACCATCACCACTGTCCCAC
TACTATCAGCATGACATCACCATCACCACCACCATCATCATTACCACCGCTACTACCAACATCACCATCA
CCACAATTCTACTGCCATCACCATTAACATTACCACCACCATCATCACTATCACCATCACCACCATCATC
ACCACTGCCATTATCACTGCCACCATCATCACTATCCTCTATATTTCCTCATCTGTATTATCATTACTAC
CACCATCACTATCACCACCATCGTCACCATCATAATCACCATCAACACCATCTCCAATACCACCATCACT
GTAACCATCATCACCACCACCATGATCACTATCACCATCATCACAATGATCACTGTAACCATCATTACTA
CCCACCACCATCACCACTACTCCACCACCATCACCATTATCATTACCATCACCATTATCACCACCATCAT
CATCACCAGCACCACCATCATCACCAGCACCACCATCACCATCACCATCATTAACACCATCACTATCACC
ATTGGTTTAATCATCACCACCATCATCATAAATAAACATCACATAACCAGGGTGTAGCTGGGTGTTGACC
CCAGAGCCCACTCACTGTTTCCTCTCTCCCACCCCCATCCACACATTTCTAACCACCATCCTGCACTGGG
CTCCCAGTCTCCTCTGGTCTCACCCACATGTCCACTGAGAAAAGGATTTTCAGAACACCAACTAGACCAG
GAGGAGCCACATACATAACTCAGGCCTGCTTATCAACTTTCTACATGTTAATAATGACATCAGATCAATG
GGTGTTCTCAGCTTCTCAGAAGGAGGTCAAAATTCTCCCCCTCTCCCCTTCATGTGTCCAGACCTTCCCG
GATTTGGATGTACCAAGTGCAGAGTGGTGTTGAGGCCAAGGGGCTCATCCATGTAAGTCTCATCTGCAAT
CACTGGGCTGATCCCGTGGCCCTGTCTCCAGGGCGCCATCAGAGAGGGCTTCAATCCTCAGGTTACCTGT
GGCCCACCCTGCCCTCAGAGGTGCCATCTCTACATTGGCCACGAGATGGCAGCACATACTCATAGACTGC
ATTAATTTCCCAGCAACTCCTGGTGGGTTTTCCCTCTTATCAGGATGTTTGCCTTGCTCAGAGAGCAAAT
CTGAGAGCAGTGACACCTAACTTAACTTTCAGCAAAATATTTTGAGAAGGGTGCCCCTTTACACATCTGT
GCAGTCCAGGTGATGCATCCCATGCCCAATGCTCGGTAGTCAGGAGGAGCTTCCTCCATGCAGCTCTGCG
GAAGAGACTCTTCCACGCTGCTCATGTAAACTCCAGATTCGGTGTCAGTTTTCTGACACCGAAGACAATG
ATCTAAGTGCAGTCAAGGGCTTTGGGGAAAGCAGGAGAGAGTGCCTCAGTTCTAGCCTGTGCCATGCTTG
CAAAGTTTTGCAAAATTCTAATGAGAGCTGGGCTTGCAACATTGGAAACTTGGATTATTTGTGAGAGCAC
TGAGAAATCCCTGGGCATGTCCATCTGGAAAAACAGCATTTCCTCTGGCACTTTAGCAGAGGTTCTGTTT
CAATTTGGCGAAGGAAATTAAGCAGTTTTTCACAAAAGAAGAACTACAACGAGGAGAATTGTCCCTAGTA
TTTCTTCTCCCTAATTGTCAAGGAAGTGTAAATTAGAAAATGAATCAGGACAATTTCCACCTACTATGTT
AGCTAATATTTTAAAAATTGAATATCACAAGGGTGAGGCAAAGTAATTGTTTTCCAGTGACATTTTCCAC
TGTCACACCCTTTTAGAGAATAATTTGGCAATGTTACTGTGAGATAGAAATATGTCTATATAATTATGGG
AACTGAGACTTCAGAAAGTAATAAGGAATAAGAATGAAATTTATGAACAAACATGTGGAAGGTTGGAAGC
AAGAGTGGGGCCAACACGCATGGGGAGGAAGCATTTGGGCAGCGACTCCGCAGACCCAGACTCAAGCTGA
GCTATACAACCTCCTTACGCCTCAGTTTCCTCAACTGAAGAACAGGAATGACAAGTGCCTGTTTCATAGG
ACCGTTGTGAGGATTAAGTGAGATATACCACATTATGAGCTTGTGCCTGGAAAGGTTGATTCTTAGTAAA
TGATGACTATTCTTTTTTATTGCAATAAAATTTATACAACATAGAGTTACTATTTTAACCATTTTTGCAG
GTACCACTGAGTGGCATTCAGTACATTCACAATGGTGTGCAACCGTCACCATATTTCCAGGACATTTTTC
TCATCCCCAAAGGAAACCTCATGCCCATTAAGCAGTCACTCCTCATTAAAATATTAGTTATGAAGACTGT
AGCATTTTTTTAAAAACTCATGATATAACATTGATTGAAAAAATCAGTATAGGAAATTGTGCATTATGAT
GTAATAGTAAAAGAAGCATATAAAAATCTGAAAAAAGTATATAAAAAGAATAGCAATTGTATTTCTCAGA
CTCTCTTTACATTGTAAAAATCATTTTGATAGCTTCAAAAGAAAAGCAAAAAGTACACAAACAACAACCA
ACCCCAAAGCAGCATGACAAAGCCCAGATTGTTGAATCCAGGTCTTGGGAACATAAAATCTTATATGACA
TTTGCACTTTAATGGGTCAGAGAGTCCAGTGGCATTGGGAGCTGCCTTGTGTTCTGCAGCCTCACGGACA
GACAGGAGGTCCAGCTCCACTGCTCTGTTCTTCTGGAATTTCCTCGTGAACAAGCTTTGGCCTCAGTAAC
CATTTCTTTCATCTTTTTAAACACAGGTACCTTTGGGACTGGCCTTCTCAAGGAAGCCCAGCTCCTTGTT
ATTGAGAATGAAGTGTGCAATCACTATAAGTATATTTGTGCTGAGCATTTGGCCAGAGGCACTGACAGTT
GCCAGGTAAGAAAAGATCAATAGATCAAAGTCTTGTGCTCTCCCGTCTCAGTCTCAGTCCCTTAGACGTC
AGTCCCAAAGTGGCAAATTCAGGAAGGTTTTGTCAGTGGAAGACCCCAGTCTAAGTGTTGCTCAGAAACT
CCCCAGATCTGTCCCTGAATGCATATTCAGATCATCTAAGGAGACGTCTTGGGGCTTGAGTTCCAGATCC
ATAGCAAGGGAGCCGTAAGTGCCATAACTACCTCAGGCCACTCACCTTCCTGGTGTGTGCTGGTCACCAG
TGACTGAAGTGGTGGCTTTTCCAGTAGAGAGGAAGGTAGAGGGTACAGGACCGAGACAAATTACACACAC
TTAACAATGATGTCCAGGCTAGCCCAGTCTAAAGGAAACACCAAGTTAGGAAGCAATGCATGCAGGATTC
ACAAGGGATTATTTTTTTTCCCAGGAAAAAACTAAGTGATGTGGTTTTGTTGAATAGACTTTGCTAAGTA
CTTAAGCACTGCAGATGCTTGAGTAATATGCTCATAAGTTCCTTTCTGATTTGAATTACTGGGAAAATGT
ACATATGGATAAGAGAAGGATGGCATCCCATATTAAAAGGTTGGCAGCTTAAAGCTCACATGAATTTTCC
CCTACCTCTGTTTAGGGTGACAGTGGAGGGCCTCTGGTTTGCTTCGAGAAGGACAAATACATTTTACAAG
GAGTCACTTCTTGGGGTCTTGGCTGTGCACGCCCCAATAAGCCTGGTGTCTATGCTCGTGTTTCAAGGTT
TGTTACTTGGATTGAGGGAATGATGAGAAATAATTAATTGGACGGGAGACAGAGTGAAGCATCAACCTAC
TTAGAAGCTGAAACGTGGGTAAGGATTTAGCATGCTGGAAATAATAGACAGCAATCAAACGAAGACACTG
TTCCCAGCTACCAGCTATGCCAAACCTTGGCATTTTTGGTATTTTTGTGTATAAGCTTTTAAGGTCTGAC
TGACAAATTCTGTATTAAGGTGTCATAGCTATGACATTTGTTAAAAATAAACTCTGCACTTATTTTGATT
TGAA.

By “adenine” or “9H-Purin-6-amine” is meant a purine nucleobase with the molecular formula C₅H₅N₅, having the structure

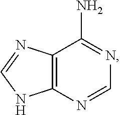

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

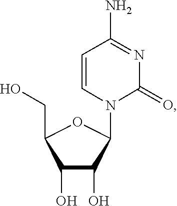

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, PCT/US2021/016827, PCT/US2022/073781, PCT/US24/34189, or PCT/US2020/028568, the full contents of which are each incorporated herein by reference in their entireties for all purposes. Further non-limiting examples of adenosine deaminases include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. Further exemplary adenosine deaminase amino acid sequences include: TadA-8e (SEQ ID NO: 470), Tad1 (SEQ ID NO: 471), Tad2 (SEQ ID NO: 472), Tad3 (SEQ ID NO: 473), Tad4 (SEQ ID NO: 474), Tad6 (SEQ ID NO: 475), Tad6-SR (SEQ ID NO: 476), TadA9 (SEQ ID NO: 477), TadA20 (SEQ ID NO: 478), Staphylococcus aureus TadA (SEQ ID NO: 479), Bacillus subtilis TadA (SEQ ID NO: 480), Salmonella typhimurium TadA (SEQ ID NO: 481), Shewanella putrefaciens (SEQ ID NO: 482), Haemophilus influenzae F3031 TadA (SEQ ID NO: 483), Caulobacter crescentus TadA (SEQ ID NO: 484), Geobacter sulfurreducens TadA (SEQ ID NO: 485), Streptococcus pyogenes TadA (SEQ ID NO: 486), Aquifex aeolicus TadA (SEQ ID NO: 487), and E. col TadA deaminase (ecTadA) (SEQ ID NO: 488).

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide. “Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrastemally. Alternatively, or concurrently, administration can be by the oral route.

By “agent” is meant any small molecule chemical compound, antibody, nucleic acid molecule, polypeptide, or fragments thereof. In an embodiment, the agent is a base editor system described herein or a component thereof.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease. In embodiments, the disease is atherosclerosis or a cardiovascular disease. Editing of an LPa gene in a subject having atherosclerosis or a cardiovascular disease may ameliorate symptoms associated with the disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and 7r-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

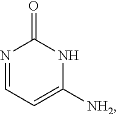

and corresponding to CAS No. 71-30-7.

By “cytidine” is meant a cytosine molecule attached to a ribose sugar via a glycosidic bond, having the structure

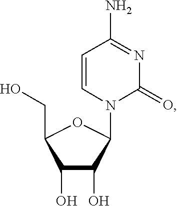

and corresponding to CAS No. 65-46-3. Its molecular formula is C₉H₁₃N₃O₅.

By “Cytidine Base Editor (CBE)” is meant a base editor comprising a cytidine deaminase. Non-limiting examples of cytidine deaminase base editor amino acid sequences include amino acid sequences for BE4max (SEQ ID NO: 553), YE1-BE4 (SEQ ID NO: 554), YE2-BE4 (SEQ ID NO: 555), YEE-BE4 (SEQ ID NO: 556), EE-BE4 (SEQ ID NO: 557), R33A-BE4 (SEQ ID NO: 558), R33A+K34A-BE4 (SEQ ID NO: 559), APOBEC3A (A3A)-BE4 (SEQ ID NO: 560), APOBEC3B (A3B)-BE4 (SEQ ID NO: 561), APOBEC3G (A3G)-BE4 (SEQ ID NO: 562), AID-BE4 (SEQ ID NO: 563), CDA-BE4 (SEQ ID NO: 564), FERNY-BE4 (SEQ ID NO: 565), evolved APOBEC3A (eA3A)-BE4 (SEQ ID NO: 566), AALN-BE4 (SEQ ID NO: 567), BE4max modified with SpCas9-NG (SEQ ID NO: 568), YE1-SpCas9-NG (YE1-NG) (SEQ ID NO: 569), YE2-SpCas9-NG (SEQ ID NO: 570), YEE-SpCas9-NG (SEQ ID NO: 571), EE-SpCas9-NG (SEQ ID NO: 572), R33A+K34A-SpCas9-NG (SEQ ID NO: 573), YE1-CP1028 (YE1-BE4-CP1028, or YE1-CP) (SEQ ID NO: 574), YE2-CP1028 (YE2-BE4-CP1028) (SEQ ID NO: 575), YEE-CP1028 (YEE-BE4-CP1028) (SEQ ID NO: 576), EE-CP1028 (EE-BE4-CP1028) (SEQ ID NO: 577), R33A+K34A-CP1028 (R33A+K34A-BE4-CP1028) (SEQ ID NO: 578), BE4max (with nickase) (SEQ ID NO: 597), BE4 (SEQ ID NO: 598), BE4 with His tag (SEQ ID NO: 599), BE4max (SEQ ID NO: 600), AncBE4max 689 (SEQ ID NO: 601), and AncBE4max 687 (SEQ ID NO: 602).

By “Cytidine Base Editor (CBE) polynucleotide” is meant a polynucleotide encoding a CBE. Non-limiting examples of polynucleotide sequences encoding cytidine deaminase base editors include those encoding BE4max (SEQ ID NO: 616), AncBE4max689 (SEQ ID NO: 617), and AncBE4max687 (SEQ ID NO: 618).

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, PCT/US2016/058344, PCT/US2020/062428, and PCT/US2019/033848, the disclosures of which are incorporated herein by reference in their entireties for all purposes.

Non-limiting examples of cytidine deaminase amino acid sequences include amino acid sequences for Rat APOBEC1 (SEQ ID NO: 579), Human APOBEC1 (SEQ ID NO: 580), Human APOBEC3 (SEQ ID NO: 581), Human APOBEC3B (SEQ ID NO: 582), Human APOBEC3G (SEQ ID NO: 583), evoAPOBEC3A(eA3A) (SEQ ID NO: 584), evoCDA (SEQ ID NO: 585), evoAPOBEC1 (SEQ ID NO: 586), YE1 (SEQ ID NO: 587), YE2 (SEQ ID NO: 588), YEE (SEQ ID NO: 589), EE (SEQ ID NO: 590), R33A (SEQ ID NO: 591), R33A+K34A (SEQ ID NO: 592), AALN (SEQ ID NO: 593), FERNY (SEQ ID NO: 594), evoFERNY (SEQ ID NO: 595), APOBEC (SEQ ID NO: 619), Anc686 APOBEC (SEQ ID NO: 620), Human APOBEC-3G D316R D317R (SEQ ID NO: 621), Human APOBEC-3G chain A (SEQ ID NO: 622), Human APOBEC3-G chain A D120R_D121R (SEQ ID NO: 623), Mouse APOBEC3 (SEQ ID NO: 624), Rat APOBEC3 (SEQ ID NO: 625), Rhesus macaque APOBEC-3G (SEQ ID NO: 626), Chimpanzee APOBEC-3G (SEQ ID NO: 627), Green Monkey APOBEC-3G (SEQ ID NO: 628), Human APOBEC-3G (SEQ ID NO: 629), Human APOBEC-3F (SEQ ID NO: 630), Human APOBEC-3B (SEQ ID NO: 631), Rat APOBEC-3B (SEQ ID NO: 632), Bovine APOBEC-3B (SEQ ID NO: 633), Chimpanzee APOBEC-3B (SEQ ID NO: 634), Gorilla APOBEC-3C (SEQ ID NO: 635), Human APOBEC-3A (SEQ ID NO: 636), Rhesus macaque APOBEC-3A (SEQ ID NO: 637), Bovine APOBEC-3A (SEQ ID NO: 638), Human APOBEC-3H (SEQ ID NO: 639), Human APOBEC-3D (SEQ ID NO: 640), Rat ABOPEC1 (SEQ ID NO: 641), Anc689 APOBEC (SEQ ID NO: 642), Anc687 APOBEC (SEQ ID NO: 643), Anc686 APOBEC (SEQ ID NO: 644), Anc655 APOBEC (SEQ ID NO: 645), and Anc733 APOBEC (SEQ ID NO: 646).

By “cytidine deaminase polynucleotide” is meant a polynucleotide encoding a cytidine deaminase. Non-limiting examples of polynucleotide sequences encoding cytidine deaminase domains include those encoding Rat APOBEC1 (SEQ ID NO: 604), Anc689 APOBEC (SEQ ID NO: 605), Anc687 APOBEC (SEQ ID NO: 606), Anc686 APOBEC (SEQ ID NO: 607), Anc655 APOBEC (SEQ ID NO: 608), Anc733 APOBEC (SEQ ID NO: 609), Rat APOBEC1 (SEQ ID NO: 610), Anc689 APOBEC (SEQ ID NO: 611), Anc687 APOBEC (SEQ ID NO: 612), Anc686 APOBEC (SEQ ID NO: 613), Anc655 APOBEC (SEQ ID NO: 614), and Anc733 APOBEC (SEQ ID NO: 615).

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

The term “detect” refers to identifying the presence, absence or amount of the analyte to be detected. In one embodiment, a sequence alteration in a polynucleotide or polypeptide is detected. In another embodiment, the presence of indels is detected. In one embodiment LPa is detected in the blood serum or a tissue of a subject.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include cardiovascular disease. Non-limiting examples of cardiovascular diseases include aortic stenosis, atherosclerotic cardiovascular disease (ASCVD), abnormal heart rhythms, or arrhythmias, aorta disease and Marfan syndrome, congenital heart disease, coronary artery disease (narrowing of the arteries), coronary heart disease (CHD), deep vein thrombosis and pulmonary embolism, heart attack, heart failure, heart muscle disease (cardiomyopathy), heart valve disease, pericardial disease, peripheral vascular disease, rheumatic heart disease, stroke, and vascular disease (blood vessel disease).

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity.

In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. Non-limiting examples of proteins having dual deaminase activity include those described in International Patent Application Publications No. WO 2024/040083 and WO 2022/204574, the disclosures of which are hereby incorporated by reference in their entireties for all purposes.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease. For example, an effective amount of a base editor system described herein is administered to a patient where the administration is associated with a decrease in serum concentrations of LPa in the patient.

The term “exonuclease” refers to a protein or polypeptide capable of removing successive nucleotides from either the 5′ or 3′ end of a polynucleotide.

The term “endonuclease” refers to a protein or polypeptide capable of catalyzing the cleavage of internal regions in a polynucleotide.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “gene” is meant a polynucleotide sequence that is transcribed as a single unit.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule. “Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In an embodiment, the level of LPa in a sample (e.g., blood, serum, plasma, tissue) is a marker of cardiovascular disease and/or atherosclerosis.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C₅-bromouridine, C₅-fluorouridine, C₅-iodouridine, C₅-propynyl-uridine, C₅-propynyl-cytidine, C₅-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
or
(SEQ ID NO: 329)
RKSGKIAAIVVKRPRKPKKKRKV.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and NI-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2cl, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2cl, Cas12c/C2c3, Cas12d/CasY, Cas12e/CasX, Cas12g, Cas12h, Cas12i, Cas12j/CasD, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October;1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine). Further non-limiting examples of nucleic acid programmable DNA binding proteins include those disclosed in International Patent Application No. PCT/US2019/047996.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “OpenCRISPR-1 polypeptide” is meant a protein with an amino acid sequence having at least about 85% amino acid sequence identity to SEQ ID NO: 463, or a fragment thereof that associates with a nucleic acid, such as a guide nucleic acid or guide polynucleotide, that guides the napDNAbp to a specific nucleic acid sequence. Further details relating to the OpenCRISPR-1 polypeptide are disclosed in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes.

By “OpenCRISPR-1 polynucleotide” is meant a nucleic acid molecule encoding an OpenCRISPR-1 polypeptide, as well as the introns, exons, 3′ untranslated regions, 5′ untranslated regions, and regulatory sequences associated with its expression, or fragments thereof. In embodiments, an OpenCRISPR-1 polynucleotide is the genomic sequence, cDNA, mRNA, or gene associated with and/or required for OpenCRISPR-1 expression. An exemplary OpenCRISPR-1 nucleotide sequence is provided at SEQ ID NO: 464.

In various embodiments, a guide RNA suitable for use in combination with an OpenCRISPR-1 polypeptide contains a scaffold having at least 85% sequence identity to a nucleotide sequence selected from the following, or fragments thereof capable of binding to an OpenCRISPR-1 polypeptide:

(SEQ ID NO: 465)
GUUUUAGAGCUGUGUUGAAAAACACAGCAAGUUAAAAUAAGGCUUUGUC
CGUAUCCAACUUGAAAAAGUGAGCACCGAUUCGGUGC;
(SEQ ID NO: 466)
GUUUUAGAGCUGGAAACAGCAAGUUAAAAUAAGGCUUUGUCCGUAUCCA
ACUUGAAAAAGUGAGCACCGAUUCGGUGC;
and
(SEQ ID NO: 467)
GUUUUAGAGCUAGAAAUAGCAAGUUAAAAUAAGGCUAGUCCGUUAUCAA
CUUGAAAAAGUGGCACCGAGUCGGUGC.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female. “Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering.

For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%. In embodiments, administration of a base editor system of the disclosure to a subject is associated with a reduction in serum levels of LPa in the subject and/or a reduced incidence of coronary heart disease in the subject.

By “reference” is meant a standard or control condition. In an embodiment, the reference is the level of LPa present in the blood of a subject not having a cardiovascular disease or atherosclerosis or having a low incidence of cardiovascular disease. In another embodiment, the reference is the level of LPa present in the blood of a subject having a cardiovascular disease or atherosclerosis or having an elevated risk for developing a cardiovascular disease. In another embodiment, a reference is an unedited cell. Another example of a reference is a base editor system lacking one or more alterations or one or more components of a base editor system of interest. In an embodiment, a reference is a base editor system containing a base editor polypeptide, or one or more polynucleotide encoding the same, and/or guide polynucleotide, or a polynucleotide encoding the same, that differs from that of a base editor system of interest. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA).

The term “single nucleotide polymorphism (SNP)” refers to a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA glycosylase
inhibitor
(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

In some embodiments, the agent inhibiting the uracil-excision repair system is a uracil stabilizing protein (USP). See, e.g., WO 2022015969 Al, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a schematic diagram showing LP(a) structure, properties, regulation, and relation to disease. Lipoprotein(a) [LP(a)]contains a lipid-rich domain, primarily cholesteryl esters, and apolipoprotein(a) [apo(a)]. Apo(a) binds to apolipoprotein B100 (apoB) via a single disulfide bond (a) at a location close the low-density lipoprotein receptor binding site of apoB (b). Apo(a) contains repeated kringle (K) structures (KIV and KV), comparable with those in plasminogen. There are 10 different subtypes of apo(a) KIV, where type 2 is present in multiple copies, resulting in a highly variable molecular mass (300-800 kDa). Apo(a) is compositionally unique among apolipoproteins with a high carbohydrate content (z28%). Proinflammatory and proatherogenic oxidized phospholipids bind to apo(a) KIV type 10 (c) and can also be found in the lipid phase. Apo(a) contains a protease domain (d) that lacks enzymatic activity. The Lp(a) concentration is heterogeneous and, to a major extent, controlled by genetics, inversely related to the copy number variation in the LPA gene. Other factors such as ethnicity and race and medical and environmental conditions also play roles in Lp(a) regulation. Lp(a) has been associated with increased risks of atherosclerosis, thrombosis, and aortic valve calcification.

FIG. 2 provides a schematic diagram showing single nucleotide polymorphisms (SNPs) reported to modify Lp(a) concentrations, including multiple variants in the KIV-2 region. The exons are numbered according to the domain that they encode (1-10: KIV-1 to KIV-10, L. leader sequence, P. protease domain, 5′: 5′ UTR, 3′: 3′ UTR). For orientation, three exons carry a superscript that reports the exon number in the genome sequence hg38. SNPs that have been associated with higher Lp(a) concentrations are shown above the gene structure. SNPs that have been associated with lower Lp(a) are shown below. SNPs that prevent protein production completely (null alleles) are underlined. FIG. 2 is taken from Coassin and Kronenberg, Atherosclerosis 349:17-35, 2022 (doi.org/10.1016/j.atherosclerosis.2022.04.003), the disclosure of which is incorporated herein by reference in its entirety for all purposes.

FIG. 3 provides a violin plot showing the distribution of serum LP(a) concentrations (mg/dL) in subjects containing 0, 1, or 2 (see x-axis) LP(a) polynucleotide variants containing the SNP chr6:160532610:A>G.

FIG. 4 provides a plot showing maximum C to T or A to G base editing frequencies measured in HEK293T cells transfected with the indicated base editor systems. In FIG. 4, the following notation is used to identify each base editor system indicated along the x-axis: the first term preceding the hyphen (“-”) indicates the guide polynucleotide of the base editor system (e.g., gRNA3512), the term “ABE” or “CBE” indicates that the base editor was an “adenosine deaminase base editor” or a “cytidine deaminase base editor,” respectively, and the last term, which follows the final underscore (“_”), indicates the PAM sequence recognized by the nucleic acid programmable DNA binding protein (napDNAbp) domain of the base editor (e.g., “NGG,” where “N” indicates A, C, T, or G). The guide polynucleotides of the base editor systems of FIG. 4 targeted the base editors to effect the introduction of a stop codon or to disrupt a splice site in an LPA polynucleotide in the HEK293T cells.

FIG. 5 provides a plot showing maximum C to T or A to G base editing frequencies measured in HEK293T cells transfected with the indicated base editor systems. In FIG. 5, the following notation is used to identify each base editor system indicated along the x-axis: the first term preceding the hyphen (“-”) indicates the guide polynucleotide of the base editor system (e.g., gRNA3420), ABE or CBE indicate that the base editor was an “adenosine deaminase base editor” or a “cytidine deaminase base editor,” respectively, and the last term, which follows the final underscore (“_”) indicates the PAM sequence recognized by the nucleic acid programmable DNA binding protein (napDNAbp) domain of the base editor (e.g., “NNNRRT,” where “N” indicates A, C, T, or G, and where “R” is A or G). The guide polynucleotides of the base editor systems of FIG. 5 targeted the base editors to effect the introduction of a stop codon or to disrupt a splice site in an LPA polynucleotide in the HEK293T cells.

### DETAILED DESCRIPTION

Provided herein are base editors and guide RNAs (gRNAs) for use in editing, modifying, or altering a target polynucleotide. In particular embodiments, a base editor of the present disclosure modifies an LPA polynucleotide. In particular embodiments, a base editor of the disclosure introduces a stop codon, protective SNP, missense mutation, or indel (e.g., a 1, 2, 3, 4, 5, 6, 7, 8, 9, or 10-nucleotide acid insertion or deletion (indel)) alteration in an LPA polynucleotide, or disrupts a splice site in the LPA polynucleotide. In embodiments, the alterations are associated with a reduction in activity or levels of an LPA polypeptide and/or polynucleotide in a cell. In some embodiments, the alterations are associated with reduced incidence of atherosclerotic cardiovascular disease (ASCVD) in a subject. In some cases, the alterations are associated with a reduction in incidence of heart attack, stroke, and/or aortic stenosis in a subject.

The various aspects and embodiments of the disclosure are based, at least in part, upon the discovery that base editing (e.g., disruption of splice acceptor or splice donor, or introduction of a stop codon, missense mutation, or indel alteration) can be used to reduce the expression of a LPA polypeptide associated with cardiovascular disease or to introduce to an LPA polypeptide single nucleotide polymorphisms (SNPs) to an LPA polynucleotide that were previously found to be associated with reduced risk of cardiovascular disease in a subject. In particular, reducing activity and/or expression of the LPA polypeptide or introducing protective SNPs to the polynucleotide encoding the LPA polypeptide in a subject diagnosed with or having a propensity to develop cardiovascular disease can be an effective treatment strategy. This reduction in activity and/or expression or introduction of an SNP can be effected using any of the base editing systems and methods provided herein. Accordingly, the disclosure features compositions and methods for editing an LPA polynucleotide. The edit to the LPA polynucleotide is associated with a reduction in expression of an LPA polypeptide in a cell of a subject and/or a reduction in symptoms associated with cardiovascular disease. In some embodiments, the edit to the LPA polynucleotide is associated with a reduction in LPA concentrations in the blood of a subject. In some instances, the LPA concentrations are reduced by about or at least about 10%, 15%, 20%, 25%, 30%, 35%, 40%, 45%, 50%, 55%, 60%, 65%, 70%, 75%, or 80%. In some embodiments, the LPA concentrations are reduced to less than 100 mg/dL, 90 mg/dL, 80 mg/dL, 70 mg/dL, 60 mg/dL, 50 mg/dL, 40 mg/dL, 30 mg/dL, or 20 mg/dL.

In embodiments, the methods of the present disclosure include disrupting splicing of an LPA polynucleotide transcript. For example, the base editors or base editor systems provided herein can be used for editing a nucleobase in the splice acceptor situated 5′ of an exon of the LPA polynucleotide. In some embodiments, the target sequence is a splice acceptor in the intron of an intron sequence adjacent to an exon of the LPA polynucleotide and is associated with a change in the splice acceptor compared to a wild-type splice acceptor. In some embodiments, the deamination of an A or C nucleobase in the splice acceptor results in disruption of splicing of the mRNA transcript during transcription. In some embodiments, the subject has or has the potential to develop cardiovascular disease.

In some instances, the methods of the present disclosure include modifying an LPA polynucleotide to introduce a stop codon, indel, or missense mutation associated with a reduction in levels or activity of the LPA polynucleotide and/or polypeptide. The alterations can be effected by a base editor system, such as those described herein.

In some cases, the methods of the present disclosure include modifying an LPA polynucleotide to introduce a single nucleotide polymorphism (SNP) known to be associated with reduced incidence of cardiovascular disease in a subject. Such SNPs may be referred to as protective alterations or variants of LPA. Non-limiting examples of such SNPs include missense variants of i significantly associated with decreased serum LPA concentrations in the United Kingdom Biobank (UKBB) (n=10) or null alleles shown previously to be associated with decreased serum LPA concentrations (n=10). In some cases, a variant may require a transversion alteration, however the corresponding transition variant may have a similar effect on serum LPA concentrations. Non-limiting examples of SNPs associated with reduced incidence of cardiovascular disease in a subject are listed in FIG. 2. Non-limiting examples of protective variants of LPA are listed in Table 8.

In some embodiments, the present disclosure provides base editors or nucleases that efficiently generate an intended mutation, such as a point mutation or indel, in a nucleic acid molecule (e.g., a nucleic acid within a genome of a subject) without generating a significant number of unintended mutations, such as unintended point mutations. In some embodiments, an intended mutation is a mutation that is generated by a specific base editor (e.g., an adenosine base editor or a cytidine base editor) bound to a guide polynucleotide (e.g., gRNA), specifically designed to generate the intended mutation. In some embodiments, the intended mutation is an adenine (A) to guanine (G) point mutation within the non-coding region of a gene. In some embodiments, the intended mutation is a cytosine (C) to thymine (T) point mutation within the non-coding region of a gene. In some embodiments, the intended mutation is a mutation of a splice acceptor in the non-coding region 5′ of an exon of a gene associated with a disease or disorder. In some instances, the intended mutation is an indel mutation. In some embodiments, the intended mutation is an adenine (A) to guanine (G) point mutation in the splice acceptor in the non-coding region 5′ of an exon of a gene associated with a disease or disorder. In some embodiments, the intended mutation is a missense mutation. The intended mutation can include the introduction of a stop codon to a polynucleotide sequence. In some embodiments, the intended mutation is a mutation that disrupts normal splicing of a complete transcript of a gene, for example, an A to G change in the splice acceptor within the non-coding region located 5′ of an exon of a disease-causing or a disease-associated gene. In some embodiments, the intended mutation is a mutation in the splice acceptor that disrupts splicing of a gene transcript and results in an alternative transcript that encodes a truncated and/or nonfunctional protein product. In some embodiments, the intended mutation is an SNP known to be associated with a reduction in incidence of cardiovascular disease in a subject.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

In some embodiments, editing of a plurality of nucleobase pairs in one or more genes using the methods provided herein results in formation of at least one intended mutation. In some embodiments, the formation of the at least one intended mutation is in the splice acceptor 5′ of an exon of a disease-associated gene and results in disruption of splicing of the mRNA transcript of a disease-associated gene. It should be appreciated that multiplex editing can be accomplished using any method or combination of methods provided herein.

The present disclosure provides methods for the treatment of a subject diagnosed with a cardiovascular disease. For example, in some embodiments, a method is provided that comprises administering to a subject having or having a propensity to develop a cardiovascular disease, an effective amount of a nucleobase editor (e.g., an adenosine deaminase base editor or a cytidine deaminase base editor) to effect an alteration in an LPA polynucleotide sequence.

### Cardiovascular Disease

Cardiovascular disease (CVD) encompasses a group of disorders of the heart and blood vessels. In many instances, CVD is associated with an increase in atherosclerosis. CVD associated diseases include coronary heart disease—a disease of the blood vessels supplying the heart muscle; cerebrovascular disease—a disease of the blood vessels supplying the brain; peripheral arterial disease—a disease of blood vessels supplying the arms and legs; rheumatic heart disease—damage to the heart muscle and heart valves from rheumatic fever, caused by streptococcal bacteria; congenital heart disease—birth defects that affect the normal development and functioning of the heart caused by malformations of the heart structure from birth; and deep vein thrombosis and pulmonary embolism—blood clots in the leg veins, which can dislodge and move to the heart and lungs. Heart attacks and strokes are usually acute events and are mainly caused by a blockage that prevents blood from flowing to the heart or brain. The most common reason for this is a build-up of fatty deposits on the inner walls of the blood vessels that supply the heart or brain. Strokes can be caused by bleeding from a blood vessel in the brain or from blood clots.

Symptoms of a heart attack include: pain or discomfort in the center of the chest; and/or pain or discomfort in the arms, the left shoulder, elbows, jaw, or back.

Symptoms of stroke include: sudden weakness of the face, arm, or leg, most often on one side of the body, numbness of the face, arm, or leg, especially on one side of the body, confusion, difficulty speaking or understanding speech, difficulty seeing with one or both eyes, difficulty walking, dizziness and/or loss of balance or coordination, severe headache with no known cause, and fainting or unconsciousness.

Rheumatic heart disease is caused by damage to the heart valves and heart muscle from the inflammation and scarring caused by rheumatic fever. Rheumatic fever is caused by an abnormal response of the body to infection with streptococcal bacteria, which usually begins as a sore throat or tonsillitis in children. Symptoms of rheumatic heart disease include: shortness of breath, fatigue, irregular heartbeats, chest pain and fainting.

Human genetic studies have indicated that plasma lipoprotein(a) (LPA) is causally associated with the risk of cardiovascular disease.

### Lipoprotein(a)

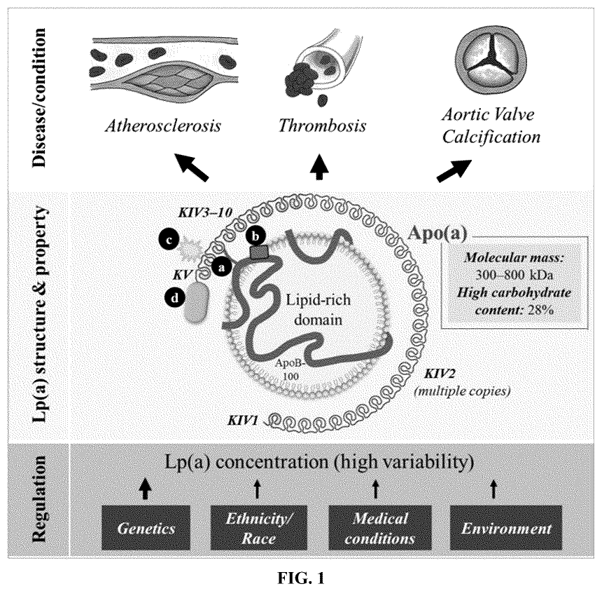

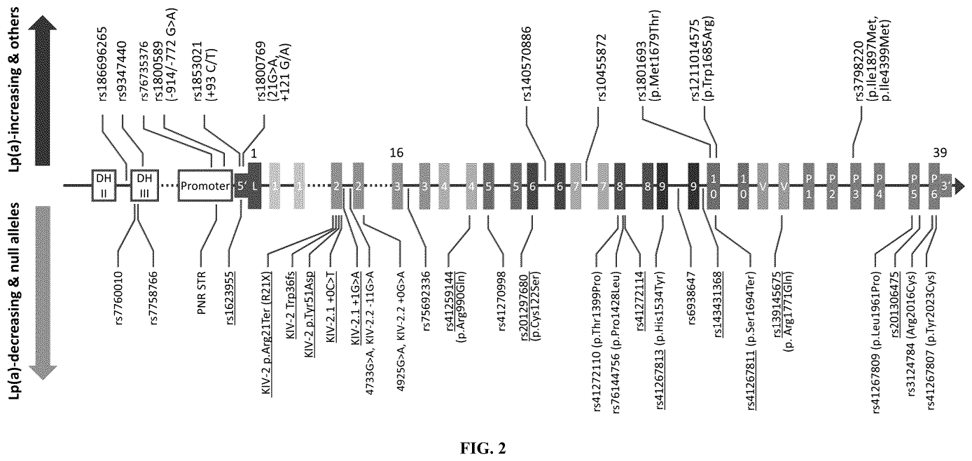

Lipoprotein(a) (LPA) is a lipoprotein that has emerged as an independent risk factor for developing vascular disease (see, e.g., FIG. 1). A number of SNPs reported to modify LPA concentrations are shown in FIG. 2. Plasma LPA levels above the common cut-off level of 300 mg/L place individuals at risk of developing heart disease, particularly if combined with other lipid and thrombogenic risk factors. In general, LPA levels have proven challenging to regulate. LPA has a high affinity for arterial walls and displays many athero-thrombogenic properties. Ongoing research continues to highlight the clinical importance of LPA in connection with cardiovascular disease. A review of LPA biology is provided by McCormick Clin Biochem Rev. 2004 February; 25(1): 69-80, which is incorporated herein in its entirety.

Elevated LPA levels increase the risk of atherosclerotic cardiovascular disease (ASCVD) in a subject. Increased lipoprotein(a) (LPA) can lead to heart attack, stroke, and aortic stenosis. About 1.4 billion people globally have elevated LP(a) levels, described as greater than about 50 mg/dL, and possibly higher in patients with established ASCVD. Elevated LPA levels can occur in patients with otherwise normal lipid levels. A UK Biobank analysis supported the current American College of Cardiology/American Heart Association cholesterol and primary prevention guidelines' recommendation to use Lp(a) as a risk-enhancing factor that, if measured, would favor statin initiation among individuals at borderline (5%-7.4%) or intermediate (7.5%-19.9%) 10-year predicted risk for ASCVD.

### Editing of Target Genes

Exemplary guide RNA sequences that can be used to produce the polynucleotide edits described above (e.g., missense mutations, stop codons, indel mutations, introduction of single nucleotide polymorphisms, splice-site disruption mutations, etc.) are listed in Tables 1A-1 to 1C-2 below. To produce the polynucleotide edits, cells (e.g., cells in or from a subject, such as hepatocytes) of a subject are contacted with one or more guide RNAs containing one or more of the spacer sequences listed in Tables 1A-2, 1B-2, or 1C-2 below, or fragments thereof, and an endonuclease or a nucleobase editor polypeptide or complex containing a nucleic acid programmable DNA binding protein (napDNAbp) and a cytidine deaminase or adenosine deaminase. In embodiments, the base editor and/or endonuclease is introduced to the cell using a polynucleotide sequence (e.g., mRNA) encoding the base editor, such as a base editor having an amino acid sequence selected from those listed in Table 2, or an amino acid sequence having at least about 85%, 90%, 95%, 99%, or greater sequence identity to a sequence listed in Table 2 or a fragment thereof (e.g., an adenosine deaminase domain or napDNAbp domain). Tables 1A-1, 1B-1, and 1C-1 below list representative guide RNA sequences that can be used in combination with the indicated editors (e.g., endonucleases or base editors). The guide polynucleotide sequences listed in Tables 1A-1, 1B-1, and 1C-1 correspond to and contain the spacer sequences listed in Tables 1A-2, 1B-2 and 1C-2, respectively, and can be used to target the target sequences listed in Tables 1A-2, 1B-2 and 1C-2 to effect the edits listed in Tables 1A-2, 1B-2 and 1C-2. In some embodiments, the gRNA comprises nucleotide analogs. These nucleotide analogs can inhibit degradation of the gRNA from cellular processes. Tables 1A-2, 1B-2, and 1C-2 list target sequences to be used for gRNAs. Further exemplary spacer sequences suitable for use in gRNA sequences for use in the methods provided herein include fragments of any of the spacers listed in Tables 1A-2, 1B-2, and 1C-2 as well as any of the spacers provided in Tables 1A-2, 1B-2, and 1C-2 modified to include an extension or truncation at the 3′ and/or 5′ end(s). In embodiments, a spacer sequence of Tables 1A-2, 1B-2, and 1C-2 can be modified to include a 1, 2, 3, 4, 5, 6, 7, 8, 9, or 10 nucleotide extension or truncation at the 3′ and/or 5′ end(s).

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

Variants of the spacer sequences of the disclosure comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

Exemplary guide polynucleotide sequences are provided below in Tables 1A-1 to 1C-2 and exemplary base editor sequences are provided in Table 2.

In the below tables, “mN” indicates a 2′-OMe modification of the nucleotide “N”, and “Ns” indicates that the nucleotide “N” is linked to the following (i.e., 3′) nucleotide by a phosphorothioate (PS).

TABLE 1A-1
Exemplary guide polynucleotide sequences for use in targeting a base editor to
introduce a single nucleotide polymorphism (SNP) to an LPA polynucleotide. In
embodiments, the SNP is associated with a reduction in incidence of cardiovascular disease
in a subject. In some cases, the SNP is associated with a reduction in serum concentrations
of LPA in a subject and/or in a reduction in risk for coronary heart disease (CHD).

| | | SEQ |
|---|---|---|
| Guide Name | Guide polynucleotide sequence | ID NO |
| CBE_NGA_20nt_4-9_006_- | mCsmAsmCsUGGCAUCAGAGGACCCCGUUUUAGAGCUAGA | 445 |
| 160577150_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_- | mCsmAsmCsUGGCAUCAGAGGACCCCGUUUUAGAGCUAGA | 446 |
| 160577151_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_- | mCsmAsmCsUGGCAUCAGAGGACCCCGUUUUAGAGCUAGA | 447 |
| 160577150_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mGsmGsmCAUCAGAGGACCCCAGAAGUUUUAGAGCUAGA | 448 |
| 9_030_−_160577145_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_- | mUsmGsmAsUACCACACUGGCAUCAGGUUUUAGAGCUAGA | 449 |
| 160577158_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_- | mGsmAsmUsACCACACUGGCAUCAGAGUUUUAGAGCUAGA | 450 |
| 160577157_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4- | mGsmGsmGsCUUUUCUCAGGUGGUGCGUUUUAGAGCUAGA | 451 |
| 9_006_+_160577276_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mGsmGsmGsCUUUUCUCAGGUGGUGCGUUUUAGAGCUAGA | 452 |
| 9_028_+_160577276_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3- | mGsmGsmGsCUUUUCUCAGGUGGUGCGUUUUAGAGCUAGA | 453 |
| 16_035_+_160577276_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20nt_4- | mAsmCsmAsGGGCUUUUCUCAGGUGGGUUUUAGAGCUAGA | 454 |
| 9_009_+_160577273_spCas9 | AAUAGCAAGUUAAAAUAAGGCUAGUCCGUUAUCAACUUGA | |
| | AAAAGUGGCACCGAGUCGGUGCmUsmUsmUSU | |
| CBE_NG_20nt_4- | mAsmCsmAsGGGCUUUUCUCAGGUGGGUUUUAGAGCUAGA | 455 |
| 9_028_+_160577273_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mAsmCsmAsGGGCUUUUCUCAGGUGGGUUUUAGAGCUAGA | 456 |
| 9_030_+_160577273_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT_21nt_3- | mAsmGsmGsGCUUUUCUCAGGUGGUGCGUUUUAGUACUCU | 457 |
| 12_015_+_160577275_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NG_20nt_4- | mCsmCsmAsCAGGGCUUUUCUCAGGUGUUUUAGAGCUAGA | 458 |
| 9_028_+_160577271_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3- | mUsmGsmGsACCACAGGGCUUUUCUCGUUUUAGAGCUAGA | 459 |
| 16_033_+_160577267_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_0nt_3- | mAsmCsmCsACAGGGCUUUUCUCAGGGUUUUAGAGCUAGA | 460 |
| 16_033_+_160577270_SpCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3-9_005_−_ | mCsmAsmCsUGGCAUCAGAGAACCACGUUUUAGAGCUAGA | 461 |
| 160589567_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3-9_027_−_ | mCsmAsmCsUGGCAUCAGAGAACCACGUUUUAGAGCUAGA | 462 |
| 160589568_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3- | mCsmAsmCsUGGCAUCAGAGAACCACGUUUUAGAGCUAGA | 647 |
| 12_019_−_160589567_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNGRRT_21nt_5- | mCsmAsmCsACUGGCAUCAGAGAACCAGUUUUAGUACUCU | 648 |
| 14_011_−_160589565_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NRCH_20nt_3- | mGsmGsmCsAUCAGAGAACCACAGAAGUUUUAGAGCUAGA | 649 |
| 9_024_−_160589562_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_13- | mUsmGsmAsCACCACACUGGCAUCAGGUUUUAGAGCUAGA | 650 |
| 16_034_−_160589575_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4-9_006_−_ | mUsmAsmCsUGCCGUAACCCUGAUGGGUUUUAGAGCUAGA | 651 |
| 160545510_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028-_ | mUsmAsmCsUGCCGUAACCCUGAUGGGUUUUAGAGCUAGA | 652 |
| 160545511_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035-_ | mUsmAsmCsUGCCGUAACCCUGAUGGGUUUUAGAGCUAGA | 653 |
| 160545510_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT 21nt_3- | mUsmGsmCsCGUAACCCUGAUGGUGACGUUUUAGUACUCU | 654 |
| 12_015_−_160545503_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NG_20nt_4-9_028_−_ | mAsmGsmUsACUGCCGUAACCCUGAUGUUUUAGAGCUAGA | 655 |
| 160545513_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mAsmCsmUsGCCGUAACCCUGAUGGUGUUUUAGAGCUAGA | 656 |
| 9_030_−_160545508_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mCsmAsmGsUACUGCCGUAACCCUGAGUUUUAGAGCUAGA | 657 |
| 160545513_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mUsmUsmUsCAGUACUGCCGUAACCCGUUUUAGAGCUAGA | 658 |
| 160545516_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_4-9_003_−_ | mGsmGsmCsUCCUUCCGAACAAGGUAGUUUUAGAGCUAGA | 659 |
| 160635116_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmGsmCsUCCUUCCGAACAAGGUAGUUUUAGAGCUAGA | 660 |
| 160635117_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mGsmGsmCsUCCUUCCGAACAAGGUAGUUUUAGAGCUAGA | 661 |
| 160635116_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_4-9_003_−_ | mGsmGsmCsUCCUUCCGAACAAGGUAGUUUUAGAGCUAGA | 662 |
| 160635116_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmGsmCsUCCUUCCGAACAAGGUAGUUUUAGAGCUAGA | 663 |
| 160635117_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mGsmGsmCsUCCUUCCGAACAAGGUAGUUUUAGAGCUAGA | 664 |
| 160635116_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4-9_006_−_ | mGsmCsmUsCCUUCCGAACAAGGUAAGUUUUAGAGCUAGA | 665 |
| 160635115_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9 028_−_ | mGsmCsmUsCCUUCCGAACAAGGUAAGUUUUAGAGCUAGA | 666 |
| 160635116_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mGsmCsmUsCCUUCCGAACAAGGUAAGUUUUAGAGCUAGA | 667 |
| 160635115_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4-9_006_−_ | mGsmCsmUsCCUUCCGAACAAGGUAAGUUUUAGAGCUAGA | 668 |
| 160635115_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmCsmUsCCUUCCGAACAAGGUAAGUUUUAGAGCUAGA | 669 |
| 160635116_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035- | mGsmCsmUsCCUUCCGAACAAGGUAAGUUUUAGAGCUAGA | 670 |
| 160635115_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNGRRT_21nt_3- | mAsmGsmGsCUCCUUCCGAACAAGGUAGUUUUAGUACUCU | 671 |
| 12_012_−_ | GUAAUGAAAAUUACAGAAUCUACUAAAACAAGGCAAAAUG | |
| 160635113_saCas9 | CCGUGUUUAUCUCGUCAACUUGUUGGCGAGAUsmUsmUsm | |
| | U | |
| CBE_NNGRRT_21nt_3- | mAsmGsmGsCUCCUUCCGAACAAGGUAGUUUUAGUACUCU | 672 |
| 12_012_−_ | GUAAUGAAAAUUACAGAAUCUACUAAAACAAGGCAAAAUG | |
| 160635113_saCas9 | CCGUGUUUAUCUCGUCAACUUGUUGGCGAGAUsmUsmUsm | |
| | U | |
| CBE_NGG_20nt_3-16_033_−_ | mCsmUsmAsGAGGCUCCUUCCGAACAGUUUUAGAGCUAGA | 673 |
| 160635121_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mCsmUsmAsGAGGCUCCUUCCGAACAGUUUUAGAGCUAGA | 674 |
| 160635121_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mUsmAsmGsAGGCUCCUUCCGAACAAGUUUUAGAGCUAGA | 675 |
| 160635121_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mCsmCsmAsAGCCUAGAGGCUCCUUCGUUUUAGAGCUAGA | 676 |
| 160635127_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_4-9_003_−_ | mUsmCsmCsACGGCUGUUUCUGAACAGUUUUAGAGCUAGA | 677 |
| 160590942_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mUsmCsmCsACGGCUGUUUCUGAACAGUUUUAGAGCUAGA | 678 |
| 160590943_spCas9 | AAUAGCAAGUUAAAAUAAGGCUAGUCCGUUAUCAACUUGA | |
| | AAAAGUGGCACCGAGUCGGUGCmUsmUsmUSU | |
| CBE_NGG_20nt_3-16_033_−_ | mUsmCsmCsACGGCUGUUUCUGAACAGUUUUAGAGCUAGA | 679 |
| 160590942_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT_21nt_3- | mAsmCsmGsUCCACGGCUGUUUCUGAAGUUUUAGUACUCU | 680 |
| 12_015_−_ | GUAAUGAAAAUUACAGAAUCUACUAAAACAAGGCAAAAUG | |
| 160590941_saCas9 | CCGUGUUUAUCUCGUCAACUUGUUGGCGAGAUsmUsmUsm | |
| | U | |
| CBE_NG_20nt_4-9_028_−_ | mCsmCsmAsCGGCUGUUUCUGAACAAGUUUUAGAGCUAGA | 681 |
| 160590942_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mGsmAsmCsGUCCACGGCUGUUUCUGGUUUUAGAGCUAGA | 682 |
| 9_030_−_160590945_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mGsmCsmGsACGUCCACGGCUGUUUCGUUUUAGAGCUAGA | 683 |
| 160590948_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mUsmAsmCsUCAUUUGGGUAGUUUUCGUUUUAGAGCUAGA | 684 |
| 9_002_+_160556021_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mUsmAsmCsUCAUUUGGGUAGUUUUCGUUUUAGAGCUAGA | 685 |
| 9_027_+_160556021_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mUsmAsmCsUCAUUUGGGUAGUUUUCGUUUUAGAGCUAGA | 686 |
| 12_018_+_160556021_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mAsmCsmUsCAUUUGGGUAGUUUUCUGUUUUAGAGCUAGA | 687 |
| 9_002_+_160556022_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mAsmCsmUsCAUUUGGGUAGUUUUCUGUUUUAGAGCUAGA | 688 |
| 9_027_+_160556022_spCas9 | AAUAGCAAGUUAAAAUAAGGCUAGUCCGUUAUCAACUUGA | |
| | AAAAGUGGCACCGAGUCGGUGCmUsmUsmUSU | |
| ABE_NGG_20nt_3- | mAsmCsmUsCAUUUGGGUAGUUUUCUGUUUUAGAGCUAGA | 689 |
| 12_018_+_160556022_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mCsmUsmCsAUUUGGGUAGUUUUCUGGUUUUAGAGCUAGA | 690 |
| 9_002_+_160556023_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mCsmUsmCsAUUUGGGUAGUUUUCUGGUUUUAGAGCUAGA | 691 |
| 9_027_+_160556023_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mCsmUsmCsAUUUGGGUAGUUUUCUGGUUUUAGAGCUAGA | 692 |
| 12_018_+_160556023_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNGRRT_21nt_5- | mAsmUsmAsCUCAUUUGGGUAGUUUUCGUUUUAGUACUCU | 693 |
| 14_011_+_160556020_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NG_20nt_3- | mUsmCsmAsUUUGGGUAGUUUUCUGGGUUUUAGAGCUAGA | 694 |
| 9_027_+_160556024_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3- | mAsmGsmUsAACAGUGGUUGCCUUCUGUUUUAGAGCUAGA | 695 |
| 9_008_+_160547881_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mAsmGsmUsAACAGUGGUUGCCUUCUGUUUUAGAGCUAGA | 696 |
| 9_027_+_160547881_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3- | mAsmGsmUsAACAGUGGUUGCCUUCUGUUUUAGAGCUAGA | 697 |
| 12_020_+_160547881_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NRCH_20nt_3- | mAsmGsmUsAACAGUGGUUGCCUUCUGUUUUAGAGCUAGA | 698 |
| 9_024_+_160547881_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_4- | mCsmUsmGsCGUCUGAGCAUUGCGUCGUUUUAGAGCUAGA | 699 |
| 9_003_+_160635188_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mCsmUsmGsCGUCUGAGCAUUGCGUCGUUUUAGAGCUAGA | 700 |
| 9_028_+_160635188_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3- | mCsmUsmGsCGUCUGAGCAUUGCGUCGUUUUAGAGCUAGA | 701 |
| 16_033_+_160635188_SpCas9 | AAUAGCAAGUUAAAAUAAGGCUAGUCCGUUAUCAACUUGA | |
| | AAAAGUGGCACCGAGUCGGUGCmUsmUsmUSU | |
| CBE_NNNRRT_21nt_3- | mCsmUsmUsCUGCGUCUGAGCAUUGCGGUUUUAGUACUCU | 702 |
| 12_015_+_160635185_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NG_20nt_4- | mCsmCsmUsUCUGCGUCUGAGCAUUGGUUUUAGAGCUAGA | 703 |
| 9_028_+_160635184_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT_21nt_3- | mAsmUsmGsUGCCUCGGUAACUCUGUCGUUUUAGUACUCU | 704 |
| 12_015_+_160599590_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21nt_3- | mUsmGsmCsCUCGGUAACUCUGUCCAUGUUUUAGUACUCU | 705 |
| 12_015_+_160599593_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21nt_3- | mCsmUsmCsGGUAACUCUGUCCAUAAUGUUUUAGUACUCU | 706 |
| 12_015_+_160599596_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NGG_20nt_3- | mCsmUsmCsGGUAACUCUGUCCAUAAGUUUUAGAGCUAGA | 707 |
| 16_033_+_160599596_SpCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mGsmGsmUsAAUGGCCAGAGUUAUCGGUUUUAGAGCUAGA | 708 |
| 12_018_−_ | AAUAGCAAGUUAAAAUAAGGCUAGUCCGUUAUCAACUUGA | |
| 160556120_spCas9 | AAAAGUGGCACCGAGUCGGUGCmUsmUsmUsU | |
| ABE_NGC_20nt_3-12_020_−_ | mGsmUsmAsAUGGCCAGAGUUAUCGAGUUUUAGAGCUAGA | 709 |
| 160556119_spCas9 | AAUAGCAAGUUAAAAUAAGGCUAGUCCGUUAUCAACUUGA | |
| | AAAAGUGGCACCGAGUCGGUGCmUsmUsmUSU | |
| ABE_NRCH_20nt_3- | mAsmAsmUsGGCCAGAGUUAUCGAGGGUUUUAGAGCUAGA | 710 |
| 9_024_−_160556116_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_13- | mAsmUsmGsGUAAUGGCCAGAGUUAUGUUUUAGAGCUAGA | 711 |
| 16_034_−_ | AAUAGCAAGUUAAAAUAAGGCUAGUCCGUUAUCAACUUGA | |
| 160556122_SpCas9 | AAAAGUGGCACCGAGUCGGUGCmUsmUsmUsU | |
| CBE_NGG_20nt_4-9_003_−_ | mCsmGsmUsCCCUCCGAAUGUUAUUCGUUUUAGAGCUAGA | 712 |
| 160600946_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mCsmGsmUsCCCUCCGAAUGUUAUUCGUUUUAGAGCUAGA | 713 |
| 160600947_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mCsmGsmUsCCCUCCGAAUGUUAUUCGUUUUAGAGCUAGA | 714 |
| 160600946_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20nt_4-9_009_−_ | mGsmUsmCsCCUCCGAAUGUUAUUCUGUUUUAGAGCUAGA | 715 |
| 160600945_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmUsmCsCCUCCGAAUGUUAUUCUGUUUUAGAGCUAGA | 716 |
| 160600946_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mGsmUsmCsCCUCCGAAUGUUAUUCUGUUUUAGAGCUAGA | 717 |
| 9_030_−_160600944_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3-12_020_−_ | mGsmUsmGsAUGGACAGAGUUAUCGAGUUUUAGAGCUAGA | 718 |
| 160585140_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NRCH_20nt_3- | mUsmGsmGsACAGAGUUAUCGAGGCAGUUUUAGAGCUAGA | 719 |
| 9_024_−_160585135_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_13- | mGsmGsmUsGAUGGACAGAGUUAUCGGUUUUAGAGCUAGA | 720 |
| 16_032_−_ | AAUAGCAAGUUAAAAUAAGGCUAGUCCGUUAUCAACUUGA | |
| 160585141_SpCas9 | AAAAGUGGCACCGAGUCGGUGCmUsmUsmUsU | |
| ABE_NGA_20nt_13- | mGsmAsmGsGUGAUGGACAGAGUUAUGUUUUAGAGCUAGA | 721 |
| 16_034_−_ | AAUAGCAAGUUAAAAUAAGGCUAGUCCGUUAUCAACUUGA | |
| 160585143_SpCas9 | AAAAGUGGCACCGAGUCGGUGCmUsmUsmUsU | |
| ABE_NGG_20nt_3-9_002_−_ | mAsmCsmAsCACUUUCUGGGCACUGCGUUUUAGAGCUAGA | 722 |
| 160664225_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3-9_027_−_ | mAsmCsmAsCACUUUCUGGGCACUGCGUUUUAGAGCUAGA | 723 |
| 160664226_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mAsmCsmAsCACUUUCUGGGCACUGCGUUUUAGAGCUAGA | 724 |
| 12_018_−_ | AAUAGCAAGUUAAAAUAAGGCUAGUCCGUUAUCAACUUGA | |
| 160664225_spCas9 | AAAAGUGGCACCGAGUCGGUGCmUsmUsmUsU | |
| ABE_NGC_20nt_3-9_008_−_ | mGsmGsmGsACACACUUUCUGGGCACGUUUUAGAGCUAGA | 725 |
| 160664228_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3-9_027_−_ | mGsmGsmGsACACACUUUCUGGGCACGUUUUAGAGCUAGA | 726 |
| 160664229_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3-12_020_−_ | mGsmGsmGsACACACUUUCUGGGCACGUUUUAGAGCUAGA | 727 |
| 160664228_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NRCH_20nt_3- | mGsmGsmGsACACACUUUCUGGGCACGUUUUAGAGCUAGA | 728 |
| 9_024_−_160664227_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mGsmGsmGsAUUGGGACACACUUUCUGUUUUAGAGCUAGA | 729 |
| 12_018_−_ | AAUAGCAAGUUAAAAUAAGGCUAGUCCGUUAUCAACUUGA | |
| 160664234_spCas9 | AAAAGUGGCACCGAGUCGGUGCmUsmUsmUsU | |
| ABE_NGC_20nt_3-12_020_−_ | mGsmGsmAsUUGGGACACACUUUCUGGUUUUAGAGCUAGA | 730 |
| 160664233_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NRCH_20nt_3- | mAsmUsmUsGGGACACACUUUCUGGGGUUUUAGAGCUAGA | 731 |
| 9_024_−_160664230_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_13- | mUsmGsmGsGAUUGGGACACACUUUCGUUUUAGAGCUAGA | 732 |
| 16_032_−_ | AAUAGCAAGUUAAAAUAAGGCUAGUCCGUUAUCAACUUGA | |
| 160664235_SpCas9 | AAAAGUGGCACCGAGUCGGUGCmUsmUsmUsU | |
| CBE_NGG_20nt_4- | mAsmUsmGsCCUCGCCCUGCUUCGGCGUUUUAGAGCUAGA | 733 |
| 9_003_+_160685562_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mAsmUsmGsCCUCGCCCUGCUUCGGCGUUUUAGAGCUAGA | 734 |
| 9_028_+_160685562_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3- | mAsmUsmGsCCUCGCCCUGCUUCGGCGUUUUAGAGCUAGA | 735 |
| 16_033_+_160685562_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20nt_4- | mUsmGsmCsCUCGCCCUGCUUCGGCUGUUUUAGAGCUAGA | 736 |
| 9_009_+_160685563_spCas9 | AAUAGCAAGUUAAAAUAAGGCUAGUCCGUUAUCAACUUGA | |
| | AAAAGUGGCACCGAGUCGGUGCmUsmUsmUSU | |
| CBE_NG_20nt_4- | mUsmGsmCsCUCGCCCUGCUUCGGCUGUUUUAGAGCUAGA | 737 |
| 9_028_+_160685563_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20nt_4- | mCsmCsmUsCGCCCUGCUUCGGCUGGGUUUUAGAGCUAGA | 738 |
| 9_009_+_160685565_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mCsmCsmUsCGCCCUGCUUCGGCUGGGUUUUAGAGCUAGA | 739 |
| 9_028_+_160685565_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mCsmCsmUsCGCCCUGCUUCGGCUGGGUUUUAGAGCUAGA | 740 |
| 9_030_+_160685565_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT_21nt_3- | mCsmUsmCsGCCCUGCUUCGGCUGGCGGUUUUAGUACUCU | 741 |
| 12_015_+_160685566_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NRCH_20nt_3- | mUsmCsmGsCCCUGCUUCGGCUGGCGGUUUUAGAGCUAGA | 742 |
| 9_030_+_160685567_spCas9 | AAUAGCAAGUUAAAAUAAGGCUAGUCCGUUAUCAACUUGA | |
| | AAAAGUGGCACCGAGUCGGUGCmUsmUsmUSU | |
| CBE_NGG_20nt_3- | mGsmGsmCsAAUGCCUCGCCCUGCUUGUUUUAGAGCUAGA | 743 |
| 16_033_+_160685558_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20nt_4-9_009_−_ | mGsmGsmUsCACUCCCACCCGAAUACGUUUUAGAGCUAGA | 744 |
| 160685456_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmGsmUsCACUCCCACCCGAAUACGUUUUAGAGCUAGA | 745 |
| 160685457_spCas9 | AAUAGCAAGUUAAAAUAAGGCUAGUCCGUUAUCAACUUGA | |
| | AAAAGUGGCACCGAGUCGGUGCmUsmUsmUSU | |
| CBE_NRCH_20nt_3- | mUsmCsmGsGGUCACUCCCACCCGAAGUUUUAGAGCUAGA | 746 |
| 9_030_−_160685458_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mAsmAsmAsAUCGGGUCACUCCCACCGUUUUAGAGCUAGA | 747 |
| 160685463_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4- | mUsmGsmGsAUUUCGGCAGUAGUUCUGUUUUAGAGCUAGA | 748 |
| 9_006_+_160601067_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mUsmGsmGsAUUUCGGCAGUAGUUCUGUUUUAGAGCUAGA | 749 |
| 9_028_+_160601067_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3- | mUsmGsmGsAUUUCGGCAGUAGUUCUGUUUUAGAGCUAGA | 750 |
| 16_035_+_160601067_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT 21nt_3- | mAsmUsmCsUGGAUUUCGGCAGUAGUUGUUUUAGUACUCU | 751 |
| 12_015_+_160601064_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NGG_20nt_3- | mGsmAsmAsCAAAGACGUACGCAUUUGUUUUAGAGCUAGA | 752 |
| 9_002_+_160599485_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mGsmAsmAsCAAAGACGUACGCAUUUGUUUUAGAGCUAGA | 753 |
| 9_027_+_160599485_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mGsmAsmAsCAAAGACGUACGCAUUUGUUUUAGAGCUAGA | 754 |
| 12_018_+_160599485_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNGRRT_21nt_5- | mAsmAsmAsGAACAAAGACGUACGCAUGUUUUAGUACUCU | 755 |
| 14_011_+_160599482_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21nt_5- | mGsmAsmAsCAAAGACGUACGCAUUUGGUUUUAGUACUCU | 756 |
| 14_014_+_160599485_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NG_20nt_3- | mAsmAsmCsAAAGACGUACGCAUUUGGUUUUAGAGCUAGA | 757 |
| 9_027_+_160599486_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mAsmAsmAsGACGUACGCAUUUGGGUGUUUUAGAGCUAGA | 758 |
| 9_027_+_160599489_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mAsmGsmAsACAAAGACGUACGCAUUGUUUUAGAGCUAGA | 759 |
| 12_018_+_160599484_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3- | mUsmAsmAsCACCAAGGACUAAUCUCGUUUUAGAGCUAGA | 760 |
| 9_008_+_160591044_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mUsmAsmAsCACCAAGGACUAAUCUCGUUUUAGAGCUAGA | 761 |
| 9_027_+_160591044_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20nt_3- | mUsmAsmAsCACCAAGGACUAAUCUCGUUUUAGAGCUAGA | 762 |
| 12_020_+_160591044_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NRCH_20nt_3- | mUsmAsmAsCACCAAGGACUAAUCUCGUUUUAGAGCUAGA | 763 |
| 9_024_+_160591044_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_13- | mGsmAsmUsCCAUGGUAUAACACCAAGUUUUAGAGCUAGA | 764 |
| 16_034_+_160591033_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_4-9_006_−_ | mGsmCsmGsAUGCUCAGACACAGAAGGUUUUAGAGCUAGA | 765 |
| 160548537_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4-9_028_−_ | mGsmCsmGsAUGCUCAGACACAGAAGGUUUUAGAGCUAGA | 766 |
| 160548538_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mGsmCsmGsAUGCUCAGACACAGAAGGUUUUAGAGCUAGA | 767 |
| 160548537_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NNNRRT_21nt_3- | mAsmUsmGsCUCAGACACAGAAGGGACGUUUUAGUACUCU | 768 |
| 12_015_−_ | GUAAUGAAAAUUACAGAAUCUACUAAAACAAGGCAAAAUG | |
| 160548530_saCas9 | CCGUGUUUAUCUCGUCAACUUGUUGGCGAGAUsmUsmUsm | |
| | U | |
| CBE_NG_20nt_4-9_028_−_ | mUsmGsmCsUCAGACACAGAAGGGACGUUUUAGAGCUAGA | 769 |
| 160548534_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mCsmGsmAsUGCUCAGACACAGAAGGGUUUUAGAGCUAGA | 770 |
| 9_030_−_160548535_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mAsmCsmGsCGAUGCUCAGACACAGAGUUUUAGAGCUAGA | 771 |
| 160548539_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mCsmGsmCsGAUGCUCAGACACAGAAGUUUUAGAGCUAGA | 772 |
| 160548538_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20nt_3-16_033_−_ | mCsmUsmCsAGACACAGAAGGGACUGGUUUUAGAGCUAGA | 773 |
| 160548531_SpCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20nt_3-16_035_−_ | mCsmUsmGsACGCGAUGCUCAGACACGUUUUAGAGCUAGA | 774 |
| 160548542_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3- | mAsmAsmCsAAGGAGCUGGGCUUCCUGUUUUAGAGCUAGA | 775 |
| 9_005_+_160532605_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3- | mAsmAsmCsAAGGAGCUGGGCUUCCUGUUUUAGAGCUAGA | 776 |
| 9_027_+_160532605_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3- | mAsmAsmCsAAGGAGCUGGGCUUCCUGUUUUAGAGCUAGA | 777 |
| 12_019_+_160532605_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3- | mCsmAsmAsGGAGCUGGGCUUCCUUGGUUUUAGAGCUAGA | 778 |
| 9 005_+_160532607_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG 20nt_3- | mCsmAsmAsGGAGCUGGGCUUCCUUGGUUUUAGAGCUAGA | 779 |
| 9 027_+_160532607_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20nt_3- | mCsmAsmAsGGAGCUGGGCUUCCUUGGUUUUAGAGCUAGA | 780 |
| 12_019_+_160532607_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_13- | mCsmAsmUsUCUCAAUAACAAGGAGCGUUUUAGAGCUAGA | 781 |
| 16_032_+_160532595_SpCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_13- | mAsmUsmUsCUCAAUAACAAGGAGCUGUUUUAGAGCUAGA | 782 |
| 16_032_+_160532596_SpCas9 | AAUAGCAAGUUAAAAUAAGGCUAGUCCGUUAUCAACUUGA | |
| | AAAAGUGGCACCGAGUCGGUGCmUsmUsmUSU | |
| CBE_NGC_20nt_4- | mUsmCsmUsUACCUGGCAACUGUCAGGUUUUAGAGCUAGA | 783 |
| 9_009_+_160532524_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mUsmCsmUsUACCUGGCAACUGUCAGGUUUUAGAGCUAGA | 784 |
| 9_028_+_160532524_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NRCH_20nt_3- | mUsmCsmUsUACCUGGCAACUGUCAGGUUUUAGAGCUAGA | 785 |
| 9_030_+_160532524_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NG_20nt_4- | mUsmUsmUsCUUACCUGGCAACUGUCGUUUUAGAGCUAGA | 786 |
| 9_028_+_160532522_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3-9_002_−_ | mGsmUsmGsUCUAUGCUCGUGUUUCAGUUUUAGAGCUAGA | 787 |
| 160531767_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NG_20nt_3-9_027_−_ | mGsmUsmGsUCUAUGCUCGUGUUUCAGUUUUAGAGCUAGA | 788 |
| 160531768_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20nt_3- | mGsmUsmGsUCUAUGCUCGUGUUUCAGUUUUAGAGCUAGA | 789 |
| 12_018_−_ | AAUAGCAAGUUAAAAUAAGGCUAGUCCGUUAUCAACUUGA | |
| 160531767_spCas9 | AAAAGUGGCACCGAGUCGGUGCmUsmUsmUsU | |
| ABE_NNNRRT_21nt_5- | mCsmUsmGsGUGUCUAUGCUCGUGUUUGUUUUAGUACUCU | 790 |
| 14_014_−_ | GUAAUGAAAAUUACAGAAUCUACUAAAACAAGGCAAAAUG | |
| 160531766_saCas9 | CCGUGUUUAUCUCGUCAACUUGUUGGCGAGAUsmUsmUsm | |
| | U | |
| ABE_NG_20nt_3-9_027_−_ | mUsmGsmUsCUAUGCUCGUGUUUCAAGUUUUAGAGCUAGA | 791 |
| 160531767_spCas9 | AAUAGCAAGUUAAAAUAAGGCUAGUCCGUUAUCAACUUGA | |
| | AAAAGUGGCACCGAGUCGGUGCmUsmUsmUsU | |
| gRNA5755-6621 | mCsmAsmCsUGGCAUCAGAGGACCCCGUUUUAGAGCUAGAAAUAGCAAG | 792 |
| | UUAAAAUAAGGCUAGUCCGUUAUCAACUUGAAAAAGUGGCACCGAGUCG | |
| | GUGCmUsmUsmUsU | |
| gRNA5756-6622 | mGsmGsmCAUCAGAGGACCCCAGAAGUUUUAGAGCUAGAAAUAGCAAG | 793 |
| | UUAAAAUAAGGCUAGUCCGUUAUCAACUUGAAAAAGUGGCACCGAGUCG | |
| | GUGCmUsmUsmUsU | |
| gRNA5757-6623 | mUsmGsmASUACCACACUGGCAUCAGGUUUUAGAGCUAGAAAUAGCAAG | 794 |
| | UUAAAAUAAGGCUAGUCCGUUAUCAACUUGAAAAAGUGGCACCGAGUCG | |
| | GUGCmUsmUsmUsU | |
| gRNA5758-6624 | mGsmAsmUsACCACACUGGCAUCAGAGUUUUAGAGCUAGAAAUAGCAAG | 795 |
| | UUAAAAUAAGGCUAGUCCGUUAUCAACUUGAAAAAGUGGCACCGAGUCG | |
| | GUGCmUsmUsmUsU | |
| gRNA5759-6625 | mGsmGsmGsCUUUUCUCAGGUGGUGCGUUUUAGAGCUAGAAAUAGCAAG | 796 |
| | UUAAAAUAAGGCUAGUCCGUUAUCAACUUGAAAAAGUGGCACCGAGUCG | |
| | GUGCmUsmUsmUsU | |
| gRNA5760-6626 | mAsmCsmAsGGGCUUUUCUCAGGUGGGUUUUAGAGCUAGAAAUAGCAAG | 797 |
| | UUAAAAUAAGGCUAGUCCGUUAUCAACUUGAAAAAGUGGCACCGAGUCG | |
| | GUGCmUsmUsmUsU | |
| gRNA5761-6627 | mAsmGsmGsGCUUUUCUCAGGUGGUGCGUUUUAGUACUCUGUAAUGAAA | 798 |
| | AUUACAGAAUCUACUAAAACAAGGCAAAAUGCCGUGUUUAUCUCGUCAA | |
| | CUUGUUGGCGAGAUsmUsmUsmU | |
| gRNA5762-6628 | mCsmCsmAsCAGGGCUUUUCUCAGGUGUUUUAGAGCUAGAAAUAGCAAG | 799 |
| | UUAAAAUAAGGCUAGUCCGUUAUCAACUUGAAAAAGUGGCACCGAGUCG | |
| | GUGCmUsmUsmUsU | |
| gRNA5763-6629 | mUsmGsmGsACCACAGGGCUUUUCUCGUUUUAGAGCUAGAAAUAGCAAG | 800 |
| | UUAAAAUAAGGCUAGUCCGUUAUCAACUUGAAAAAGUGGCACCGAGUCG | |
| | GUGCmUsmUsmUsU | |
| gRNA5764-6630 | mAsmCsmCsACAGGGCUUUUCUCAGGGUUUUAGAGCUAGAAAUAGCAAG | 801 |
| | UUAAAAUAAGGCUAGUCCGUUAUCAACUUGAAAAAGUGGCACCGAGUCG | |
| | GUGCmUsmUsmUsU | |
| gRNA5765-6631 | mCsmAsmCsUGGCAUCAGAGAACCACGUUUUAGAGCUAGAAAUAGCAAG | 802 |
| | UUAAAAUAAGGCUAGUCCGUUAUCAACUUGAAAAAGUGGCACCGAGUCG | |
| | GUGCmUsmUsmUsU | |
| gRNA5766-6632 | mCsmAsmCsACUGGCAUCAGAGAACCAGUUUUAGUACUCUGUAAUGAAA | 803 |
| | AUUACAGAAUCUACUAAAACAAGGCAAAAUGCCGUGUUUAUCUCGUCAA | |
| | CUUGUUGGCGAGAUsmUsmUsmU | |
| gRNA5767-6633 | mGsmGsmCsAUCAGAGAACCACAGAAGUUUUAGAGCUAGAAAUAGCAAG | 804 |
| | UUAAAAUAAGGCUAGUCCGUUAUCAACUUGAAAAAGUGGCACCGAGUCG | |
| | GUGCmUsmUsmUsU | |
| gRNA5768-6634 | mUsmGsmAsCACCACACUGGCAUCAGGUUUUAGAGCUAGAAAUAGCAAG | 805 |
| | UUAAAAUAAGGCUAGUCCGUUAUCAACUUGAAAAAGUGGCACCGAGUCG | |
| | GUGCmUsmUsmUsU | |
| gRNA5769-6635 | mUsmAsmCsUGCCGUAACCCUGAUGGGUUUUAGAGCUAGAAAUAGCAAG | 806 |
| | UUAAAAUAAGGCUAGUCCGUUAUCAACUUGAAAAAGUGGCACCGAGUCG | |
| | GUGCmUsmUsmUsU | |
| gRNA5770-6636 | mUsmGsmCsCGUAACCCUGAUGGUGACGUUUUAGUACUCUGUAAUGAAA | 807 |
| | AUUACAGAAUCUACUAAAACAAGGCAAAAUGCCGUGUUUAUCUCGUCAA | |
| | CUUGUUGGCGAGAUsmUsmUsmU | |
| gRNA5771-6637 | mAsmGsmUsACUGCCGUAACCCUGAUGUUUUAGAGCUAGAAAUAGCAAG | 808 |
| | UUAAAAUAAGGCUAGUCCGUUAUCAACUUGAAAAAGUGGCACCGAGUCG | |
| | GUGCmUsmUsmUsU | |
| gRNA5772-6638 | mAsmCsmUsGCCGUAACCCUGAUGGUGUUUUAGAGCUAGAAAUAGCAAG | 809 |
| | UUAAAAUAAGGCUAGUCCGUUAUCAACUUGAAAAAGUGGCACCGAGUCG | |
| | GUGCmUsmUsmUsU | |
| gRNA5773-6639 | mCsmAsmGsUACUGCCGUAACCCUGAGUUUUAGAGCUAGAAAUAGCAAG | 810 |
| | UUAAAAUAAGGCUAGUCCGUUAUCAACUUGAAAAAGUGGCACCGAGUCG | |
| | GUGCmUsmUsmUsU | |
| gRNA5774-6640 | mUsmUsmUsCAGUACUGCCGUAACCCGUUUUAGAGCUAGAAAUAGCAAG | 811 |
| | UUAAAAUAAGGCUAGUCCGUUAUCAACUUGAAAAAGUGGCACCGAGUCG | |
| | GUGCmUsmUsmUsU | |
| gRNA5775-6641 | mGsmGsmCsUCCUUCCGAACAAGGUAGUUUUAGAGCUAGAAAUAGCAAG | 812 |
| | UUAAAAUAAGGCUAGUCCGUUAUCAACUUGAAAAAGUGGCACCGAGUCG | |
| | GUGCmUsmUsmUsU | |
| gRNA5776-6642 | mGsmCsmUsCCUUCCGAACAAGGUAAGUUUUAGAGCUAGAAAUAGCAAG | 813 |
| | UUAAAAUAAGGCUAGUCCGUUAUCAACUUGAAAAAGUGGCACCGAGUCG | |
| | GUGCmUsmUsmUsU | |
| gRNA5777-6643 | mAsmGsmGsCUCCUUCCGAACAAGGUAGUUUUAGUACUCUGUAAUGAAA | 814 |
| | AUUACAGAAUCUACUAAAACAAGGCAAAAUGCCGUGUUUAUCUCGUCAA | |
| | CUUGUUGGCGAGAUsmUsmUsmU | |
| gRNA5778-6644 | mCsmUsmAsGAGGCUCCUUCCGAACAGUUUUAGAGCUAGAAAUAGCAAG | 815 |
| | UUAAAAUAAGGCUAGUCCGUUAUCAACUUGAAAAAGUGGCACCGAGUCG | |
| | GUGCmUsmUsmUsU | |
| gRNA5779-6645 | mUsmAsmGsAGGCUCCUUCCGAACAAGUUUUAGAGCUAGAAAUAGCAAG | 816 |
| | UUAAAAUAAGGCUAGUCCGUUAUCAACUUGAAAAAGUGGCACCGAGUCG | |
| | GUGCmUsmUsmUsU | |
| gRNA5780-6646 | mCsmCsmAsAGCCUAGAGGCUCCUUCGUUUUAGAGCUAGAAAUAGCAAG | 817 |
| | UUAAAAUAAGGCUAGUCCGUUAUCAACUUGAAAAAGUGGCACCGAGUCG | |
| | GUGCmUsmUsmUsU | |
| gRNA5781-6647 | mUsmCsmCsACGGCUGUUUCUGAACAGUUUUAGAGCUAGAAAUAGCAAG | 818 |
| | UUAAAAUAAGGCUAGUCCGUUAUCAACUUGAAAAAGUGGCACCGAGUCG | |
| | GUGCmUsmUsmUsU | |
| gRNA5782-6648 | mAsmCsmGsUCCACGGCUGUUUCUGAAGUUUUAGUACUCUGUAAUGAAA | 819 |
| | AUUACAGAAUCUACUAAAACAAGGCAAAAUGCCGUGUUUAUCUCGUCAA | |
| | CUUGUUGGCGAGAUsmUsmUsmU | |
| gRNA5783-6649 | mCsmCsmAsCGGCUGUUUCUGAACAAGUUUUAGAGCUAGAAAUAGCAAG | 820 |
| | UUAAAAUAAGGCUAGUCCGUUAUCAACUUGAAAAAGUGGCACCGAGUCG | |
| | GUGCmUsmUsmUsU | |
| gRNA5784-6650 | mGsmAsmCsGUCCACGGCUGUUUCUGGUUUUAGAGCUAGAAAUAGCAAG | 821 |
| | UUAAAAUAAGGCUAGUCCGUUAUCAACUUGAAAAAGUGGCACCGAGUCG | |
| | GUGCmUsmUsmUsU | |
| gRNA5785-6651 | mGsmCsmGsACGUCCACGGCUGUUUCGUUUUAGAGCUAGAAAUAGCAAG | 822 |
| | UUAAAAUAAGGCUAGUCCGUUAUCAACUUGAAAAAGUGGCACCGAGUCG | |
| | GUGCmUsmUsmUsU | |
| gRNA5786-6652 | mUsmAsmCsUCAUUUGGGUAGUUUUCGUUUUAGAGCUAGAAAUAGCAAG | 823 |
| | UUAAAAUAAGGCUAGUCCGUUAUCAACUUGAAAAAGUGGCACCGAGUCG | |
| | GUGCmUsmUsmUsU | |
| gRNA5787-6653 | mAsmCsmUsCAUUUGGGUAGUUUUCUGUUUUAGAGCUAGAAAUAGCAAG | 824 |
| | UUAAAAUAAGGCUAGUCCGUUAUCAACUUGAAAAAGUGGCACCGAGUCG | |
| | GUGCmUsmUsmUsU | |
| gRNA5788-6654 | mCsmUsmCsAUUUGGGUAGUUUUCUGGUUUUAGAGCUAGAAAUAGCAAG | 825 |
| | UUAAAAUAAGGCUAGUCCGUUAUCAACUUGAAAAAGUGGCACCGAGUCG | |
| | GUGCmUsmUsmUsU | |
| gRNA5789-6655 | mAsmUsmAsCUCAUUUGGGUAGUUUUCGUUUUAGUACUCUGUAAUGAAA | 826 |
| | AUUACAGAAUCUACUAAAACAAGGCAAAAUGCCGUGUUUAUCUCGUCAA | |
| | CUUGUUGGCGAGAUsmUsmUsmU | |
| gRNA5790-6656 | mUsmCsmAsUUUGGGUAGUUUUCUGGGUUUUAGAGCUAGAAAUAGCAAG | 827 |
| | UUAAAAUAAGGCUAGUCCGUUAUCAACUUGAAAAAGUGGCACCGAGUCG | |
| | GUGCmUsmUsmUsU | |
| gRNA5791-6657 | mAsmGsmUsAACAGUGGUUGCCUUCUGUUUUAGAGCUAGAAAUAGCAAG | 828 |
| | UUAAAAUAAGGCUAGUCCGUUAUCAACUUGAAAAAGUGGCACCGAGUCG | |
| | GUGCmUsmUsmUsU | |
| gRNA5792-6658 | mCsmUsmGsCGUCUGAGCAUUGCGUCGUUUUAGAGCUAGAAAUAGCAAG | 829 |
| | UUAAAAUAAGGCUAGUCCGUUAUCAACUUGAAAAAGUGGCACCGAGUCG | |
| | GUGCmUsmUsmUsU | |
| gRNA5793-6659 | mCsmUsmUsCUGCGUCUGAGCAUUGCGGUUUUAGUACUCUGUAAUGAAA | 830 |
| | AUUACAGAAUCUACUAAAACAAGGCAAAAUGCCGUGUUUAUCUCGUCAA | |
| | CUUGUUGGCGAGAUsmUsmUsmU | |
| gRNA5794-6660 | mCsmCsmUsUCUGCGUCUGAGCAUUGGUUUUAGAGCUAGAAAUAGCAAG | 831 |
| | UUAAAAUAAGGCUAGUCCGUUAUCAACUUGAAAAAGUGGCACCGAGUCG | |
| | GUGCmUsmUsmUsU | |
| gRNA5795-6661 | mAsmUsmGsUGCCUCGGUAACUCUGUCGUUUUAGUACUCUGUAAUGAAA | 832 |
| | AUUACAGAAUCUACUAAAACAAGGCAAAAUGCCGUGUUUAUCUCGUCAA | |
| | CUUGUUGGCGAGAUsmUsmUsmU | |
| gRNA5796-6662 | mUsmGsmCsCUCGGUAACUCUGUCCAUGUUUUAGUACUCUGUAAUGAAA | 833 |
| | AUUACAGAAUCUACUAAAACAAGGCAAAAUGCCGUGUUUAUCUCGUCAA | |
| | CUUGUUGGCGAGAUsmUsmUsmU | |
| gRNA5797-6663 | mCsmUsmCsGGUAACUCUGUCCAUAAUGUUUUAGUACUCUGUAAUGAAA | 834 |
| | AUUACAGAAUCUACUAAAACAAGGCAAAAUGCCGUGUUUAUCUCGUCAA | |
| | CUUGUUGGCGAGAUsmUsmUsmU | |
| gRNA5798-6664 | mCsmUsmCsGGUAACUCUGUCCAUAAGUUUUAGAGCUAGAAAUAGCAAG | 835 |
| | UUAAAAUAAGGCUAGUCCGUUAUCAACUUGAAAAAGUGGCACCGAGUCG | |
| | GUGCmUsmUsmUsU | |
| gRNA5799-6665 | mGsmGsmUsAAUGGCCAGAGUUAUCGGUUUUAGAGCUAGAAAUAGCAAG | 836 |
| | UUAAAAUAAGGCUAGUCCGUUAUCAACUUGAAAAAGUGGCACCGAGUCG | |
| | GUGCmUsmUsmUsU | |
| gRNA5800-6666 | mGsmUsmAsAUGGCCAGAGUUAUCGAGUUUUAGAGCUAGAAAUAGCAAG | 837 |
| | UUAAAAUAAGGCUAGUCCGUUAUCAACUUGAAAAAGUGGCACCGAGUCG | |
| | GUGCmUsmUsmUsU | |
| gRNA5801-6667 | mAsmAsmUsGGCCAGAGUUAUCGAGGGUUUUAGAGCUAGAAAUAGCAAG | 838 |
| | UUAAAAUAAGGCUAGUCCGUUAUCAACUUGAAAAAGUGGCACCGAGUCG | |
| | GUGCmUsmUsmUsU | |
| gRNA5802-6668 | mAsmUsmGsGUAAUGGCCAGAGUUAUGUUUUAGAGCUAGAAAUAGCAAG | 839 |
| | UUAAAAUAAGGCUAGUCCGUUAUCAACUUGAAAAAGUGGCACCGAGUCG | |
| | GUGCmUsmUsmUsU | |
| gRNA5803-6669 | mCsmGsmUsCCCUCCGAAUGUUAUUCGUUUUAGAGCUAGAAAUAGCAAG | 840 |
| | UUAAAAUAAGGCUAGUCCGUUAUCAACUUGAAAAAGUGGCACCGAGUCG | |
| | GUGCmUsmUsmUsU | |
| gRNA5804-6670 | mGsmUsmCsCCUCCGAAUGUUAUUCUGUUUUAGAGCUAGAAAUAGCAAG | 841 |
| | UUAAAAUAAGGCUAGUCCGUUAUCAACUUGAAAAAGUGGCACCGAGUCG | |
| | GUGCmUsmUsmUsU | |
| gRNA5805-6671 | mGsmUsmGsAUGGACAGAGUUAUCGAGUUUUAGAGCUAGAAAUAGCAAG | 842 |
| | UUAAAAUAAGGCUAGUCCGUUAUCAACUUGAAAAAGUGGCACCGAGUCG | |
| | GUGCmUsmUsmUsU | |
| gRNA5806-6672 | mUsmGsmGsACAGAGUUAUCGAGGCAGUUUUAGAGCUAGAAAUAGCAAG | 843 |
| | UUAAAAUAAGGCUAGUCCGUUAUCAACUUGAAAAAGUGGCACCGAGUCG | |
| | GUGCmUsmUsmUsU | |
| gRNA5807-6673 | mGsmGsmUsGAUGGACAGAGUUAUCGGUUUUAGAGCUAGAAAUAGCAAG | 844 |
| | UUAAAAUAAGGCUAGUCCGUUAUCAACUUGAAAAAGUGGCACCGAGUCG | |
| | GUGCmUsmUsmUsU | |
| gRNA5808-6674 | mGsmAsmGsGUGAUGGACAGAGUUAUGUUUUAGAGCUAGAAAUAGCAAG | 845 |
| | UUAAAAUAAGGCUAGUCCGUUAUCAACUUGAAAAAGUGGCACCGAGUCG | |
| | GUGCmUsmUsmUsU | |
| gRNA5809-6675 | mAsmCsmAsCACUUUCUGGGCACUGCGUUUUAGAGCUAGAAAUAGCAAG | 846 |
| | UUAAAAUAAGGCUAGUCCGUUAUCAACUUGAAAAAGUGGCACCGAGUCG | |
| | GUGCmUsmUsmUsU | |
| gRNA5810-6676 | mGsmGsmGsACACACUUUCUGGGCACGUUUUAGAGCUAGAAAUAGCAAG | 847 |
| | UUAAAAUAAGGCUAGUCCGUUAUCAACUUGAAAAAGUGGCACCGAGUCG | |
| | GUGCmUsmUsmUsU | |
| gRNA5811-6677 | mGsmGsmGsAUUGGGACACACUUUCUGUUUUAGAGCUAGAAAUAGCAAG | 848 |
| | UUAAAAUAAGGCUAGUCCGUUAUCAACUUGAAAAAGUGGCACCGAGUCG | |
| | GUGCmUsmUsmUsU | |
| gRNA5812-6678 | mGsmGsmAsUUGGGACACACUUUCUGGUUUUAGAGCUAGAAAUAGCAAG | 849 |
| | UUAAAAUAAGGCUAGUCCGUUAUCAACUUGAAAAAGUGGCACCGAGUCG | |
| | GUGCmUsmUsmUsU | |
| gRNA5813-6679 | mAsmUsmUsGGGACACACUUUCUGGGGUUUUAGAGCUAGAAAUAGCAAG | 850 |
| | UUAAAAUAAGGCUAGUCCGUUAUCAACUUGAAAAAGUGGCACCGAGUCG | |
| | GUGCmUsmUsmUsU | |
| gRNA5814-6680 | mUsmGsmGsGAUUGGGACACACUUUCGUUUUAGAGCUAGAAAUAGCAAG | 851 |
| | UUAAAAUAAGGCUAGUCCGUUAUCAACUUGAAAAAGUGGCACCGAGUCG | |
| | GUGCmUsmUsmUsU | |
| gRNA5815-6681 | mAsmUsmGsCCUCGCCCUGCUUCGGCGUUUUAGAGCUAGAAAUAGCAAG | 852 |
| | UUAAAAUAAGGCUAGUCCGUUAUCAACUUGAAAAAGUGGCACCGAGUCG | |
| | GUGCmUsmUsmUsU | |
| gRNA5816-6682 | mUsmGsmCsCUCGCCCUGCUUCGGCUGUUUUAGAGCUAGAAAUAGCAAG | 853 |
| | UUAAAAUAAGGCUAGUCCGUUAUCAACUUGAAAAAGUGGCACCGAGUCG | |
| | GUGCmUsmUsmUsU | |
| gRNA5817-6683 | mCsmCsmUsCGCCCUGCUUCGGCUGGGUUUUAGAGCUAGAAAUAGCAAG | 854 |
| | UUAAAAUAAGGCUAGUCCGUUAUCAACUUGAAAAAGUGGCACCGAGUCG | |
| | GUGCmUsmUsmUsU | |
| gRNA5818-6684 | mCsmUsmCsGCCCUGCUUCGGCUGGCGGUUUUAGUACUCUGUAAUGAAA | 855 |
| | AUUACAGAAUCUACUAAAACAAGGCAAAAUGCCGUGUUUAUCUCGUCAA | |
| | CUUGUUGGCGAGAUsmUsmUsmU | |
| gRNA5819-6685 | mUsmCsmGsCCCUGCUUCGGCUGGCGGUUUUAGAGCUAGAAAUAGCAAG | 856 |
| | UUAAAAUAAGGCUAGUCCGUUAUCAACUUGAAAAAGUGGCACCGAGUCG | |
| | GUGCmUsmUsmUsU | |
| gRNA5820-6686 | mGsmGsmCsAAUGCCUCGCCCUGCUUGUUUUAGAGCUAGAAAUAGCAAG | 857 |
| | UUAAAAUAAGGCUAGUCCGUUAUCAACUUGAAAAAGUGGCACCGAGUCG | |
| | GUGCmUsmUsmUsU | |
| gRNA5821-6687 | mGsmGsmUsCACUCCCACCCGAAUACGUUUUAGAGCUAGAAAUAGCAAG | 858 |
| | UUAAAAUAAGGCUAGUCCGUUAUCAACUUGAAAAAGUGGCACCGAGUCG | |
| | GUGCmUsmUsmUsU | |
| gRNA5822-6688 | mUsmCsmGsGGUCACUCCCACCCGAAGUUUUAGAGCUAGAAAUAGCAAG | 859 |
| | UUAAAAUAAGGCUAGUCCGUUAUCAACUUGAAAAAGUGGCACCGAGUCG | |
| | GUGCmUsmUsmUsU | |
| gRNA5823-6689 | mAsmAsmAsAUCGGGUCACUCCCACCGUUUUAGAGCUAGAAAUAGCAAG | 860 |
| | UUAAAAUAAGGCUAGUCCGUUAUCAACUUGAAAAAGUGGCACCGAGUCG | |
| | GUGCmUsmUsmUsU | |
| gRNA5824-6690 | mUsmGsmGsAUUUCGGCAGUAGUUCUGUUUUAGAGCUAGAAAUAGCAAG | 861 |
| | UUAAAAUAAGGCUAGUCCGUUAUCAACUUGAAAAAGUGGCACCGAGUCG | |
| | GUGCmUsmUsmUsU | |
| gRNA5825-6691 | mAsmUsmCsUGGAUUUCGGCAGUAGUUGUUUUAGUACUCUGUAAUGAAA | 862 |
| | AUUACAGAAUCUACUAAAACAAGGCAAAAUGCCGUGUUUAUCUCGUCAA | |
| | CUUGUUGGCGAGAUsmUsmUsmU | |
| gRNA5826-6692 | mGsmAsmAsCAAAGACGUACGCAUUUGUUUUAGAGCUAGAAAUAGCAAG | 863 |
| | UUAAAAUAAGGCUAGUCCGUUAUCAACUUGAAAAAGUGGCACCGAGUCG | |
| | GUGCmUsmUsmUsU | |
| gRNA5827-6693 | mAsmAsmAsGAACAAAGACGUACGCAUGUUUUAGUACUCUGUAAUGAAA | 864 |
| | AUUACAGAAUCUACUAAAACAAGGCAAAAUGCCGUGUUUAUCUCGUCAA | |
| | CUUGUUGGCGAGAUsmUsmUsmU | |
| gRNA5828-6694 | mGsmAsmAsCAAAGACGUACGCAUUUGGUUUUAGUACUCUGUAAUGAAA | 865 |
| | AUUACAGAAUCUACUAAAACAAGGCAAAAUGCCGUGUUUAUCUCGUCAA | |
| | CUUGUUGGCGAGAUsmUsmUsmU | |
| gRNA5829-6695 | mAsmAsmCsAAAGACGUACGCAUUUGGUUUUAGAGCUAGAAAUAGCAAG | 866 |
| | UUAAAAUAAGGCUAGUCCGUUAUCAACUUGAAAAAGUGGCACCGAGUCG | |
| | GUGCmUsmUsmUsU | |
| gRNA5830-6696 | mAsmAsmAsGACGUACGCAUUUGGGUGUUUUAGAGCUAGAAAUAGCAAG | 867 |
| | UUAAAAUAAGGCUAGUCCGUUAUCAACUUGAAAAAGUGGCACCGAGUCG | |
| | GUGCmUsmUsmUsU | |
| gRNA5831-6697 | mAsmGsmAsACAAAGACGUACGCAUUGUUUUAGAGCUAGAAAUAGCAAG | 868 |
| | UUAAAAUAAGGCUAGUCCGUUAUCAACUUGAAAAAGUGGCACCGAGUCG | |
| | GUGCmUsmUsmUsU | |
| gRNA3489-6698 | mUsmAsmAsCACCAAGGACUAAUCUCGUUUUAGAGCUAGAAAUAGCAAG | 869 |
| | UUAAAAUAAGGCUAGUCCGUUAUCAACUUGAAAAAGUGGCACCGAGUCG | |
| | GUGCmUsmUsmUsU | |
| gRNA5832-6699 | mGsmAsmUsCCAUGGUAUAACACCAAGUUUUAGAGCUAGAAAUAGCAAG | 870 |
| | UUAAAAUAAGGCUAGUCCGUUAUCAACUUGAAAAAGUGGCACCGAGUCG | |
| | GUGCmUsmUsmUsU | |
| gRNA5833-6700 | mGsmCsmGsAUGCUCAGACACAGAAGGUUUUAGAGCUAGAAAUAGCAAG | 871 |
| | UUAAAAUAAGGCUAGUCCGUUAUCAACUUGAAAAAGUGGCACCGAGUCG | |
| | GUGCmUsmUsmUsU | |
| gRNA5834-6701 | mAsmUsmGsCUCAGACACAGAAGGGACGUUUUAGUACUCUGUAAUGAAA | 872 |
| | AUUACAGAAUCUACUAAAACAAGGCAAAAUGCCGUGUUUAUCUCGUCAA | |
| | CUUGUUGGCGAGAUsmUsmUsmU | |
| gRNA4122-6702 | mUsmGsmCsUCAGACACAGAAGGGACGUUUUAGAGCUAGAAAUAGCAAG | 873 |
| | UUAAAAUAAGGCUAGUCCGUUAUCAACUUGAAAAAGUGGCACCGAGUCG | |
| | GUGCmUsmUsmUsU | |
| gRNA5835-6703 | mCsmGsmAsUGCUCAGACACAGAAGGGUUUUAGAGCUAGAAAUAGCAAG | 874 |
| | UUAAAAUAAGGCUAGUCCGUUAUCAACUUGAAAAAGUGGCACCGAGUCG | |
| | GUGCmUsmUsmUsU | |
| gRNA3431-6704 | mAsmCsmGsCGAUGCUCAGACACAGAGUUUUAGAGCUAGAAAUAGCAAG | 875 |
| | UUAAAAUAAGGCUAGUCCGUUAUCAACUUGAAAAAGUGGCACCGAGUCG | |
| | GUGCmUsmUsmUsU | |
| gRNA5836-6705 | mCsmGsmCsGAUGCUCAGACACAGAAGUUUUAGAGCUAGAAAUAGCAAG | 876 |
| | UUAAAAUAAGGCUAGUCCGUUAUCAACUUGAAAAAGUGGCACCGAGUCG | |
| | GUGCmUsmUsmUsU | |
| gRNA5837-6706 | mCsmUsmCsAGACACAGAAGGGACUGGUUUUAGAGCUAGAAAUAGCAAG | 877 |
| | UUAAAAUAAGGCUAGUCCGUUAUCAACUUGAAAAAGUGGCACCGAGUCG | |
| | GUGCmUsmUsmUsU | |
| gRNA3454-6707 | mCsmUsmGsACGCGAUGCUCAGACACGUUUUAGAGCUAGAAAUAGCAAG | 878 |
| | UUAAAAUAAGGCUAGUCCGUUAUCAACUUGAAAAAGUGGCACCGAGUCG | |
| | GUGCmUsmUsmUsU | |
| gRNA5838-6708 | mAsmAsmCsAAGGAGCUGGGCUUCCUGUUUUAGAGCUAGAAAUAGCAAG | 879 |
| | UUAAAAUAAGGCUAGUCCGUUAUCAACUUGAAAAAGUGGCACCGAGUCG | |
| | GUGCmUsmUsmUsU | |
| gRNA5839-6709 | mCsmAsmAsGGAGCUGGGCUUCCUUGGUUUUAGAGCUAGAAAUAGCAAG | 880 |
| | UUAAAAUAAGGCUAGUCCGUUAUCAACUUGAAAAAGUGGCACCGAGUCG | |
| | GUGCmUsmUsmUsU | |
| gRNA5840-6710 | mCsmAsmUsUCUCAAUAACAAGGAGCGUUUUAGAGCUAGAAAUAGCAAG | 881 |
| | UUAAAAUAAGGCUAGUCCGUUAUCAACUUGAAAAAGUGGCACCGAGUCG | |
| | GUGCmUsmUsmUsU | |
| gRNA5841-6711 | mAsmUsmUsCUCAAUAACAAGGAGCUGUUUUAGAGCUAGAAAUAGCAAG | 882 |
| | UUAAAAUAAGGCUAGUCCGUUAUCAACUUGAAAAAGUGGCACCGAGUCG | |
| | GUGCmUsmUsmUsU | |
| gRNA5842-6712 | mUsmCsmUsUACCUGGCAACUGUCAGGUUUUAGAGCUAGAAAUAGCAAG | 883 |
| | UUAAAAUAAGGCUAGUCCGUUAUCAACUUGAAAAAGUGGCACCGAGUCG | |
| | GUGCmUsmUsmUsU | |
| gRNA5843-6713 | mUsmUsmUsCUUACCUGGCAACUGUCGUUUUAGAGCUAGAAAUAGCAAG | 884 |
| | UUAAAAUAAGGCUAGUCCGUUAUCAACUUGAAAAAGUGGCACCGAGUCG | |
| | GUGCmUsmUsmUsU | |
| gRNA5844-6714 | mGsmUsmGsUCUAUGCUCGUGUUUCAGUUUUAGAGCUAGAAAUAGCAAG | 885 |
| | UUAAAAUAAGGCUAGUCCGUUAUCAACUUGAAAAAGUGGCACCGAGUCG | |
| | GUGCmUsmUsmUsU | |
| gRNA5845-6715 | mCsmUsmGsGUGUCUAUGCUCGUGUUUGUUUUAGUACUCUGUAAUGAAA | 886 |
| | AUUACAGAAUCUACUAAAACAAGGCAAAAUGCCGUGUUUAUCUCGUCAA | |
| | CUUGUUGGCGAGAUsmUsmUsmU | |
| gRNA5846-6716 | mUsmGsmUsCUAUGCUCGUGUUUCAAGUUUUAGAGCUAGAAAUAGCAAG | 887 |
| | UUAAAAUAAGGCUAGUCCGUUAUCAACUUGAAAAAGUGGCACCGAGUCG | |
| | GUGCmUsmUsmUsU | |

TABLE 1A-2
Exemplary spacer sequences and their corresponding target sequences for use
in targeting a base editor to introduce a single nucleotide polymorphism (SNP) to an LPA
polynucleotide. In embodiments, the SNP is associated with a reduction in incidence of
cardiovascular disease in a subject. In some cases, the SNP is associated with a reduction
in serum concentrations of LPA in a subject and/or in a reduction in risk for coronary heart
disease (CHD).

| | | | | PAM | PAM |
|---|---|---|---|---|---|
| | Target SNP | Base Editor | napDN | Sequence | Se- |
| Guide Name | Introduction | Name | ANbp | Family | quence |
| CBE_NGA_20nt_4-9_006_- | chr6: 1605771 | spCas9 VRQR | spCas9 | NGA | AGA |
| 160577150_spCas9 | 67: G > A | CBE | | | |
| CBE_NG_20nt_4-9_028_- | chr6: 1605771 | spCas9 NG | spCas9 | NG | AG |
| 160577151_spCas9 | 67: G > A | CBE | | | |
| CBE_NGA_20nt_3-16_035_- | chr6: 1605771 | spCas9 IBE | spCas9 | NGA | AGA |
| 160577150_spCas9 | 67: G > A | extended CBE | | | |
| CBE_NRCH_20nt_3-9_030_- | chr6: 1605771 | spCas9 NRCH | spCas9 | NRCH | AACT |
| 160577145_spCas9 | 67: G > A | CBE | | | |
| CBE_NGG_20nt_3-16_033_- | chr6: 1605771 | SpCas9 IBE | SpCas9 | NGG | AGG |
| 160577158_SpCas9 | 67: G > A | CBE | | | |
| CBE_NGA_20nt_3-16_035_- | chr6: 1605771 | spCas9 IBE | spCas9 | NGA | GGA |
| 160577157_spCas9 | 67: G > A | extended CBE | | | |
| CBE_NGA_20nt_4- | chr6: 1605772 | spCas9 VRQR | spCas9 | NGA | TGA |
| 9_006_+_160577276_spCas9 | 80: C > T | CBE | | | |
| CBE_NG_20nt_4- | chr6: 1605772 | spCas9 NG | spCas9 | NG | TG |
| 9_028_+_160577276_spCas9 | 80: C > T | CBE | | | |
| CBE_NGA_20nt_3- | chr6: 1605772 | spCas9 IBE | spCas9 | NGA | TGA |
| 16_035_+_160577276_spCas9 | 80: C > T | extended CBE | | | |
| CBE_NGC_20nt_4- | chr6: 1605772 | spCas9 NGC | spCas9 | NGC | TGC |
| 9_009_+_160577273_spCas9 | 80: C > T | CBE | | | |
| CBE_NG_20nt_4- | chr6: 1605772 | spCas9 NG | spCas9 | NG | TG |
| 9_028_+_160577273_spCas9 | 80: C > T | CBE | | | |
| CBE_NRCH_20nt_3- | chr6: 1605772 | spCas9 NRCH | spCas9 | NRCH | TGCT |
| 9_030_+_160577273_spCas9 | 80: C > T | CBE | | | |
| CBE_NNNRRT_21nt_3- | chr6: 1605772 | saCas9 KKH | saCas9 | NNNRRT | TGAAAT |
| 12_015_+_160577275_saCas9 | 80: C > T | CBE | | | |
| CBE_NG_20nt_4- | chr6: 1605772 | spCas9 NG | spCas9 | NG | GG |
| 9_028_+_160577271_spCas9 | 80: C > T | CBE | | | |
| CBE_NGG_20nt_3- | chr6: 1605772 | SpCas9 IBE | SpCas9 | NGG | AGG |
| 16_033_+_160577267_SpCas9 | 80: C > T | CBE | | | |
| CBE_NGG_20nt_3- | chr6: 1605772 | SpCas9 IBE | SpCas9 | NGG | TGG |
| 16_033_+_160577270_SpCas9 | 80: C > T | CBE | | | |
| ABE_NGA_20nt_3-9_005_- | chr6: 1605895 | spCas9 VRQR | spCas9 | NGA | AGA |
| 160589567_spCas9 | 83: T > C | ABE | | | |
| ABE_NG_20nt_3-9_027_- | chr6: 1605895 | spCas9 NG | spCas9 | NG | AG |
| 160589568_spCas9 | 83: T > C | ABE | | | |
| ABE_NGA_20nt_3-12_019_- | chr6: 1605895 | spCas9 VRQR | spCas9 | NGA | AGA |
| 160589567_spCas9 | 83: T > C | IBE | | | |
| ABE_NNGRRT_21nt_5- | chr6: 1605895 | saCas9 ABE | saCas9 | NNGRRT | CAGAAT |
| 14_011_−_160589565_saCas9 | 83: T > C | | | | |
| ABE_NRCH_20nt_3-9_024_- | chr6: 1605895 | spCas9 NRCH | spCas9 | NRCH | TACT |
| 160589562_spCas9 | 83: T > C | ABE | | | |
| ABE_NGA_20nt_13-16_034_- | chr6: 1605895 | SpCas9 IBE | SpCas9 | NGA | AGA |
| 160589575_SpCas9 | 83: T > C | expanded ABE | | | |
| CBE_NGA_20nt_4-9_006_- | chr6: 1605455 | spCas9 VRQR | spCas9 | NGA | TGA |
| 160545510_spCas9 | 27: G > A | CBE | | | |
| CBE_NG_20nt_4-9_028_- | chr6: 1605455 | spCas9 NG | spCas9 | NG | TG |
| 160545511_spCas9 | 27: G > A | CBE | | | |
| CBE_NGA_20nt_3-16_035_- | chr6: 1605455 | spCas9 IBE | spCas9 | NGA | TGA |
| 160545510_spCas9 | 27: G > A | extended CBE | | | |
| CBE_NNNRRT_21nt_3- | chr6: 1605455 | saCas9 KKH | saCas9 | NNNRRT | ATCAAT |
| 12_015_−_160545503_saCas9 | 27: G > A | CBE | | | |
| CBE_NG_20nt_4-9_028_- | chr6: 1605455 | spCas9 NG | spCas9 | NG | GG |
| 160545513_spCas9 | 27: G > A | CBE | | | |
| CBE_NRCH_20nt_3-9_030_- | chr6: 1605455 | spCas9 NRCH | spCas9 | NRCH | GACA |
| 160545508_spCas9 | 27: G > A | CBE | | | |
| CBE_NGG_20nt_3-16_033_- | chr6: 1605455 | SpCas9 IBE | SpCas9 | NGG | TGG |
| 160545513_SpCas9 | 27: G > A | CBE | | | |
| CBE_NGA_20nt_3-16_035_- | chr6: 1605455 | spCas9 IBE | spCas9 | NGA | TGA |
| 160545516_spCas9 | 27: G > A | extended CBE | | | |
| CBE_NGG_20nt_4-9_003_- | chr6: 1606351 | spCas9 CBE | spCas9 | NGG | AGG |
| 160635116_spCas9 | 34: G > A | | | | |
| CBE_NG_20nt_4-9_028_- | chr6: 1606351 | spCas9 NG | spCas9 | NG | AG |
| 160635117_spCas9 | 34: G > A | CBE | | | |
| CBE_NGG_20nt_3-16_033_- | chr6: 1606351 | SpCas9 IBE | SpCas9 | NGG | AGG |
| 160635116_SpCas9 | 34: G > A | CBE | | | |
| CBE_NGG_20nt_4-9_003_- | chr6: 1606351 | spCas9 CBE | spCas9 | NGG | AGG |
| 160635116_spCas9 | 35: G > A | | | | |
| CBE_NG_20nt_4-9_028_- | chr6: 1606351 | spCas9 NG | spCas9 | NG | AG |
| 160635117_spCas9 | 35: G > A | CBE | | | |
| CBE_NGG_20nt_3-16_033_- | chr6: 1606351 | SpCas9 IBE | SpCas9 | NGG | AGG |
| 160635116_SpCas9 | 35: G > A | CBE | | | |
| CBE_NGA_20nt_4-9_006_- | chr6: 1606351 | spCas9 VRQR | spCas9 | NGA | GGA |
| 160635115_spCas9 | 34: G > A | CBE | | | |
| CBE_NG_20nt_4-9_028_- | chr6: 1606351 | spCas9 NG | spCas9 | NG | GG |
| 160635116_spCas9 | 34: G > A | CBE | | | |
| CBE_NGA_20nt_3-16_035_- | chr6: 1606351 | spCas9 IBE | spCas9 | NGA | GGA |
| 160635115_spCas9 | 34: G > A | extended CBE | | | |
| CBE_NGA_20nt_4-9_006_- | chr6: 1606351 | spCas9 VRQR | spCas9 | NGA | GGA |
| 160635115_spCas9 | 35: G > A | CBE | | | |
| CBE_NG_20nt_4-9_028_- | chr6: 1606351 | spCas9 NG | spCas9 | NG | GG |
| 160635116_spCas9 | 35: G > A | CBE | | | |
| CBE_NGA_20nt_3-16_035_- | chr6: 1606351 | spCas9 IBE | spCas9 | NGA | GGA |
| 160635115_spCas9 | 35: G > A | extended CBE | | | |
| CBE_NNGRRT_21nt_3- | chr6: 1606351 | saCas9 CBE | saCas9 | NNGRRT | AGGAGT |
| 12_012_−_160635113_saCas9 | 34: G > A | | | | |
| CBE_NNGRRT_21nt_3- | chr6: 1606351 | saCas9 CBE | saCas9 | NNGRRT | AGGAGT |
| 12_012_−_160635113_saCas9 | 35: G > A | | | | |
| CBE_NGG_20nt_3-16_033_- | chr6: 1606351 | SpCas9 IBE | SpCas9 | NGG | AGG |
| 160635121_SpCas9 | 34: G > A | CBE | | | |
| CBE_NGG_20nt_3-16_033_- | chr6: 1606351 | SpCas9 IBE | SpCas9 | NGG | AGG |
| 160635121_SpCas9 | 35: G > A | CBE | | | |
| CBE_NG_20nt_4-9_028_- | chr6: 1606351 | spCas9 NG | spCas9 | NG | GG |
| 160635121_spCas9 | 35: G > A | CBE | | | |
| CBE_NGA_20nt_3-16_035_- | chr6: 1606351 | spCas9 IBE | spCas9 | NGA | CGA |
| 160635127_spCas9 | 35: G > A | extended CBE | | | |
| CBE_NGG_20nt_4-9_003_- | chr6: 1605909 | spCas9 CBE | spCas9 | NGG | AGG |
| 160590942_spCas9 | 61: G > A | | | | |
| CBE_NG_20nt_4-9_028_- | chr6: 1605909 | spCas9 NG | spCas9 | NG | AG |
| 160590943_spCas9 | 61: G > A | CBE | | | |
| CBE_NGG_20nt_3-16_033_- | chr6: 1605909 | SpCas9 IBE | SpCas9 | NGG | AGG |
| 160590942_SpCas9 | 61: G > A | CBE | | | |
| CBE_NNNRRT_21nt_3- | chr6: 1605909 | saCas9 KKH | saCas9 | NNNRRT | CAAGGT |
| 12_015_−_160590941_saCas9 | 61: G > A | CBE | | | |
| CBE_NG_20nt_4-9_028_- | chr6: 1605909 | spCas9 NG | spCas9 | NG | GG |
| 160590942_spCas9 | 61: G > A | CBE | | | |
| CBE_NRCH_20nt_3-9_030_- | chr6: 1605909 | spCas9 NRCH | spCas9 | NRCH | AACA |
| 160590945_spCas9 | 61: G > A | CBE | | | |
| CBE_NGA_20nt_3-16_035_- | chr6: 1605909 | spCas9 IBE | spCas9 | NGA | TGA |
| 160590948_spCas9 | 61: G > A | extended CBE | | | |
| ABE_NGG_20nt_3- | chr6: 1605560 | spCas9 ABE | spCas9 | NGG | TGG |
| 9_002_+_160556021_spCas9 | 27: A > G | | | | |
| ABE_NG_20nt_3- | chr6: 1605560 | spCas9 NG | spCas9 | NG | TG |
| 9_027_+_160556021_spCas9 | 27: A > G | ABE | | | |
| ABE_NGG_20nt_3- | chr6: 1605560 | spCas9 IBE | spCas9 | NGG | TGG |
| 12_018_+_160556021_spCas9 | 27: A > G | | | | |
| ABE_NGG_20nt_3- | chr6: 1605560 | spCas9 ABE | spCas9 | NGG | GGG |
| 9_002_+_160556022_spCas9 | 27: A > G | | | | |
| ABE_NG_20nt_3- | chr6: 1605560 | spCas9 NG | spCas9 | NG | GG |
| 9_027_+_160556022_spCas9 | 27: A > G | ABE | | | |
| ABE_NGG_20nt_3- | chr6: 1605560 | spCas9 IBE | spCas9 | NGG | GGG |
| 12_018_+_160556022_spCas9 | 27: A > G | | | | |
| ABE_NGG_20nt_3- | chr6: 1605560 | spCas9 ABE | spCas9 | NGG | GGG |
| 9_002_+_160556023_spCas9 | 27: A > G | | | | |
| ABE_NG_20nt_3- | chr6: 1605560 | spCas9 NG | spCas9 | NG | GG |
| 9_027_+_160556023_spCas9 | 27: A > G | ABE | | | |
| ABE_NGG_20nt_3- | chr6: 1605560 | spCas9 IBE | spCas9 | NGG | GGG |
| 12_018_+_160556023_spCas9 | 27: A > G | | | | |
| ABE_NNGRRT_21nt_5- | chr6: 1605560 | saCas9 ABE | saCas9 | NNGRRT | TGGGGT |
| 14_011_+_160556020_saCas9 | 27: A > G | | | | |
| ABE_NG_20nt_3- | chr6: 1605560 | spCas9 NG | spCas9 | NG | GG |
| 9_027_+_160556024_spCas9 | 27: A > G | ABE | | | |
| ABE_NGC_20nt_3- | chr6: 1605478 | spCas9 NGC | spCas9 | NGC | TGC |
| 9_008_+_160547881_spCas9 | 86: A > G | ABE | | | |
| ABE_NG_20nt_3- | chr6: 1605478 | spCas9 NG | spCas9 | NG | TG |
| 9_027_+_160547881_spCas9 | 86: A > G | ABE | | | |
| ABE_NGC_20nt_3- | chr6: 1605478 | spCas9 NGC | spCas9 | NGC | TGC |
| 12_020_+_160547881_spCas9 | 86: A > G | IBE | | | |
| ABE_NRCH_20nt_3- | chr6: 1605478 | spCas9 NRCH | spCas9 | NRCH | TGCC |
| 9_024_+_160547881_spCas9 | 86: A > G | ABE | | | |
| CBE_NGG_20nt_4- | chr6: 1606351 | spCas9 CBE | spCas9 | NGG | AGG |
| 9_003_+_160635188_spCas9 | 92: C > T | | | | |
| CBE_NG_20nt_4- | chr6: 1606351 | spCas9 NG | spCas9 | NG | AG |
| 9_028_+_160635188_spCas9 | 92: C > T | CBE | | | |
| CBE_NGG_20nt_3- | chr6: 1606351 | SpCas9 IBE | SpCas9 | NGG | AGG |
| 16_033_+_160635188_SpCas9 | 92: C > T | CBE | | | |
| CBE_NNNRRT_21nt_3- | chr6: 1606351 | saCas9 KKH | saCas9 | NNNRRT | TCAGGT |
| 12_015_+_160635185_saCas9 | 92: C > T | CBE | | | |
| CBE_NG_20nt_4- | chr6: 1606351 | spCas9 NG | spCas9 | NG | CG |
| 9_028_+_160635184_spCas9 | 92: C > T | CBE | | | |
| CBE_NNNRRT_21nt_3- | chr6: 1605995 | saCas9 KKH | saCas9 | NNNRRT | CATAAT |
| 12_015_+_160599590_saCas9 | 99: C > T | CBE | | | |
| CBE_NNNRRT_21nt_3- | chr6: 1605995 | saCas9 KKH | saCas9 | NNNRRT | AATGGT |
| 12_015_+_160599593_saCas9 | 99: C > T | CBE | | | |
| CBE_NNNRRT_21nt_3- | chr6: 1605995 | saCas9 KKH | saCas9 | NNNRRT | GGTAGT |
| 12_015_+_160599596_saCas9 | 99: C > T | CBE | | | |
| CBE_NGG_20nt_3- | chr6: 1605995 | SpCas9 IBE | SpCas9 | NGG | TGG |
| 16_033_+_160599596_SpCas9 | 99: C > T | CBE | | | |
| ABE_NGG_20nt_3-12_018_- | chr6: 1605561 | spCas9 IBE | spCas9 | NGG | AGG |
| 160556120_spCas9 | 33: T > C | | | | |
| ABE_NGC_20nt_3-12_020_- | chr6: 1605561 | spCas9 NGC | spCas9 | NGC | GGC |
| 160556119_spCas9 | 33: T > C | IBE | | | |
| ABE_NRCH_20nt_3-9_024_- | chr6: 1605561 | spCas9 NRCH | spCas9 | NRCH | CACA |
| 160556116_spCas9 | 33: T > C | ABE | | | |
| ABE_NGA_20nt_13-16_034_- | chr6: 1605561 | SpCas9 IBE | SpCas9 | NGA | CGA |
| 160556122_SpCas9 | 33: T > C | expanded ABE | | | |
| CBE_NGG_20nt_4-9_003_- | chr6: 1606009 | spCas9 CBE | spCas9 | NGG | TGG |
| 160600946_spCas9 | 61: G > A | | | | |
| CBE_NG_20nt_4-9_028_- | chr6: 1606009 | spCas9 NG | spCas9 | NG | TG |
| 160600947_spCas9 | 61: G > A | CBE | | | |
| CBE_NGG_20nt_3-16_033_- | chr6: 1606009 | SpCas9 IBE | SpCas9 | NGG | TGG |
| 160600946_SpCas9 | 61: G > A | CBE | | | |
| CBE_NGC_20nt_4-9_009_- | chr6: 1606009 | spCas9 NGC | spCas9 | NGC | GGC |
| 160600945_spCas9 | 61: G > A | CBE | | | |
| CBE_NG_20nt_4-9_028_- | chr6: 1606009 | spCas9 NG | spCas9 | NG | GG |
| 160600946_spCas9 | 61: G > A | CBE | | | |
| CBE_NRCH_20nt_3-9_030_- | chr6: 1606009 | spCas9 NRCH | spCas9 | NRCH | GGCT |
| 160600944_spCas9 | 61: G > A | CBE | | | |
| ABE_NGC_20nt_3-12_020_- | chr6: 1605851 | spCas9 NGC | spCas9 | NGC | GGC |
| 160585140_spCas9 | 52: T > C | IBE | | | |
| ABE_NRCH_20nt_3-9_024_- | chr6: 1605851 | spCas9 NRCH | spCas9 | NRCH | CACT |
| 160585135_spCas9 | 52: T > C | ABE | | | |
| ABE_NGG_20nt_13-16_032_- | chr6: 1605851 | SpCas9 | SpCas9 | NGG | AGG |
| 160585141_SpCas9 | 52: T > C | IBE_expanded | | | |
| | | ABE | | | |
| ABE_NGA_20nt_13-16_034_- | chr6: 1605851 | SpCas9 IBE | SpCas9 | NGA | CGA |
| 160585143_SpCas9 | 52: T > C | expanded ABE | | | |
| ABE_NGG_20nt_3-9_002_- | chr6: 1606642 | spCas9 ABE | spCas9 | NGG | TGG |
| 160664225_spCas9 | 46: T > C | | | | |
| ABE_NG_20nt_3-9_027_- | chr6: 1606642 | spCas9 NG | spCas9 | NG | TG |
| 160664226_spCas9 | 46: T > C | ABE | | | |
| ABE_NGG_20nt_3-12_018_- | chr6: 1606642 | spCas9 IBE | spCas9 | NGG | TGG |
| 160664225_spCas9 | 46: T > C | | | | |
| ABE_NGC_20nt_3-9_008_- | chr6: 1606642 | spCas9 NGC | spCas9 | NGC | TGC |
| 160664228_spCas9 | 46: T > C | ABE | | | |
| ABE_NG_20nt_3-9_027_- | chr6: 1606642 | spCas9 NG | spCas9 | NG | TG |
| 160664229_spCas9 | 46: T > C | ABE | | | |
| ABE_NGC_20nt_3-12_020_- | chr6: 1606642 | spCas9 NGC | spCas9 | NGC | TGC |
| 160664228_spCas9 | 46: T > C | IBE | | | |
| ABE_NRCH_20nt_3-9_024_- | chr6: 1606642 | spCas9 NRCH | spCas9 | NRCH | TGCT |
| 160664227_spCas9 | 46: T > C | ABE | | | |
| ABE_NGG_20nt_3-12_018_- | chr6: 1606642 | spCas9 IBE | spCas9 | NGG | GGG |
| 160664234_spCas9 | 46: T > C | | | | |
| ABE_NGC_20nt_3-12_020_- | chr6: 1606642 | spCas9 NGC | spCas9 | NGC | GGC |
| 160664233_spCas9 | 46: T > C | IBE | | | |
| ABE_NRCH_20nt_3-9_024_- | chr6: 1606642 | spCas9 NRCH | spCas9 | NRCH | CACT |
| 160664230_spCas9 | 46: T > C | ABE | | | |
| ABE_NGG_20nt_13-16_032_- | chr6: 1606642 | SpCas9 | SpCas9 | NGG | TGG |
| 160664235_SpCas9 | 46: T > C | IBE_expanded | | | |
| | | ABE | | | |
| CBE_NGG_20nt_4- | chr6: 1606855 | spCas9 CBE | spCas9 | NGG | TGG |
| 9_003_+_160685562_spCas9 | 71: C > T | | | | |
| CBE_NG_20nt_4- | chr6: 1606855 | spCas9 NG | spCas9 | NG | TG |
| 9_028_+_160685562_spCas9 | 71: C > T | CBE | | | |
| CBE_NGG_20nt_3- | chr6: 1606855 | SpCas9 IBE | SpCas9 | NGG | TGG |
| 16_033_+_160685562_SpCas9 | 71: C > T | CBE | | | |
| CBE_NGC_20nt_4- | chr6: 1606855 | spCas9 NGC | spCas9 | NGC | GGC |
| 9_009_+_160685563_spCas9 | 71: C > T | CBE | | | |
| CBE_NG_20nt_4- | chr6: 1606855 | spCas9 NG | spCas9 | NG | GG |
| 9_028_+_160685563_spCas9 | 71: C > T | CBE | | | |
| CBE_NGC_20nt_4- | chr6: 1606855 | spCas9 NGC | spCas9 | NGC | CGC |
| 9_009_+_160685565_spCas9 | 71: C > T | CBE | | | |
| CBE_NG_20nt_4- | chr6: 1606855 | spCas9 NG | spCas9 | NG | CG |
| 9_028_+_160685565_spCas9 | 71: C > T | CBE | | | |
| CBE_NRCH_20nt_3- | chr6: 1606855 | spCas9 NRCH | spCas9 | NRCH | CGCA |
| 9_030_+_160685565_spCas9 | 71: C > T | CBE | | | |
| CBE_NNNRRT_21nt_3- | chr6: 1606855 | saCas9 KKH | saCas9 | NNNRRT | CACAGT |
| 12_015_+_160685566_saCas9 | 71: C > T | CBE | | | |
| CBE_NRCH_20nt_3- | chr6: 1606855 | spCas9 NRCH | spCas9 | NRCH | CACA |
| 9_030_+_160685567_spCas9 | 71: C > T | CBE | | | |
| CBE_NGG_20nt_3- | chr6: 1606855 | SpCas9 IBE | SpCas9 | NGG | CGG |
| 16_033_+_160685558_SpCas9 | 71: C > T | CBE | | | |
| CBE_NGC_20nt_4-9_009_- | chr6: 1606854 | spCas9 NGC | spCas9 | NGC | TGC |
| 160685456_spCas9 | 76: G > A | CBE | | | |
| CBE_NG_20nt_4-9_028_- | chr6: 1606854 | spCas9 NG | spCas9 | NG | TG |
| 160685457_spCas9 | 76: G > A | CBE | | | |
| CBE_NRCH_20nt_3-9_030_- | chr6: 1606854 | spCas9 NRCH | spCas9 | NRCH | TACT |
| 160685458_spCas9 | 76: G > A | CBE | | | |
| CBE_NGA_20nt_3-16_035_- | chr6: 1606854 | spCas9 IBE | spCas9 | NGA | CGA |
| 160685463_spCas9 | 76: G > A | extended CBE | | | |
| CBE_NGA_20nt_4- | chr6: 1606010 | spCas9 VRQR | spCas9 | NGA | TGA |
| 9_006_+_160601067_spCas9 | 75: C > T | CBE | | | |
| CBE_NG_20nt_4- | chr6: 1606010 | spCas9 NG | spCas9 | NG | TG |
| 9_028_+_160601067_spCas9 | 75: C > T | CBE | | | |
| CBE_NGA_20nt_3- | chr6: 1606010 | spCas9 IBE | spCas9 | NGA | TGA |
| 16_035_+_160601067_spCas9 | 75: C > T | extended CBE | | | |
| CBE_NNNRRT_21nt_3- | chr6: 1606010 | saCas9 KKH | saCas9 | NNNRRT | CTTGAT |
| 12_015_+_160601064_saCas9 | 75: C > T | CBE | | | |
| ABE_NGG_20nt_3- | chr6: 1605994 | spCas9 ABE | spCas9 | NGG | GGG |
| 9_002_+_160599485_spCas9 | 94: A > G | | | | |
| ABE_NG_20nt_3- | chr6: 1605994 | spCas9 NG | spCas9 | NG | GG |
| 9_027_+_160599485_spCas9 | 94: A > G | ABE | | | |
| ABE_NGG_20nt_3- | chr6: 1605994 | spCas9 IBE | spCas9 | NGG | GGG |
| 12_018_+_160599485_spCas9 | 94: A > G | | | | |
| ABE_NNGRRT_21nt_5- | chr6: 1605994 | saCas9 ABE | saCas9 | NNGRRT | TTGGGT |
| 14_011_+_160599482_saCas9 | 94: A > G | | | | |
| ABE_NNNRRT_21nt_5- | chr6: 1605994 | saCas9 KKH | saCas9 | NNNRRT | GGTAGT |
| 14_014_+_160599485_saCas9 | 94: A > G | ABE | | | |
| ABE_NG_20nt_3- | chr6: 1605994 | spCas9 NG | spCas9 | NG | GG |
| 9_027_+_160599486_spCas9 | 94: A > G | ABE | | | |
| ABE_NG_20nt_3- | chr6: 1605994 | spCas9 NG | spCas9 | NG | AG |
| 9_027_+_160599489_spCas9 | 94: A > G | ABE | | | |
| ABE_NGG_20nt_3- | chr6: 1605994 | spCas9 IBE | spCas9 | NGG | TGG |
| 12_018_+_160599484_spCas9 | 94: A > G | | | | |
| ABE_NGC_20nt_3- | chr6: 1605910 | spCas9 NGC | spCas9 | NGC | AGC |
| 9_008_+_160591044_spCas9 | 49: A > G | ABE | | | |
| ABE_NG_20nt_3- | chr6: 1605910 | spCas9 NG | spCas9 | NG | AG |
| 9_027_+_160591044_spCas9 | 49: A > G | ABE | | | |
| ABE_NGC_20nt_3- | chr6: 1605910 | spCas9 NGC | spCas9 | NGC | AGC |
| 12_020_+_160591044_spCas9 | 49: A > G | IBE | | | |
| ABE_NRCH_20nt_3- | chr6: 1605910 | spCas9 NRCH | spCas9 | NRCH | AGCA |
| 9_024_+_160591044_spCas9 | 49: A > G | ABE | | | |
| ABE_NGA_20nt_13- | chr6: 1605910 | SpCas9 IBE | SpCas9 | NGA | GGA |
| 16_034_+_160591033_SpCas9 | 49: A > G | expanded ABE | | | |
| CBE_NGA_20nt_4-9_006_- | chr6: 1605485 | spCas9 VRQR | spCas9 | NGA | GGA |
| 160548537_spCas9 | 52: G > A | CBE | | | |
| CBE_NG_20nt_4-9_028_- | chr6: 1605485 | spCas9 NG | spCas9 | NG | GG |
| 160548538_spCas9 | 52: G > A | CBE | | | |
| CBE_NGA_20nt_3-16_035_- | chr6: 1605485 | spCas9 IBE | spCas9 | NGA | GGA |
| 160548537_spCas9 | 52: G > A | extended CBE | | | |
| CBE_NNNRRT_21nt_3- | chr6: 1605485 | saCas9 KKH | saCas9 | NNNRRT | TGTGGT |
| 12_015_−_160548530_saCas9 | 52: G > A | CBE | | | |
| CBE_NG_20nt_4-9_028_- | chr6: 1605485 | spCas9 NG | spCas9 | NG | TG |
| 160548534_spCas9 | 52: G > A | CBE | | | |
| CBE_NRCH_20nt_3-9_030_- | chr6: 1605485 | spCas9 NRCH | spCas9 | NRCH | GACT |
| 160548535_spCas9 | 52: G > A | CBE | | | |
| CBE_NGG_20nt_3-16_033_- | chr6: 1605485 | SpCas9 IBE | SpCas9 | NGG | AGG |
| 160548539_SpCas9 | 52: G > A | CBE | | | |
| CBE_NGG_20nt_3-16_033_- | chr6: 1605485 | SpCas9 IBE | SpCas9 | NGG | GGG |
| 160548538_SpCas9 | 52: G > A | CBE | | | |
| CBE_NGG_20nt_3-16_033_- | chr6: 1605485 | SpCas9 IBE | SpCas9 | NGG | TGG |
| 160548531_SpCas9 | 52: G > A | CBE | | | |
| CBE_NGA_20nt_3-16_035_- | chr6: 1605485 | spCas9 IBE | spCas9 | NGA | AGA |
| 160548542_spCas9 | 52: G > A | extended CBE | | | |
| ABE_NGA_20nt_3- | chr6: 1605326 | spCas9 VRQR | spCas9 | NGA | TGA |
| 9_005_+_160532605_spCas9 | 10: A > G | ABE | | | |
| ABE_NG_20nt_3- | chr6: 1605326 | spCas9 NG | spCas9 | NG | TG |
| 9_027_+_160532605_spCas9 | 10: A > G | ABE | | | |
| ABE_NGA_20nt_3- | chr6: 1605326 | spCas9 VRQR | spCas9 | NGA | TGA |
| 12_019_+_160532605_spCas9 | 10: A > G | IBE | | | |
| ABE_NGA_20nt_3- | chr6: 1605326 | spCas9 VRQR | spCas9 | NGA | AGA |
| 9_005_+_160532607_spCas9 | 10: A > G | ABE | | | |
| ABE_NG_20nt_3- | chr6: 1605326 | spCas9 NG | spCas9 | NG | AG |
| 9_027_+_160532607_spCas9 | 10: A > G | ABE | | | |
| ABE_NGA_20nt_3- | chr6: 1605326 | spCas9 VRQR | spCas9 | NGA | AGA |
| 12_019_+_160532607_spCas9 | 10: A > G | IBE | | | |
| ABE_NGG_20nt_13- | chr6: 1605326 | SpCas9 | SpCas9 | NGG | TGG |
| 16_032_+_160532595_SpCas9 | 10: A > G | IBE_expanded | | | |
| | | ABE | | | |
| ABE_NGG_20nt_13- | chr6: 1605326 | SpCas9 | SpCas9 | NGG | GGG |
| 16_032_+_160532596_SpCas9 | 10: A > G | IBE_expanded | | | |
| | | ABE | | | |
| CBE_NGC_20nt_4- | chr6: 1605325 | spCas9 NGC | spCas9 | NGC | TGC |
| 9_009_+_160532524_spCas9 | 31: C > T | CBE | | | |
| CBE_NG_20nt_4- | chr6: 1605325 | spCas9 NG | spCas9 | NG | TG |
| 9_028_+_160532524_spCas9 | 31: C > T | CBE | | | |
| CBE_NRCH_20nt_3- | chr6: 1605325 | spCas9 NRCH | spCas9 | NRCH | TGCC |
| 9_030_+_160532524_spCas9 | 31: C > T | CBE | | | |
| CBE_NG_20nt_4- | chr6: 1605325 | spCas9 NG | spCas9 | NG | AG |
| 9_028_+_160532522_spCas9 | 31: C > T | CBE | | | |
| ABE_NGG_20nt_3-9_002_- | chr6: 1605317 | spCas9 ABE | spCas9 | NGG | AGG |
| 160531767_spCas9 | 84: T > C | | | | |
| ABE_NG_20nt_3-9_027_- | chr6: 1605317 | spCas9 NG | spCas9 | NG | AG |
| 160531768_spCas9 | 84: T > C | ABE | | | |
| ABE_NGG_20nt_3-12_018_- | chr6: 1605317 | spCas9 IBE | spCas9 | NGG | AGG |
| 160531767_spCas9 | 84: T > C | | | | |
| ABE_NNNRRT_21nt_5- | chr6: 1605317 | saCas9 KKH | saCas9 | NNNRRT | CAAGGT |
| 14_014_−_160531766_saCas9 | 84: T > C | ABE | | | |
| ABE_NG_20nt_3-9_027_- | chr6: 1605317 | spCas9 NG | spCas9 | NG | GG |
| 160531767_spCas9 | 84: T > C | ABE | | | |
| | | | Target | | |
| | | | Nucleo- | | |
| | | | tide | | |
| | | | Posi- | | |
| | | | tion | Location of | |
| | | | in | target site | |
| | Target | | Target | on Chr6 | |
| | Sequence | SEQ | Sequence | (hg38.2bit | |
| | Including | ID | (from 5' | genome sequence) | |
| Guide Name | PAM | NO | end) | start | end |
| CBE_NGA_20nt_4- | CACTGGCATCA | 888 | 7 | 160577150 | 160577173 |
| 9_006_- | GAGGACCCCAG | | | | |
| 160577150_spCas9 | A | | | | |
| CBE_NG_20nt_4- | CACTGGCATCA | 889 | 7 | 160577151 | 160577173 |
| 9_028_- | GAGGACCCCAG | | | | |
| 160577151_spCas9 | | | | | |
| CBE_NGA_20nt_3- | CACTGGCATCA | 890 | 7 | 160577150 | 160577173 |
| 16_035_- | GAGGACCCCAG | | | | |
| 160577150_spCas9 | A | | | | |
| CBE_NRCH_20nt_3 | GGCATCAGAGG | 891 | 3 | 160577145 | 160577169 |
| -9_030_- | ACCCCAGAAAA | | | | |
| 160577145_spCas9 | CT | | | | |
| CBE_NGG_20nt_3- | TGATACCACAC | 892 | 15 | 160577158 | 160577181 |
| 16_033_- | TGGCATCAGAG | | | | |
| 160577158_SpCas9 | G | | | | |
| CBE_NGA_20nt_3- | GATACCACACT | 893 | 14 | 160577157 | 160577180 |
| 16_035_- | GGCATCAGAGG | | | | |
| 160577157_spCas9 | A | | | | |
| CBE_NGA_20nt_4- | GGGCTTTTCTC | 894 | 4 | 160577276 | 160577299 |
| 9_006_+_16057727 | AGGTGGTGCTG | | | | |
| 6_spCas9 | A | | | | |
| CBE_NG_20nt_4- | GGGCTTTTCTC | 895 | 4 | 160577276 | 160577298 |
| 9_028_+_16057727 | AGGTGGTGCTG | | | | |
| 6_spCas9 | | | | | |
| CBE_NGA_20nt_3- | GGGCTTTTCTC | 896 | 4 | 160577276 | 160577299 |
| 16_035_+_1605772 | AGGTGGTGCTG | | | | |
| 76_spCas9 | A | | | | |
| CBE_NGC_20nt_4- | ACAGGGCTTTT | 897 | 7 | 160577273 | 160577296 |
| 9_009_+_16057727 | CTCAGGTGGTG | | | | |
| 3_spCas9 | C | | | | |
| CBE_NG_20nt_4- | ACAGGGCTTTT | 898 | 7 | 160577273 | 160577295 |
| 9_028_+_16057727 | CTCAGGTGGTG | | | | |
| 3_spCas9 | | | | | |
| CBE_NRCH_20nt_3 | ACAGGGCTTTT | 899 | 7 | 160577273 | 160577297 |
| - | CTCAGGTGGTG | | | | |
| 9_030_+_16057727 | CT | | | | |
| 3_spCas9 | | | | | |
| CBE_NNNRRT_21n | AGGGCTTTTCT | 900 | 5 | 160577275 | 160577302 |
| t_3- | CAGGTGGTGCT | | | | |
| 12_015_+_1605772 | GAAAT | | | | |
| 75_saCas9 | | | | | |
| CBE_NG_20nt_4- | CCACAGGGCTT | 901 | 9 | 160577271 | 160577293 |
| 9_028_+_16057727 | TTCTCAGGTGG | | | | |
| 1_spCas9 | | | | | |
| CBE_NGG_20nt_3- | TGGACCACAGG | 902 | 13 | 160577267 | 160577290 |
| 16_033_+_1605772 | GCTTTTCTCAG | | | | |
| 67_SpCas9 | G | | | | |
| CBE_NGG_20nt_3- | ACCACAGGGCT | 903 | 10 | 160577270 | 160577293 |
| 16_033_+_1605772 | TTTCTCAGGTG | | | | |
| 70_SpCas9 | G | | | | |
| ABE_NGA_20nt_3- | CACTGGCATCA | 904 | 8 | 160589567 | 160589590 |
| 9_005_- | GAGAACCACAG | | | | |
| 160589567_spCas9 | A | | | | |
| ABE_NG_20nt_3- | CACTGGCATCA | 905 | 8 | 160589568 | 160589590 |
| 9_027_- | GAGAACCACAG | | | | |
| 160589568_spCas9 | | | | | |
| ABE_NGA_20nt_3- | CACTGGCATCA | 906 | 8 | 160589567 | 160589590 |
| 12_019_- | GAGAACCACAG | | | | |
| 160589567_spCas9 | A | | | | |
| ABE_NNGRRT_21 | CACACTGGCAT | 907 | 10 | 160589565 | 160589592 |
| nt_5-14_011_- | CAGAGAACCAC | | | | |
| 160589565_saCas9 | AGAAT | | | | |
| ABE_NRCH_20nt_3 | GGCATCAGAGA | 908 | 4 | 160589562 | 160589586 |
| -9_024 | ACCACAGAATA | | | | |
| 160589562_spCas9 | CT | | | | |
| ABE_NGA_20nt_13 | TGACACCACAC | 909 | 16 | 160589575 | 160589598 |
| -16_034_- | TGGCATCAGAG | | | | |
| 160589575_SpCas9 | A | | | | |
| CBE_NGA_20nt_4- | TACTGCCGTAA | 910 | 7 | 160545510 | 160545533 |
| 9_006_- | CCCTGATGGTG | | | | |
| 160545510_spCas9 | A | | | | |
| CBE_NG_20nt_4- | TACTGCCGTAA | 911 | 7 | 160545511 | 160545533 |
| 9_028_- | CCCTGATGGTG | | | | |
| 160545511_spCas9 | | | | | |
| CBE_NGA_20nt_3- | TACTGCCGTAA | 912 | 7 | 160545510 | 160545533 |
| 16_035_- | CCCTGATGGTG | | | | |
| 160545510_spCas9 | A | | | | |
| CBE_NNNRRT_21n | TGCCGTAACCC | 913 | 4 | 160545503 | 160545530 |
| t_3-12_015_- | TGATGGTGACA | | | | |
| 160545503_saCas9 | TCAAT | | | | |
| CBE_NG_20nt_4- | AGTACTGCCGT | 914 | 9 | 160545513 | 160545535 |
| 9_028_- | AACCCTGATGG | | | | |
| 160545513_spCas9 | | | | | |
| CBE_NRCH_20nt_3 | ACTGCCGTAAC | 915 | 6 | 160545508 | 160545532 |
| -9_030_- | CCTGATGGTGA | | | | |
| 160545508_spCas9 | CA | | | | |
| CBE_NGG_20nt_3- | CAGTACTGCCG | 916 | 10 | 160545513 | 160545536 |
| 16_033_- | TAACCCTGATG | | | | |
| 160545513_SpCas9 | G | | | | |
| CBE_NGA_20nt_3- | TTTCAGTACTG | 917 | 13 | 160545516 | 160545539 |
| 16_035_- | CCGTAACCCTG | | | | |
| 160545516_spCas9 | A | | | | |
| CBE_NGG_20nt_4- | GGCTCCTTCCG | 918 | 6 | 160635116 | 160635139 |
| 9_003_- | AACAAGGTAAG | | | | |
| 160635116_spCas9 | G | | | | |
| CBE_NG_20nt_4- | GGCTCCTTCCG | 919 | 6 | 160635117 | 160635139 |
| 9_028_- | AACAAGGTAAG | | | | |
| 160635117_spCas9 | | | | | |
| CBE_NGG_20nt_3- | GGCTCCTTCCG | 920 | 6 | 160635116 | 160635139 |
| 16_033_- | AACAAGGTAAG | | | | |
| 160635116_SpCas9 | G | | | | |
| CBE_NGG_20nt_4- | GGCTCCTTCCG | 921 | 5 | 160635116 | 160635139 |
| 9_003_- | AACAAGGTAAG | | | | |
| 160635116_spCas9 | G | | | | |
| CBE_NG_20nt_4- | GGCTCCTTCCG | 922 | 5 | 160635117 | 160635139 |
| 9_028_- | AACAAGGTAAG | | | | |
| 160635117_spCas9 | | | | | |
| CBE_NGG_20nt_3- | GGCTCCTTCCG | 923 | 5 | 160635116 | 160635139 |
| 16_033_- | AACAAGGTAAG | | | | |
| 160635116_SpCas9 | G | | | | |
| CBE_NGA_20nt_4- | GCTCCTTCCGA | 924 | 5 | 160635115 | 160635138 |
| 9_006_- | ACAAGGTAAGG | | | | |
| 160635115_spCas9 | A | | | | |
| CBE_NG_20nt_4- | GCTCCTTCCGA | 925 | 5 | 160635116 | 160635138 |
| 9_028_- | ACAAGGTAAGG | | | | |
| 160635116_spCas9 | | | | | |
| CBE_NGA_20nt_3- | GCTCCTTCCGA | 926 | 5 | 160635115 | 160635138 |
| 16_035_- | ACAAGGTAAGG | | | | |
| 160635115_spCas9 | A | | | | |
| CBE_NGA_20nt_4- | GCTCCTTCCGA | 927 | 4 | 160635115 | 160635138 |
| 9_006_- | ACAAGGTAAGG | | | | |
| 160635115_spCas9 | A | | | | |
| CBE_NG_20nt_4- | GCTCCTTCCGA | 928 | 4 | 160635116 | 160635138 |
| 9_028_- | ACAAGGTAAGG | | | | |
| 160635116_spCas9 | | | | | |
| CBE_NGA_20nt_3- | GCTCCTTCCGA | 929 | 4 | 160635115 | 160635138 |
| 16_035_- | ACAAGGTAAGG | | | | |
| 160635115_spCas9 | A | | | | |
| CBE_NNGRRT_21nt | AGGCTCCTTCC | 930 | 7 | 160635113 | 160635140 |
| _3-12_012_- | GAACAAGGTAA | | | | |
| 160635113_saCas9 | GGAGT | | | | |
| CBE_NNGRRT_21nt | AGGCTCCTTCC | 931 | 6 | 160635113 | 160635140 |
| _3-12_012_- | GAACAAGGTAA | | | | |
| 160635113_saCas9 | GGAGT | | | | |
| CBE_NGG_20nt_3- | CTAGAGGCTCC | 932 | 11 | 160635121 | 160635144 |
| 16_033_- | TTCCGAACAAG | | | | |
| 160635121_SpCas9 | G | | | | |
| CBE_NGG_20nt_3- | CTAGAGGCTCC | 933 | 10 | 160635121 | 160635144 |
| 16_033_- | TTCCGAACAAG | | | | |
| 160635121_SpCas9 | G | | | | |
| CBE_NG_20nt_4- | TAGAGGCTCCT | 934 | 9 | 160635121 | 160635143 |
| 9_028_- | TCCGAACAAGG | | | | |
| 160635121_spCas9 | | | | | |
| CBE_NGA_20nt_3- | CCAAGCCTAGA | 935 | 16 | 160635127 | 160635150 |
| 16_035_- | GGCTCCTTCCG | | | | |
| 160635127_spCas9 | A | | | | |
| CBE_NGG_20nt_4- | TCCACGGCTGT | 936 | 5 | 160590942 | 160590965 |
| 9_003_- | TTCTGAACAAG | | | | |
| 160590942_spCas9 | G | | | | |
| CBE_NG_20nt_4- | TCCACGGCTGT | 937 | 5 | 160590943 | 160590965 |
| 9_028_- | TTCTGAACAAG | | | | |
| 160590943_spCas9 | | | | | |
| CBE_NGG_20nt_3- | TCCACGGCTGT | 938 | 5 | 160590942 | 160590965 |
| 16_033_- | TTCTGAACAAG | | | | |
| 160590942_SpCas9 | G | | | | |
| CBE_NNNRRT_21n | ACGTCCACGGC | 939 | 8 | 160590941 | 160590968 |
| t_3-12_015_- | TGTTTCTGAAC | | | | |
| 160590941_saCas9 | AAGGT | | | | |
| CBE_NG_20nt_4- | CCACGGCTGTT | 940 | 4 | 160590942 | 160590964 |
| 9_028_- | TCTGAACAAGG | | | | |
| 160590942_spCas9 | | | | | |
| CBE_NRCH_20nt_3 | GACGTCCACGG | 941 | 9 | 160590945 | 160590969 |
| -9_030_- | CTGTTTCTGAA | | | | |
| 160590945_spCas9 | CA | | | | |
| CBE_NGA_20nt_3- | GCGACGTCCAC | 942 | 11 | 160590948 | 160590971 |
| 16_035_- | GGCTGTTTCTG | | | | |
| 160590948_spCas9 | A | | | | |
| ABE_NGG_20nt_3- | TACTCATTTGG | 943 | 6 | 160556021 | 160556044 |
| 9_002_+_16055602 | GTAGTTTTCTG | | | | |
| 1_spCas9 | G | | | | |
| ABE_NG_20nt_3- | TACTCATTTGG | 944 | 6 | 160556021 | 160556043 |
| 9_027_+_16055602 | GTAGTTTTCTG | | | | |
| 1_spCas9 | | | | | |
| ABE_NGG_20nt_3- | TACTCATTTGG | 945 | 6 | 160556021 | 160556044 |
| 12_018_+_1605560 | GTAGTTTTCTG | | | | |
| 21_spCas9 | G | | | | |
| ABE_NGG_20nt_3- | ACTCATTTGGG | 946 | 5 | 160556022 | 160556045 |
| 9_002_+_16055602 | TAGTTTTCTGG | | | | |
| 2_spCas9 | G | | | | |
| ABE_NG_20nt_3- | ACTCATTTGGG | 947 | 5 | 160556022 | 160556044 |
| 9_027_+_16055602 | TAGTTTTCTGG | | | | |
| 2_spCas9 | | | | | |
| ABE_NGG_20nt_3- | ACTCATTTGGG | 948 | 5 | 160556022 | 160556045 |
| 12_018_+_1605560 | TAGTTTTCTGG | | | | |
| 22_spCas9 | G | | | | |
| ABE_NGG_20nt_3- | CTCATTTGGGT | 949 | 4 | 160556023 | 160556046 |
| 9_002_+_16055602 | AGTTTTCTGGG | | | | |
| 3_spCas9 | G | | | | |
| ABE_NG_20nt_3- | CTCATTTGGGT | 950 | 4 | 160556023 | 160556045 |
| 9_027_+_16055602 | AGTTTTCTGGG | | | | |
| 3_spCas9 | | | | | |
| ABE_NGG_20nt_3- | CTCATTTGGGT | 951 | 4 | 160556023 | 160556046 |
| 12_018_+_1605560 | AGTTTTCTGGG | | | | |
| 23_spCas9 | G | | | | |
| ABE_NNGRRT_21 | ATACTCATTTG | 952 | 7 | 160556020 | 160556047 |
| nt_5- | GGTAGTTTTCT | | | | |
| 14_011_+_1605560 | GGGGT | | | | |
| 20_saCas9 | | | | | |
| ABE_NG_20nt_3- | TCATTTGGGTA | 953 | 3 | 160556024 | 160556046 |
| 9_027_+_16055602 | GTTTTCTGGGG | | | | |
| 4_spCas9 | | | | | |
| ABE_NGC_20nt_3- | AGTAACAGTGG | 954 | 5 | 160547881 | 160547904 |
| 9_008_+_16054788 | TTGCCTTCTTG | | | | |
| 1_spCas9 | C | | | | |
| ABE_NG_20nt_3- | AGTAACAGTGG | 955 | 5 | 160547881 | 160547903 |
| 9_027_+_16054788 | TTGCCTTCTTG | | | | |
| 1_spCas9 | | | | | |
| ABE_NGC_20nt_3- | AGTAACAGTGG | 956 | 5 | 160547881 | 160547904 |
| 12_020_+_1605478 | TTGCCTTCTTG | | | | |
| 81_spCas9 | C | | | | |
| ABE_NRCH_20nt_3 | AGTAACAGTGG | 957 | 5 | 160547881 | 160547905 |
| - | TTGCCTTCTTG | | | | |
| 9_024_+_16054788 | CC | | | | |
| 1_spCas9 | | | | | |
| CBE_NGG_20nt_4- | CTGCGTCTGAG | 958 | 4 | 160635188 | 160635211 |
| 9_003_+_16063518 | CATTGCGTCAG | | | | |
| 8_spCas9 | G | | | | |
| CBE_NG_20nt_4- | CTGCGTCTGAG | 959 | 4 | 160635188 | 160635210 |
| 9_028_+_16063518 | CATTGCGTCAG | | | | |
| 8_spCas9 | | | | | |
| CBE_NGG_20nt_3- | CTGCGTCTGAG | 960 | 4 | 160635188 | 160635211 |
| 16_033_+_1606351 | CATTGCGTCAG | | | | |
| 88_SpCas9 | G | | | | |
| CBE_NNNRRT_21n | CTTCTGCGTCT | 961 | 7 | 160635185 | 160635212 |
| t_3- | GAGCATTGCGT | | | | |
| 12_015_+_1606351 | CAGGT | | | | |
| 85_saCas9 | | | | | |
| CBE_NG_20nt_4- | CCTTCTGCGTC | 962 | 8 | 160635184 | 160635206 |
| 9_028_+_16063518 | TGAGCATTGCG | | | | |
| 4_spCas9 | | | | | |
| CBE_NNNRRT_21n | ATGTGCCTCGG | 963 | 9 | 160599590 | 160599617 |
| t_3- | TAACTCTGTCC | | | | |
| 12_015_+_1605995 | ATAAT | | | | |
| 90_saCas9 | | | | | |
| CBE_NNNRRT_21n | TGCCTCGGTAA | 964 | 6 | 160599593 | 160599620 |
| t_3- | CTCTGTCCATA | | | | |
| 12_015_+_1605995 | ATGGT | | | | |
| 93_saCas9 | | | | | |
| CBE_NNNRRT_ | CTCGGTAACTC | 965 | 3 | 160599596 | 160599623 |
| 21nt_3- | TGTCCATAATG | | | | |
| 12_015_+_1605995 | GTAGT | | | | |
| 96_saCas9 | | | | | |
| CBE_NGG_20nt_3- | CTCGGTAACTC | 966 | 3 | 160599596 | 160599619 |
| 16_033_+_1605995 | TGTCCATAATG | | | | |
| 96_SpCas9 | G | | | | |
| ABE_NGG_20nt_3- | GGTAATGGCCA | 967 | 11 | 160556120 | 160556143 |
| 12_018_- | GAGTTATCGAG | | | | |
| 160556120_spCas9 | G | | | | |
| ABE_NGC_20nt_3- | GTAATGGCCAG | 968 | 10 | 160556119 | 160556142 |
| 12_020_- | AGTTATCGAGG | | | | |
| 160556119_spCas9 | C | | | | |
| ABE_NRCH_20nt_3 | AATGGCCAGAG | 969 | 8 | 160556116 | 160556140 |
| -9_024_- | TTATCGAGGCA | | | | |
| 160556116_spCas9 | CA | | | | |
| ABE_NGA_20nt_13 | ATGGTAATGGC | 970 | 13 | 160556122 | 160556145 |
| -16_034_- | CAGAGTTATCG | | | | |
| 160556122_SpCas9 | A | | | | |
| CBE_NGG_20nt_4- | CGTCCCTCCGA | 971 | 9 | 160600946 | 160600969 |
| 9_003_- | ATGTTATTCTG | | | | |
| 160600946_spCas9 | G | | | | |
| CBE_NG_20nt_4- | CGTCCCTCCGA | 972 | 9 | 160600947 | 160600969 |
| 9_028_- | ATGTTATTCTG | | | | |
| 160600947_spCas9 | | | | | |
| CBE_NGG_20nt_3- | CGTCCCTCCGA | 973 | 9 | 160600946 | 160600969 |
| 16_033_- | ATGTTATTCTG | | | | |
| 160600946_SpCas9 | G | | | | |
| CBE_NGC_20nt_4- | GTCCCTCCGAA | 974 | 8 | 160600945 | 160600968 |
| 9_009_- | TGTTATTCTGG | | | | |
| 160600945_spCas9 | C | | | | |
| CBE_NG_20nt_4- | GTCCCTCCGAA | 975 | 8 | 160600946 | 160600968 |
| 9_028_- | TGTTATTCTGG | | | | |
| 160600946_spCas9 | | | | | |
| CBE_NRCH_20nt_3 | GTCCCTCCGAA | 976 | 8 | 160600944 | 160600968 |
| -9_030_- | TGTTATTCTGG | | | | |
| 160600944_spCas9 | CT | | | | |
| ABE_NGC_20nt_3- | GTGATGGACAG | 977 | 12 | 160585140 | 160585163 |
| 12_020_- | AGTTATCGAGG | | | | |
| 160585140_spCas9 | C | | | | |
| ABE_NRCH_20nt_3 | TGGACAGAGTT | 978 | 8 | 160585135 | 160585159 |
| -9_024_- | ATCGAGGCACA | | | | |
| 160585135_spCas9 | CT | | | | |
| ABE_NGG_20nt_13 | GGTGATGGACA | 979 | 13 | 160585141 | 160585164 |
| -16_032_- | GAGTTATCGAG | | | | |
| 160585141_SpCas9 | G | | | | |
| ABE_NGA_20nt_13 | GAGGTGATGGA | 980 | 15 | 160585143 | 160585166 |
| -16_034_- | CAGAGTTATCG | | | | |
| 160585143_SpCas9 | A | | | | |
| ABE_NGG_20nt_3- | ACACACTTTCT | 981 | 3 | 160664225 | 160664248 |
| 9_002_- | GGGCACTGCTG | | | | |
| 160664225_spCas9 | G | | | | |
| ABE_NG_20nt_3- | ACACACTTTCT | 982 | 3 | 160664226 | 160664248 |
| 9_027_- | GGGCACTGCTG | | | | |
| 160664226_spCas9 | | | | | |
| ABE_NGG_20nt_3- | ACACACTTTCT | 983 | 3 | 160664225 | 160664248 |
| 12_018_- | GGGCACTGCTG | | | | |
| 160664225_spCas9 | G | | | | |
| ABE_NGC_20nt_3- | GGGACACACTT | 984 | 6 | 160664228 | 160664251 |
| 9_008_- | TCTGGGCACTG | | | | |
| 160664228_spCas9 | C | | | | |
| ABE_NG_20nt_3- | GGGACACACTT | 985 | 6 | 160664229 | 160664251 |
| 9_027_- | TCTGGGCACTG | | | | |
| 160664229_spCas9 | | | | | |
| ABE_NGC_20nt_3- | GGGACACACTT | 986 | 6 | 160664228 | 160664251 |
| 12_020_- | TCTGGGCACTG | | | | |
| 160664228_spCas9 | C | | | | |
| ABE_NRCH_20nt_3 | GGGACACACTT | 987 | 6 | 160664227 | 160664251 |
| -9_024_- | TCTGGGCACTG | | | | |
| 160664227_spCas9 | CT | | | | |
| ABE_NGG_20nt_3- | GGGATTGGGAC | 988 | 12 | 160664234 | 160664257 |
| 12_018 | ACACTTTCTGG | | | | |
| 160664234_spCas9 | G | | | | |
| ABE_NGC_20nt_3- | GGATTGGGACA | 989 | 11 | 160664233 | 160664256 |
| 12_020_- | CACTTTCTGGG | | | | |
| 160664233_spCas9 | C | | | | |
| ABE_NRCH_20nt_3 | ATTGGGACACA | 990 | 9 | 160664230 | 160664254 |
| -9_024_- | CTTTCTGGGCA | | | | |
| 160664230_spCas9 | CT | | | | |
| ABE_NGG_20nt_13 | TGGGATTGGGA | 991 | 13 | 160664235 | 160664258 |
| -16_032_- | CACACTTTCTG | | | | |
| 160664235_SpCas9 | G | | | | |
| CBE_NGG_20nt_4- | ATGCCTCGCCC | 992 | 9 | 160685562 | 160685585 |
| 9_003_+_16068556 | TGCTTCGGCTG | | | | |
| 2_spCas9 | G | | | | |
| CBE_NG_20nt_4- | ATGCCTCGCCC | 993 | 9 | 160685562 | 160685584 |
| 9_028_+_16068556 | TGCTTCGGCTG | | | | |
| 2_spCas9 | | | | | |
| CBE_NGG_20nt_3- | ATGCCTCGCCC | 994 | 9 | 160685562 | 160685585 |
| 16_033_+_1606855 | TGCTTCGGCTG | | | | |
| 62_SpCas9 | G | | | | |
| CBE_NGC_20nt_4- | TGCCTCGCCCT | 995 | 8 | 160685563 | 160685586 |
| 9_009_+_16068556 | GCTTCGGCTGG | | | | |
| 3_spCas9 | C | | | | |
| CBE_NG_20nt_4- | TGCCTCGCCCT | 996 | 8 | 160685563 | 160685585 |
| 9_028_+_16068556 | GCTTCGGCTGG | | | | |
| 3_spCas9 | | | | | |
| CBE_NGC_20nt_4- | CCTCGCCCTGC | 997 | 6 | 160685565 | 160685588 |
| 9_009_+_16068556 | TTCGGCTGGCG | | | | |
| 5_spCas9 | C | | | | |
| CBE_NG_20nt_4- | CCTCGCCCTGC | 998 | 6 | 160685565 | 160685587 |
| 9_028_+_16068556 | TTCGGCTGGCG | | | | |
| 5_spCas9 | | | | | |
| CBE_NRCH_20nt_3 | CCTCGCCCTGC | 999 | 6 | 160685565 | 160685589 |
| 9_030_+_16068556 | TTCGGCTGGCG | | | | |
| 5_spCas9 | CA | | | | |
| CBE_NNNRRT_21n | CTCGCCCTGCT | 1000 | 5 | 160685566 | 160685593 |
| t_3- | TCGGCTGGCGC | | | | |
| 12_015_+_1606855 | ACAGT | | | | |
| 66_saCas9 | | | | | |
| CBE_NRCH_20nt_3 | TCGCCCTGCTT | 1001 | 4 | 160685567 | 160685591 |
| 9_030_+_16068556 | CGGCTGGCGCA | | | | |
| 7_spCas9 | CA | | | | |
| CBE_NGG_20nt_3- | GGCAATGCCTC | 1002 | 13 | 160685558 | 160685581 |
| 16_033_+_1606855 | GCCCTGCTTCG | | | | |
| 58_SpCas9 | G | | | | |
| CBE_NGC_20nt_4- | GGTCACTCCCA | 1003 | 4 | 160685456 | 160685479 |
| 9_009_- | CCCGAATACTG | | | | |
| 160685456_spCas9 | C | | | | |
| CBE_NG_20nt_4- | GGTCACTCCCA | 1004 | 4 | 160685457 | 160685479 |
| 9_028_- | CCCGAATACTG | | | | |
| 160685457_spCas9 | | | | | |
| CBE_NRCH_20nt_3 | TCGGGTCACTC | 1005 | 7 | 160685458 | 160685482 |
| -9_030_- | CCACCCGAATA | | | | |
| 160685458_spCas9 | CT | | | | |
| CBE_NGA_20nt_3- | AAAATCGGGTC | 1006 | 11 | 160685463 | 160685486 |
| 16_035_- | ACTCCCACCCG | | | | |
| 160685463_spCas9 | A | | | | |
| CBE_NGA_20nt_4- | TGGATTTCGGC | 1007 | 8 | 160601067 | 160601090 |
| 9_006_+_16060106 | AGTAGTTCTTG | | | | |
| 7_spCas9 | A | | | | |
| CBE_NG_20nt_4- | TGGATTTCGGC | 1008 | 8 | 160601067 | 160601089 |
| 9_028_+_16060106 | AGTAGTTCTTG | | | | |
| 7_spCas9 | | | | | |
| CBE_NGA_20nt_3- | TGGATTTCGGC | 1009 | 8 | 160601067 | 160601090 |
| 16_035_+_1606010 | AGTAGTTCTTG | | | | |
| 67_spCas9 | A | | | | |
| CBE_NNNRRT_21n | ATCTGGATTTC | 1010 | 11 | 160601064 | 160601091 |
| t_3- | GGCAGTAGTTC | | | | |
| 12_015_+_1606010 | TTGAT | | | | |
| 64_saCas9 | | | | | |
| ABE_NGG_20nt_3- | GAACAAAGACG | 1011 | 9 | 160599485 | 160599508 |
| 9_002_+_16059948 | TACGCATTTGG | | | | |
| 5_spCas9 | G | | | | |
| ABE_NG_20nt_3- | GAACAAAGACG | 1012 | 9 | 160599485 | 160599507 |
| 9_027_+_16059948 | TACGCATTTGG | | | | |
| 5_spCas9 | | | | | |
| ABE_NGG_20nt_3- | GAACAAAGACG | 1013 | 9 | 160599485 | 160599508 |
| 12_018_+_1605994 | TACGCATTTGG | | | | |
| 85_spCas9 | G | | | | |
| ABE_NNGRRT_21 | AAAGAACAAAG | 1014 | 12 | 160599482 | 160599509 |
| nt_5- | ACGTACGCATT | | | | |
| 14_011_+_1605994 | TGGGT | | | | |
| 82_saCas9 | | | | | |
| ABE_NNNRRT_21 | GAACAAAGACG | 1015 | 9 | 160599485 | 160599512 |
| nt_5- | TACGCATTTGG | | | | |
| 14_014_+_1605994 | GTAGT | | | | |
| 85_saCas9 | | | | | |
| ABE_NG_20nt_3- | AACAAAGACGT | 1016 | 8 | 160599486 | 160599508 |
| 9_027_+_16059948 | ACGCATTTGGG | | | | |
| 6_spCas9 | | | | | |
| ABE_NG_20nt_3- | AAAGACGTACG | 1017 | 5 | 160599489 | 160599511 |
| 9_027_+_16059948 | CATTTGGGTAG | | | | |
| 9_spCas9 | | | | | |
| ABE_NGG_20nt_3- | AGAACAAAGAC | 1018 | 10 | 160599484 | 160599507 |
| 12_018_+_1605994 | GTACGCATTTG | | | | |
| 84_spCas9 | G | | | | |
| ABE_NGC_20nt_3- | TAACACCAAGG | 1019 | 5 | 160591044 | 160591067 |
| 9_008_+_16059104 | ACTAATCTCAG | | | | |
| 4_spCas9 | C | | | | |
| ABE_NG_20nt_3- | TAACACCAAGG | 1020 | 5 | 160591044 | 160591066 |
| 9_027_+_16059104 | ACTAATCTCAG | | | | |
| 4_spCas9 | | | | | |
| ABE_NGC_20nt_3- | TAACACCAAGG | 1021 | 5 | 160591044 | 160591067 |
| 12_020_+_1605910 | ACTAATCTCAG | | | | |
| 44_spCas9 | C | | | | |
| ABE_NRCH_20nt_3 | TAACACCAAGG | 1022 | 5 | 160591044 | 160591068 |
| - | ACTAATCTCAG | | | | |
| 9_024_+_16059104 | CA | | | | |
| 4_spCas9 | | | | | |
| ABE_NGA_20nt_13 | GATCCATGGTA | 1023 | 16 | 160591033 | 160591056 |
| 16_034_+_1605910 | TAACACCAAGG | | | | |
| 33_SpCas9 | A | | | | |
| CBE_NGA_20nt_4- | GCGATGCTCAG | 1024 | 9 | 160548537 | 160548560 |
| 9_006_- | ACACAGAAGGG | | | | |
| 160548537_spCas9 | A | | | | |
| CBE_NG_20nt_4- | GCGATGCTCAG | 1025 | 9 | 160548538 | 160548560 |
| 9_028_- | ACACAGAAGGG | | | | |
| 160548538_spCas9 | | | | | |
| CBE_NGA_20nt_3- | GCGATGCTCAG | 1026 | 9 | 160548537 | 160548560 |
| 16_035_- | ACACAGAAGGG | | | | |
| 160548537_spCas9 | A | | | | |
| CBE_NNNRRT_21n | ATGCTCAGACA | 1027 | 6 | 160548530 | 160548557 |
| t_3-12_015_- | CAGAAGGGACT | | | | |
| 160548530_saCas9 | GTGGT | | | | |
| CBE_NG_20nt_4- | TGCTCAGACAC | 1028 | 5 | 160548534 | 160548556 |
| 9_028_- | AGAAGGGACTG | | | | |
| 160548534_spCas9 | | | | | |
| CBE_NRCH_20nt_3 | CGATGCTCAGA | 1029 | 8 | 160548535 | 160548559 |
| -9_030_- | CACAGAAGGGA | | | | |
| 160548535_spCas9 | CT | | | | |
| CBE_NGG_20nt_3- | ACGCGATGCTC | 1030 | 11 | 160548539 | 160548562 |
| 16_033_- | AGACACAGAAG | | | | |
| 160548539_SpCas9 | G | | | | |
| CBE_NGG_20nt_3- | CGCGATGCTCA | 1031 | 10 | 160548538 | 160548561 |
| 16_033_- | GACACAGAAGG | | | | |
| 160548538_SpCas9 | G | | | | |
| CBE_NGG_20nt_3- | CTCAGACACAG | 1032 | 3 | 160548531 | 160548554 |
| 16_033_- | AAGGGACTGTG | | | | |
| 160548531_SpCas9 | G | | | | |
| CBE_NGA_20nt_3- | CTGACGCGATG | 1033 | 14 | 160548542 | 160548565 |
| 16_035_- | CTCAGACACAG | | | | |
| 160548542_spCas9 | A | | | | |
| ABE_NGA_20nt_3- | AACAAGGAGCT | 1034 | 5 | 160532605 | 160532628 |
| 9_005_+_16053260 | GGGCTTCCTTG | | | | |
| 5_spCas9 | A | | | | |
| ABE_NG_20nt_3- | AACAAGGAGCT | 1035 | 5 | 160532605 | 160532627 |
| 9_027_+_16053260 | GGGCTTCCTTG | | | | |
| 5_spCas9 | | | | | |
| ABE_NGA_20nt_3- | AACAAGGAGCT | 1036 | 5 | 160532605 | 160532628 |
| 12_019_+_1605326 | GGGCTTCCTTG | | | | |
| 05_spCas9 | A | | | | |
| ABE_NGA_20nt_3- | CAAGGAGCTGG | 1037 | 3 | 160532607 | 160532630 |
| 9_005_+_16053260 | GCTTCCTTGAG | | | | |
| 7_spCas9 | A | | | | |
| ABE_NG_20nt_3- | CAAGGAGCTGG | 1038 | 3 | 160532607 | 160532629 |
| 9_027_+_16053260 | GCTTCCTTGAG | | | | |
| 7_spCas9 | | | | | |
| ABE_NGA_20nt_3- | CAAGGAGCTGG | 1039 | 3 | 160532607 | 160532630 |
| 12_019_+_1605326 | GCTTCCTTGAG | | | | |
| 07_spCas9 | A | | | | |
| ABE_NGG_20nt_13- | CATTCTCAATA | 1040 | 15 | 160532595 | 160532618 |
| 16_032_+_1605325 | ACAAGGAGCTG | | | | |
| 95_SpCas9 | G | | | | |
| ABE_NGG_20nt_13- | ATTCTCAATAA | 1041 | 14 | 160532596 | 160532619 |
| 16_032_+_1605325 | CAAGGAGCTGG | | | | |
| 96_SpCas9 | G | | | | |
| CBE_NGC_20nt_4- | TCTTACCTGGC | 1042 | 7 | 160532524 | 160532547 |
| 9_009_+_16053252 | AACTGTCAGTG | | | | |
| 4_spCas9 | C | | | | |
| CBE_NG_20nt_4- | TCTTACCTGGC | 1043 | 7 | 160532524 | 160532546 |
| 9_028_+_16053252 | AACTGTCAGTG | | | | |
| 4_spCas9 | | | | | |
| CBE_NRCH_20nt_ | TCTTACCTGGC | 1044 | 7 | 160532524 | 160532548 |
| 3- | AACTGTCAGTG | | | | |
| 9_030_+_16053252 | CC | | | | |
| 4_spCas9 | | | | | |
| CBE_NG_20nt_4- | TTTCTTACCTG | 1045 | 9 | 160532522 | 160532544 |
| 9_028_+_16053252 | GCAACTGTCAG | | | | |
| 2_spCas9 | | | | | |
| ABE_NGG_20nt_3- | GTGTCTATGCT | 1046 | 7 | 160531767 | 160531790 |
| 9_002_- | CGTGTTTCAAG | | | | |
| 160531767_spCas9 | G | | | | |
| ABE_NG_20nt_3- | GTGTCTATGCT | 1047 | 7 | 160531768 | 160531790 |
| 9_027_- | CGTGTTTCAAG | | | | |
| 160531768_spCas9 | | | | | |
| ABE_NGG_20nt_3- | GTGTCTATGCT | 1048 | 7 | 160531767 | 160531790 |
| 12_018_- | CGTGTTTCAAG | | | | |
| 160531767_spCas9 | G | | | | |
| ABE_NNNRRT_21 | CTGGTGTCTAT | 1049 | 10 | 160531766 | 160531793 |
| nt_5-14_014_- | GCTCGTGTTTC | | | | |
| 160531766_saCas9 | AAGGT | | | | |
| ABE_NG_20nt_3- | TGTCTATGCTC | 1050 | 6 | 160531767 | 160531789 |
| 9_027_- | GTGTTTCAAGG | | | | |
| 160531767_spCas9 | | | | | |

| | | SEQ ID |
|---|---|---|
| Guide Name | Spacer sequence | NO |
| CBE_NGA_20nt_ | CACUGGCAUCAGAGGACCCC | 1051 |
| 4-9_006_−_ | | |
| 160577150_spCas9 | | |
| CBE_NG_20nt_ | CACUGGCAUCAGAGGACCCC | 1052 |
| 4-9_028_−_ | | |
| 160577151_spCas9 | | |
| CBE_NGA_20nt_ | CACUGGCAUCAGAGGACCCC | 1053 |
| 3-16_035_−_ | | |
| 160577150_spCas9 | | |
| CBE_NRCH_20nt_ | GGCAUCAGAGGACCCCAGAA | 1054 |
| 3-9_030_−_ | | |
| 160577145_spCas9 | | |
| CBE_NGG_20nt_ | UGAUACCACACUGGCAUCAG | 1055 |
| 3-16_033_−_ | | |
| 160577158_SpCas9 | | |
| CBE_NGA_20nt_ | GAUACCACACUGGCAUCAGA | 1056 |
| 3-16_035_−_ | | |
| 160577157_spCas9 | | |
| CBE_NGA_20nt_ | GGGCUUUUCUCAGGUGGUGC | 1057 |
| 4-9_006_+_ | | |
| 160577276_spCas9 | | |
| CBE_NG_20nt_ | GGGCUUUUCUCAGGUGGUGC | 1058 |
| 4-9_028_+_ | | |
| 160577276_spCas9 | | |
| CBE_NGA_20nt_ | GGGCUUUUCUCAGGUGGUGC | 1059 |
| 3-16_035_+_ | | |
| 160577276_spCas9 | | |
| CBE_NGC_20nt_ | ACAGGGCUUUUCUCAGGUGG | 1060 |
| 4-9_009_+_ | | |
| 160577273_spCas9 | | |
| CBE_NG_20nt_ | ACAGGGCUUUUCUCAGGUGG | 1061 |
| 4-9_028_+_ | | |
| 160577273_spCas9 | | |
| CBE_NRCH_20nt_ | ACAGGGCUUUUCUCAGGUGG | 1062 |
| 3-9_030_+_ | | |
| 160577273_spCas9 | | |
| CBE_NNNRRT_21nt_ | AGGGCUUUUCUCAGGUGGUGC | 1063 |
| 3- | | |
| 12_015_+_ | | |
| 160577275_saCas9 | | |
| CBE_NG_20nt_ | CCACAGGGCUUUUCUCAGGU | 1064 |
| 4-9_028_+_ | | |
| 160577271_spCas9 | | |
| CBE_NGG_20nt_ | UGGACCACAGGGCUUUUCUC | 1065 |
| 3-16_033_+_ | | |
| 160577267_SpCas9 | | |
| CBE_NGG_20nt_ | ACCACAGGGCUUUUCUCAGG | 1066 |
| 3-16_033_+_ | | |
| 160577270_SpCas9 | | |
| ABE_NGA_20nt_ | CACUGGCAUCAGAGAACCAC | 1067 |
| 3-9_005_−_ | | |
| 160589567_spCas9 | | |
| ABE_NG_20nt_ | CACUGGCAUCAGAGAACCAC | 1068 |
| 3-9_027_−_ | | |
| 160589568_spCas9 | | |
| ABE_NGA_20nt_ | CACUGGCAUCAGAGAACCAC | 1069 |
| 3-12_019_−_ | | |
| 160589567_spCas9 | | |
| ABE_NNGRRT_21nt_ | CACACUGGCAUCAGAGAACCA | 1070 |
| 5-14_011_- | | |
| 160589565_saCas9 | | |
| ABE_NRCH_20nt_ | GGCAUCAGAGAACCACAGAA | 1071 |
| 3-9_024_−_ | | |
| 160589562_spCas9 | | |
| ABE_NGA_20nt_ | UGACACCACACUGGCAUCAG | 1072 |
| 13-16_034_−_ | | |
| 160589575_SpCas9 | | |
| CBE_NGA_20nt_ | UACUGCCGUAACCCUGAUGG | 1073 |
| 4-9_006_−_ | | |
| 160545510_spCas9 | | |
| CBE_NG_20nt_ | UACUGCCGUAACCCUGAUGG | 1074 |
| 4-9_028_−_ | | |
| 160545511_spCas9 | | |
| CBE_NGA_20nt_ | UACUGCCGUAACCCUGAUGG | 1075 |
| 3-16_035_−_ | | |
| 160545510_spCas9 | | |
| CBE_NNNRRT_21nt_ | UGCCGUAACCCUGAUGGUGAC | 1076 |
| 3-12_015_- | | |
| 160545503_saCas9 | | |
| CBE_NG_20nt_ | AGUACUGCCGUAACCCUGAU | 1077 |
| 4-9_028_−_ | | |
| 160545513_spCas9 | | |
| CBE_NRCH_20nt_ | ACUGCCGUAACCCUGAUGGU | 1078 |
| 3-9_030_−_ | | |
| 160545508_spCas9 | | |
| CBE_NGG_20nt_ | CAGUACUGCCGUAACCCUGA | 1079 |
| 3-16_033_−_ | | |
| 160545513_SpCas9 | | |
| CBE_NGA_20nt_ | UUUCAGUACUGCCGUAACCC | 1080 |
| 3-16_035_−_ | | |
| 160545516_spCas9 | | |
| CBE_NGG_20nt_ | GGCUCCUUCCGAACAAGGUA | 1081 |
| 4-9_003_−_ | | |
| 160635116_spCas9 | | |
| CBE_NG_20nt_ | GGCUCCUUCCGAACAAGGUA | 1082 |
| 4-9_028_−_ | | |
| 160635117_spCas9 | | |
| CBE_NGG_20nt_ | GGCUCCUUCCGAACAAGGUA | 1083 |
| 3-16_033_−_ | | |
| 160635116_SpCas9 | | |
| CBE_NGG_20nt_ | GGCUCCUUCCGAACAAGGUA | 1084 |
| 4-9_003_−_ | | |
| 160635116_spCas9 | | |
| CBE_NG_20nt_ | GGCUCCUUCCGAACAAGGUA | 1085 |
| 4-9_028_−_ | | |
| 160635117_spCas9 | | |
| CBE_NGG_20nt_ | GGCUCCUUCCGAACAAGGUA | 1086 |
| 3-16_033_−_ | | |
| 160635116_SpCas9 | | |
| CBE_NGA_20nt_ | GCUCCUUCCGAACAAGGUAA | 1087 |
| 4-9_006_−_ | | |
| 160635115_spCas9 | | |
| CBE_NG_20nt_ | GCUCCUUCCGAACAAGGUAA | 1088 |
| 4-9_028_−_ | | |
| 160635116_spCas9 | | |
| CBE_NGA_20nt_ | GCUCCUUCCGAACAAGGUAA | 1089 |
| 3-16_035_−_ | | |
| 160635115_spCas9 | | |
| CBE_NGA_20nt_ | GCUCCUUCCGAACAAGGUAA | 1090 |
| 4-9_006_−_ | | |
| 160635115_spCas9 | | |
| CBE_NG_20nt_ | GCUCCUUCCGAACAAGGUAA | 1091 |
| 4-9_028_−_ | | |
| 160635116_spCas9 | | |
| CBE_NGA_20nt_ | GCUCCUUCCGAACAAGGUAA | 1092 |
| 3-16_035_−_ | | |
| 160635115_spCas9 | | |
| CBE_NNGRRT_21nt_ | AGGCUCCUUCCGAACAAGGUA | 1093 |
| 3-12_012_- | | |
| 160635113_saCas9 | | |
| CBE_NNGRRT_21nt_ | AGGCUCCUUCCGAACAAGGUA | 1094 |
| 3-12_012_- | | |
| 160635113_saCas9 | | |
| CBE_NGG_20nt_ | CUAGAGGCUCCUUCCGAACA | 1095 |
| 3-16_033_−_ | | |
| 160635121_SpCas9 | | |
| CBE_NGG_20nt_ | CUAGAGGCUCCUUCCGAACA | 1096 |
| 3-16_033_−_ | | |
| 160635121_SpCas9 | | |
| CBE_NG_20nt_ | UAGAGGCUCCUUCCGAACAA | 1097 |
| 4-9_028_−_ | | |
| 160635121_spCas9 | | |
| CBE_NGA_20nt_ | CCAAGCCUAGAGGCUCCUUC | 1098 |
| 3-16_035_−_ | | |
| 160635127_spCas9 | | |
| CBE_NGG_20nt_ | UCCACGGCUGUUUCUGAACA | 1099 |
| 4-9_003_−_ | | |
| 160590942_spCas9 | | |
| CBE_NG_20nt_ | UCCACGGCUGUUUCUGAACA | 1100 |
| 4-9_028_−_ | | |
| 160590943_spCas9 | | |
| CBE_NGG_20nt_ | UCCACGGCUGUUUCUGAACA | 1101 |
| 3-16_033_−_ | | |
| 160590942_SpCas9 | | |
| CBE_NNNRRT_21nt_ | ACGUCCACGGCUGUUUCUGAA | 1102 |
| 3-12_015_- | | |
| 160590941_saCas9 | | |
| CBE_NG_20nt_ | CCACGGCUGUUUCUGAACAA | 1103 |
| 4-9_028_−_ | | |
| 160590942_spCas9 | | |
| CBE_NRCH_20nt_ | GACGUCCACGGCUGUUUCUG | 1104 |
| 3-9_030_−_ | | |
| 160590945_spCas9 | | |
| CBE_NGA_20nt_ | GCGACGUCCACGGCUGUUUC | 1105 |
| 3-16_035_−_ | | |
| 160590948_spCas9 | | |
| ABE_NGG_20nt_ | UACUCAUUUGGGUAGUUUUC | 1106 |
| 3-9_002_+_ | | |
| 160556021_spCas9 | | |
| ABE_NG_20nt_ | UACUCAUUUGGGUAGUUUUC | 1107 |
| 3-9_027_+_ | | |
| 160556021_spCas9 | | |
| ABE_NGG_20nt_ | UACUCAUUUGGGUAGUUUUC | 1108 |
| 3-12_018_+_ | | |
| 160556021_spCas9 | | |
| ABE_NGG_20nt_ | ACUCAUUUGGGUAGUUUUCU | 1109 |
| 3-9_002_+_ | | |
| 160556022_spCas9 | | |
| ABE_NG_20nt_ | ACUCAUUUGGGUAGUUUUCU | 1110 |
| 3-9_027_+_ | | |
| 160556022_spCas9 | | |
| ABE_NGG_20nt_ | ACUCAUUUGGGUAGUUUUCU | 1111 |
| 3-12_018_+_ | | |
| 160556022_spCas9 | | |
| ABE_NGG_20nt_ | CUCAUUUGGGUAGUUUUCUG | 1112 |
| 3-9_002_+_ | | |
| 160556023_spCas9 | | |
| ABE_NG_20nt_ | CUCAUUUGGGUAGUUUUCUG | 1113 |
| 3-9_027_+_ | | |
| 160556023_spCas9 | | |
| ABE_NGG_20nt_ | CUCAUUUGGGUAGUUUUCUG | 1114 |
| 3-12_018_+_ | | |
| 160556023_spCas9 | | |
| ABE_NNGRRT_21nt_ | AUACUCAUUUGGGUAGUUUUC | 1115 |
| 5- | | |
| 14_011_+_ | | |
| 160556020_saCas9 | | |
| ABE_NG_20nt_ | UCAUUUGGGUAGUUUUCUGG | 1116 |
| 3-9_027_+_ | | |
| 160556024_spCas9 | | |
| ABE_NGC_20nt_ | AGUAACAGUGGUUGCCUUCU | 1117 |
| 3-9_008_+_ | | |
| 160547881_spCas9 | | |
| ABE_NG_20nt_ | AGUAACAGUGGUUGCCUUCU | 1118 |
| 3-9_027_+_ | | |
| 160547881_spCas9 | | |
| ABE_NGC_20nt_ | AGUAACAGUGGUUGCCUUCU | 1119 |
| 3-12_020_+_ | | |
| 160547881_spCas9 | | |
| ABE_NRCH_20nt_ | AGUAACAGUGGUUGCCUUCU | 1120 |
| 3-9_024_+_ | | |
| 160547881_spCas9 | | |
| CBE_NGG_20nt_ | CUGCGUCUGAGCAUUGCGUC | 1121 |
| 4-9_003_+_ | | |
| 160635188_spCas9 | | |
| CBE_NG_20nt_ | CUGCGUCUGAGCAUUGCGUC | 1122 |
| 4-9_028_+_ | | |
| 160635188_spCas9 | | |
| CBE_NGG_20nt_ | CUGCGUCUGAGCAUUGCGUC | 1123 |
| 3-16_033_+_ | | |
| 160635188_SpCas9 | | |
| CBE_NNNRRT_21nt_ | CUUCUGCGUCUGAGCAUUGCG | 1124 |
| 3- | | |
| 12_015_+_ | | |
| 160635185_saCas9 | | |
| CBE_NG_20nt_ | CCUUCUGCGUCUGAGCAUUG | 1125 |
| 4-9_028_+_ | | |
| 160635184_spCas9 | | |
| CBE_NNNRRT_21nt_ | AUGUGCCUCGGUAACUCUGUC | 1126 |
| 3- | | |
| 12_015_+_ | | |
| 160599590_saCas9 | | |
| CBE_NNNRRT_21nt_ | UGCCUCGGUAACUCUGUCCAU | 1127 |
| 3- | | |
| 12_015_+_ | | |
| 160599593_saCas9 | | |
| CBE_NNNRRT_21nt_ | CUCGGUAACUCUGUCCAUAAU | 1128 |
| 3- | | |
| 12_015_+_ | | |
| 160599596_saCas9 | | |
| CBE_NGG_20nt_ | CUCGGUAACUCUGUCCAUAA | 1129 |
| 3-16_033_+_ | | |
| 160599596_SpCas9 | | |
| ABE_NGG_20nt_ | GGUAAUGGCCAGAGUUAUCG | 1130 |
| 3-12_018_−_ | | |
| 160556120_spCas9 | | |
| ABE_NGC_20nt_ | GUAAUGGCCAGAGUUAUCGA | 1131 |
| 3-12_020_−_ | | |
| 160556119_spCas9 | | |
| ABE_NRCH_20nt_ | AAUGGCCAGAGUUAUCGAGG | 1132 |
| 3-9_024_−_ | | |
| 160556116_spCas9 | | |
| ABE_NGA_20nt_ | AUGGUAAUGGCCAGAGUUAU | 1133 |
| 13-16_034_−_ | | |
| 160556122_SpCas9 | | |
| CBE_NGG_20nt_ | CGUCCCUCCGAAUGUUAUUC | 1134 |
| 4-9_003_−_ | | |
| 160600946_spCas9 | | |
| CBE_NG_20nt_ | CGUCCCUCCGAAUGUUAUUC | 1135 |
| 4-9_028_−_ | | |
| 160600947_spCas9 | | |
| CBE_NGG_20nt_ | CGUCCCUCCGAAUGUUAUUC | 1136 |
| 3-16_033_−_ | | |
| 160600946_SpCas9 | | |
| CBE_NGC_20nt_ | GUCCCUCCGAAUGUUAUUCU | 1137 |
| 4-9_009_−_ | | |
| 160600945_spCas9 | | |
| CBE_NG_20nt_ | GUCCCUCCGAAUGUUAUUCU | 1138 |
| 4-9_028_−_ | | |
| 160600946_spCas9 | | |
| CBE_NRCH_20nt_ | GUCCCUCCGAAUGUUAUUCU | 1139 |
| 3-9_030_−_ | | |
| 160600944_spCas9 | | |
| ABE_NGC_20nt_ | GUGAUGGACAGAGUUAUCGA | 1140 |
| 3-12_020_−_ | | |
| 160585140_spCas9 | | |
| ABE_NRCH_20nt_ | UGGACAGAGUUAUCGAGGCA | 1141 |
| 3-9_024_−_ | | |
| 160585135_spCas9 | | |
| ABE_NGG_20nt_ | GGUGAUGGACAGAGUUAUCG | 1142 |
| 13-16_032_−_ | | |
| 160585141_SpCas9 | | |
| ABE_NGA_20nt_ | GAGGUGAUGGACAGAGUUAU | 1143 |
| 13-16_034_−_ | | |
| 160585143_SpCas9 | | |
| ABE_NGG_20nt_ | ACACACUUUCUGGGCACUGC | 1144 |
| 3-9_002_−_ | | |
| 160664225_spCas9 | | |
| ABE_NG_20nt_ | ACACACUUUCUGGGCACUGC | 1145 |
| 3-9_027_−_ | | |
| 160664226_spCas9 | | |
| ABE_NGG_20nt_ | ACACACUUUCUGGGCACUGC | 1146 |
| 3-12_018_−_ | | |
| 160664225_spCas9 | | |
| ABE_NGC_20nt_ | GGGACACACUUUCUGGGCAC | 1147 |
| 3-9_008_−_ | | |
| 160664228_spCas9 | | |
| ABE_NG_20nt_ | GGGACACACUUUCUGGGCAC | 1148 |
| 3-9_027_−_ | | |
| 160664229_spCas9 | | |
| ABE_NGC_20nt_ | GGGACACACUUUCUGGGCAC | 1149 |
| 3-12_020_−_ | | |
| 160664228_spCas9 | | |
| ABE_NRCH_20nt_ | GGGACACACUUUCUGGGCAC | 1150 |
| 3-9_024_−_ | | |
| 160664227_spCas9 | | |
| ABE_NGG_20nt_ | GGGAUUGGGACACACUUUCU | 1151 |
| 3-12_018_−_ | | |
| 160664234_spCas9 | | |
| ABE_NGC_20nt_ | GGAUUGGGACACACUUUCUG | 1152 |
| 3-12_020_−_ | | |
| 160664233_spCas9 | | |
| ABE_NRCH_20nt_ | AUUGGGACACACUUUCUGGG | 1153 |
| 3-9_024_−_ | | |
| 160664230_spCas9 | | |
| ABE_NGG_20nt_ | UGGGAUUGGGACACACUUUC | 1154 |
| 13-16_032_−_ | | |
| 160664235_SpCas9 | | |
| CBE_NGG_20nt_ | AUGCCUCGCCCUGCUUCGGC | 1155 |
| 4-9_003_+_ | | |
| 160685562_spCas9 | | |
| CBE_NG_20nt_ | AUGCCUCGCCCUGCUUCGGC | 1156 |
| 4-9_028_+_ | | |
| 160685562_spCas9 | | |
| CBE_NGG_20nt_ | AUGCCUCGCCCUGCUUCGGC | 1157 |
| 3-16_033_+_ | | |
| 160685562_SpCas9 | | |
| CBE_NGC_20nt_ | UGCCUCGCCCUGCUUCGGCU | 1158 |
| 4-9_009_+_ | | |
| 160685563_spCas9 | | |
| CBE_NG_20nt_ | UGCCUCGCCCUGCUUCGGCU | 1159 |
| 4-9_028_+_ | | |
| 160685563_spCas9 | | |
| CBE_NGC_20nt_ | CCUCGCCCUGCUUCGGCUGG | 1160 |
| 4-9_009_+_ | | |
| 160685565_spCas9 | | |
| CBE_NG_20nt_ | CCUCGCCCUGCUUCGGCUGG | 1161 |
| 4-9_028_+_ | | |
| 160685565_spCas9 | | |
| CBE_NRCH_20nt_ | CCUCGCCCUGCUUCGGCUGG | 1162 |
| 3-9_030_+_ | | |
| 160685565_spCas9 | | |
| CBE_NNNRRT_21nt_ | CUCGCCCUGCUUCGGCUGGCG | 1163 |
| 3- | | |
| 12_015_+_ | | |
| 160685566_saCas9 | | |
| CBE_NRCH_20nt_ | UCGCCCUGCUUCGGCUGGCG | 1164 |
| 3-9_030_+_ | | |
| 160685567_spCas9 | | |
| CBE_NGG_20nt_ | GGCAAUGCCUCGCCCUGCUU | 1165 |
| 3-16_033_+_ | | |
| 160685558_SpCas9 | | |
| CBE_NGC_20nt_ | GGUCACUCCCACCCGAAUAC | 1166 |
| 4-9_009_−_ | | |
| 160685456_spCas9 | | |
| CBE_NG_20nt_ | GGUCACUCCCACCCGAAUAC | 1167 |
| 4-9_028_−_ | | |
| 160685457_spCas9 | | |
| CBE_NRCH_20nt_ | UCGGGUCACUCCCACCCGAA | 1168 |
| 3-9_030_−_ | | |
| 160685458_spCas9 | | |
| CBE_NGA_20nt_ | AAAAUCGGGUCACUCCCACC | 1169 |
| 3-16_035_−_ | | |
| 160685463_spCas9 | | |
| CBE_NGA_20nt_ | UGGAUUUCGGCAGUAGUUCU | 1170 |
| 4-9_006_+_ | | |
| 160601067_spCas9 | | |
| CBE_NG_20nt_ | UGGAUUUCGGCAGUAGUUCU | 1171 |
| 4-9_028_+_ | | |
| 160601067_spCas9 | | |
| CBE_NGA_20nt_ | UGGAUUUCGGCAGUAGUUCU | 1172 |
| 3-16_035_+_ | | |
| 160601067_spCas9 | | |
| CBE_NNNRRT_21nt_ | AUCUGGAUUUCGGCAGUAGUU | 1173 |
| 3- | | |
| 12_015_+_ | | |
| 160601064_saCas9 | | |
| ABE_NGG_20nt_ | GAACAAAGACGUACGCAUUU | 1174 |
| 3-9_002_+_ | | |
| 160599485_spCas9 | | |
| ABE_NG_20nt_ | GAACAAAGACGUACGCAUUU | 1175 |
| 3-9_027_+_ | | |
| 160599485_spCas9 | | |
| ABE_NGG_20nt_ | GAACAAAGACGUACGCAUUU | 1176 |
| 3-12_018_+_ | | |
| 160599485_spCas9 | | |
| ABE_NNGRRT_21nt_ | AAAGAACAAAGACGUACGCAU | 1177 |
| 5- | | |
| 14_011_+_ | | |
| 160599482_saCas9 | | |
| ABE_NNNRRT_21nt_ | GAACAAAGACGUACGCAUUUG | 1178 |
| 5- | | |
| 14_014_+_ | | |
| 160599485_saCas9 | | |
| ABE_NG_20nt_ | AACAAAGACGUACGCAUUUG | 1179 |
| 3-9_027_+_ | | |
| 160599486_spCas9 | | |
| ABE_NG_20nt_ | AAAGACGUACGCAUUUGGGU | 1180 |
| 3-9_027_+_ | | |
| 160599489_spCas9 | | |
| ABE_NGG_20nt_ | AGAACAAAGACGUACGCAUU | 1181 |
| 3-12_018_+_ | | |
| 160599484_spCas9 | | |
| ABE_NGC_20nt_ | UAACACCAAGGACUAAUCUC | 1182 |
| 3-9_008_+_ | | |
| 160591044_spCas9 | | |
| ABE_NG_20nt_ | UAACACCAAGGACUAAUCUC | 1183 |
| 3-9_027_+_ | | |
| 160591044_spCas9 | | |
| ABE_NGC_20nt_ | UAACACCAAGGACUAAUCUC | 1184 |
| 3-12_020_+_ | | |
| 160591044_spCas9 | | |
| ABE_NRCH_20nt_ | UAACACCAAGGACUAAUCUC | 1185 |
| 3-9_024_+_ | | |
| 160591044_spCas9 | | |
| ABE_NGA_20nt_ | GAUCCAUGGUAUAACACCAA | 1186 |
| 13-16_034_+_ | | |
| 160591033_SpCas9 | | |
| CBE_NGA_20nt_ | GCGAUGCUCAGACACAGAAG | 1187 |
| 4-9_006_−_ | | |
| 160548537_spCas9 | | |
| CBE_NG_20nt_ | GCGAUGCUCAGACACAGAAG | 1188 |
| 4-9_028_−_ | | |
| 160548538_spCas9 | | |
| CBE_NGA_20nt_ | GCGAUGCUCAGACACAGAAG | 1189 |
| 3-16_035_−_ | | |
| 160548537_spCas9 | | |
| CBE_NNNRRT_21nt_ | AUGCUCAGACACAGAAGGGAC | 1190 |
| 3-12_015_- | | |
| 160548530_saCas9 | | |
| CBE_NG_20nt_ | UGCUCAGACACAGAAGGGAC | 1191 |
| 4-9_028_−_ | | |
| 160548534_spCas9 | | |
| CBE_NRCH_20nt_ | CGAUGCUCAGACACAGAAGG | 1192 |
| 3-9_030_−_ | | |
| 160548535_spCas9 | | |
| CBE_NGG_20nt_ | ACGCGAUGCUCAGACACAGA | 1193 |
| 3-16_033_−_ | | |
| 160548539_SpCas9 | | |
| CBE_NGG_20nt_ | CGCGAUGCUCAGACACAGAA | 1194 |
| 3-16_033_−_ | | |
| 160548538_SpCas9 | | |
| CBE_NGG_20nt_ | CUCAGACACAGAAGGGACUG | 1195 |
| 3-16_033_−_ | | |
| 160548531_SpCas9 | | |
| CBE_NGA_20nt_ | CUGACGCGAUGCUCAGACAC | 1196 |
| 3-16_035_−_ | | |
| 160548542_spCas9 | | |
| ABE_NGA_20nt_ | AACAAGGAGCUGGGCUUCCU | 1197 |
| 3-9_005_+_ | | |
| 160532605_spCas9 | | |
| ABE_NG_20nt_ | AACAAGGAGCUGGGCUUCCU | 1198 |
| 3-9_027_+_ | | |
| 160532605_spCas9 | | |
| ABE_NGA_20nt_ | AACAAGGAGCUGGGCUUCCU | 1199 |
| 3-12_019_+_ | | |
| 160532605_spCas9 | | |
| ABE_NGA_20nt_ | CAAGGAGCUGGGCUUCCUUG | 1200 |
| 3-9_005_+_ | | |
| 160532607_spCas9 | | |
| ABE_NG_20nt_ | CAAGGAGCUGGGCUUCCUUG | 1201 |
| 3-9_027_+_ | | |
| 160532607_spCas9 | | |
| ABE_NGA_20nt_ | CAAGGAGCUGGGCUUCCUUG | 1202 |
| 3-12_019_+_ | | |
| 160532607_spCas9 | | |
| ABE_NGG_20nt_ | CAUUCUCAAUAACAAGGAGC | 1203 |
| 13-16_032_+_ | | |
| 160532595_SpCas9 | | |
| ABE_NGG_20nt_ | AUUCUCAAUAACAAGGAGCU | 1204 |
| 13-16_032_+_ | | |
| 160532596_SpCas9 | | |
| CBE_NGC_20nt_ | UCUUACCUGGCAACUGUCAG | 1205 |
| 4-9_009_+_ | | |
| 160532524_spCas9 | | |
| CBE_NG_20nt_ | UCUUACCUGGCAACUGUCAG | 1206 |
| 4-9_028_+_ | | |
| 160532524_spCas9 | | |
| CBE_NRCH_20nt_ | UCUUACCUGGCAACUGUCAG | 1207 |
| 3-9_030_+_ | | |
| 160532524_spCas9 | | |
| CBE_NG_20nt_ | UUUCUUACCUGGCAACUGUC | 1208 |
| 4-9_028_+_ | | |
| 160532522_spCas9 | | |
| ABE_NGG_20nt_ | GUGUCUAUGCUCGUGUUUCA | 1209 |
| 3-9_002_−_ | | |
| 160531767_spCas9 | | |
| ABE_NG_20nt_ | GUGUCUAUGCUCGUGUUUCA | 1210 |
| 3-9_027_−_ | | |
| 160531768_spCas9 | | |
| ABE_NGG_20nt_ | GUGUCUAUGCUCGUGUUUCA | 1211 |
| 3-12_018_−_ | | |
| 160531767_spCas9 | | |
| ABE_NNNRRT_21nt_ | CUGGUGUCUAUGCUCGUGUUU | 1212 |
| 5-14_014_- | | |
| 160531766_saCas9 | | |
| ABE_NG_20nt_ | UGUCUAUGCUCGUGUUUCAA | 1213 |
| 3-9_027_−_ | | |
| 160531767_spCas9 | | |

TABLE 1B-1
Exemplary guide polynucleotide sequences for use in targeting a base editor to
disrupt a splice site or introduce a start codon in an LPA polynucleotide.

| | | SEQ |
|---|---|---|
| Guide Name | Guide Polynucleotide Sequence | ID NO |
| ABE_NGA_20 nt_3- | mAsmAsmAsCGUACUUCUUCAAGCAGGUUUUAGAGCU | 1216 |
| 9_005_+_160541098_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmAsmAsCGUACUUCUUCAAGCAGGUUUUAGAGCU | 1217 |
| 9_006_+_160541098_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mAsmAsmAsACACCAAGGGCCUGUAUGUUUUAGAGCU | 1214 |
| 9_003_+_160548601_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mAsmAsmAsCACCAAGGGCCUGUAUCGUUUUAGAGCU | 1215 |
| 9_009_+_160548602_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3-9_002_−_ | mAsmAsmCsACAGGUACCUUUGGGACGUUUUAGAGCU | 1218 |
| 160532633_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmAsmCsACCAAGGGGCUGCCACAGUUUUAGAGCU | 1219 |
| 9_006_+_160601042_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmAsmGsCCACUGGAAAUUCCAAAGUUUUAGAGCU | 1220 |
| 9_006_+_160601092_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmAsmGsGCAGUCCAUUCUGCAUCGUUUUAGAGCU | 1221 |
| 9_006_+_160600969_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3- | mAsmAsmUsUUCUUACCUUGUUCAGAGUUUUAGAGCU | 1222 |
| 9_002_+_160578512_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3-9_008_−_ | mAsmCsmAsCAGGUACCUUUGGGACUGUUUUAGAGCU | 1223 |
| 160532632_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mAsmCsmAsGUCCAGGACUGCUACCAGUUUUAGAGCU | 1224 |
| 160589669_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mAsmCsmGsCGAUGCUCAGACACAGAGUUUUAGAGCU | 1225 |
| 160548539_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3- | mAsmCsmGsUACUUCUUCAAGCAGUGGUUUUAGAGCU | 1226 |
| 9_008_+_160541100_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mAsmCsmGsUACUUCUUCAAGCAGUGGUUUUAGAGCU | 1227 |
| 9_009_+_160541100_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mAsmGsmCsCCAGCUCCUUGUUAUUGGUUUUAGAGCU | 1228 |
| 160532598_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mAsmGsmGsAUGCUGUGGCACAAGGUGUUUUAGAGCU | 1229 |
| 9_003_+_160542802_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mAsmGsmGsCCUGUAAGGAAAGUAUUGUUUUAGAGCU | 1230 |
| 9_009_+_160537957_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmGsmUsACUCCCACCUCACACACGUUUUAGAGCU | 1231 |
| 9_006_+_160557482_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mAsmGsmUsGCUGAAAUUAAAACAGAGUUUUAGAGCU | 1232 |
| 9_006_+_160599655_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mAsmUsmGsUCAAUCUUGGUCAUCCAGUUUUAGAGCU | 1233 |
| 160556067_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mAsmUsmUsAUGGACAGAGUUACCGAGUUUUAGAGCU | 1234 |
| 160599594_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mCsmAsmCsACAAGCAGAUAUUGCCUGUUUUAGAGCU | 1235 |
| 160540055_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmAsmCsCAGCAUAGUCGGACCCCGUUUUAGAGCU | 1236 |
| 160599514_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mCsmAsmGsUACUCCCACCUCACACAGUUUUAGAGCU | 1237 |
| 9_003_+_160557481_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmAsmUsCAACAUAAUAGGACCACGUUUUAGAGCU | 1238 |
| 160650352_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mCsmAsmUsCCAGGUUCCAAGCCUAGGUUUUAGAGCU | 1239 |
| 160548492_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mCsmCsmAsAACCUAGAAAGAAAACAGUUUUAGAGCU | 1240 |
| 9_009_+_160541175_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mCsmCsmUsGCCCAUUUAUUUGUCCCGUUUUAGAGCU | 1241 |
| 9_003_+_160547801_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mCsmGsmAsUGCCAAUGUGGUGUCAUGUUUUAGAGCU | 1242 |
| 9_006_+_160585072_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mCsmGsmGsCAGUGCUACCAUGGUAAGUUUUAGAGCU | 1243 |
| 160556135_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mCsmUsmCsCCACCUCACACACGGAUGUUUUAGAGCU | 1244 |
| 9_003_+_160557486_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsACACAAUGCCUGGUGACGUUUUAGAGCU | 1245 |
| 160595418_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsACACAAUGCUCAGAAACGUUUUAGAGCU | 1246 |
| 160557454_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsACACGAUGCUCAGAUGCGUUUUAGAGCU | 1247 |
| 160600981_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsACGCAAUGUCCAGUGAUGUUUUAGAGCU | 1248 |
| 160586513_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsACGCGAUGCUCAGACACGUUUUAGAGCU | 1249 |
| 160548542_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mCsmUsmGsCCCAUUUAUUUGUCCCUGUUUUAGAGCU | 1250 |
| 9_006_+_160547802_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mCsmUsmGsCCGAAAUCCAGAUCCUGGUUUUAGAGCU | 1251 |
| 160601057_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsCUACCGAGGUGAUGGACGUUUUAGAGCU | 1252 |
| 160585151_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsGGGUCCAGGACUGCUACGUUUUAGAGCU | 1253 |
| 160585164_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mCsmUsmGsUCAAUCUUGGUCAUCUAGUUUUAGAGCU | 1254 |
| 160577178_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mCsmUsmGsUCACCCUAAACAGAGGUGUUUUAGAGCU | 1255 |
| 9_003_+_160531882_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3-9_002_−_ | mCsmUsmGsUUUAGGGUGACAGUGGAGUUUUAGAGCU | 1256 |
| 160531875_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mGsmAsmAsAUGGACAGAGUUAUCAAGUUUUAGAGCU | 1257 |
| 160605140_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mGsmAsmAsCAAGGUAAGAAGUCUCUGUUUUAGAGCU | 1258 |
| 160590927_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmAsmAsGCCCAGCUCCUUGUUAUGUUUUAGAGCU | 1259 |
| 160532600_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3- | mGsmAsmCsUUCCUACCUUCUUCAGAGUUUUAGAGCU | 1260 |
| 9_005_+_160595343_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3- | mGsmAsmCsUUCUUACCUUGUUCAGAGUUUUAGAGCU | 1261 |
| 9_002_+_160548467_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmAsmGsCAAAGCCAUGUGGUCCAGUUUUAGAGCU | 1262 |
| 160650466_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmAsmGsCAAAGCCCCACAGUCCAGUUUUAGAGCU | 1263 |
| 160589681_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmAsmGsCAAAGCCCCGGGGUCCAGUUUUAGAGCU | 1264 |
| 160594086_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mGsmAsmGsGAUGCUGUGGCACAAGGGUUUUAGAGCU | 1265 |
| 9_003_+_160542801_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3-9_005_−_ | mGsmCsmCsACAGCAUCCUCUUCAUUGUUUUAGAGCU | 1266 |
| 160542792_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3- | mGsmCsmUsCCUUACCUUGUUCAGAAGUUUUAGAGCU | 1267 |
| 9_005_+_160606467_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmCsmUsCCUUACCUUGUUCAGAAGUUUUAGAGCU | 1268 |
| 9_006_+_160606467_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmCsmUsGCUAAAAUUAAAACAGAGUUUUAGAGCU | 1269 |
| 9_006_+_160650493_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3- | mGsmCsmUsUCUUACCUUCUUCAGAAGUUUUAGAGCU | 1270 |
| 9_005_+_160586439_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmCsmUsUCUUACCUUCUUCAGAAGUUUUAGAGCU | 1271 |
| 9_006_+_160586439_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mGsmGsmAsGCAAAGCCCCACAGUCCGUUUUAGAGCU | 1272 |
| 160589682_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mGsmGsmAsGCAAAGCCCCGGGGUCCGUUUUAGAGCU | 1273 |
| 160594087_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmGsmAsUGCUGUGGCACAAGGUGGUUUUAGAGCU | 1274 |
| 9_006_+_160542803_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mGsmGsmCsAGUCCAUUCUGCAUCUGGUUUUAGAGCU | 1275 |
| 9_009_+_160600971_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3- | mGsmGsmCsUCCUUACCUUGUUCAGAGUUUUAGAGCU | 1276 |
| 9_002_+_160606466_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3- | mGsmGsmCsUUCUUACCUUCUUCAGAGUUUUAGAGCU | 1277 |
| 9_002_+_160586438_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mGsmGsmGsGUCCAGGACUGCUACCGGUUUUAGAGCU | 1278 |
| 160585162_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmGsmGsUGCAGGAGUGCUACCACGUUUUAGAGCU | 1279 |
| 160605161_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmGsmUsGCUGAAAUGAAAAGAAAGUUUUAGAGCU | 1280 |
| 9_006_+_160605201_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmGsmUsGCUGCUAAAAUUAAAACGUUUUAGAGCU | 1281 |
| 9_006_+_160650490_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mGsmUsmCsACCCUAAACAGAGGUAGGUUUUAGAGCU | 1282 |
| 9_003_+_160531884_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mGsmUsmCsAGUGCUGAAAUUAAAACGUUUUAGAGCU | 1283 |
| 9_006_+_160599652_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mGsmUsmCsAUCCAGGUUCCAAGCCUGUUUUAGAGCU | 1284 |
| 160548494_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4-9_006_−_ | mGsmUsmCsCAGGACUGCUACCGAGGGUUUUAGAGCU | 1285 |
| 160585159_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mUsmAsmAsCACCAAGGACUAAUCUCGUUUUAGAGCU | 1286 |
| 9_009_+_160591044_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mUsmAsmAsCACCAAGGGGCUGCCACGUUUUAGAGCU | 1287 |
| 9_003_+_160601041_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mUsmAsmAsCACCAGGGUUGUUUCCCGUUUUAGAGCU | 1288 |
| 9_006_+_160557514_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3-9_005_−_ | mUsmAsmCsAGGCCUGCCGUCAUCACGUUUUAGAGCU | 1289 |
| 160537943_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGA_20 nt_4- | mUsmCsmAsCCCUAAACAGAGGUAGGGUUUUAGAGCU | 1290 |
| 9_006_+_160531885_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mUsmCsmAsUCCAGGUUCCAAGCCUAGUUUUAGAGCU | 1291 |
| 160548493_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mUsmGsmAsACAAGGUAAGAAAUUUGGUUUUAGAGCU | 1292 |
| 160578507_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mUsmGsmAsACAAGGUAAGAAGUCUCGUUUUAGAGCU | 1293 |
| 160590928_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mUsmGsmAsACAAGGUAAGGAGCCUGGUUUUAGAGCU | 1294 |
| 160606461_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4-9_003_−_ | mUsmGsmAsGCAAAGCCAUGUGGUCCGUUUUAGAGCU | 1295 |
| 160650467_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mUsmGsmCsCGAAAUCCAGAUCCUGUGUUUUAGAGCU | 1296 |
| 160601056_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGG_20 nt_4- | mUsmGsmUsCACCCUAAACAGAGGUAGUUUUAGAGCU | 1297 |
| 9_003_+_160531883_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3-9_008_−_ | mUsmGsmUsUUAGGGUGACAGUGGAGGUUUUAGAGCU | 1298 |
| 160531874_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3-9_005_−_ | mUsmUsmAsAUUUCAGCACUGACUGAGUUUUAGAGCU | 1299 |
| 160599646_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3- | mUsmUsmCsCUACCUUCUUCAGAAGAGUUUUAGAGCU | 1300 |
| 9_008_+_160595346_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mUsmUsmCsCUACCUUCUUCAGAAGAGUUUUAGAGCU | 1301 |
| 9_009_+_160595346_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGA_20 nt_3-9_005_−_ | mUsmUsmCsUACAGACUGUAUGUUUGGUUUUAGAGCU | 1302 |
| 160547922_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3- | mUsmUsmCsUUACCUGCUUCAGAAUGGUUUUAGAGCU | 1303 |
| 9_008_+_160557382_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mUsmUsmCsUUACCUGCUUCAGAAUGGUUUUAGAGCU | 1304 |
| 9_009_+_160557382_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3- | mUsmUsmCsUUACCUUGUUCAAAAAAGUUUUAGAGCU | 1305 |
| 9_008_+_160600909_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4- | mUsmUsmCsUUACCUUGUUCAAAAAAGUUUUAGAGCU | 1306 |
| 9_009_+_160600909_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| CBE_NGC_20 nt_4-9_009_−_ | mUsmUsmGsAACAAGGUAAGAAGUUGGUUUUAGAGCU | 1307 |
| 160600902_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3-9_008_−_ | mUsmUsmUsCAGCACCAACUGAAAACGUUUUAGAGCU | 1308 |
| 160585188_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3-9_008_−_ | mUsmUsmUsCAGCACCACCUGAGAAAGUUUUAGAGCU | 1309 |
| 160577278_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3-9_002_−_ | mUsmUsmUsCUACAGACUGUAUGUUUGUUUUAGAGCU | 1310 |
| 160547923_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGC_20 nt_3-9_008_−_ | mUsmUsmUSUAGCAGCACCUGAGCAAGUUUUAGAGCU | 1311 |
| 160650480_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |
| ABE_NGG_20 nt_3-9_002_−_ | mUsmUsmUsUCUACAGACUGUAUGUUGUUUUAGAGCU | 1312 |
| 160547924_spCas9 | AGAAAUAGCAAGUUAAAAUAAGGCUAGUCCGUUAUCA | |
| | ACUUGAAAAAGUGGCACCGAGUCGGUGCmUsmUsmUs | |
| | U | |

| | Alternative | Target | | SEQ |
|---|---|---|---|---|
| Guide | Guide | Site | | ID |
| Name | Name | Name | Guide Polynucleotide Sequence | NO |
| gRNA3423- | gRNA3423 | TSBTx6282 | mAsmAsmAsCGUACUUCUUCAAGCAGGUUUUAGAGC | 1315 |
| 3114 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3421- | gRNA3421 | TSBTx6280 | mAsmAsmAsACACCAAGGGCCUGUAUGUUUUAGAGC | 1313 |
| 3112 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3422- | gRNA3422 | TSBTx6281 | mAsmAsmAsCACCAAGGGCCUGUAUCGUUUUAGAGC | 1314 |
| 3113 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3424- | gRNA3424 | TSBTx6283 | mAsmAsmCsACAGGUACCUUUGGGACGUUUUAGAGC | 1316 |
| 3115 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3425- | gRNA3425 | TSBTx6284 | mAsmAsmCsACCAAGGGGCUGCCACAGUUUUAGAGC | 1317 |
| 3116 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3426- | gRNA3426 | TSBTx6285 | mAsmAsmGsCCACUGGAAAUUCCAAAGUUUUAGAGC | 1318 |
| 3117 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3427- | gRNA3427 | TSBTx6286 | mAsmAsmGsGCAGUCCAUUCUGCAUCGUUUUAGAGC | 1319 |
| 3118 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3428- | gRNA3428 | TSBTx6287 | mAsmAsmUsUUCUUACCUUGUUCAGAGUUUUAGAGC | 1320 |
| 3119 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3429- | gRNA3429 | TSBTx6288 | mAsmCsmAsCAGGUACCUUUGGGACUGUUUUAGAGC | 1321 |
| 3120 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3430- | gRNA3430 | TSBTx6289 | mAsmCsmAsGUCCAGGACUGCUACCAGUUUUAGAGC | 1322 |
| 3121 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3431- | gRNA3431 | TSBTx6290 | mAsmCsmGsCGAUGCUCAGACACAGAGUUUUAGAGC | 1323 |
| 3122 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3432- | gRNA3432 | TSBTx6291 | mAsmCsmGsUACUUCUUCAAGCAGUGGUUUUAGAGC | 1324 |
| 3123 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3433- | gRNA3433 | TSBTx6292 | mAsmGsmCsCCAGCUCCUUGUUAUUGGUUUUAGAGC | 1325 |
| 3124 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3434- | gRNA3434 | TSBTx6293 | mAsmGsmGsAUGCUGUGGCACAAGGUGUUUUAGAGC | 1326 |
| 3125 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3435- | gRNA3435 | TSBTx6294 | mAsmGsmGsCCUGUAAGGAAAGUAUUGUUUUAGAGC | 1327 |
| 3126 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3436- | gRNA3436 | TSBTx6295 | mAsmGsmUsACUCCCACCUCACACACGUUUUAGAGC | 1328 |
| 3127 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3437- | gRNA3437 | TSBTx6296 | mAsmGsmUsGCUGAAAUUAAAACAGAGUUUUAGAGC | 1329 |
| 3128 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3438- | gRNA3438 | TSBTx6297 | mAsmUsmGsUCAAUCUUGGUCAUCCAGUUUUAGAGC | 1330 |
| 3129 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3439- | gRNA3439 | TSBTx6298 | mAsmUsmUsAUGGACAGAGUUACCGAGUUUUAGAGC | 1331 |
| 3130 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3440- | gRNA3440 | TSBTx6299 | mCsmAsmCsACAAGCAGAUAUUGCCUGUUUUAGAGC | 1332 |
| 3131 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3441- | gRNA3441 | TSBTx6300 | mCsmAsmCsCAGCAUAGUCGGACCCCGUUUUAGAGC | 1333 |
| 3132 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3442- | gRNA3442 | TSBTx6301 | mCsmAsmGsUACUCCCACCUCACACAGUUUUAGAGC | 1334 |
| 3133 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3443- | gRNA3443 | TSBTx6302 | mCsmAsmUsCAACAUAAUAGGACCACGUUUUAGAGC | 1335 |
| 3134 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3444- | gRNA3444 | TSBTx6303 | mCsmAsmUsCCAGGUUCCAAGCCUAGGUUUUAGAGC | 1336 |
| 3135 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3445- | gRNA3445 | TSBTx6304 | mCsmCsmAsAACCUAGAAAGAAAACAGUUUUAGAGC | 1337 |
| 3136 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3446- | gRNA3446 | TSBTx6305 | mCsmCsmUsGCCCAUUUAUUUGUCCCGUUUUAGAGC | 1338 |
| 3137 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3447- | gRNA3447 | TSBTx6306 | mCsmGsmAsUGCCAAUGUGGUGUCAUGUUUUAGAGC | 1339 |
| 3138 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3448- | gRNA3448 | TSBTx6307 | mCsmGsmGsCAGUGCUACCAUGGUAAGUUUUAGAGC | 1340 |
| 3139 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3449- | gRNA3449 | TSBTx6308 | mCsmUsmCsCCACCUCACACACGGAUGUUUUAGAGC | 1341 |
| 3140 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3450- | gRNA3450 | TSBTx6309 | mCsmUsmGsACACAAUGCCUGGUGACGUUUUAGAGC | 1342 |
| 3141 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3451- | gRNA3451 | TSBTx6310 | mCsmUsmGsACACAAUGCUCAGAAACGUUUUAGAGC | 1343 |
| 3142 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3452- | gRNA3452 | TSBTx6311 | mCsmUsmGsACACGAUGCUCAGAUGCGUUUUAGAGC | 1344 |
| 3143 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3453- | gRNA3453 | TSBTx6312 | mCsmUsmGsACGCAAUGUCCAGUGAUGUUUUAGAGC | 1345 |
| 3144 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3454- | gRNA3454 | TSBTx6313 | mCsmUsmGsACGCGAUGCUCAGACACGUUUUAGAGC | 1346 |
| 3145 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3455- | gRNA3455 | TSBTx6314 | mCsmUsmGsCCCAUUUAUUUGUCCCUGUUUUAGAGC | 1347 |
| 3146 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3456- | gRNA3456 | TSBTx6315 | mCsmUsmGsCCGAAAUCCAGAUCCUGGUUUUAGAGC | 1348 |
| 3147 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3457- | gRNA3457 | TSBTx6316 | mCsmUsmGsCUACCGAGGUGAUGGACGUUUUAGAGC | 1349 |
| 3148 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3458- | gRNA3458 | TSBTx6317 | mCsmUsmGsGGGUCCAGGACUGCUACGUUUUAGAGC | 1350 |
| 3149 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3459- | gRNA3459 | TSBTx6318 | mCsmUsmGsUCAAUCUUGGUCAUCUAGUUUUAGAGC | 1351 |
| 3150 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3460- | gRNA3460 | TSBTx6319 | mCsmUsmGsUCACCCUAAACAGAGGUGUUUUAGAGC | 1352 |
| 3151 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3461- | gRNA3461 | TSBTx6320 | mCsmUsmGsUUUAGGGUGACAGUGGAGUUUUAGAGC | 1353 |
| 3152 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3462- | gRNA3462 | TSBTx6321 | mGsmAsmAsAUGGACAGAGUUAUCAAGUUUUAGAGC | 1354 |
| 3153 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3463- | gRNA3463 | TSBTx6322 | mGsmAsmAsCAAGGUAAGAAGUCUCUGUUUUAGAGC | 1355 |
| 3154 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3464- | gRNA3464 | TSBTx6323 | mGsmAsmAsGCCCAGCUCCUUGUUAUGUUUUAGAGC | 1356 |
| 3155 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3465- | gRNA3465 | TSBTx6324 | mGsmAsmCsUUCCUACCUUCUUCAGAGUUUUAGAGC | 1357 |
| 3156 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3466- | gRNA3466 | TSBTx6325 | mGsmAsmCsUUCUUACCUUGUUCAGAGUUUUAGAGC | 1358 |
| 3157 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3467- | gRNA3467 | TSBTx6326 | mGsmAsmGsCAAAGCCAUGUGGUCCAGUUUUAGAGC | 1359 |
| 3158 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3468- | gRNA3468 | TSBTx6327 | mGsmAsmGsCAAAGCCCCACAGUCCAGUUUUAGAGC | 1360 |
| 3159 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3469- | gRNA3469 | TSBTx6328 | mGsmAsmGsCAAAGCCCCGGGGUCCAGUUUUAGAGC | 1361 |
| 3160 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3470- | gRNA3470 | TSBTx6329 | mGsmAsmGsGAUGCUGUGGCACAAGGGUUUUAGAGC | 1362 |
| 3161 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3471- | gRNA3471 | TSBTx6330 | mGsmCsmCsACAGCAUCCUCUUCAUUGUUUUAGAGC | 1363 |
| 3162 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3472- | gRNA3472 | TSBTx6331 | mGsmCsmUsCCUUACCUUGUUCAGAAGUUUUAGAGC | 1364 |
| 3163 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3473- | gRNA3473 | TSBTx6332 | mGsmCsmUsGCUAAAAUUAAAACAGAGUUUUAGAGC | 1365 |
| 3164 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3474- | gRNA3474 | TSBTx6333 | mGsmCsmUsUCUUACCUUCUUCAGAAGUUUUAGAGC | 1366 |
| 3165 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3475- | gRNA3475 | TSBTx6334 | mGsmGsmAsGCAAAGCCCCACAGUCCGUUUUAGAGC | 1367 |
| 3166 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3476- | gRNA3476 | TSBTx6335 | mGsmGsmAsGCAAAGCCCCGGGGUCCGUUUUAGAGC | 1368 |
| 3167 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3477- | gRNA3477 | TSBTx6336 | mGsmGsmAsUGCUGUGGCACAAGGUGGUUUUAGAGC | 1369 |
| 3168 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3478- | gRNA3478 | TSBTx6337 | mGsmGsmCsAGUCCAUUCUGCAUCUGGUUUUAGAGC | 1370 |
| 3169 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3479- | gRNA3479 | TSBTx6338 | mGsmGsmCsUCCUUACCUUGUUCAGAGUUUUAGAGC | 1371 |
| 3170 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3480- | gRNA3480 | TSBTx6339 | mGsmGsmCsUUCUUACCUUCUUCAGAGUUUUAGAGC | 1372 |
| 3171 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3481- | gRNA3481 | TSBTx6340 | mGsmGsmGsGUCCAGGACUGCUACCGGUUUUAGAGC | 1373 |
| 3172 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3482- | gRNA3482 | TSBTx6341 | mGsmGsmGsUGCAGGAGUGCUACCACGUUUUAGAGC | 1374 |
| 3173 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3483- | gRNA3483 | TSBTx6342 | mGsmGsmUsGCUGAAAUGAAAAGAAAGUUUUAGAGC | 1375 |
| 3174 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3484- | gRNA3484 | TSBTx6343 | mGsmGsmUsGCUGCUAAAAUUAAAACGUUUUAGAGC | 1376 |
| 3175 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3485- | gRNA3485 | TSBTx6344 | mGsmUsmCsACCCUAAACAGAGGUAGGUUUUAGAGC | 1377 |
| 3176 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3486- | gRNA3486 | TSBTx6345 | mGsmUsmCsAGUGCUGAAAUUAAAACGUUUUAGAGC | 1378 |
| 3177 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3487- | gRNA3487 | TSBTx6346 | mGsmUsmCsAUCCAGGUUCCAAGCCUGUUUUAGAGC | 1379 |
| 3178 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3488- | gRNA3488 | TSBTx6347 | mGsmUsmCsCAGGACUGCUACCGAGGGUUUUAGAGC | 1380 |
| 3179 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3489- | gRNA3489 | TSBTx6348 | mUsmAsmAsCACCAAGGACUAAUCUCGUUUUAGAGC | 1381 |
| 3180 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3490- | gRNA3490 | TSBTx6349 | mUsmAsmAsCACCAAGGGGCUGCCACGUUUUAGAGC | 1382 |
| 3181 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3491- | gRNA3491 | TSBTx6350 | mUsmAsmAsCACCAGGGUUGUUUCCCGUUUUAGAGC | 1383 |
| 3182 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3492- | gRNA3492 | TSBTx6351 | mUsmAsmCsAGGCCUGCCGUCAUCACGUUUUAGAGC | 1384 |
| 3183 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3493- | gRNA3493 | TSBTx6352 | mUsmCsmAsCCCUAAACAGAGGUAGGGUUUUAGAGC | 1385 |
| 3184 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3494- | gRNA3494 | TSBTx6353 | mUsmCsmAsUCCAGGUUCCAAGCCUAGUUUUAGAGC | 1386 |
| 3185 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3495- | gRNA3495 | TSBTx6354 | mUsmGsmAsACAAGGUAAGAAAUUUGGUUUUAGAGC | 1387 |
| 3186 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3496- | gRNA3496 | TSBTx6355 | mUsmGsmAsACAAGGUAAGAAGUCUCGUUUUAGAGC | 1388 |
| 3187 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3497- | gRNA3497 | TSBTx6356 | mUsmGsmAsACAAGGUAAGGAGCCUGGUUUUAGAGC | 1389 |
| 3188 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3498- | gRNA3498 | TSBTx6357 | mUsmGsmAsGCAAAGCCAUGUGGUCCGUUUUAGAGC | 1390 |
| 3189 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3499- | gRNA3499 | TSBTx6358 | mUsmGsmCsCGAAAUCCAGAUCCUGUGUUUUAGAGC | 1391 |
| 3190 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3500- | gRNA3500 | TSBTx6359 | mUsmGsmUsCACCCUAAACAGAGGUAGUUUUAGAGC | 1392 |
| 3191 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3501- | gRNA3501 | TSBTx6360 | mUsmGsmUsUUAGGGUGACAGUGGAGGUUUUAGAGC | 1393 |
| 3192 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3502- | gRNA3502 | TSBTx6361 | mUsmUsmAsAUUUCAGCACUGACUGAGUUUUAGAGC | 1394 |
| 3193 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3503- | gRNA3503 | TSBTx6362 | mUsmUsmCsCUACCUUCUUCAGAAGAGUUUUAGAGC | 1395 |
| 3194 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3504- | gRNA3504 | TSBTx6363 | mUsmUsmCsUACAGACUGUAUGUUUGGUUUUAGAGC | 1396 |
| 3195 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3505- | gRNA3505 | TSBTx6364 | mUsmUsmCsUUACCUGCUUCAGAAUGGUUUUAGAGC | 1397 |
| 3196 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3506- | gRNA3506 | TSBTx6365 | mUsmUsmCsUUACCUUGUUCAAAAAAGUUUUAGAGC | 1398 |
| 3197 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3507- | gRNA3507 | TSBTx6366 | mUsmUsmGsAACAAGGUAAGAAGUUGGUUUUAGAGC | 1399 |
| 3198 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3508- | gRNA3508 | TSBTx6367 | mUsmUsmUsCAGCACCAACUGAAAACGUUUUAGAGC | 1400 |
| 3199 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3509- | gRNA3509 | TSBTx6368 | mUsmUsmUsCAGCACCACCUGAGAAAGUUUUAGAGC | 1401 |
| 3200 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |
| gRNA3510- | gRNA3510 | TSBTx6369 | mUsmUsmUsCUACAGACUGUAUGUUUGUUUUAGAGC | 1402 |
| 3201 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3511- | gRNA3511 | TSBTx6370 | mUsmUsmUSUAGCAGCACCUGAGCAAGUUUUAGAGC | 1403 |
| 3202 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| gRNA3512- | gRNA3512 | TSBTx6371 | mUsmUsmUsUCUACAGACUGUAUGUUGUUUUAGAGC | 1404 |
| 3203 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUsU | |
| sgRNA_088- | sgRNA_088 | TSBTx904 | mCsmAsmGsGAUCCGCACAGACUCCAGUUUUAGAGC | 1405 |
| 3329 | | | UAGAAAUAGCAAGUUAAAAUAAGGCUAGUCCGUUAU | |
| | | | CAACUUGAAAAAGUGGCACCGAGUCGGUGCmUsmUs | |
| | | | mUSU | |

TABLE 1B-2
Exemplary spacer sequences and their corresponding target sequences for use
in targeting a base editor to disrupt a splice site or introduce a stop codon in an LPA
polynucleotide.

| | Description of | Base | | PAM | |
|---|---|---|---|---|---|
| | the Target | Editor | | Sequence | PAM |
| Guide Name | Alteration | Name | napDNAbp | Family | Sequence |
| ABE_NGA_20 nt_3- | Splice Site | spCas9 | spCas9 | NGA | TGA |
| 9_005_+_160541098_ | Disruption | VRQR | | | |
| spCas9 | | ABE | | | |
| CBE_NGA_20 nt_4- | Splice Site | spCas9 | spCas9 | NGA | TGA |
| 9_006_+_160541098_ | Disruption | VRQR | | | |
| spCas9 | | CBE | | | |
| CBE_NGG_20 nt_4- | Introduction of a | spCas9 | spCas9 | NGG | CGG |
| 9_003_+_160548601_ | Stop Codon | CBE | | | |
| spCas9 | | | | | |
| CBE_NGC_20 nt_4- | Introduction of a | spCas9 | spCas9 | NGC | GGC |
| 9_009_+_160548602_ | Stop Codon | NGC CBE | | | |
| spCas9 | | | | | |
| ABE_NGG_20 nt_3-9_002_−_ | Splice Site | spCas9 | spCas9 | NGG | TGG |
| 160532633_spCas9 | Disruption | ABE | | | |
| CBE_NGA_20 nt_4- | Introduction of a | spCas9 | spCas9 | NGA | GGA |
| 9_006_+_160601042_ | Stop Codon | VRQR | | | |
| spCas9 | | CBE | | | |
| CBE_NGA_20 nt_4- | Splice Site | spCas9 | spCas9 | NGA | AGA |
| 9_006_+_160601092_ | Disruption | VRQR | | | |
| spCas9 | | CBE | | | |
| CBE_NGA_20 nt_4- | Introduction of a | spCas9 | spCas9 | NGA | TGA |
| 9_006_+_160600969_ | Stop Codon | VRQR | | | |
| spCas9 | | CBE | | | |
| ABE_NGG_20 nt_3- | Splice Site | spCas9 | spCas9 | NGG | AGG |
| 9_002_+_160578512_ | Disruption | ABE | | | |
| spCas9 | | | | | |
| ABE_NGC_20 nt_3-9_008_−_ | Splice Site | spCas9 | spCas9 | NGC | GGC |
| 160532632_spCas9 | Disruption | NGC | | | |
| | | ABE | | | |
| CBE_NGG_20 nt_4-9_003_−_ | Introduction of a | spCas9 | spCas9 | NGG | TGG |
| 160589669_spCas9 | Stop Codon | CBE | | | |
| CBE_NGG_20 nt_4-9_003_−_ | Introduction of a | spCas9 | spCas9 | NGG | AGG |
| 160548539_spCas9 | Stop Codon | CBE | | | |
| ABE_NGC_20 nt_3- | Splice Site | spCas9 | spCas9 | NGC | AGC |
| 9_008_+_160541100_ | Disruption | NGC | | | |
| spCas9 | | ABE | | | |
| CBE_NGC_20 nt_4- | Splice Site | spCas9 | spCas9 | NGC | AGC |
| 9_009_+_160541100_ | Disruption | NGC CBE | | | |
| spCas9 | | | | | |
| CBE_NGA_20 nt_4-9_006_−_ | Introduction of a | spCas9 | spCas9 | NGA | AGA |
| 160532598_spCas9 | Stop Codon | VRQR | | | |
| | | CBE | | | |
| CBE_NGG_20 nt_4- | Splice Site | spCas9 | spCas9 | NGG | GGG |
| 9_003_+_160542802_ | Disruption | CBE | | | |
| spCas9 | | | | | |
| CBE_NGC_20 nt_4- | Splice Site | spCas9 | spCas9 | NGC | AGC |
| 9_009_+_160537957_ | Disruption | NGC CBE | | | |
| spCas9 | | | | | |
| CBE_NGA_20 nt_4- | Introduction of a | spCas9 | spCas9 | NGA | GGA |
| 9_006_+_160557482_ | Stop Codon | VRQR | | | |
| spCas9 | | CBE | | | |
| CBE_NGA_20 nt_4- | Splice Site | spCas9 | spCas9 | NGA | AGA |
| 9_006_+_160599655_ | Disruption | VRQR | | | |
| spCas9 | | CBE | | | |
| CBE_NGA_20 nt_4-9_006_−_ | Introduction of a | spCas9 | spCas9 | NGA | TGA |
| 160556067_spCas9 | Stop Codon | VRQR | | | |
| | | CBE | | | |
| CBE_NGC_20 nt_4-9_009_−_ | Introduction of a | spCas9 | spCas9 | NGC | GGC |
| 160599594_spCas9 | Stop Codon | NGC CBE | | | |
| CBE_NGC_20 nt_4-9_009_−_ | Introduction of a | spCas9 | spCas9 | NGC | TGC |
| 160540055_spCas9 | Stop Codon | NGC CBE | | | |
| CBE_NGA_20 nt_4-9_006_−_ | Introduction of a | spCas9 | spCas9 | NGA | AGA |
| 160599514_spCas9 | Stop Codon | VRQR | | | |
| | | CBE | | | |
| CBE_NGG_20 nt_4- | Introduction of a | spCas9 | spCas9 | NGG | CGG |
| 9_003_+_160557481_ | Stop Codon | CBE | | | |
| spCas9 | | | | | |
| CBE_NGA_20 nt_4-9_006_−_ | Introduction of a | spCas9 | spCas9 | NGA | AGA |
| 160650352_spCas9 | Stop Codon | VRQR | | | |
| | | CBE | | | |
| CBE_NGC_20 nt_4-9_009_−_ | Introduction of a | spCas9 | spCas9 | NGC | GGC |
| 160548492_spCas9 | Stop Codon | NGC CBE | | | |
| CBE_NGC_20 nt_4- | Splice Site | spCas9 | spCas9 | NGC | TGC |
| 9_009_+_160541175_ | Disruption | NGC CBE | | | |
| spCas9 | | | | | |
| CBE_NGG_20 nt_4- | Introduction of a | spCas9 | spCas9 | NGG | TGG |
| 9_003_+_160547801_ | Stop Codon | CBE | | | |
| spCas9 | | | | | |
| CBE_NGA_20 nt_4- | Introduction of a | spCas9 | spCas9 | NGA | AGA |
| 9_006_+_160585072_ | Stop Codon | VRQR | | | |
| spCas9 | | CBE | | | |
| CBE_NGG_20 nt_4-9_003_−_ | Introduction of a | spCas9 | spCas9 | NGG | TGG |
| 160556135_spCas9 | Stop Codon | CBE | | | |
| CBE_NGG_20 nt_4- | Introduction of a | spCas9 | spCas9 | NGG | CGG |
| 9_003_+_160557486_ | Stop Codon | CBE | | | |
| spCas9 | | | | | |
| CBE_NGA_20 nt_4-9_006_−_ | Introduction of a | spCas9 | spCas9 | NGA | AGA |
| 160595418_spCas9 | Stop Codon | VRQR | | | |
| | | CBE | | | |
| CBE_NGA_20 nt_4-9_006_−_ | Introduction of a | spCas9 | spCas9 | NGA | AGA |
| 160557454_spCas9 | Stop Codon | VRQR | | | |
| | | CBE | | | |
| CBE_NGA_20 nt_4-9_006_−_ | Introduction of a | spCas9 | spCas9 | NGA | AGA |
| 160600981_spCas9 | Stop Codon | VRQR | | | |
| | | CBE | | | |
| CBE_NGA_20 nt_4-9_006_−_ | Introduction of a | spCas9 | spCas9 | NGA | GGA |
| 160586513_spCas9 | Stop Codon | VRQR | | | |
| | | CBE | | | |
| CBE_NGA_20 nt_4-9_006_−_ | Introduction of a | spCas9 | spCas9 | NGA | AGA |
| 160548542_spCas9 | Stop Codon | VRQR | | | |
| | | CBE | | | |
| CBE_NGA_20 nt_4- | Introduction of a | spCas9 | spCas9 | NGA | GGA |
| 9_006_+_160547802_ | Stop Codon | VRQR | | | |
| spCas9 | | CBE | | | |
| CBE_NGG_20 nt_4-9_003_−_ | Introduction of a | spCas9 | spCas9 | NGG | TGG |
| 160601057_spCas9 | Stop Codon | CBE | | | |
| CBE_NGA_20 nt_4-9_006_−_ | Introduction of a | spCas9 | spCas9 | NGA | AGA |
| 160585151_spCas9 | Stop Codon | VRQR | | | |
| | | CBE | | | |
| CBE_NGA_20 nt_4-9_006_−_ | Introduction of a | spCas9 | spCas9 | NGA | CGA |
| 160585164_spCas9 | Stop Codon | VRQR | | | |
| | | CBE | | | |
| CBE_NGA_20 nt_4-9_006_−_ | Introduction of a | spCas9 | spCas9 | NGA | TGA |
| 160577178_spCas9 | Stop Codon | VRQR | | | |
| | | CBE | | | |
| CBE_NGG_20 nt_4- | Splice Site | spCas9 | spCas9 | NGG | AGG |
| 9_003_+_160531882_ | Disruption | CBE | | | |
| spCas9 | | | | | |
| ABE_NGG_20 nt_3-9_002_−_ | Splice Site | spCas9 | spCas9 | NGG | GGG |
| 160531875_spCas9 | Disruption | ABE | | | |
| CBE_NGC_20 nt_4-9_009_−_ | Introduction of a | spCas9 | spCas9 | NGC | GGC |
| 160605140_spCas9 | Stop Codon | NGC CBE | | | |
| CBE_NGC_20 nt_4-9_009_−_ | Introduction of a | spCas9 | spCas9 | NGC | GGC |
| 160590927_spCas9 | Stop Codon | NGC CBE | | | |
| CBE_NGA_20 nt_4-9_006_−_ | Introduction of a | spCas9 | spCas9 | NGA | TGA |
| 160532600_spCas9 | Stop Codon | VRQR | | | |
| | | CBE | | | |
| ABE_NGA_20 nt_3- | Splice Site | spCas9 | spCas9 | NGA | AGA |
| 9_005_+_160595343_ | Disruption | VRQR | | | |
| spCas9 | | ABE | | | |
| ABE_NGG_20 nt_3- | Splice Site | spCas9 | spCas9 | NGG | AGG |
| 9_002_+_160548467_ | Disruption | ABE | | | |
| spCas9 | | | | | |
| CBE_NGA_20 nt_4-9_006_−_ | Introduction of a | spCas9 | spCas9 | NGA | GGA |
| 160650466_spCas9 | Stop Codon | VRQR | | | |
| | | CBE | | | |
| CBE_NGA_20 nt_4-9_006_−_ | Introduction of a | spCas9 | spCas9 | NGA | GGA |
| 160589681_spCas9 | Stop Codon | VRQR | | | |
| | | CBE | | | |
| CBE_NGA_20 nt_4-9_006_−_ | Introduction of a | spCas9 | spCas9 | NGA | GGA |
| 160594086_spCas9 | Stop Codon | VRQR | | | |
| | | CBE | | | |
| CBE_NGG_20 nt_4- | Splice Site | spCas9 | spCas9 | NGG | TGG |
| 9_003_+_160542801_ | Disruption | CBE | | | |
| spCas9 | | | | | |
| ABE_NGA_20 nt_3-9_005_−_ | Splice Site | spCas9 | spCas9 | NGA | TGA |
| 160542792_spCas9 | Disruption | VRQR | | | |
| | | ABE | | | |
| ABE_NGA_20 nt_3- | Splice Site | spCas9 | spCas9 | NGA | GGA |
| 9_005_+_160606467_ | Disruption | VRQR | | | |
| spCas9 | | ABE | | | |
| CBE_NGA_20 nt_4- | Splice Site | spCas9 | spCas9 | NGA | GGA |
| 9_006_+_160606467_ | Disruption | VRQR | | | |
| spCas9 | | CBE | | | |
| CBE_NGA_20 nt_4- | Splice Site | spCas9 | spCas9 | NGA | AGA |
| 9_006_+_160650493_ | Disruption | VRQR | | | |
| spCas9 | | CBE | | | |
| ABE_NGA_20 nt_3- | Splice Site | spCas9 | spCas9 | NGA | GGA |
| 9_005_+_160586439_ | Disruption | VRQR | | | |
| spCas9 | | ABE | | | |
| CBE_NGA_20 nt_4- | Splice Site | spCas9 | spCas9 | NGA | GGA |
| 9_006_+_160586439_ | Disruption | VRQR | | | |
| spCas9 | | CBE | | | |
| CBE_NGG_20 nt_4-9_003_−_ | Introduction of a | spCas9 | spCas9 | NGG | AGG |
| 160589682_spCas9 | Stop Codon | CBE | | | |
| CBE_NGG_20 nt_4-9_003_−_ | Introduction of a | spCas9 | spCas9 | NGG | AGG |
| 160594087_spCas9 | Stop Codon | CBE | | | |
| CBE_NGA_20 nt_4- | Splice Site | spCas9 | spCas9 | NGA | GGA |
| 9_006_+_160542803_ | Disruption | VRQR | | | |
| spCas9 | | CBE | | | |
| CBE_NGC_20 nt_4- | Introduction of a | spCas9 | spCas9 | NGC | AGC |
| 9_009_+_160600971_ | Stop Codon | NGC CBE | | | |
| spCas9 | | | | | |
| ABE_NGG_20 nt_3- | Splice Site | spCas9 | spCas9 | NGG | AGG |
| 9_002_+_160606466_ | Disruption | ABE | | | |
| spCas9 | | | | | |
| ABE_NGG_20 nt_3- | Splice Site | spCas9 | spCas9 | NGG | AGG |
| 9_002_+_160586438_ | Disruption | ABE | | | |
| spCas9 | | | | | |
| CBE_NGG_20 nt_4-9_003_−_ | Introduction of a | spCas9 | spCas9 | NGG | AGG |
| 160585162_spCas9 | Stop Codon | CBE | | | |
| CBE_NGA_20 nt_4-9_006_−_ | Introduction of a | spCas9 | spCas9 | NGA | GGA |
| 160605161_spCas9 | Stop Codon | VRQR | | | |
| | | CBE | | | |
| CBE_NGA_20 nt_4- | Splice Site | spCas9 | spCas9 | NGA | AGA |
| 9_006_+_160605201_ | Disruption | VRQR | | | |
| spCas9 | | CBE | | | |
| CBE_NGA_20 nt_4- | Splice Site | spCas9 | spCas9 | NGA | AGA |
| 9_006_+_160650490_ | Disruption | VRQR | | | |
| spCas9 | | CBE | | | |
| CBE_NGG_20 nt_4- | Splice Site | spCas9 | spCas9 | NGG | GGG |
| 9_003_+_160531884_ | Disruption | CBE | | | |
| spCas9 | | | | | |
| CBE_NGA_20 nt_4- | Splice Site | spCas9 | spCas9 | NGA | AGA |
| 9_006_+_160599652_ | Disruption | VRQR | | | |
| spCas9 | | CBE | | | |
| CBE_NGG_20 nt_4-9_003_−_ | Introduction of a | spCas9 | spCas9 | NGG | AGG |
| 160548494_spCas9 | Stop Codon | CBE | | | |
| CBE_NGA_20 nt_4-9_006_−_ | Introduction of a | spCas9 | spCas9 | NGA | TGA |
| 160585159_spCas9 | Stop Codon | VRQR | | | |
| | | CBE | | | |
| CBE_NGC_20 nt_4- | Introduction of a | spCas9 | spCas9 | NGC | AGC |
| 9_009_+_160591044_ | Stop Codon | NGC CBE | | | |
| spCas9 | | | | | |
| CBE_NGG_20 nt_4- | Introduction of a | spCas9 | spCas9 | NGG | AGG |
| 9_003_+_160601041_ | Stop Codon | CBE | | | |
| spCas9 | | | | | |
| CBE_NGA_20 nt_4- | Introduction of a | spCas9 | spCas9 | NGA | AGA |
| 9_006_+_160557514_ | Stop Codon | VRQR | | | |
| spCas9 | | CBE | | | |
| ABE_NGA_20 nt_3-9_005_−_ | Splice Site | spCas9 | spCas9 | NGA | TGA |
| 160537943_spCas9 | Disruption | VRQR | | | |
| | | ABE | | | |
| CBE_NGA_20 nt_4- | Splice Site | spCas9 | spCas9 | NGA | GGA |
| 9_006_+_160531885_ | Disruption | VRQR | | | |
| spCas9 | | CBE | | | |
| CBE_NGG_20 nt_4-9_003_−_ | Introduction of a | spCas9 | spCas9 | NGG | GGG |
| 160548493_spCas9 | Stop Codon | CBE | | | |
| CBE_NGG_20 nt_4-9_003_−_ | Introduction of a | spCas9 | spCas9 | NGG | TGG |
| 160578507_spCas9 | Stop Codon | CBE | | | |
| CBE_NGG_20 nt_4-9_003_−_ | Introduction of a | spCas9 | spCas9 | NGG | TGG |
| 160590928_spCas9 | Stop Codon | CBE | | | |
| CBE_NGG_20 nt_4-9_003_−_ | Introduction of a | spCas9 | spCas9 | NGG | TGG |
| 160606461_spCas9 | Stop Codon | CBE | | | |
| CBE_NGG_20 nt_4-9_003_−_ | Introduction of a | spCas9 | spCas9 | NGG | AGG |
| 160650467_spCas9 | Stop Codon | CBE | | | |
| CBE_NGC_20 nt_4-9_009_−_ | Introduction of a | spCas9 | spCas9 | NGC | GGC |
| 160601056_spCas9 | Stop Codon | NGC CBE | | | |
| CBE_NGG_20 nt_4- | Splice Site | spCas9 | spCas9 | NGG | GGG |
| 9_003_+_160531883_ | Disruption | CBE | | | |
| spCas9 | | | | | |
| ABE_NGC_20 nt_3-9_008_−_ | Splice Site | spCas9 | spCas9 | NGC | GGC |
| 160531874_spCas9 | Disruption | NGC | | | |
| | | ABE | | | |
| ABE_NGA_20 nt_3-9_005_−_ | Splice Site | spCas9 | spCas9 | NGA | GGA |
| 160599646_spCas9 | Disruption | VRQR | | | |
| | | ABE | | | |
| ABE_NGC_20 nt_3- | Splice Site | spCas9 | spCas9 | NGC | AGC |
| 9_008_+_160595346_ | Disruption | NGC | | | |
| spCas9 | | ABE | | | |
| CBE_NGC_20 nt_4- | Splice Site | spCas9 | spCas9 | NGC | AGC |
| 9_009_+_160595346_ | Disruption | NGC CBE | | | |
| spCas9 | | | | | |
| ABE_NGA_20 nt_3-9_005_−_ | Splice Site | spCas9 | spCas9 | NGA | GGA |
| 160547922_spCas9 | Disruption | VRQR | | | |
| | | ABE | | | |
| ABE_NGC_20 nt_3- | Splice Site | spCas9 | spCas9 | NGC | AGC |
| 9_008_+_160557382_ | Disruption | NGC | | | |
| spCas9 | | ABE | | | |
| CBE_NGC_20 nt_4- | Splice Site | spCas9 | spCas9 | NGC | AGC |
| 9_009_+_160557382_ | Disruption | NGC CBE | | | |
| spCas9 | | | | | |
| ABE_NGC_20 nt_3- | Splice Site | spCas9 | spCas9 | NGC | AGC |
| 9_008_+_160600909_ | Disruption | NGC | | | |
| spCas9 | | ABE | | | |
| CBE_NGC_20 nt_4- | Splice Site | spCas9 | spCas9 | NGC | AGC |
| 9_009_+_160600909_ | Disruption | NGC CBE | | | |
| spCas9 | | | | | |
| CBE_NGC_20 nt_4-9_009_−_ | Introduction of a | spCas9 | spCas9 | NGC | TGC |
| 160600902_spCas9 | Stop Codon | NGC CBE | | | |
| ABE_NGC_20 nt_3-9_008_−_ | Splice Site | spCas9 | spCas9 | NGC | AGC |
| 160585188_spCas9 | Disruption | NGC | | | |
| | | ABE | | | |
| ABE_NGC_20 nt_3-9_008_−_ | Splice Site | spCas9 | spCas9 | NGC | AGC |
| 160577278_spCas9 | Disruption | NGC | | | |
| | | ABE | | | |
| ABE_NGG_20 nt_3-9_002_−_ | Splice Site | spCas9 | spCas9 | NGG | GGG |
| 160547923_spCas9 | Disruption | ABE | | | |
| ABE_NGC_20 nt_3-9_008_−_ | Splice Site | spCas9 | spCas9 | NGC | AGC |
| 160650480_spCas9 | Disruption | NGC | | | |
| | | ABE | | | |
| ABE_NGG_20 nt_3-9_002_−_ | Splice Site | spCas9 | spCas9 | NGG | TGG |
| 160547924_spCas9 | Disruption | ABE | | | |
| | | | Target Nucleotide | Location of target site | |
| | | SEQ | Position in Target | on Chr6 (hg38.2bit | |
| | | ID | Sequence (from 5′ | genome sequence) | |
| Guide Name | Target Sequence | NO | end) | start | end |
| ABE_NGA_20 nt_3- | AAACGTACTTCTT | 1408 | 7 | 160541098 | 160541121 |
| 9_005_+_160541098_ | CAAGCAGTGA | | | | |
| spCas9 | | | | | |
| CBE_NGA_20 nt_4- | AAACGTACTTCTT | 1409 | 8 | 160541098 | 160541121 |
| 9_006_+_160541098_ | CAAGCAGTGA | | | | |
| spCas9 | | | | | |
| CBE_NGG_20 nt_4- | AAAACACCAAGGG | 1406 | 8, 7 | 160548601 | 160548624 |
| 9_003_+_160548601_ | CCTGTATCGG | | | | |
| spCas9 | | | | | |
| CBE_NGC_20 nt_4- | AAACACCAAGGGC | 1407 | 7, 6 | 160548602 | 160548625 |
| 9_009_+_160548602_ | CTGTATCGGC | | | | |
| spCas9 | | | | | |
| ABE_NGG_20 nt_3- | AACACAGGTACCT | 1410 | 6 | 160532633 | 160532656 |
| 9_002_−_ | TTGGGACTGG | | | | |
| 160532633_spCas9 | | | | | |
| CBE_NGA_20 nt_4- | AACACCAAGGGGC | 1411 | 6, 5 | 160601042 | 160601065 |
| 9_006_+_160601042_ | TGCCACAGGA | | | | |
| spCas9 | | | | | |
| CBE_NGA_20 nt_4- | AAGCCACTGGAAA | 1412 | 7 | 160601092 | 160601115 |
| 9_006_+_160601092_ | TTCCAAAAGA | | | | |
| _spCas9 | | | | | |
| CBE_NGA_20 nt_4- | AAGGCAGTCCATT | 1413 | 9 | 160600969 | 160600992 |
| 9_006_+_160600969_ | CTGCATCTGA | | | | |
| spCas9 | | | | | |
| ABE_NGG_20 nt_3- | AATTTCTTACCTT | 1414 | 9 | 160578512 | 160578535 |
| 9_002_+_160578512_ | GTTCAGAAGG | | | | |
| spCas9 | | | | | |
| ABE_NGC_20 nt_3- | ACACAGGTACCTT | 1415 | 5 | 160532632 | 160532655 |
| 9_008_−_ | TGGGACTGGC | | | | |
| 160532632_spCas9 | | | | | |
| CBE_NGG_20 nt_4- | ACAGTCCAGGACT | 1416 | 7 | 160589669 | 160589692 |
| 9_003_−_ | GCTACCATGG | | | | |
| 160589669_spCas9 | | | | | |
| CBE_NGG_20 nt_4- | ACGCGATGCTCAG | 1417 | 4 | 160548539 | 160548562 |
| 9_003_−_ | ACACAGAAGG | | | | |
| 160548539_spCas9 | | | | | |
| ABE_NGC_20 nt_3- | ACGTACTTCTTCA | 1418 | 5 | 160541100 | 160541123 |
| 9_008_+_160541100_ | AGCAGTGAGC | | | | |
| spCas9 | | | | | |
| CBE_NGC_20 nt_4- | ACGTACTTCTTCA | 1419 | 6 | 160541100 | 160541123 |
| 9_009_+_160541100_ | AGCAGTGAGC | | | | |
| spCas9 | | | | | |
| CBE_NGA_20 nt_4- | AGCCCAGCTCCTT | 1420 | 5 | 160532598 | 160532621 |
| 9_006_−_ | GTTATTGAGA | | | | |
| 160532598_spCas9 | | | | | |
| CBE_NGG_20 nt_4- | AGGATGCTGTGGC | 1421 | 7 | 160542802 | 160542825 |
| 9_003_+_160542802_ | ACAAGGTGGG | | | | |
| spCas9 | | | | | |
| CBE_NGC_20 nt_4- | AGGCCTGTAAGGA | 1422 | 5 | 160537957 | 160537980 |
| 9_009_+_160537957_ | AAGTATTAGC | | | | |
| spCas9 | | | | | |
| CBE_NGA_20 nt_4- | AGTACTCCCACCT | 1423 | 9, 8 | 160557482 | 160557505 |
| 9_006_+_160557482_ | CACACACGGA | | | | |
| spCas9 | | | | | |
| CBE_NGA_20 nt_4- | AGTGCTGAAATTA | 1424 | 5 | 160599655 | 160599678 |
| 9_006_+_160599655_ | AAACAGAAGA | | | | |
| spCas9 | | | | | |
| CBE_NGA_20 nt_4- | ATGTCAATCTTGG | 1425 | 5 | 160556067 | 160556090 |
| 9_006_−_ | TCATCCATGA | | | | |
| 160556067_spCas9 | | | | | |
| CBE_NGC_20 nt_4- | ATTATGGACAGAG | 1426 | 9 | 160599594 | 160599617 |
| 9_009_−_ | TTACCGAGGC | | | | |
| 160599594_spCas9 | | | | | |
| CBE_NGC_20 nt_4- | CACACAAGCAGAT | 1427 | 5 | 160540055 | 160540078 |
| 9_009_−_ | ATTGCCTTGC | | | | |
| 160540055_spCas9 | | | | | |
| CBE_NGA_20 nt_4- | CACCAGCATAGTC | 1428 | 4 | 160599514 | 160599537 |
| 9_006_−_ | GGACCCCAGA | | | | |
| 160599514_spCas9 | | | | | |
| CBE_NGG_20 nt_4- | CAGTACTCCCACC | 1429 | 9 | 160557481 | 160557504 |
| 9_003_+_160557481_ | TCACACACGG | | | | |
| spCas9 | | | | | |
| CBE_NGA_20 nt_4- | CATCAACATAATA | 1430 | 4 | 160650352 | 160650375 |
| 9_006_−_ | GGACCACAGA | | | | |
| 160650352_spCas9 | | | | | |
| CBE_NGC_20 nt_4- | CATCCAGGTTCCA | 1431 | 5 | 160548492 | 160548515 |
| 9_009_−_ | AGCCTAGGGC | | | | |
| 160548492_spCas9 | | | | | |
| CBE_NGC_20 nt_4- | CCAAACCTAGAAA | 1432 | 7 | 160541175 | 160541198 |
| 9_009_+_160541175_ | GAAAACATGC | | | | |
| spCas9 | | | | | |
| CBE_NGG_20 nt_4- | CCTGCCCATTTAT | 1433 | 7, 6 | 160547801 | 160547824 |
| 9_003_+_160547801_ | TTGTCCCTGG | | | | |
| spCas9 | | | | | |
| CBE_NGA_20 nt_4- | CGATGCCAATGTG | 1434 | 7, 6 | 160585072 | 160585095 |
| 9_006_+_160585072_ | GTGTCATAGA | | | | |
| spCas9 | | | | | |
| CBE_NGG_20 nt_4- | CGGCAGTGCTACC | 1435 | 4 | 160556135 | 160556158 |
| 9_003_−_ | ATGGTAATGG | | | | |
| 160556135_spCas9 | | | | | |
| CBE_NGG_20 nt_4- | CTCCCACCTCACA | 1436 | 5, 4 | 160557486 | 160557509 |
| 9_003_+_160557486_ | CACGGATCGG | | | | |
| spCas9 | | | | | |
| CBE_NGA_20 nt_4- | CTGACACAATGCC | 1437 | 7 | 160595418 | 160595441 |
| 9_006_−_ | TGGTGACAGA | | | | |
| 160595418_spCas9 | | | | | |
| CBE_NGA_20 nt_4- | CTGACACAATGCT | 1438 | 7 | 160557454 | 160557477 |
| 9_006_−_ | CAGAAACAGA | | | | |
| 160557454_spCas9 | | | | | |
| CBE_NGA_20 nt_4- | CTGACACGATGCT | 1439 | 7 | 160600981 | 160601004 |
| 9_006_−_ | CAGATGCAGA | | | | |
| 160600981_spCas9 | | | | | |
| CBE_NGA_20 nt_4- | CTGACGCAATGTC | 1440 | 7 | 160586513 | 160586536 |
| 9_006_−_ | CAGTGATGGA | | | | |
| 160586513_spCas9 | | | | | |
| CBE_NGA_20 nt_4- | CTGACGCGATGCT | 1441 | 7 | 160548542 | 160548565 |
| 9_006_−_ | CAGACACAGA | | | | |
| 160548542_spCas9 | | | | | |
| CBE_NGA_20 nt_4- | CTGCCCATTTATT | 1442 | 6, 5 | 160547802 | 160547825 |
| 9_006_+_160547802_ | TGTCCCTGGA | | | | |
| spCas9 | | | | | |
| CBE_NGG_20 nt_4- | CTGCCGAAATCCA | 1443 | 5 | 160601057 | 160601080 |
| 9_003_−_ | GATCCTGTGG | | | | |
| 160601057_spCas9 | | | | | |
| CBE_NGA_20 nt_4- | CTGCTACCGAGGT | 1444 | 8 | 160585151 | 160585174 |
| 9_006_−_ | GATGGACAGA | | | | |
| 160585151_spCas9 | | | | | |
| CBE_NGA_20 nt_4- | CTGGGGTCCAGGA | 1445 | 9 | 160585164 | 160585187 |
| 9_006_−_ | CTGCTACCGA | | | | |
| 160585164_spCas9 | | | | | |
| CBE_NGA_20 nt_4- | CTGTCAATCTTGG | 1446 | 5 | 160577178 | 160577201 |
| 9_006_−_ | TCATCTATGA | | | | |
| 160577178_spCas9 | | | | | |
| CBE_NGG_20 nt_4- | CTGTCACCCTAAA | 1447 | 9 | 160531882 | 160531905 |
| 9_003_+_160531882_ | CAGAGGTAGG | | | | |
| spCas9 | | | | | |
| ABE_NGG_20 nt_3- | CTGTTTAGGGTGA | 1448 | 7 | 160531875 | 160531898 |
| 9_002_−_ | CAGTGGAGGG | | | | |
| 160531875_spCas9 | | | | | |
| CBE_NGC_20 nt_4- | GAAATGGACAGAG | 1449 | 9 | 160605140 | 160605163 |
| 9_009_−_ | TTATCAAGGC | | | | |
| 160605140_spCas9 | | | | | |
| CBE_NGC_20 nt_4- | GAACAAGGTAAGA | 1450 | 4 | 160590927 | 160590950 |
| 9_009_−_ | AGTCTCTGGC | | | | |
| 160590927_spCas9 | | | | | |
| CBE_NGA_20 nt_4- | GAAGCCCAGCTCC | 1451 | 7 | 160532600 | 160532623 |
| 9_006_−_ | TTGTTATTGA | | | | |
| 160532600_spCas9 | | | | | |
| ABE_NGA_20 nt_3- | GACTTCCTACCTT | 1452 | 9 | 160595343 | 160595366 |
| 9_005_+_160595343_ | CTTCAGAAGA | | | | |
| spCas9 | | | | | |
| ABE_NGG_20 nt_3- | GACTTCTTACCTT | 1453 | 9 | 160548467 | 160548490 |
| 9_002_+_160548467_ | GTTCAGAAGG | | | | |
| spCas9 | | | | | |
| CBE_NGA_20 nt_4- | GAGCAAAGCCATG | 1454 | 4 | 160650466 | 160650489 |
| 9_006_−_ | TGGTCCAGGA | | | | |
| 160650466_spCas9 | | | | | |
| CBE_NGA_20 nt_4- | GAGCAAAGCCCCA | 1455 | 4 | 160589681 | 160589704 |
| 9_006_−_ | CAGTCCAGGA | | | | |
| 160589681_spCas9 | | | | | |
| CBE_NGA_20 nt_4- | GAGCAAAGCCCCG | 1456 | 4 | 160594086 | 160594109 |
| 9_006_−_ | GGGTCCAGGA | | | | |
| 160594086_spCas9 | | | | | |
| CBE_NGG_20 nt_4- | GAGGATGCTGTGG | 1457 | 8 | 160542801 | 160542824 |
| 9_003_+_160542801_ | CACAAGGTGG | | | | |
| spCas9 | | | | | |
| ABE_NGA_20 nt_3- | GCCACAGCATCCT | 1458 | 6 | 160542792 | 160542815 |
| 9_005_−_ | CTTCATTTGA | | | | |
| 160542792_spCas9 | | | | | |
| ABE_NGA_20 nt_3- | GCTCCTTACCTTG | 1459 | 8 | 160606467 | 160606490 |
| 9_005_+_160606467_ | TTCAGAAGGA | | | | |
| spCas9 | | | | | |
| CBE_NGA_20 nt_4- | GCTCCTTACCTTG | 1460 | 9 | 160606467 | 160606490 |
| 9_006_+_160606467_ | TTCAGAAGGA | | | | |
| spCas9 | | | | | |
| CBE_NGA_20 nt_4- | GCTGCTAAAATTA | 1461 | 5 | 160650493 | 160650516 |
| 9_006_+_160650493_ | AAACAGAAGA | | | | |
| spCas9 | | | | | |
| ABE_NGA_20 nt_3- | GCTTCTTACCTTC | 1462 | 8 | 160586439 | 160586462 |
| 9_005_+_160586439_ | TTCAGAAGGA | | | | |
| spCas9 | | | | | |
| CBE_NGA_20 nt_4- | GCTTCTTACCTTC | 1463 | 9 | 160586439 | 160586462 |
| 9_006_+_160586439_ | TTCAGAAGGA | | | | |
| spCas9 | | | | | |
| CBE_NGG_20 nt_4- | GGAGCAAAGCCCC | 1464 | 5 | 160589682 | 160589705 |
| 9_003_−_ | ACAGTCCAGG | | | | |
| 160589682_spCas9 | | | | | |
| CBE_NGG_20 nt_4- | GGAGCAAAGCCCC | 1465 | 5 | 160594087 | 160594110 |
| 9_003_−_ | GGGGTCCAGG | | | | |
| 160594087_spCas9 | | | | | |
| CBE_NGA_20 nt_4- | GGATGCTGTGGCA | 1466 | 6 | 160542803 | 160542826 |
| 9_006_+_160542803_ | CAAGGTGGGA | | | | |
| spCas9 | | | | | |
| CBE_NGC_20 nt_4- | GGCAGTCCATTCT | 1467 | 8, 7 | 160600971 | 160600994 |
| 9_009_+_160600971_ | GCATCTGAGC | | | | |
| spCas9 | | | | | |
| ABE_NGG_20 nt_3- | GGCTCCTTACCTT | 1468 | 9 | 160606466 | 160606489 |
| 9_002_+_160606466_ | GTTCAGAAGG | | | | |
| spCas9 | | | | | |
| ABE_NGG_20 nt_3- | GGCTTCTTACCTT | 1469 | 9 | 160586438 | 160586461 |
| 9_002_+_160586438_ | CTTCAGAAGG | | | | |
| spCas9 | | | | | |
| CBE_NGG_20 nt_4- | GGGGTCCAGGACT | 1470 | 7 | 160585162 | 160585185 |
| 9_003_−_ | GCTACCGAGG | | | | |
| 160585162_spCas9 | | | | | |
| CBE_NGA_20 nt_4- | GGGTGCAGGAGTG | 1471 | 6 | 160605161 | 160605184 |
| 9_006_−_ | CTACCACGGA | | | | |
| 160605161_spCas9 | | | | | |
| CBE_NGA_20 nt_4- | GGTGCTGAAATGA | 1472 | 5 | 160605201 | 160605224 |
| 9_006_+_160605201_ | AAAGAAAAGA | | | | |
| spCas9 | | | | | |
| CBE_NGA_20 nt_4- | GGTGCTGCTAAAA | 1473 | 8 | 160650490 | 160650513 |
| 9_006_+_160650490_ | TTAAAACAGA | | | | |
| spCas9 | | | | | |
| CBE_NGG_20 nt_4- | GTCACCCTAAACA | 1474 | 7 | 160531884 | 160531907 |
| 9_003_+_160531884_ | GAGGTAGGGG | | | | |
| spCas9 | | | | | |
| CBE_NGA_20 nt_4- | GTCAGTGCTGAAA | 1475 | 8 | 160599652 | 160599675 |
| 9_006_+_16059965 | TTAAAACAGA | | | | |
| 2_spCas9 | | | | | |
| CBE_NGG_20 nt_4- | GTCATCCAGGTTC | 1476 | 7 | 160548494 | 160548517 |
| 9_003_−_ | CAAGCCTAGG | | | | |
| 160548494_spCas9 | | | | | |
| CBE_NGA_20 nt_4- | GTCCAGGACTGCT | 1477 | 4 | 160585159 | 160585182 |
| 9_006_−_ | ACCGAGGTGA | | | | |
| 160585159_spCas9 | | | | | |
| CBE_NGC_20 nt_4- | TAACACCAAGGAC | 1478 | 7, 6 | 160591044 | 160591067 |
| 9_009_+_160591044_ | TAATCTCAGC | | | | |
| spCas9 | | | | | |
| CBE_NGG_20 nt_4- | TAACACCAAGGGG | 1479 | 7, 6 | 160601041 | 160601064 |
| 9_003_+_160601041_ | CTGCCACAGG | | | | |
| spCas9 | | | | | |
| CBE_NGA_20 nt_4- | TAACACCAGGGTT | 1480 | 7, 6 | 160557514 | 160557537 |
| 9_006_+_160557514_ | GTTTCCCAGA | | | | |
| spCas9 | | | | | |
| ABE_NGA_20 nt_3- | TACAGGCCTGCCG | 1481 | 4 | 160537943 | 160537966 |
| 9_005_−_ | TCATCACTGA | | | | |
| 160537943_spCas9 | | | | | |
| CBE_NGA_20 nt_4- | TCACCCTAAACAG | 1482 | 6 | 160531885 | 160531908 |
| 9_006_+_160531885_ | AGGTAGGGGA | | | | |
| spCas9 | | | | | |
| CBE_NGG_20 nt_4- | TCATCCAGGTTCC | 1483 | 6 | 160548493 | 160548516 |
| 9_003_−_ | AAGCCTAGGG | | | | |
| 160548493_spCas9 | | | | | |
| CBE_NGG_20 nt_4- | TGAACAAGGTAAG | 1484 | 5 | 160578507 | 160578530 |
| 9_003_−_ | AAATTTGTGG | | | | |
| 160578507_spCas9 | | | | | |
| CBE_NGG_20 nt_4- | TGAACAAGGTAAG | 1485 | 5 | 160590928 | 160590951 |
| 9_003_−_ | AAGTCTCTGG | | | | |
| 160590928_spCas9 | | | | | |
| CBE_NGG_20 nt_4- | TGAACAAGGTAAG | 1486 | 5 | 160606461 | 160606484 |
| 9_003_−_ | GAGCCTGTGG | | | | |
| 160606461_spCas9 | | | | | |
| CBE_NGG_20 nt_4- | TGAGCAAAGCCAT | 1487 | 5 | 160650467 | 160650490 |
| 9_003_−_ | GTGGTCCAGG | | | | |
| 160650467_spCas9 | | | | | |
| CBE_NGC_20 nt_4- | TGCCGAAATCCAG | 1488 | 4 | 160601056 | 160601079 |
| 9_009_−_ | ATCCTGTGGC | | | | |
| 160601056_spCas9 | | | | | |
| CBE_NGG_20 nt_4- | TGTCACCCTAAAC | 1489 | 8 | 160531883 | 160531906 |
| 9_003_+_160531883_ | AGAGGTAGGG | | | | |
| spCas9 | | | | | |
| ABE_NGC_20 nt_3- | TGTTTAGGGTGAC | 1490 | 6 | 160531874 | 160531897 |
| 9_008_−_ | AGTGGAGGGC | | | | |
| 160531874_spCas9 | | | | | |
| ABE_NGA_20 nt_3- | TTAATTTCAGCAC | 1491 | 9 | 160599646 | 160599669 |
| 9_005_−_ | TGACTGAGGA | | | | |
| 160599646_spCas9 | | | | | |
| ABE_NGC_20 nt_3- | TTCCTACCTTCTT | 1492 | 6 | 160595346 | 160595369 |
| 9_008_+_160595346_ | CAGAAGAAGC | | | | |
| spCas9 | | | | | |
| CBE_NGC_20 nt_4- | TTCCTACCTTCTT | 1493 | 7 | 160595346 | 160595369 |
| 9_009_+_160595346_ | CAGAAGAAGC | | | | |
| spCas9 | | | | | |
| ABE_NGA_20 nt_3- | TTCTACAGACTGT | 1494 | 7 | 160547922 | 160547945 |
| 9_005_−_ | ATGTTTGGGA | | | | |
| 160547922_spCas9 | | | | | |
| ABE_NGC_20 nt_3- | TTCTTACCTGCTT | 1495 | 6 | 160557382 | 160557405 |
| 9_008_+_160557382_ | CAGAATGAGC | | | | |
| spCas9 | | | | | |
| CBE_NGC_20 nt_4- | TTCTTACCTGCTT | 1496 | 7 | 160557382 | 160557405 |
| 9_009_+_160557382_ | CAGAATGAGC | | | | |
| spCas9 | | | | | |
| ABE_NGC_20 nt_3- | TTCTTACCTTGTT | 1497 | 6 | 160600909 | 160600932 |
| 9_008_+_160600909_ | CAAAAAAAGC | | | | |
| spCas9 | | | | | |
| CBE_NGC_20 nt_4- | TTCTTACCTTGTT | 1498 | 7 | 160600909 | 160600932 |
| 9_009_+_160600909_ | CAAAAAAAGC | | | | |
| spCas9 | | | | | |
| CBE_NGC_20 nt_4- | TTGAACAAGGTAA | 1499 | 6 | 160600902 | 160600925 |
| 9_009_−_ | GAAGTTGTGC | | | | |
| 160600902_spCas9 | | | | | |
| ABE_NGC_20 nt_3- | TTTCAGCACCAAC | 1500 | 5 | 160585188 | 160585211 |
| 9_008_−_ | TGAAAACAGC | | | | |
| 160585188_spCas9 | | | | | |
| ABE_NGC_20 nt_3- | TTTCAGCACCACC | 1501 | 5 | 160577278 | 160577301 |
| 9_008_−_ | TGAGAAAAGC | | | | |
| 160577278_spCas9 | | | | | |
| ABE_NGG_20 nt_3- | TTTCTACAGACTG | 1502 | 8 | 160547923 | 160547946 |
| 9_002_−_ | TATGTTTGGG | | | | |
| 160547923_spCas9 | | | | | |
| ABE_NGC_20 nt_3- | TTTTAGCAGCACC | 1503 | 5 | 160650480 | 160650503 |
| 9_008_−_ | TGAGCAAAGC | | | | |
| 160650480_spCas9 | | | | | |
| ABE_NGG_20 nt_3- | TTTTCTACAGACT | 1504 | 9 | 160547924 | 160547947 |
| 9_002_−_ | GTATGTTTGG | | | | |
| 160547924_spCas9 | | | | | |

| | | SEQ ID |
|---|---|---|
| Guide Name | Spacer Sequence | NO |
| ABE_NGA_20 nt_3-9_005_+_160541098_spCas9 | AAACGUACUUCUUCAAGCAG | 1507 |
| CBE_NGA_20 nt_4-9_006_+_160541098_spCas9 | AAACGUACUUCUUCAAGCAG | 1508 |
| CBE_NGG_20 nt_4-9_003_+_160548601_spCas9 | AAAACACCAAGGGCCUGUAU | 1505 |
| CBE_NGC_20 nt_4-9_009_+_160548602_spCas9 | AAACACCAAGGGCCUGUAUC | 1506 |
| ABE_NGG_20 nt_3-9_002_−_160532633_spCas9 | AACACAGGUACCUUUGGGAC | 1509 |
| CBE_NGA_20 nt_4-9_006_+_160601042_spCas9 | AACACCAAGGGGCUGCCACA | 1510 |
| CBE_NGA_20 nt_4-9_006_+_160601092_spCas9 | AAGCCACUGGAAAUUCCAAA | 1511 |
| CBE_NGA_20 nt_4-9_006_+_160600969_spCas9 | AAGGCAGUCCAUUCUGCAUC | 1512 |
| ABE_NGG_20 nt_3-9_002_+_160578512_spCas9 | AAUUUCUUACCUUGUUCAGA | 1513 |
| ABE_NGC_20 nt_3-9_008_−_160532632_spCas9 | ACACAGGUACCUUUGGGACU | 1514 |
| CBE_NGG_20 nt_4-9_003_−_160589669_spCas9 | ACAGUCCAGGACUGCUACCA | 1515 |
| CBE_NGG_20 nt_4-9_003_−_160548539_spCas9 | ACGCGAUGCUCAGACACAGA | 1516 |
| ABE_NGC_20 nt_3-9_008_+_160541100_spCas9 | ACGUACUUCUUCAAGCAGUG | 1517 |
| CBE_NGC_20 nt_4-9_009_+_160541100_spCas9 | ACGUACUUCUUCAAGCAGUG | 1518 |
| CBE_NGA_20 nt_4-9_006_−_160532598_spCas9 | AGCCCAGCUCCUUGUUAUUG | 1519 |
| CBE_NGG_20 nt_4-9_003_+_160542802_spCas9 | AGGAUGCUGUGGCACAAGGU | 1520 |
| CBE_NGC_20 nt_4-9_009_+_160537957_spCas9 | AGGCCUGUAAGGAAAGUAUU | 1521 |
| CBE_NGA_20 nt_4-9_006_+_160557482_spCas9 | AGUACUCCCACCUCACACAC | 1522 |
| CBE_NGA_20 nt_4-9_006_+_160599655_spCas9 | AGUGCUGAAAUUAAAACAGA | 1523 |
| CBE_NGA_20 nt_4-9_006_−_160556067_spCas9 | AUGUCAAUCUUGGUCAUCCA | 1524 |
| CBE_NGC_20 nt_4-9_009_−_160599594_spCas9 | AUUAUGGACAGAGUUACCGA | 1525 |
| CBE_NGC_20 nt_4-9_009_−_160540055_spCas9 | CACACAAGCAGAUAUUGCCU | 1526 |
| CBE_NGA_20 nt_4-9_006_−_160599514_spCas9 | CACCAGCAUAGUCGGACCCC | 1527 |
| CBE_NGG_20 nt_4-9_003_+_160557481_spCas9 | CAGUACUCCCACCUCACACA | 1528 |
| CBE_NGA_20 nt_4-9_006_−_160650352_spCas9 | CAUCAACAUAAUAGGACCAC | 1529 |
| CBE_NGC_20 nt_4-9_009_−_160548492_spCas9 | CAUCCAGGUUCCAAGCCUAG | 1530 |
| CBE_NGC_20 nt_4-9_009_+_160541175_spCas9 | CCAAACCUAGAAAGAAAACA | 1531 |
| CBE_NGG_20 nt_4-9_003_+_160547801_spCas9 | CCUGCCCAUUUAUUUGUCCC | 1532 |
| CBE_NGA_20 nt_4-9_006_+_160585072_spCas9 | CGAUGCCAAUGUGGUGUCAU | 1533 |
| CBE_NGG_20 nt_4-9_003_−_160556135_spCas9 | CGGCAGUGCUACCAUGGUAA | 1534 |
| CBE_NGG_20 nt_4-9_003_+_160557486_spCas9 | CUCCCACCUCACACACGGAU | 1535 |
| CBE_NGA_20 nt_4-9_006_−_160595418_spCas9 | CUGACACAAUGCCUGGUGAC | 1536 |
| CBE_NGA_20 nt_4-9_006_−_160557454_spCas9 | CUGACACAAUGCUCAGAAAC | 1537 |
| CBE_NGA_20 nt_4-9_006_−_160600981_spCas9 | CUGACACGAUGCUCAGAUGC | 1538 |
| CBE_NGA_20 nt_4-9_006_−_160586513_spCas9 | CUGACGCAAUGUCCAGUGAU | 1539 |
| CBE_NGA_20 nt_4-9_006_−_160548542_spCas9 | CUGACGCGAUGCUCAGACAC | 1540 |
| CBE_NGA_20 nt_4-9_006_+_160547802_spCas9 | CUGCCCAUUUAUUUGUCCCU | 1541 |
| CBE_NGG_20 nt_4-9_003_−_160601057_spCas9 | CUGCCGAAAUCCAGAUCCUG | 1542 |
| CBE_NGA_20 nt_4-9_006_−_160585151_spCas9 | CUGCUACCGAGGUGAUGGAC | 1543 |
| CBE_NGA_20 nt_4-9_006_−_160585164_spCas9 | CUGGGGUCCAGGACUGCUAC | 1544 |
| CBE_NGA_20 nt_4-9_006_−_160577178_spCas9 | CUGUCAAUCUUGGUCAUCUA | 1545 |
| CBE_NGG_20 nt_4-9_003_+_160531882_spCas9 | CUGUCACCCUAAACAGAGGU | 1546 |
| ABE_NGG_20 nt_3-9_002_−_160531875_spCas9 | CUGUUUAGGGUGACAGUGGA | 1547 |
| CBE_NGC_20 nt_4-9_009_−_160605140_spCas9 | GAAAUGGACAGAGUUAUCAA | 1548 |
| CBE_NGC_20 nt_4-9_009_−_160590927_spCas9 | GAACAAGGUAAGAAGUCUCU | 1549 |
| CBE_NGA_20 nt_4-9_006_−_160532600_spCas9 | GAAGCCCAGCUCCUUGUUAU | 1550 |
| ABE_NGA_20 nt_3-9_005_+_160595343_spCas9 | GACUUCCUACCUUCUUCAGA | 1551 |
| ABE_NGG_20 nt_3-9_002_+_160548467_spCas9 | GACUUCUUACCUUGUUCAGA | 1552 |
| CBE_NGA_20 nt_4-9_006_−_160650466_spCas9 | GAGCAAAGCCAUGUGGUCCA | 1553 |
| CBE_NGA_20 nt_4-9_006_−_160589681_spCas9 | GAGCAAAGCCCCACAGUCCA | 1554 |
| CBE_NGA_20 nt_4-9_006_−_160594086_spCas9 | GAGCAAAGCCCCGGGGUCCA | 1555 |
| CBE_NGG_20 nt_4-9_003_+_160542801_spCas9 | GAGGAUGCUGUGGCACAAGG | 1556 |
| ABE_NGA_20 nt_3-9_005_−_160542792_spCas9 | GCCACAGCAUCCUCUUCAUU | 1557 |
| ABE_NGA_20 nt_3-9_005_+_160606467_spCas9 | GCUCCUUACCUUGUUCAGAA | 1558 |
| CBE_NGA_20 nt_4-9_006_+_160606467_spCas9 | GCUCCUUACCUUGUUCAGAA | 1559 |
| CBE_NGA_20 nt_4-9_006_+_160650493_spCas9 | GCUGCUAAAAUUAAAACAGA | 1560 |
| ABE_NGA_20 nt_3-9_005_+_160586439_spCas9 | GCUUCUUACCUUCUUCAGAA | 1561 |
| CBE_NGA_20 nt_4-9_006_+_160586439_spCas9 | GCUUCUUACCUUCUUCAGAA | 1562 |
| CBE_NGG_20 nt_4-9_003_−_160589682_spCas9 | GGAGCAAAGCCCCACAGUCC | 1563 |
| CBE_NGG_20 nt_4-9_003_−_160594087_spCas9 | GGAGCAAAGCCCCGGGGUCC | 1564 |
| CBE_NGA_20 nt_4-9_006_+_160542803_spCas9 | GGAUGCUGUGGCACAAGGUG | 1565 |
| CBE_NGC_20 nt_4-9_009_+_160600971_spCas9 | GGCAGUCCAUUCUGCAUCUG | 1566 |
| ABE_NGG_20 nt_3-9_002_+_160606466_spCas9 | GGCUCCUUACCUUGUUCAGA | 1567 |
| ABE_NGG_20 nt_3-9_002_+_160586438_spCas9 | GGCUUCUUACCUUCUUCAGA | 1568 |
| CBE_NGG_20 nt_4-9_003_−_160585162_spCas9 | GGGGUCCAGGACUGCUACCG | 1569 |
| CBE_NGA_20 nt_4-9_006_−_160605161_spCas9 | GGGUGCAGGAGUGCUACCAC | 1570 |
| CBE_NGA_20 nt_4-9_006_+_160605201_spCas9 | GGUGCUGAAAUGAAAAGAAA | 1571 |
| CBE_NGA_20 nt_4-9_006_+_160650490_spCas9 | GGUGCUGCUAAAAUUAAAAC | 1572 |
| CBE_NGG_20 nt_4-9_003_+_160531884_spCas9 | GUCACCCUAAACAGAGGUAG | 1573 |
| CBE_NGA_20 nt_4-9_006_+_160599652_spCas9 | GUCAGUGCUGAAAUUAAAAC | 1574 |
| CBE_NGG_20 nt_4-9_003_−_160548494_spCas9 | GUCAUCCAGGUUCCAAGCCU | 1575 |
| CBE_NGA_20 nt_4-9_006_−_160585159_spCas9 | GUCCAGGACUGCUACCGAGG | 1576 |
| CBE_NGC_20 nt_4-9_009_+_160591044_spCas9 | UAACACCAAGGACUAAUCUC | 1577 |
| CBE_NGG_20 nt_4-9_003_+_160601041_spCas9 | UAACACCAAGGGGCUGCCAC | 1578 |
| CBE_NGA_20 nt_4-9_006_+_160557514_spCas9 | UAACACCAGGGUUGUUUCCC | 1579 |
| ABE_NGA_20 nt_3-9_005_−_160537943_spCas9 | UACAGGCCUGCCGUCAUCAC | 1580 |
| CBE_NGA_20 nt_4-9_006_+_160531885_spCas9 | UCACCCUAAACAGAGGUAGG | 1581 |
| CBE_NGG_20 nt_4-9_003_−_160548493_spCas9 | UCAUCCAGGUUCCAAGCCUA | 1582 |
| CBE_NGG_20 nt_4-9_003_−_160578507_spCas9 | UGAACAAGGUAAGAAAUUUG | 1583 |
| CBE_NGG_20 nt_4-9_003_−_160590928_spCas9 | UGAACAAGGUAAGAAGUCUC | 1584 |
| CBE_NGG_20 nt_4-9_003_−_160606461_spCas9 | UGAACAAGGUAAGGAGCCUG | 1585 |
| CBE_NGG_20 nt_4-9_003_−_160650467_spCas9 | UGAGCAAAGCCAUGUGGUCC | 1586 |
| CBE_NGC_20 nt_4-9_009_−_160601056_spCas9 | UGCCGAAAUCCAGAUCCUGU | 1587 |
| CBE_NGG_20 nt_4-9_003_+_160531883_spCas9 | UGUCACCCUAAACAGAGGUA | 1588 |
| ABE_NGC_20 nt_3-9_008_−_160531874_spCas9 | UGUUUAGGGUGACAGUGGAG | 1589 |
| ABE_NGA_20 nt_3-9_005_−_160599646_spCas9 | UUAAUUUCAGCACUGACUGA | 1590 |
| ABE_NGC_20 nt_3-9_008_+_160595346_spCas9 | UUCCUACCUUCUUCAGAAGA | 1591 |
| CBE_NGC_20 nt_4-9_009_+_160595346_spCas9 | UUCCUACCUUCUUCAGAAGA | 1592 |
| ABE_NGA_20 nt_3-9_005_−_160547922_spCas9 | UUCUACAGACUGUAUGUUUG | 1593 |
| ABE_NGC_20 nt_3-9_008_+_160557382_spCas9 | UUCUUACCUGCUUCAGAAUG | 1594 |
| CBE_NGC_20 nt_4-9_009_+_160557382_spCas9 | UUCUUACCUGCUUCAGAAUG | 1595 |
| ABE_NGC_20 nt_3-9_008_+_160600909_spCas9 | UUCUUACCUUGUUCAAAAAA | 1596 |
| CBE_NGC_20 nt_4-9_009_+_160600909_spCas9 | UUCUUACCUUGUUCAAAAAA | 1597 |
| CBE_NGC_20 nt_4-9_009_−_160600902_spCas9 | UUGAACAAGGUAAGAAGUUG | 1598 |
| ABE_NGC_20 nt_3-9_008_−_160585188_spCas9 | UUUCAGCACCAACUGAAAAC | 1599 |
| ABE_NGC_20 nt_3-9_008_−_160577278_spCas9 | UUUCAGCACCACCUGAGAAA | 1600 |
| ABE_NGG_20 nt_3-9_002_−_160547923_spCas9 | UUUCUACAGACUGUAUGUUU | 1601 |
| ABE_NGC_20 nt_3-9_008_−_160650480_spCas9 | UUUUAGCAGCACCUGAGCAA | 1602 |
| ABE_NGG_20 nt_3-9_002_−_160547924_spCas9 | UUUUCUACAGACUGUAUGUU | 1603 |

TABLE 1C-1
Exemplary guide polynucleotide sequences for use in targeting a base editor to
disrupt a promoter region of an LPA polynucleotide.

| | | SEQ |
|---|---|---|
| Guide Name | Guide Polynucleotide Sequence | ID NO |
| ABE_NNNRRT_21 nt_5- | mUsmAsmUsUUUAUAAGACUCUAUAUUGUUUUAGUACUCU | 1604 |
| 14_014_−_ | GUAAUGAAAAUUACAGAAUCUACUAAAACAAGGCAAAAUG | |
| 160664368_saCas9 | CCGUGUUUAUCUCGUCAACUUGUUGGCGAGAUsmUsmUsm | |
| | U | |
| CBE_NNNRRT_21 nt_3- | mUsmAsmUsUUUAUAAGACUCUAUAUUGUUUUAGUACUCU | 1605 |
| 12_015_−_ | GUAAUGAAAAUUACAGAAUCUACUAAAACAAGGCAAAAUG | |
| 160664368_saCas9 | CCGUGUUUAUCUCGUCAACUUGUUGGCGAGAUsmUsmUsm | |
| | U | |
| ABE_NGG_20 nt_3-9_002_−_ | mAsmGsmGsCAAUGUGGAGCAGCUGAGUUUUAGAGCUAGA | 1606 |
| 160664446_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mAsmGsmGsCAAUGUGGAGCAGCUGAGUUUUAGAGCUAGA | 1607 |
| 160664446_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4- | mAsmUsmGsAUUCUCUCAGAGACCCAGUUUUAGAGCUAGA | 1608 |
| 9_009_+_160664319_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3- | mAsmUsmGsAUUCUCUCAGAGACCCAGUUUUAGAGCUAGA | 1609 |
| 9_008_+_160664319_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mGsmCsmAsAUGUGGAGCAGCUGAGGGUUUUAGAGCUAGA | 1610 |
| 160664444_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mGsmCsmAsAUGUGGAGCAGCUGAGGGUUUUAGAGCUAGA | 1611 |
| 160664444_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4-9_006_−_ | mGsmGsmAsGGAAACAAGACUAAUCAGUUUUAGAGCUAGA | 1612 |
| 160664512_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mGsmGsmAsGGAAACAAGACUAAUCAGUUUUAGAGCUAGA | 1613 |
| 160664512_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mAsmUsmCsAGGAAAGAUGAAGGUCUGUUUUAGAGCUAGA | 1614 |
| 160664496_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mAsmUsmCsAGGAAAGAUGAAGGUCUGUUUUAGAGCUAGA | 1615 |
| 160664496_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mAsmAsmUsUUGACUAUCUGGUUUGUGUUUUAGAGCUAGA | 1616 |
| 160664290_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mAsmAsmUsUUGACUAUCUGGUUUGUGUUUUAGAGCUAGA | 1617 |
| 160664290_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mUsmCsmAsGGAAAGAUGAAGGUCUAGUUUUAGAGCUAGA | 1618 |
| 160664495_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mUsmCsmAsGGAAAGAUGAAGGUCUAGUUUUAGAGCUAGA | 1619 |
| 160664495_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4- | mAsmAsmGsCCAUUUCCCCCCUCAGCGUUUUAGAGCUAGA | 1620 |
| 9_009_+_160664434_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3- | mAsmAsmGsCCAUUUCCCCCCUCAGCGUUUUAGAGCUAGA | 1621 |
| 9_008_+_160664434_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mGsmAsmAsGGAGUAAGGAGACAUAAAGUUUUAGUACUCU | 1622 |
| 14_014_−_ | GUAAUGAAAAUUACAGAAUCUACUAAAACAAGGCAAAAUG | |
| 160664462_saCas9 | CCGUGUUUAUCUCGUCAACUUGUUGGCGAGAUsmUsmUsm | |
| | U | |
| CBE_NNNRRT_21 nt_3- | mGsmAsmAsGGAGUAAGGAGACAUAAAGUUUUAGUACUCU | 1623 |
| 12_015_−_ | GUAAUGAAAAUUACAGAAUCUACUAAAACAAGGCAAAAUG | |
| 160664462_saCas9 | CCGUGUUUAUCUCGUCAACUUGUUGGCGAGAUsmUsmUsm | |
| | U | |
| ABE_NGG_20 nt_3-9_002_−_ | mCsmAsmGsGAAAGAUGAAGGUCUAGGUUUUAGAGCUAGA | 1624 |
| 160664494_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mCsmAsmGsGAAAGAUGAAGGUCUAGGUUUUAGAGCUAGA | 1625 |
| 160664494_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mUsmAsmAsUUUGACUAUCUGGUUUGGUUUUAGAGCUAGA | 1626 |
| 160664291_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mUsmAsmAsUUUGACUAUCUGGUUUGGUUUUAGAGCUAGA | 1627 |
| 160664291_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNGRRT_21 nt_5- | mUsmUsmUsUAUUUUAUCCAAAAGAAAGUUUUAGUACUCU | 1628 |
| 14_011_−_ | GUAAUGAAAAUUACAGAAUCUACUAAAACAAGGCAAAAUG | |
| 160664546_saCas9 | CCGUGUUUAUCUCGUCAACUUGUUGGCGAGAUsmUsmUsm | |
| | U | |
| CBE_NNGRRT_21 nt_3- | mUsmUsmUsUAUUUUAUCCAAAAGAAAGUUUUAGUACUCU | 1629 |
| 12_012_−_ | GUAAUGAAAAUUACAGAAUCUACUAAAACAAGGCAAAAUG | |
| 160664546_saCas9 | CCGUGUUUAUCUCGUCAACUUGUUGGCGAGAUsmUsmUsm | |
| | U | |
| CBE_NGC_20 nt_4-9_009_−_ | mUsmUsmGsACUAUCUGGUUUGUGGGGUUUUAGAGCUAGA | 1630 |
| 160664287_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3-9_008_−_ | mUsmUsmGsACUAUCUGGUUUGUGGGGUUUUAGAGCUAGA | 1631 |
| 160664287_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4- | mUsmAsmAsGUUAAUGAUUCUCUCAGGUUUUAGAGCUAGA | 1632 |
| 9_006_+_160664312_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3- | mUsmAsmAsGUUAAUGAUUCUCUCAGGUUUUAGAGCUAGA | 1633 |
| 9_005_+_160664312_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4- | mGsmAsmGsAGUGCAAUGUCAAUAGAGUUUUAGAGCUAGA | 1634 |
| 9_009_+_160664398_spCas9 | AAUAGCAAGUUAAAAUAAGGCUAGUCCGUUAUCAACUUGA | |
| | AAAAGUGGCACCGAGUCGGUGCmUsmUsmUSU | |
| ABE_NGC_20 nt_3- | mGsmAsmGsAGUGCAAUGUCAAUAGAGUUUUAGAGCUAGA | 1635 |
| 9_008_+_160664398_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3- | mUsmGsmUsCAAUAGAUGCUGGGAAGGUUUUAGAGCUAGA | 1636 |
| 9_002_+_160664408_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4- | mUsmGsmUsCAAUAGAUGCUGGGAAGGUUUUAGAGCUAGA | 1637 |
| 9_003_+_160664408_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mAsmCsmCsCUGCUGAGCCAGUGGCAGUUUUAGAGCUAGA | 1638 |
| 160664336_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mAsmCsmCsCUGCUGAGCCAGUGGCAGUUUUAGAGCUAGA | 1639 |
| 160664336_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4-9_009_−_ | mUsmCsmAsAGGUAAUGUUUGAACCCGUUUUAGAGCUAGA | 1640 |
| 160664352_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3-9_008_−_ | mUsmCsmAsAGGUAAUGUUUGAACCCGUUUUAGAGCUAGA | 1641 |
| 160664352_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4-9_006_−_ | mGsmAsmAsAGAUGAAGGUCUAGGGGGUUUUAGAGCUAGA | 1642 |
| 160664491_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mGsmAsmAsAGAUGAAGGUCUAGGGGGUUUUAGAGCUAGA | 1643 |
| 160664491_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3-9_002_−_ | mAsmAsmGsAUGAAGGUCUAGGGGUGGUUUUAGAGCUAGA | 1644 |
| 160664489_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mAsmAsmGsAUGAAGGUCUAGGGGUGGUUUUAGAGCUAGA | 1645 |
| 160664489_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mUsmAsmAsAAUAUUUGAGAGUGCAAUGUUUUAGUACUCU | 1646 |
| 14_014_+_160664388_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| CBE_NNNRRT_21 nt_3- | mUsmAsmAsAAUAUUUGAGAGUGCAAUGUUUUAGUACUCU | 1647 |
| 12_015_+_160664388_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| ABE_NGG_20 nt_3-9_002_−_ | mGsmGsmGsAGGAAACAAGACUAAUCGUUUUAGAGCUAGA | 1648 |
| 160664513_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mGsmGsmGsAGGAAACAAGACUAAUCGUUUUAGAGCUAGA | 1649 |
| 160664513_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3- | mCsmCsmCsAUGCCACUGGCUCAGCAGUUUUAGAGCUAGA | 1650 |
| 9_002_+_160664335_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4- | mCsmCsmCsAUGCCACUGGCUCAGCAGUUUUAGAGCUAGA | 1651 |
| 9_003_+_160664335_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4- | mUsmGsmCsAAUGUCAAUAGAUGCUGGUUUUAGAGCUAGA | 1652 |
| 9_006_+_160664403_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3- | mUsmGsmCsAAUGUCAAUAGAUGCUGGUUUUAGAGCUAGA | 1653 |
| 9_005_+_160664403_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4-9_006_−_ | mUsmAsmAsAGGCAAUGUGGAGCAGCGUUUUAGAGCUAGA | 1654 |
| 160664449_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mUsmAsmAsAGGCAAUGUGGAGCAGCGUUUUAGAGCUAGA | 1655 |
| 160664449_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mGsmUsmCsUUAUAAAAUAUUUGAGAGGUUUUAGUACUCU | 1656 |
| 14_014_+_160664382_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| CBE_NNNRRT_21 nt_3- | mGsmUsmCsUUAUAAAAUAUUUGAGAGGUUUUAGUACUCU | 1657 |
| 12_015_+_160664382_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| ABE_NGG_20 nt_3-9_002_−_ | mUsmUsmUsGAACCCUGCUGAGCCAGGUUUUAGAGCUAGA | 1658 |
| 160664341_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4-9_003_−_ | mUsmUsmUsGAACCCUGCUGAGCCAGGUUUUAGAGCUAGA | 1659 |
| 160664341_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4-9_009_−_ | mGsmUsmAsAUGUUUGAACCCUGCUGGUUUUAGAGCUAGA | 1660 |
| 160664347_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3-9_008_−_ | mGsmUsmAsAUGUUUGAACCCUGCUGGUUUUAGAGCUAGA | 1661 |
| 160664347_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGC_20 nt_4-9_009_−_ | mUsmUsmGsAACCCUGCUGAGCCAGUGUUUUAGAGCUAGA | 1662 |
| 160664340_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGC_20 nt_3-9_008_−_ | mUsmUsmGsAACCCUGCUGAGCCAGUGUUUUAGAGCUAGA | 1663 |
| 160664340_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4-9_006_−_ | mGsmCsmCsAGUGGCAUGGGUCUCUGGUUUUAGAGCUAGA | 1664 |
| 160664326_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mGsmCsmCsAGUGGCAUGGGUCUCUGGUUUUAGAGCUAGA | 1665 |
| 160664326_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mUsmUsmUsAUAAGACUCUAUAUUCAAGUUUUAGUACUCU | 1666 |
| 14_014_−_ | GUAAUGAAAAUUACAGAAUCUACUAAAACAAGGCAAAAUG | |
| 160664365_saCas9 | CCGUGUUUAUCUCGUCAACUUGUUGGCGAGAUsmUsmUsm | |
| | U | |
| CBE_NNNRRT_21 nt_3- | mUsmUsmUsAUAAGACUCUAUAUUCAAGUUUUAGUACUCU | 1667 |
| 12_015_−_ | GUAAUGAAAAUUACAGAAUCUACUAAAACAAGGCAAAAUG | |
| 160664365_saCas9 | CCGUGUUUAUCUCGUCAACUUGUUGGCGAGAUsmUsmUsm | |
| | U | |
| CBE_NGA_20 nt_4-9_006_−_ | mGsmAsmGsCCAGUGGCAUGGGUCUCGUUUUAGAGCUAGA | 1668 |
| 160664328_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mGsmAsmGsCCAGUGGCAUGGGUCUCGUUUUAGAGCUAGA | 1669 |
| 160664328_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mAsmUsmCsAUUAACUUAAUUUGACUAGUUUUAGUACUCU | 1670 |
| 14_014_−_ | GUAAUGAAAAUUACAGAAUCUACUAAAACAAGGCAAAAUG | |
| 160664297_saCas9 | CCGUGUUUAUCUCGUCAACUUGUUGGCGAGAUsmUsmUsm | |
| | U | |
| CBE_NNNRRT_21 nt_3- | mAsmUsmCsAUUAACUUAAUUUGACUAGUUUUAGUACUCU | 1671 |
| 12_015_−_ | GUAAUGAAAAUUACAGAAUCUACUAAAACAAGGCAAAAUG | |
| 160664297_saCas9 | CCGUGUUUAUCUCGUCAACUUGUUGGCGAGAUsmUsmUsm | |
| | U | |
| CBE_NGA_20 nt_4- | mCsmCsmCsUAGACCUUCAUCUUUCCGUUUUAGAGCUAGA | 1672 |
| 9_006_+_160664495_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3- | mCsmCsmCsUAGACCUUCAUCUUUCCGUUUUAGAGCUAGA | 1673 |
| 9_005_+_160664495_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mCsmAsmAsACCAGAUAGUCAAAUUAAGUUUUAGUACUCU | 1674 |
| 14_014_+_160664294_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| CBE_NNNRRT_21 nt_3- | mCsmAsmAsACCAGAUAGUCAAAUUAAGUUUUAGUACUCU | 1675 |
| 12_015_+_160664294_saCas | GUAAUGAAAAUUACAGAAUCUACUAAAACAAGGCAAAAUG | |
| 9 | CCGUGUUUAUCUCGUCAACUUGUUGGCGAGAUsmUsmUsm | |
| | U | |
| CBE_NGA_20 nt_4- | mUsmUsmUsGAGAGUGCAAUGUCAAUGUUUUAGAGCUAGA | 1676 |
| 9_006_+_160664395_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3- | mUsmUsmUsGAGAGUGCAAUGUCAAUGUUUUAGAGCUAGA | 1677 |
| 9_005_+_160664395_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4-9_006_−_ | mAsmGsmGsUAAUGUUUGAACCCUGCGUUUUAGAGCUAGA | 1678 |
| 160664349_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGA_20 nt_3-9_005_−_ | mAsmGsmGsUAAUGUUUGAACCCUGCGUUUUAGAGCUAGA | 1679 |
| 160664349_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NGG_20 nt_3- | mAsmGsmUsGCAAUGUCAAUAGAUGCGUUUUAGAGCUAGA | 1680 |
| 9_002_+_160664401_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGG_20 nt_4- | mAsmGsmUsGCAAUGUCAAUAGAUGCGUUUUAGAGCUAGA | 1681 |
| 9_003_+_160664401_spCas9 | AAUAGCAAGUUAAAAUAAGGCUAGUCCGUUAUCAACUUGA | |
| | AAAAGUGGCACCGAGUCGGUGCmUsmUsmUsU | |
| CBE_NGA_20 nt_4- | mGsmCsmAsGGGUUCAAACAUUACCUGUUUUAGAGCUAGA | 1682 |
| 9_006_+_160664352_spCas9 | AAUAGCAAGUUAAAAUAAGGCUAGUCCGUUAUCAACUUGA | |
| | AAAAGUGGCACCGAGUCGGUGCmUsmUsmUSU | |
| ABE_NGA_20 nt_3- | mGsmCsmAsGGGUUCAAACAUUACCUGUUUUAGAGCUAGA | 1683 |
| 9_005_+_160664352_spCas9 | AAUAGCAAGUUAAAAUAAGGCUAGUCCGUUAUCAACUUGA | |
| | AAAAGUGGCACCGAGUCGGUGCmUsmUsmUsU | |
| ABE_NNNRRT_21 nt_5- | mAsmCsmCsAGAUAGUCAAAUUAAGUUGUUUUAGUACUCU | 1684 |
| 14_014_+_160664297_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mAsmCsmCsAGAUAGUCAAAUUAAGUUGUUUUAGUACUCU | 1685 |
| 12_015_+_160664297_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mGsmAsmGsGAAACAAGACUAAUCAGGGUUUUAGUACUCU | 1686 |
| 14_014_−_ | GUAAUGAAAAUUACAGAAUCUACUAAAACAAGGCAAAAUG | |
| 160664507_saCas9 | CCGUGUUUAUCUCGUCAACUUGUUGGCGAGAUsmUsmUsm | |
| | U | |
| CBE_NNNRRT_21 nt_3- | mGsmAsmGsGAAACAAGACUAAUCAGGGUUUUAGUACUCU | 1687 |
| 12_015_−_ | GUAAUGAAAAUUACAGAAUCUACUAAAACAAGGCAAAAUG | |
| 160664507_saCas9 | CCGUGUUUAUCUCGUCAACUUGUUGGCGAGAUsmUsmUsm | |
| | U | |
| ABE_NNNRRT_21 nt_5- | mGsmGsmUsCUCUGAGAGAAUCAUUAAGUUUUAGUACUCU | 1688 |
| 14_014_−_ | GUAAUGAAAAUUACAGAAUCUACUAAAACAAGGCAAAAUG | |
| 160664310_saCas9 | CCGUGUUUAUCUCGUCAACUUGUUGGCGAGAUsmUsmUsm | |
| | U | |
| CBE_NNNRRT_21 nt_3- | mGsmGsmUsCUCUGAGAGAAUCAUUAAGUUUUAGUACUCU | 1689 |
| 12_015_−_ | GUAAUGAAAAUUACAGAAUCUACUAAAACAAGGCAAAAUG | |
| 160664310_saCas9 | CCGUGUUUAUCUCGUCAACUUGUUGGCGAGAUsmUsmUsm | |
| | U | |
| ABE_NNNRRT_21 nt_5- | mCsmCsmCsACAAACCAGAUAGUCAAAGUUUUAGUACUCU | 1690 |
| 14_014_+_160664290_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mCsmCsmCsACAAACCAGAUAGUCAAAGUUUUAGUACUCU | 1691 |
| 12_015_+_160664290_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNGRRT_21 nt_5- | mAsmUsmAsGAGUCUUAUAAAAUAUUUGUUUUAGUACUCU | 1692 |
| 14_011_+_160664377_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNGRRT_21 nt_3- | mAsmUsmAsGAGUCUUAUAAAAUAUUUGUUUUAGUACUCU | 1693 |
| 12_012_+_160664377_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mAsmUsmAsUUUGAGAGUGCAAUGUCAGUUUUAGUACUCU | 1694 |
| 14_014_+_160664392_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mAsmUsmAsUUUGAGAGUGCAAUGUCAGUUUUAGUACUCU | 1695 |
| 12_015_+_160664392_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mCsmAsmAsUGUCAAUAGAUGCUGGGAGUUUUAGUACUCU | 1696 |
| 14_014_+_160664405_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mCsmAsmAsUGUCAAUAGAUGCUGGGAGUUUUAGUACUCU | 1697 |
| 12_015_+_160664405_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mCsmAsmCsCCCUAGACCUUCAUCUUUGUUUUAGUACUCU | 1698 |
| 14_014_+_160664492_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mCsmAsmCsCCCUAGACCUUCAUCUUUGUUUUAGUACUCU | 1699 |
| 12_015_+_160664492_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mCsmCsmUsAGACCUUCAUCUUUCCUGGUUUUAGUACUCU | 1700 |
| 14_014_+_160664496_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mCsmCsmUsAGACCUUCAUCUUUCCUGGUUUUAGUACUCU | 1701 |
| 12_015_+_160664496_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mUsmAsmCsCUUGAAUAUAGAGUCUUAGUUUUAGUACUCU | 1702 |
| 14_014_+_160664367_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mUsmAsmCsCUUGAAUAUAGAGUCUUAGUUUUAGUACUCU | 1703 |
| 12_015_+_160664367_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNGRRT_21 nt_5- | mGsmAsmAsCCCUGCUGAGCCAGUGGCGUUUUAGUACUCU | 1704 |
| 14_011_−_ | GUAAUGAAAAUUACAGAAUCUACUAAAACAAGGCAAAAUG | |
| 160664334_saCas9 | CCGUGUUUAUCUCGUCAACUUGUUGGCGAGAUsmUsmUsm | |
| | U | |
| CBE_NNGRRT_21 nt_3- | mGsmAsmAsCCCUGCUGAGCCAGUGGCGUUUUAGUACUCU | 1705 |
| 12_012_−_ | GUAAUGAAAAUUACAGAAUCUACUAAAACAAGGCAAAAUG | |
| 160664334_saCas9 | CCGUGUUUAUCUCGUCAACUUGUUGGCGAGAUsmUsmUsm | |
| | U | |
| ABE_NNNRRT_21 nt_5- | mGsmUsmGsCAAUGUCAAUAGAUGCUGGUUUUAGUACUCU | 1706 |
| 14_014_+_160664402_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNNRRT_21 nt_3- | mGsmUsmGsCAAUGUCAAUAGAUGCUGGUUUUAGUACUCU | 1707 |
| 12_015_+_160664402_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNGRRT_21 nt_5- | mAsmAsmUsCAGGAAAGAUGAAGGUCUGUUUUAGUACUCU | 1708 |
| 14_011_−_ | GUAAUGAAAAUUACAGAAUCUACUAAAACAAGGCAAAAUG | |
| 160664493_saCas9 | CCGUGUUUAUCUCGUCAACUUGUUGGCGAGAUsmUsmUsm | |
| | U | |
| CBE_NNGRRT_21 nt_3- | mAsmAsmUsCAGGAAAGAUGAAGGUCUGUUUUAGUACUCU | 1709 |
| 12_012_−_ | GUAAUGAAAAUUACAGAAUCUACUAAAACAAGGCAAAAUG | |
| 160664493_saCas9 | CCGUGUUUAUCUCGUCAACUUGUUGGCGAGAUsmUsmUsm | |
| | U | |
| ABE_NNGRRT_21 nt_5- | mCsmUsmUsAAUUUGACUAUCUGGUUUGUUUUAGUACUCU | 1710 |
| 14_011_−_ | GUAAUGAAAAUUACAGAAUCUACUAAAACAAGGCAAAAUG | |
| 160664289_saCas9 | CCGUGUUUAUCUCGUCAACUUGUUGGCGAGAUsmUsmUsm | |
| | U | |
| CBE_NNGRRT_21 nt_3- | mCsmUsmUsAAUUUGACUAUCUGGUUUGUUUUAGUACUCU | 1711 |
| 12_012_−_ | GUAAUGAAAAUUACAGAAUCUACUAAAACAAGGCAAAAUG | |
| 160664289_saCas9 | CCGUGUUUAUCUCGUCAACUUGUUGGCGAGAUsmUsmUsm | |
| | U | |
| ABE_NNGRRT_21 nt_5- | mGsmUsmUsCAAACAUUACCUUGAAUAGUUUUAGUACUCU | 1712 |
| 14_011_+_160664357_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNGRRT_21 nt_3- | mGsmUsmUsCAAACAUUACCUUGAAUAGUUUUAGUACUCU | 1713 |
| 12_012_+_160664357_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNGRRT_21 nt_5- | mGsmCsmCsAGUGGCAUGGGUCUCUGAGUUUUAGUACUCU | 1714 |
| 14_011_−_ | GUAAUGAAAAUUACAGAAUCUACUAAAACAAGGCAAAAUG | |
| 160664322_saCas9 | CCGUGUUUAUCUCGUCAACUUGUUGGCGAGAUsmUsmUsm | |
| | U | |
| CBE_NNGRRT_21 nt_3- | mGsmCsmCsAGUGGCAUGGGUCUCUGAGUUUUAGUACUCU | 1715 |
| 12_012_−_ | GUAAUGAAAAUUACAGAAUCUACUAAAACAAGGCAAAAUG | |
| 160664322_saCas9 | CCGUGUUUAUCUCGUCAACUUGUUGGCGAGAUsmUsmUsm | |
| | U | |
| ABE_NNGRRT_21 nt_5- | mAsmGsmAsCCCAUGCCACUGGCUCAGGUUUUAGUACUCU | 1716 |
| 14_011_+_160664332_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNGRRT_21 nt_3- | mAsmGsmAsCCCAUGCCACUGGCUCAGGUUUUAGUACUCU | 1717 |
| 12_012_+_160664332_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNGRRT_21 nt_5- | mCsmAsmGsCAGGGUUCAAACAUUACCGUUUUAGUACUCU | 1718 |
| 14_011_+_160664350_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| CBE_NNGRRT_21 nt_3- | mCsmAsmGsCAGGGUUCAAACAUUACCGUUUUAGUACUCU | 1719 |
| 12_012_+_160664350_saCas9 | GUAAUGAAAAUUACAGAAUCUACUAAAACAAGGCAAAAUG | |
| | CCGUGUUUAUCUCGUCAACUUGUUGGCGAGAUsmUsmUsm | |
| | U | |
| ABE_NNNRRT_21 nt_5- | mGsmCsmAsUCUAUUGACAUUGCACUCGUUUUAGUACUCU | 1720 |
| 14_014_−_ | GUAAUGAAAAUUACAGAAUCUACUAAAACAAGGCAAAAUG | |
| 160664394_saCas9 | CCGUGUUUAUCUCGUCAACUUGUUGGCGAGAUsmUsmUsm | |
| | U | |
| CBE_NNNRRT_21 nt_3- | mGsmCsmAsUCUAUUGACAUUGCACUCGUUUUAGUACUCU | 1721 |
| 12_015_−_ | GUAAUGAAAAUUACAGAAUCUACUAAAACAAGGCAAAAUG | |
| 160664394_saCas9 | CCGUGUUUAUCUCGUCAACUUGUUGGCGAGAUsmUsmUsm | |
| | U | |

| | Alternative | | | SEQ |
|---|---|---|---|---|
| Guide | Guide | Target Site | | ID |
| Name | Name | Name | Guide Polynucleotide Sequence | NO |
| gRNA3362- | gRNA3362 | TSBTx6221 | mUsmAsmUsUUUAUAAGACUCUAUAUUGUUUU | 1722 |
| 3053 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3363- | gRNA3363 | TSBTx6222 | mAsmGsmGsCAAUGUGGAGCAGCUGAGUUUUA | 1723 |
| 3054 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3364- | gRNA3364 | TSBTx6223 | mAsmUsmGsAUUCUCUCAGAGACCCAGUUUUA | 1724 |
| 3055 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3365- | gRNA3365 | TSBTx6224 | mGsmCsmAsAUGUGGAGCAGCUGAGGGUUUUA | 1725 |
| 3056 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3366- | gRNA3366 | TSBTx6225 | mGsmGsmAsGGAAACAAGACUAAUCAGUUUUA | 1726 |
| 3057 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3367- | gRNA3367 | TSBTx6226 | mAsmUsmCsAGGAAAGAUGAAGGUCUGUUUUA | 1727 |
| 3058 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3368- | gRNA3368 | TSBTx6227 | mAsmAsmUsUUGACUAUCUGGUUUGUGUUUUA | 1728 |
| 3059 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3369- | gRNA3369 | TSBTx6228 | mUsmCsmAsGGAAAGAUGAAGGUCUAGUUUUA | 1729 |
| 3060 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3370- | gRNA3370 | TSBTx6229 | mAsmAsmGsCCAUUUCCCCCCUCAGCGUUUUA | 1730 |
| 3061 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3371- | gRNA3371 | TSBTx6230 | mGsmAsmAsGGAGUAAGGAGACAUAAAGUUUU | 1731 |
| 3062 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3372- | gRNA3372 | TSBTx6231 | mCsmAsmGsGAAAGAUGAAGGUCUAGGUUUUA | 1732 |
| 3063 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3373- | gRNA3373 | TSBTx6232 | mUsmAsmAsUUUGACUAUCUGGUUUGGUUUUA | 1733 |
| 3064 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3374- | gRNA3374 | TSBTx6233 | mUsmUsmUsUAUUUUAUCCAAAAGAAAGUUUU | 1734 |
| 3065 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3375- | gRNA3375 | TSBTx6234 | mUsmUsmGsACUAUCUGGUUUGUGGGGUUUUA | 1735 |
| 3066 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3376- | gRNA3376 | TSBTx6235 | mUsmAsmAsGUUAAUGAUUCUCUCAGGUUUUA | 1736 |
| 3067 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3377- | gRNA3377 | TSBTx6236 | mGsmAsmGsAGUGCAAUGUCAAUAGAGUUUUA | 1737 |
| 3068 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3378- | gRNA3378 | TSBTx6237 | mUsmGsmUsCAAUAGAUGCUGGGAAGGUUUUA | 1738 |
| 3069 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3379- | gRNA3379 | TSBTx6238 | mAsmCsmCsCUGCUGAGCCAGUGGCAGUUUUA | 1739 |
| 3070 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3380- | gRNA3380 | TSBTx6239 | mUsmCsmAsAGGUAAUGUUUGAACCCGUUUUA | 1740 |
| 3071 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3381- | gRNA3381 | TSBTx6240 | mGsmAsmAsAGAUGAAGGUCUAGGGGGUUUUA | 1741 |
| 3072 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3382- | gRNA3382 | TSBTx6241 | mAsmAsmGsAUGAAGGUCUAGGGGUGGUUUUA | 1742 |
| 3073 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3383- | gRNA3383 | TSBTx6242 | mUsmAsmAsAAUAUUUGAGAGUGCAAUGUUUU | 1743 |
| 3074 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3384- | gRNA3384 | TSBTx6243 | mGsmGsmGsAGGAAACAAGACUAAUCGUUUUA | 1744 |
| 3075 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3385- | gRNA3385 | TSBTx6244 | mCsmCsmCsAUGCCACUGGCUCAGCAGUUUUA | 1745 |
| 3076 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3386- | gRNA3386 | TSBTx6245 | mUsmGsmCsAAUGUCAAUAGAUGCUGGUUUUA | 1746 |
| 3077 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3387- | gRNA3387 | TSBTx6246 | mUsmAsmAsAGGCAAUGUGGAGCAGCGUUUUA | 1747 |
| 3078 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3388- | gRNA3388 | TSBTx6247 | mGsmUsmCsUUAUAAAAUAUUUGAGAGGUUUU | 1748 |
| 3079 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3389- | gRNA3389 | TSBTx6248 | mUsmUsmUsGAACCCUGCUGAGCCAGGUUUUA | 1749 |
| 3080 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3390- | gRNA3390 | TSBTx6249 | mGsmUsmAsAUGUUUGAACCCUGCUGGUUUUA | 1750 |
| 3081 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3391- | gRNA3391 | TSBTx6250 | mUsmUsmGsAACCCUGCUGAGCCAGUGUUUUA | 1751 |
| 3082 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3392- | gRNA3392 | TSBTx6251 | mGsmCsmCsAGUGGCAUGGGUCUCUGGUUUUA | 1752 |
| 3083 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3393- | gRNA3393 | TSBTx6252 | mUsmUsmUsAUAAGACUCUAUAUUCAAGUUUU | 1753 |
| 3084 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3394- | gRNA3394 | TSBTx6253 | mGsmAsmGsCCAGUGGCAUGGGUCUCGUUUUA | 1754 |
| 3085 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3395- | gRNA3395 | TSBTx6254 | mAsmUsmCsAUUAACUUAAUUUGACUAGUUUU | 1755 |
| 3086 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3396- | gRNA3396 | TSBTx6255 | mCsmCsmCsUAGACCUUCAUCUUUCCGUUUUA | 1756 |
| 3087 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3397- | gRNA3397 | TSBTx6256 | mCsmAsmAsACCAGAUAGUCAAAUUAAGUUUU | 1757 |
| 3088 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3398- | gRNA3398 | TSBTx6257 | mUsmUsmUsGAGAGUGCAAUGUCAAUGUUUUA | 1758 |
| 3089 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3399- | gRNA3399 | TSBTx6258 | mAsmGsmGsUAAUGUUUGAACCCUGCGUUUUA | 1759 |
| 3090 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3400- | gRNA3400 | TSBTx6259 | mAsmGsmUsGCAAUGUCAAUAGAUGCGUUUUA | 1760 |
| 3091 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3401- | gRNA3401 | TSBTx6260 | mGsmCsmAsGGGUUCAAACAUUACCUGUUUUA | 1761 |
| 3092 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |
| gRNA3402- | gRNA3402 | TSBTx6261 | mAsmCsmCsAGAUAGUCAAAUUAAGUUGUUUU | 1762 |
| 3093 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3403- | gRNA3403 | TSBTx6262 | mGsmAsmGsGAAACAAGACUAAUCAGGGUUUU | 1763 |
| 3094 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3404- | gRNA3404 | TSBTx6263 | mGsmGsmUsCUCUGAGAGAAUCAUUAAGUUUU | 1764 |
| 3095 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3405- | gRNA3405 | TSBTx6264 | mCsmCsmCsACAAACCAGAUAGUCAAAGUUUU | 1765 |
| 3096 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3406- | gRNA3406 | TSBTx6265 | mAsmUsmAsGAGUCUUAUAAAAUAUUUGUUUU | 1766 |
| 3097 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3407- | gRNA3407 | TSBTx6266 | mAsmUsmAsUUUGAGAGUGCAAUGUCAGUUUU | 1767 |
| 3098 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3408- | gRNA3408 | TSBTx6267 | mCsmAsmAsUGUCAAUAGAUGCUGGGAGUUUU | 1768 |
| 3099 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3409- | gRNA3409 | TSBTx6268 | mCsmAsmCsCCCUAGACCUUCAUCUUUGUUUU | 1769 |
| 3100 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3410- | gRNA3410 | TSBTx6269 | mCsmCsmUsAGACCUUCAUCUUUCCUGGUUUU | 1770 |
| 3101 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3411- | gRNA3411 | TSBTx6270 | mUsmAsmCsCUUGAAUAUAGAGUCUUAGUUUU | 1771 |
| 3102 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3412- | gRNA3412 | TSBTx6271 | mGsmAsmAsCCCUGCUGAGCCAGUGGCGUUUU | 1772 |
| 3103 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3413- | gRNA3413 | TSBTx6272 | mGsmUsmGsCAAUGUCAAUAGAUGCUGGUUUU | 1773 |
| 3104 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3414- | gRNA3414 | TSBTx6273 | mAsmAsmUsCAGGAAAGAUGAAGGUCUGUUUU | 1774 |
| 3105 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3415- | gRNA3415 | TSBTx6274 | mCsmUsmUsAAUUUGACUAUCUGGUUUGUUUU | 1775 |
| 3106 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3416- | gRNA3416 | TSBTx6275 | mGsmUsmUsCAAACAUUACCUUGAAUAGUUUU | 1776 |
| 3107 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3417- | gRNA3417 | TSBTx6276 | mGsmCsmCsAGUGGCAUGGGUCUCUGAGUUUU | 1777 |
| 3108 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3418- | gRNA3418 | TSBTx6277 | mAsmGsmAsCCCAUGCCACUGGCUCAGGUUUU | 1778 |
| 3109 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3419- | gRNA3419 | TSBTx6278 | mCsmAsmGsCAGGGUUCAAACAUUACCGUUUU | 1779 |
| 3110 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| gRNA3420- | gRNA3420 | TSBTx6279 | mGsmCsmAsUCUAUUGACAUUGCACUCGUUUU | 1780 |
| 3111 | | | AGUACUCUGUAAUGAAAAUUACAGAAUCUACU | |
| | | | AAAACAAGGCAAAAUGCCGUGUUUAUCUCGUC | |
| | | | AACUUGUUGGCGAGAUsmUsmUsmU | |
| sgRNA_088- | sgRNA_088 | TSBTx904 | mCsmAsmGsGAUCCGCACAGACUCCAGUUUUA | 1781 |
| 3327 | | | GAGCUAGAAAUAGCAAGUUAAAAUAAGGCUAG | |
| | | | UCCGUUAUCAACUUGAAAAAGUGGCACCGAGU | |
| | | | CGGUGCmUsmUsmUsU | |

TABLE 1C-2
Exemplary spacer sequences and their corresponding target sequences for use
in targeting a base editor to disrupt a promoter region of an LPA polynucleotide.

| | | | PAM | |
|---|---|---|---|---|
| | | | Sequence | PAM |
| Guide Name | Base Editor Name | napDNAbp | Family | Sequence |
| ABE_NNNRRT_21 nt_5-14_014_−_ | saCas9 KKH ABE | saCas9 | NNNRRT | CAAGGT |
| 160664368_saCas9 | | | | |
| CBE_NNNRRT_21 nt_3-12_015_−_ | saCas9 KKH CBE | saCas9 | NNNRRT | CAAGGT |
| 160664368_saCas9 | | | | |
| ABE_NGG_20 nt_3-9_002_−_ | spCas9 ABE | spCas9 | NGG | GGG |
| 160664446_spCas9 | | | | |
| CBE_NGG_20 nt_4-9_003_−_ | spCas9 CBE | spCas9 | NGG | GGG |
| 160664446_spCas9 | | | | |
| CBE_NGC_20 nt_4- | spCas9 NGC CBE | spCas9 | NGC | TGC |
| 9_009_+_160664319_spCas9 | | | | |
| ABE_NGC_20 nt_3- | spCas9 NGC ABE | spCas9 | NGC | TGC |
| 9_008_+_160664319_spCas9 | | | | |
| ABE_NGG_20 nt_3-9_002_−_ | spCas9 ABE | spCas9 | NGG | GGG |
| 160664444_spCas9 | | | | |
| CBE_NGG_20 nt_4-9_003_−_ | spCas9 CBE | spCas9 | NGG | GGG |
| 160664444_spCas9 | | | | |
| CBE_NGA_20 nt_4-9_006_−_ | spCas9 VRQR CBE | spCas9 | NGA | GGA |
| 160664512_spCas9 | | | | |
| ABE_NGA_20 nt_3-9_005_−_ | spCas9 VRQR ABE | spCas9 | NGA | GGA |
| 160664512_spCas9 | | | | |
| ABE_NGG_20 nt_3-9_002_−_ | spCas9 ABE | spCas9 | NGG | AGG |
| 160664496_spCas9 | | | | |
| CBE_NGG_20 nt_4-9_003_−_ | spCas9 CBE | spCas9 | NGG | AGG |
| 160664496_spCas9 | | | | |
| ABE_NGG_20 nt_3-9_002_−_ | spCas9 ABE | spCas9 | NGG | GGG |
| 160664290_spCas9 | | | | |
| CBE_NGG_20 nt_4-9_003_−_ | spCas9 CBE | spCas9 | NGG | GGG |
| 160664290_spCas9 | | | | |
| ABE_NGG_20 nt_3-9_002_−_ | spCas9 ABE | spCas9 | NGG | GGG |
| 160664495_spCas9 | | | | |
| CBE_NGG_20 nt_4-9_003_−_ | spCas9 CBE | spCas9 | NGG | GGG |
| 160664495_spCas9 | | | | |
| CBE_NGC_20 nt_4- | spCas9 NGC CBE | spCas9 | NGC | TGC |
| 9_009_+_160664434_spCas9 | | | | |
| ABE_NGC_20 nt_3- | spCas9 NGC ABE | spCas9 | NGC | TGC |
| 9_008_+_160664434_spCas9 | | | | |
| ABE_NNNRRT_21 nt_5-14_014_−_ | saCas9 KKH ABE | saCas9 | NNNRRT | GGCAAT |
| 160664462_saCas9 | | | | |
| CBE_NNNRRT_21 nt_3-12_015_−_ | saCas9 KKH CBE | saCas9 | NNNRRT | GGCAAT |
| 160664462_saCas9 | | | | |
| ABE_NGG_20 nt_3-9_002_−_ | spCas9 ABE | spCas9 | NGG | GGG |
| 160664494_spCas9 | | | | |
| CBE_NGG_20 nt_4-9_003_−_ | spCas9 CBE | spCas9 | NGG | GGG |
| 160664494_spCas9 | | | | |
| ABE_NGG_20 nt_3-9_002_−_ | spCas9 ABE | spCas9 | NGG | TGG |
| 160664291_spCas9 | | | | |
| CBE_NGG_20 nt_4-9_003_−_ | spCas9 CBE | spCas9 | NGG | TGG |
| 160664291_spCas9 | | | | |
| ABE_NNGRRT_21 nt_5-14_011_−_ | saCas9 ABE | saCas9 | NNGRRT | GAGAAT |
| 160664546_saCas9 | | | | |
| CBE_NNGRRT_21 nt_3-12_012_−_ | saCas9 CBE | saCas9 | NNGRRT | GAGAAT |
| 160664546_saCas9 | | | | |
| CBE_NGC_20 nt_4-9_009_−_ | spCas9 NGC CBE | spCas9 | NGC | TGC |
| 160664287_spCas9 | | | | |
| ABE_NGC_20 nt_3-9_008_−_ | spCas9 NGC ABE | spCas9 | NGC | TGC |
| 160664287_spCas9 | | | | |
| CBE_NGA_20 nt_4- | spCas9 VRQR CBE | spCas9 | NGA | AGA |
| 9_006_+_160664312_spCas9 | | | | |
| ABE_NGA_20 nt_3- | spCas9 VRQR ABE | spCas9 | NGA | AGA |
| 9_005_+_160664312_spCas9 | | | | |
| CBE_NGC_20 nt_4- | spCas9 NGC CBE | spCas9 | NGC | TGC |
| 9_009_+_160664398_spCas9 | | | | |
| ABE_NGC_20 nt_3- | spCas9 NGC ABE | spCas9 | NGC | TGC |
| 9_008_+_160664398_spCas9 | | | | |
| ABE_NGG_20 nt_3- | spCas9 ABE | spCas9 | NGG | TGG |
| 9_002_+_160664408_spCas9 | | | | |
| CBE_NGG_20 nt_4- | spCas9 CBE | spCas9 | NGG | TGG |
| 9_003_+_160664408_spCas9 | | | | |
| ABE_NGG_20 nt_3-9_002_−_ | spCas9 ABE | spCas9 | NGG | TGG |
| 160664336_spCas9 | | | | |
| CBE_NGG_20 nt_4-9_003_−_ | spCas9 CBE | spCas9 | NGG | TGG |
| 160664336_spCas9 | | | | |
| CBE_NGC_20 nt_4-9_009_−_ | spCas9 NGC CBE | spCas9 | NGC | TGC |
| 160664352_spCas9 | | | | |
| ABE_NGC_20 nt_3-9_008_−_ | spCas9 NGC ABE | spCas9 | NGC | TGC |
| 160664352_spCas9 | | | | |
| CBE_NGA_20 nt_4-9_006_−_ | spCas9 VRQR CBE | spCas9 | NGA | TGA |
| 160664491_spCas9 | | | | |
| ABE_NGA_20 nt_3-9_005_−_ | spCas9 VRQR ABE | spCas9 | NGA | TGA |
| 160664491_spCas9 | | | | |
| ABE_NGG_20 nt_3-9_002_−_ | spCas9 ABE | spCas9 | NGG | AGG |
| 160664489_spCas9 | | | | |
| CBE_NGG_20 nt_4-9_003_−_ | spCas9 CBE | spCas9 | NGG | AGG |
| 160664489_spCas9 | | | | |
| ABE_NNNRRT_21 nt_5- | saCas9 KKH ABE | saCas9 | NNNRRT | GTCAAT |
| 14_014_+_160664388_saCas9 | | | | |
| CBE_NNNRRT_21 nt_3- | saCas9 KKH CBE | saCas9 | NNNRRT | GTCAAT |
| 12_015_+_160664388_saCas9 | | | | |
| ABE_NGG_20 nt_3-9_002_−_ | spCas9 ABE | spCas9 | NGG | AGG |
| 160664513_spCas9 | | | | |
| CBE_NGG_20 nt_4-9_003_−_ | spCas9 CBE | spCas9 | NGG | AGG |
| 160664513_spCas9 | | | | |
| ABE_NGG_20 nt_3- | spCas9 ABE | spCas9 | NGG | GGG |
| 9_002_+_160664335_spCas9 | | | | |
| CBE_NGG_20 nt_4- | spCas9 CBE | spCas9 | NGG | GGG |
| 9_003_+_160664335_spCas9 | | | | |
| CBE_NGA_20 nt_4- | spCas9 VRQR CBE | spCas9 | NGA | GGA |
| 9_006_+_160664403_spCas9 | | | | |
| ABE_NGA_20 nt_3- | spCas9 VRQR ABE | spCas9 | NGA | GGA |
| 9_005_+_160664403_spCas9 | | | | |
| CBE_NGA_20 nt_4-9_006_−_ | spCas9 VRQR CBE | spCas9 | NGA | TGA |
| 160664449_spCas9 | | | | |
| ABE_NGA_20 nt_3-9_005_−_ | spCas9 VRQR ABE | spCas9 | NGA | TGA |
| 160664449_spCas9 | | | | |
| ABE_NNNRRT_21 nt_5- | saCas9 KKH ABE | saCas9 | NNNRRT | TGCAAT |
| 14_014_+_160664382_saCas9 | | | | |
| CBE_NNNRRT_21 nt_3- | saCas9 KKH CBE | saCas9 | NNNRRT | TGCAAT |
| 12_015_+_160664382_saCas9 | | | | |
| ABE_NGG_20 nt_3-9_002_−_ | spCas9 ABE | spCas9 | NGG | TGG |
| 160664341_spCas9 | | | | |
| CBE_NGG_20 nt_4-9_003_−_ | spCas9 CBE | spCas9 | NGG | TGG |
| 160664341_spCas9 | | | | |
| CBE_NGC_20 nt_4-9_009_−_ | spCas9 NGC CBE | spCas9 | NGC | AGC |
| 160664347_spCas9 | | | | |
| ABE_NGC_20 nt_3-9_008_−_ | spCas9 NGC ABE | spCas9 | NGC | AGC |
| 160664347_spCas9 | | | | |
| CBE_NGC_20 nt_4-9_009_−_ | spCas9 NGC CBE | spCas9 | NGC | GGC |
| 160664340_spCas9 | | | | |
| ABE_NGC_20 nt_3-9_008_−_ | spCas9 NGC ABE | spCas9 | NGC | GGC |
| 160664340_spCas9 | | | | |
| CBE_NGA_20 nt_4-9_006_−_ | spCas9 VRQR CBE | spCas9 | NGA | AGA |
| 160664326_spCas9 | | | | |
| ABE_NGA_20 nt_3-9_005_−_ | spCas9 VRQR ABE | spCas9 | NGA | AGA |
| 160664326_spCas9 | | | | |
| ABE_NNNRRT_21 nt_5-14_014_−_ | saCas9 KKH ABE | saCas9 | NNNRRT | GGTAAT |
| 160664365_saCas9 | | | | |
| CBE_NNNRRT_21 nt_3-12_015_−_ | saCas9 KKH CBE | saCas9 | NNNRRT | GGTAAT |
| 160664365_saCas9 | | | | |
| CBE_NGA_20 nt_4-9_006_−_ | spCas9 VRQR CBE | spCas9 | NGA | TGA |
| 160664328_spCas9 | | | | |
| ABE_NGA_20 nt_3-9_005_−_ | spCas9 VRQR ABE | spCas9 | NGA | TGA |
| 160664328_spCas9 | | | | |
| ABE_NNNRRT_21 nt_5-14_014_−_ | saCas9 KKH ABE | saCas9 | NNNRRT | TCTGGT |
| 160664297_saCas9 | | | | |
| CBE_NNNRRT_21 nt_3-12_015_−_ | saCas9 KKH CBE | saCas9 | NNNRRT | TCTGGT |
| 160664297_saCas9 | | | | |
| CBE_NGA_20 nt_4- | spCas9 VRQR CBE | spCas9 | NGA | TGA |
| 9_006_+_160664495_spCas9 | | | | |
| ABE_NGA_20 nt_3- | spCas9 VRQR ABE | spCas9 | NGA | TGA |
| 9_005_+_160664495_spCas9 | | | | |
| ABE_NNNRRT_21 nt_5- | saCas9 KKH ABE | saCas9 | NNNRRT | GTTAAT |
| 14_014_+_160664294_saCas9 | | | | |
| CBE_NNNRRT_21 nt_3- | saCas9 KKH CBE | saCas9 | NNNRRT | GTTAAT |
| 12_015_+_160664294_saCas9 | | | | |
| CBE_NGA_20 nt_4- | spCas9 VRQR CBE | spCas9 | NGA | AGA |
| 9_006_+_160664395_spCas9 | | | | |
| ABE_NGA_20 nt_3- | spCas9 VRQR ABE | spCas9 | NGA | AGA |
| 9_005_+_160664395_spCas9 | | | | |
| CBE_NGA_20 nt_4-9_006_−_ | spCas9 VRQR CBE | spCas9 | NGA | TGA |
| 160664349_spCas9 | | | | |
| ABE_NGA_20 nt_3-9_005_−_ | spCas9 VRQR ABE | spCas9 | NGA | TGA |
| 160664349_spCas9 | | | | |
| ABE_NGG_20 nt_3- | spCas9 ABE | spCas9 | NGG | TGG |
| 9_002_+_160664401_spCas9 | | | | |
| CBE_NGG_20 nt_4- | spCas9 CBE | spCas9 | NGG | TGG |
| 9_003_+_160664401_spCas9 | | | | |
| CBE_NGA_20 nt_4- | spCas9 VRQR CBE | spCas9 | NGA | TGA |
| 9_006_+_160664352_spCas9 | | | | |
| ABE_NGA_20 nt_3- | spCas9 VRQR ABE | spCas9 | NGA | TGA |
| 9_005_+_160664352_spCas9 | | | | |
| ABE_NNNRRT_21 nt_5- | saCas9 KKH ABE | saCas9 | NNNRRT | AATGAT |
| 14_014_+_160664297_saCas9 | | | | |
| CBE_NNNRRT_21 nt_3- | saCas9 KKH CBE | saCas9 | NNNRRT | AATGAT |
| 12_015_+_160664297_saCas9 | | | | |
| ABE_NNNRRT_21 nt_5-14_014_−_ | saCas9 KKH ABE | saCas9 | NNNRRT | AAAGAT |
| 160664507_saCas9 | | | | |
| CBE_NNNRRT_21 nt_3-12_015_−_ | saCas9 KKH CBE | saCas9 | NNNRRT | AAAGAT |
| 160664507_saCas9 | | | | |
| ABE_NNNRRT_21 nt_5-14_014_−_ | saCas9 KKH ABE | saCas9 | NNNRRT | CTTAAT |
| 160664310_saCas9 | | | | |
| CBE_NNNRRT_21 nt_3-12_015_−_ | saCas9 KKH CBE | saCas9 | NNNRRT | CTTAAT |
| 160664310_saCas9 | | | | |
| ABE_NNNRRT_21 nt_5- | saCas9 KKH ABE | saCas9 | NNNRRT | TTAAGT |
| 14_014_+_160664290_saCas9 | | | | |
| CBE_NNNRRT_21 nt_3- | saCas9 KKH CBE | saCas9 | NNNRRT | TTAAGT |
| 12_015_+_160664290_saCas9 | | | | |
| ABE_NNGRRT_21 nt_5- | saCas9 ABE | saCas9 | NNGRRT | GAGAGT |
| 14_011_+_160664377_saCas9 | | | | |
| CBE_NNGRRT_21 nt_3- | saCas9 CBE | saCas9 | NNGRRT | GAGAGT |
| 12_012_+_160664377_saCas9 | | | | |
| ABE_NNNRRT_21 nt_5- | saCas9 KKH ABE | saCas9 | NNNRRT | ATAGAT |
| 14_014_+_160664392_saCas9 | | | | |
| CBE_NNNRRT_21 nt_3- | saCas9 KKH CBE | saCas9 | NNNRRT | ATAGAT |
| 12_015_+_160664392_saCas9 | | | | |
| ABE_NNNRRT_21 nt_5- | saCas9 KKH ABE | saCas9 | NNNRRT | AGTGGT |
| 14_014_+_160664405_saCas9 | | | | |
| CBE_NNNRRT_21 nt_3- | saCas9 KKH CBE | saCas9 | NNNRRT | AGTGGT |
| 12_015_+_160664405_saCas9 | | | | |
| ABE_NNNRRT_21 nt_5- | saCas9 KKH ABE | saCas9 | NNNRRT | CCTGAT |
| 14_014_+_160664492_saCas9 | | | | |
| CBE_NNNRRT_21 nt_3- | saCas9 KKH CBE | saCas9 | NNNRRT | CCTGAT |
| 12_015_+_160664492_saCas9 | | | | |
| ABE_NNNRRT_21 nt_5- | saCas9 KKH ABE | saCas9 | NNNRRT | ATTAGT |
| 14_014_+_160664496_saCas9 | | | | |
| CBE_NNNRRT_21 nt_3- | saCas9 KKH CBE | saCas9 | NNNRRT | ATTAGT |
| 12_015_+_160664496_saCas9 | | | | |
| ABE_NNNRRT_21 nt_5- | saCas9 KKH ABE | saCas9 | NNNRRT | TAAAAT |
| 14_014_+_160664367_saCas9 | | | | |
| CBE_NNNRRT_21 nt_3- | saCas9 KKH CBE | saCas9 | NNNRRT | TAAAAT |
| 12_015_+_160664367_saCas9 | | | | |
| ABE_NNGRRT_21 nt_5-14_011_−_ | saCas9 ABE | saCas9 | NNGRRT | ATGGGT |
| 160664334_saCas9 | | | | |
| CBE_NNGRRT_21 nt_3-12_012_−_ | saCas9 CBE | saCas9 | NNGRRT | ATGGGT |
| 160664334_saCas9 | | | | |
| ABE_NNNRRT_21 nt_5- | saCas9 KKH ABE | saCas9 | NNNRRT | GGAAGT |
| 14_014_+_160664402_saCas9 | | | | |
| CBE_NNNRRT_21 nt_3- | saCas9 KKH CBE | saCas9 | NNNRRT | GGAAGT |
| 12_015_+_160664402_saCas9 | | | | |
| ABE_NNGRRT_21 nt_5-14_011_−_ | saCas9 ABE | saCas9 | NNGRRT | AGGGGT |
| 160664493_saCas9 | | | | |
| CBE_NNGRRT_21 nt_3-12_012_−_ | saCas9 CBE | saCas9 | NNGRRT | AGGGGT |
| 160664493_saCas9 | | | | |
| ABE_NNGRRT_21 nt_5-14_011_−_ | saCas9 ABE | saCas9 | NNGRRT | GTGGGT |
| 160664289_saCas9 | | | | |
| CBE_NNGRRT_21 nt_3-12_012_−_ | saCas9 CBE | saCas9 | NNGRRT | GTGGGT |
| 160664289_saCas9 | | | | |
| ABE_NNGRRT_21 nt_5- | saCas9 ABE | saCas9 | NNGRRT | TAGAGT |
| 14_011_+_160664357_saCas9 | | | | |
| CBE_NNGRRT_21 nt_3- | saCas9 CBE | saCas9 | NNGRRT | TAGAGT |
| 12_012_+_160664357_saCas9 | | | | |
| ABE_NNGRRT_21 nt_5-14_011_−_ | saCas9 ABE | saCas9 | NNGRRT | GAGAAT |
| 160664322_saCas9 | | | | |
| CBE_NNGRRT_21 nt_3-12_012_−_ | saCas9 CBE | saCas9 | NNGRRT | GAGAAT |
| 160664322_saCas9 | | | | |
| ABE_NNGRRT_21 nt_5- | saCas9 ABE | saCas9 | NNGRRT | CAGGGT |
| 14_011_+_160664332_saCas9 | | | | |
| CBE_NNGRRT_21 nt_3- | saCas9 CBE | saCas9 | NNGRRT | CAGGGT |
| 12_012_+_160664332_saCas9 | | | | |
| ABE_NNGRRT_21 nt_5- | saCas9 ABE | saCas9 | NNGRRT | TTGAAT |
| 14_011_+_160664350_saCas9 | | | | |
| CBE_NNGRRT_21 nt_3- | saCas9 CBE | saCas9 | NNGRRT | TTGAAT |
| 12_012_+_160664350_saCas9 | | | | |
| ABE_NNNRRT_21 nt_5-14_014_−_ | saCas9 KKH ABE | saCas9 | NNNRRT | TCAAAT |
| 160664394_saCas9 | | | | |
| CBE_NNNRRT_21 nt_3-12_015_−_ | saCas9 KKH CBE | saCas9 | NNNRRT | TCAAAT |
| 160664394_saCas9 | | | | |
| | | | Location of target | |
| | | | site on Chr6 | |
| | | SEQ | (hg38.2bit genome | |
| | | ID | sequence) | |
| Guide_Name | Target Sequence | NO | start | end |
| ABE_NNNRRT 21_nt_5- | TATTTTATAAGACTCTATATTCAA | 1782 | 160664368 | 160664395 |
| 14_014_−_160664368_saCas9 | GGT | | | |
| CBE_NNNRRT 21_nt_3- | TATTTTATAAGACTCTATATTCAA | 1783 | 160664368 | 160664395 |
| 12_015_−_160664368_saCas9 | GGT | | | |
| ABE_NGG_20 nt_3-9_002_−_ | AGGCAATGTGGAGCAGCTGAGGG | 1784 | 160664446 | 160664469 |
| 160664446_spCas9 | | | | |
| CBE_NGG_20 nt_4-9_003_−_ | AGGCAATGTGGAGCAGCTGAGGG | 1785 | 160664446 | 160664469 |
| 160664446_spCas9 | | | | |
| CBE_NGC_20 nt_4- | ATGATTCTCTCAGAGACCCATGC | 1786 | 160664319 | 160664342 |
| 9_009_+_160664319_spCas9 | | | | |
| ABE_NGC_20 nt_3- | ATGATTCTCTCAGAGACCCATGC | 1787 | 160664319 | 160664342 |
| 9_008_+_160664319_spCas9 | | | | |
| ABE_NGG_20 nt_3-9_002_−_ | GCAATGTGGAGCAGCTGAGGGGG | 1788 | 160664444 | 160664467 |
| 160664444_spCas9 | | | | |
| CBE_NGG_20 nt_4-9_003_−_ | GCAATGTGGAGCAGCTGAGGGGG | 1789 | 160664444 | 160664467 |
| 160664444_spCas9 | | | | |
| CBE_NGA_20 nt_4-9_006_−_ | GGAGGAAACAAGACTAATCAGGA | 1790 | 160664512 | 160664535 |
| 160664512_spCas9 | | | | |
| ABE_NGA_20 nt_3-9_005_−_ | GGAGGAAACAAGACTAATCAGGA | 1791 | 160664512 | 160664535 |
| 160664512_spCas9 | | | | |
| ABE_NGG_20 nt_3-9_002_−_ | ATCAGGAAAGATGAAGGTCTAGG | 1792 | 160664496 | 160664519 |
| 160664496_spCas9 | | | | |
| CBE_NGG_20 nt_4-9_003_−_ | ATCAGGAAAGATGAAGGTCTAGG | 1793 | 160664496 | 160664519 |
| 160664496_spCas9 | | | | |
| ABE_NGG_20 nt_3-9_002_−_ | AATTTGACTATCTGGTTTGTGGG | 1794 | 160664290 | 160664313 |
| 160664290_spCas9 | | | | |
| CBE_NGG_20 nt_4-9_003_−_ | AATTTGACTATCTGGTTTGTGGG | 1795 | 160664290 | 160664313 |
| 160664290_spCas9 | | | | |
| ABE_NGG_20 nt_3-9_002_−_ | TCAGGAAAGATGAAGGTCTAGGG | 1796 | 160664495 | 160664518 |
| 160664495_spCas9 | | | | |
| CBE_NGG_20 nt_4-9_003_−_ | TCAGGAAAGATGAAGGTCTAGGG | 1797 | 160664495 | 160664518 |
| 160664495_spCas9 | | | | |
| CBE_NGC_20 nt_4- | AAGCCATTTCCCCCCTCAGCTGC | 1798 | 160664434 | 160664457 |
| 9_009_+_160664434_spCas9 | | | | |
| ABE_NGC_20 nt_3- | AAGCCATTTCCCCCCTCAGCTGC | 1799 | 160664434 | 160664457 |
| 9_008_+_160664434_spCas9 | | | | |
| ABE_NNNRRT_21 nt_5- | GAAGGAGTAAGGAGACATAAAGGC | 1800 | 160664462 | 160664489 |
| 14_014_−_160664462_saCas9 | AAT | | | |
| CBE_NNNRRT_21 nt_3- | GAAGGAGTAAGGAGACATAAAGGC | 1801 | 160664462 | 160664489 |
| 12_015_−_160664462_saCas9 | AAT | | | |
| ABE_NGG_20 nt_3-9_002_−_ | CAGGAAAGATGAAGGTCTAGGGG | 1802 | 160664494 | 160664517 |
| 160664494_spCas9 | | | | |
| CBE_NGG_20 nt_4-9_003_−_ | CAGGAAAGATGAAGGTCTAGGGG | 1803 | 160664494 | 160664517 |
| 160664494_spCas9 | | | | |
| ABE_NGG_20 nt_3-9_002_−_ | TAATTTGACTATCTGGTTTGTGG | 1804 | 160664291 | 160664314 |
| 160664291_spCas9 | | | | |
| CBE_NGG_20 nt_4-9_003_−_ | TAATTTGACTATCTGGTTTGTGG | 1805 | 160664291 | 160664314 |
| 160664291_spCas9 | | | | |
| ABE_NNGRRT_21 nt_5- | TTTTATTTTATCCAAAAGAAAGAG | 1806 | 160664546 | 160664573 |
| 14_011_−_160664546_saCas9 | AAT | | | |
| CBE_NNGRRT_21 nt_3- | TTTTATTTTATCCAAAAGAAAGAG | 1807 | 160664546 | 160664573 |
| 12_012_−_160664546_saCas9 | AAT | | | |
| CBE_NGC_20 nt_4-9_009_−_ | TTGACTATCTGGTTTGTGGGTGC | 1808 | 160664287 | 160664310 |
| 160664287_spCas9 | | | | |
| ABE_NGC_20 nt_3-9_008_−_ | TTGACTATCTGGTTTGTGGGTGC | 1809 | 160664287 | 160664310 |
| 160664287_spCas9 | | | | |
| CBE_NGA_20 nt_4- | TAAGTTAATGATTCTCTCAGAGA | 1810 | 160664312 | 160664335 |
| 9_006_+_160664312_spCas9 | | | | |
| ABE_NGA_20 nt_3- | TAAGTTAATGATTCTCTCAGAGA | 1811 | 160664312 | 160664335 |
| 9_005_+_160664312_spCas9 | | | | |
| CBE_NGC_20 nt_4- | GAGAGTGCAATGTCAATAGATGC | 1812 | 160664398 | 160664421 |
| 9_009_+_160664398_spCas9 | | | | |
| ABE_NGC_20 nt_3- | GAGAGTGCAATGTCAATAGATGC | 1813 | 160664398 | 160664421 |
| 9_008_+_160664398_spCas9 | | | | |
| ABE_NGG_20 nt_3- | TGTCAATAGATGCTGGGAAGTGG | 1814 | 160664408 | 160664431 |
| 9_002_+_160664408_spCas9 | | | | |
| CBE_NGG_20 nt_4- | TGTCAATAGATGCTGGGAAGTGG | 1815 | 160664408 | 160664431 |
| 9_003_+_160664408_spCas9 | | | | |
| ABE_NGG_20 nt_3-9_002_−_ | ACCCTGCTGAGCCAGTGGCATGG | 1816 | 160664336 | 160664359 |
| 160664336_spCas9 | | | | |
| CBE_NGG_20 nt_4-9_003_−_ | ACCCTGCTGAGCCAGTGGCATGG | 1817 | 160664336 | 160664359 |
| 160664336_spCas9 | | | | |
| CBE_NGC_20 nt_4-9_009_−_ | TCAAGGTAATGTTTGAACCCTGC | 1818 | 160664352 | 160664375 |
| 160664352_spCas9 | | | | |
| ABE_NGC_20 nt_3-9_008_−_ | TCAAGGTAATGTTTGAACCCTGC | 1819 | 160664352 | 160664375 |
| 160664352_spCas9 | | | | |
| CBE_NGA_20 nt_4-9_006_−_ | GAAAGATGAAGGTCTAGGGGTGA | 1820 | 160664491 | 160664514 |
| 160664491_spCas9 | | | | |
| ABE_NGA_20 nt_3-9_005_−_ | GAAAGATGAAGGTCTAGGGGTGA | 1821 | 160664491 | 160664514 |
| 160664491_spCas9 | | | | |
| ABE_NGG_20 nt_3-9_002_−_ | AAGATGAAGGTCTAGGGGTGAGG | 1822 | 160664489 | 160664512 |
| 160664489_spCas9 | | | | |
| CBE_NGG_20 nt_4-9_003_−_ | AAGATGAAGGTCTAGGGGTGAGG | 1823 | 160664489 | 160664512 |
| 160664489_spCas9 | | | | |
| ABE_NNNRRT_21 nt_5- | TAAAATATTTGAGAGTGCAATGTC | 1824 | 160664388 | 160664415 |
| 14_014_+_160664388_saCas9 | AAT | | | |
| CBE_NNNRRT_21 nt_3- | TAAAATATTTGAGAGTGCAATGTC | 1825 | 160664388 | 160664415 |
| 12_015_+_160664388_saCas9 | AAT | | | |
| ABE_NGG_20 nt_3-9_002_−_ | GGGAGGAAACAAGACTAATCAGG | 1826 | 160664513 | 160664536 |
| 160664513_spCas9 | | | | |
| CBE_NGG_20 nt_4-9_003_−_ | GGGAGGAAACAAGACTAATCAGG | 1827 | 160664513 | 160664536 |
| 160664513_spCas9 | | | | |
| ABE_NGG_20 nt_3- | CCCATGCCACTGGCTCAGCAGGG | 1828 | 160664335 | 160664358 |
| 9_002_+_160664335_spCas9 | | | | |
| CBE_NGG_20 nt_4- | CCCATGCCACTGGCTCAGCAGGG | 1829 | 160664335 | 160664358 |
| 9_003_+_160664335_spCas9 | | | | |
| CBE_NGA_20 nt_4- | TGCAATGTCAATAGATGCTGGGA | 1830 | 160664403 | 160664426 |
| 9_006_+_160664403_spCas9 | | | | |
| ABE_NGA_20 nt_3- | TGCAATGTCAATAGATGCTGGGA | 1831 | 160664403 | 160664426 |
| 9_005_+_160664403_spCas9 | | | | |
| CBE_NGA_20 nt_4-9_006_−_ | TAAAGGCAATGTGGAGCAGCTGA | 1832 | 160664449 | 160664472 |
| 160664449_spCas9 | | | | |
| ABE_NGA_20 nt_3-9_005_−_ | TAAAGGCAATGTGGAGCAGCTGA | 1833 | 160664449 | 160664472 |
| 160664449_spCas9 | | | | |
| ABE_NNNRRT_21 nt_5- | GTCTTATAAAATATTTGAGAGTGC | 1834 | 160664382 | 160664409 |
| 14_014_+_160664382_saCas9 | AAT | | | |
| CBE_NNNRRT_21 nt_3- | GTCTTATAAAATATTTGAGAGTGC | 1835 | 160664382 | 160664409 |
| 12_015_+_160664382_saCas9 | AAT | | | |
| ABE_NGG_20 nt_3-9_002_−_ | TTTGAACCCTGCTGAGCCAGTGG | 1836 | 160664341 | 160664364 |
| 160664341_spCas9 | | | | |
| CBE_NGG_20 nt_4-9_003_−_ | TTTGAACCCTGCTGAGCCAGTGG | 1837 | 160664341 | 160664364 |
| 160664341_spCas9 | | | | |
| CBE_NGC_20 nt_4-9_009_−_ | GTAATGTTTGAACCCTGCTGAGC | 1838 | 160664347 | 160664370 |
| 160664347_spCas9 | | | | |
| ABE_NGC_20 nt_3-9_008_−_ | GTAATGTTTGAACCCTGCTGAGC | 1839 | 160664347 | 160664370 |
| 160664347_spCas9 | | | | |
| CBE_NGC_20 nt_4-9_009_−_ | TTGAACCCTGCTGAGCCAGTGGC | 1840 | 160664340 | 160664363 |
| 160664340_spCas9 | | | | |
| ABE_NGC_20 nt_3-9_008_−_ | TTGAACCCTGCTGAGCCAGTGGC | 1841 | 160664340 | 160664363 |
| 160664340_spCas9 | | | | |
| CBE_NGA_20 nt_4-9_006_−_ | GCCAGTGGCATGGGTCTCTGAGA | 1842 | 160664326 | 160664349 |
| 160664326_spCas9 | | | | |
| ABE_NGA_20 nt_3-9_005_−_ | GCCAGTGGCATGGGTCTCTGAGA | 1843 | 160664326 | 160664349 |
| 160664326_spCas9 | | | | |
| ABE_NNNRRT_21 nt_5- | TTTATAAGACTCTATATTCAAGGT | 1844 | 160664365 | 160664392 |
| 14_014_−_160664365_saCas9 | AAT | | | |
| CBE_NNNRRT_21 nt_3- | TTTATAAGACTCTATATTCAAGGT | 1845 | 160664365 | 160664392 |
| 12_015_−_160664365_saCas9 | AAT | | | |
| CBE_NGA_20 nt_4-9_006_−_ | GAGCCAGTGGCATGGGTCTCTGA | 1846 | 160664328 | 160664351 |
| 160664328_spCas9 | | | | |
| ABE_NGA_20 nt_3-9_005_−_ | GAGCCAGTGGCATGGGTCTCTGA | 1847 | 160664328 | 160664351 |
| 160664328_spCas9 | | | | |
| ABE_NNNRRT_21 nt_5- | ATCATTAACTTAATTTGACTATCT | 1848 | 160664297 | 160664324 |
| 14_014_−_160664297_saCas9 | GGT | | | |
| CBE_NNNRRT_21 nt_3- | ATCATTAACTTAATTTGACTATCT | 1849 | 160664297 | 160664324 |
| 12_015_−_160664297_saCas9 | GGT | | | |
| CBE_NGA_20 nt_4- | CCCTAGACCTTCATCTTTCCTGA | 1850 | 160664495 | 160664518 |
| 9_006_+_160664495_spCas9 | | | | |
| ABE_NGA_20 nt_3- | CCCTAGACCTTCATCTTTCCTGA | 1851 | 160664495 | 160664518 |
| 9_005_+_160664495_spCas9 | | | | |
| ABE_NNNRRT_21 nt_5- | CAAACCAGATAGTCAAATTAAGTT | 1852 | 160664294 | 160664321 |
| 14_014_+_160664294_saCas9 | AAT | | | |
| CBE_NNNRRT_21 nt_3- | CAAACCAGATAGTCAAATTAAGTT | 1853 | 160664294 | 160664321 |
| 12_015_+_160664294_saCas9 | AAT | | | |
| CBE_NGA_20 nt_4- | TTTGAGAGTGCAATGTCAATAGA | 1854 | 160664395 | 160664418 |
| 9_006_+_160664395_spCas9 | | | | |
| ABE_NGA_20 nt_3- | TTTGAGAGTGCAATGTCAATAGA | 1855 | 160664395 | 160664418 |
| 9_005_+_160664395_spCas9 | | | | |
| CBE_NGA_20 nt_4-9_006_−_ | AGGTAATGTTTGAACCCTGCTGA | 1856 | 160664349 | 160664372 |
| 160664349_spCas9 | | | | |
| ABE_NGA_20 nt_3-9_005_−_ | AGGTAATGTTTGAACCCTGCTGA | 1857 | 160664349 | 160664372 |
| 160664349_spCas9 | | | | |
| ABE_NGG_20 nt_3- | AGTGCAATGTCAATAGATGCTGG | 1858 | 160664401 | 160664424 |
| 9_002_+_160664401_spCas9 | | | | |
| CBE_NGG_20 nt_4- | AGTGCAATGTCAATAGATGCTGG | 1859 | 160664401 | 160664424 |
| 9_003_+_160664401_spCas9 | | | | |
| CBE_NGA_20 nt_4- | GCAGGGTTCAAACATTACCTTGA | 1860 | 160664352 | 160664375 |
| 9_006_+_160664352_spCas9 | | | | |
| ABE_NGA_20 nt_3- | GCAGGGTTCAAACATTACCTTGA | 1861 | 160664352 | 160664375 |
| 9_005_+_160664352_spCas9 | | | | |
| ABE_NNNRRT_21 nt_5- | ACCAGATAGTCAAATTAAGTTAAT | 1862 | 160664297 | 160664324 |
| 14_014_+_160664297_saCas9 | GAT | | | |
| CBE_NNNRRT_21 nt_3- | ACCAGATAGTCAAATTAAGTTAAT | 1863 | 160664297 | 160664324 |
| 12_015_+_160664297_saCas9 | GAT | | | |
| ABE_NNNRRT_21 nt_5- | GAGGAAACAAGACTAATCAGGAAA | 1864 | 160664507 | 160664534 |
| 14_014_−_160664507_saCas9 | GAT | | | |
| CBE_NNNRRT_21 nt_3- | GAGGAAACAAGACTAATCAGGAAA | 1865 | 160664507 | 160664534 |
| 12_015_−_160664507_saCas9 | GAT | | | |
| ABE_NNNRRT_21 nt_5- | GGTCTCTGAGAGAATCATTAACTT | 1866 | 160664310 | 160664337 |
| 14_014_−_160664310_saCas9 | AAT | | | |
| CBE_NNNRRT_21 nt_3- | GGTCTCTGAGAGAATCATTAACTT | 1867 | 160664310 | 160664337 |
| 12_015_−_160664310_saCas9 | AAT | | | |
| ABE_NNNRRT_21 nt_5- | CCCACAAACCAGATAGTCAAATTA | 1868 | 160664290 | 160664317 |
| 14_014_+_160664290_saCas9 | AGT | | | |
| CBE_NNNRRT_21 nt_3- | CCCACAAACCAGATAGTCAAATTA | 1869 | 160664290 | 160664317 |
| 12_015_+_160664290_saCas9 | AGT | | | |
| ABE_NNGRRT_21 nt_5- | ATAGAGTCTTATAAAATATTTGAG | 1870 | 160664377 | 160664404 |
| 14_011_+_160664377_saCas9 | AGT | | | |
| CBE_NNGRRT_21 nt_3- | ATAGAGTCTTATAAAATATTTGAG | 1871 | 160664377 | 160664404 |
| 12_012_+_160664377_saCas9 | AGT | | | |
| ABE_NNNRRT_21 nt_5- | ATATTTGAGAGTGCAATGTCAATA | 1872 | 160664392 | 160664419 |
| 14_014_+_160664392_saCas9 | GAT | | | |
| CBE_NNNRRT_21 nt_3- | ATATTTGAGAGTGCAATGTCAATA | 1873 | 160664392 | 160664419 |
| 12_015_+_160664392_saCas9 | GAT | | | |
| ABE_NNNRRT_21 nt_5- | CAATGTCAATAGATGCTGGGAAGT | 1874 | 160664405 | 160664432 |
| 14_014_+_160664405_saCas9 | GGT | | | |
| CBE_NNNRRT_21 nt_3- | CAATGTCAATAGATGCTGGGAAGT | 1875 | 160664405 | 160664432 |
| 12_015_+_160664405_saCas9 | GGT | | | |
| ABE_NNNRRT_21 nt_5- | CACCCCTAGACCTTCATCTTTCCT | 1876 | 160664492 | 160664519 |
| 14_014_+_160664492_saCas9 | GAT | | | |
| CBE_NNNRRT_21 nt_3- | CACCCCTAGACCTTCATCTTTCCT | 1877 | 160664492 | 160664519 |
| 12_015_+_160664492_saCas9 | GAT | | | |
| ABE_NNNRRT_21 nt_5- | CCTAGACCTTCATCTTTCCTGATT | 1878 | 160664496 | 160664523 |
| 14_014_+_160664496_saCas9 | AGT | | | |
| CBE_NNNRRT_21 nt_3- | CCTAGACCTTCATCTTTCCTGATT | 1879 | 160664496 | 160664523 |
| 12_015_+_160664496_saCas9 | AGT | | | |
| ABE_NNNRRT_21 nt_5- | TACCTTGAATATAGAGTCTTATAA | 1880 | 160664367 | 160664394 |
| 14_014_+_160664367_saCas9 | AAT | | | |
| CBE_NNNRRT_21 nt_3- | TACCTTGAATATAGAGTCTTATAA | 1881 | 160664367 | 160664394 |
| 12_015_+_160664367_saCas9 | AAT | | | |
| ABE_NNGRRT_21 nt_5- | GAACCCTGCTGAGCCAGTGGCATG | 1882 | 160664334 | 160664361 |
| 14_011_−_160664334_saCas9 | GGT | | | |
| CBE_NNGRRT_21 nt_3- | GAACCCTGCTGAGCCAGTGGCATG | 1883 | 160664334 | 160664361 |
| 12_012_−_160664334_saCas9 | GGT | | | |
| ABE_NNNRRT_21 nt_5- | GTGCAATGTCAATAGATGCTGGGA | 1884 | 160664402 | 160664429 |
| 14_014_+_160664402_saCas9 | AGT | | | |
| CBE_NNNRRT_21 nt_3- | GTGCAATGTCAATAGATGCTGGGA | 1885 | 160664402 | 160664429 |
| 12_015_+_160664402_saCas9 | AGT | | | |
| ABE_NNGRRT_21 nt_5- | AATCAGGAAAGATGAAGGTCTAGG | 1886 | 160664493 | 160664520 |
| 14_011_−_160664493_saCas9 | GGT | | | |
| CBE_NNGRRT_21 nt_3- | AATCAGGAAAGATGAAGGTCTAGG | 1887 | 160664493 | 160664520 |
| 12_012_−_160664493_saCas9 | GGT | | | |
| ABE_NNGRRT_21 nt_5- | CTTAATTTGACTATCTGGTTTGTG | 1888 | 160664289 | 160664316 |
| 14_011_−_160664289_saCas9 | GGT | | | |
| CBE_NNGRRT_21 nt_3- | CTTAATTTGACTATCTGGTTTGTG | 1889 | 160664289 | 160664316 |
| 12_012_−_160664289_saCas9 | GGT | | | |
| ABE_NNGRRT_21 nt_5- | GTTCAAACATTACCTTGAATATAG | 1890 | 160664357 | 160664384 |
| 14_011_+_160664357_saCas9 | AGT | | | |
| CBE_NNGRRT_21 nt_3- | GTTCAAACATTACCTTGAATATAG | 1891 | 160664357 | 160664384 |
| 12_012_+_160664357_saCas9 | AGT | | | |
| ABE_NNGRRT_21 nt_5- | GCCAGTGGCATGGGTCTCTGAGAG | 1892 | 160664322 | 160664349 |
| 14_011_−_160664322_saCas9 | AAT | | | |
| CBE_NNGRRT_21 nt_3- | GCCAGTGGCATGGGTCTCTGAGAG | 1893 | 160664322 | 160664349 |
| 12_012_−_160664322_saCas9 | AAT | | | |
| ABE_NNGRRT_21 nt_5- | AGACCCATGCCACTGGCTCAGCAG | 1894 | 160664332 | 160664359 |
| 14_011_+_160664332_saCas9 | GGT | | | |
| CBE_NNGRRT_21 nt_3- | AGACCCATGCCACTGGCTCAGCAG | 1895 | 160664332 | 160664359 |
| 12_012_+_160664332_saCas9 | GGT | | | |
| ABE_NNGRRT_21 nt_5- | CAGCAGGGTTCAAACATTACCTTG | 1896 | 160664350 | 160664377 |
| 14_011_+_160664350_saCas9 | AAT | | | |
| CBE_NNGRRT_21 nt_3- | CAGCAGGGTTCAAACATTACCTTG | 1897 | 160664350 | 160664377 |
| 12_012_+_160664350_saCas9 | AAT | | | |
| ABE_NNNRRT_21 nt_5- | GCATCTATTGACATTGCACTCTCA | 1898 | 160664394 | 160664421 |
| 14_014_−_160664394_saCas9 | AAT | | | |
| CBE_NNNRRT_21 nt_3- | GCATCTATTGACATTGCACTCTCA | 1899 | 160664394 | 160664421 |
| 12_015_−_160664394_saCas9 | AAT | | | |

| | | SEQ ID |
|---|---|---|
| Guide_Name | Spacer Sequence | NO |
| ABE_NNNRRT_21 nt_5-14_014_−_ | UAUUUUAUAAGACUCUAUAUU | 1900 |
| 160664368_saCas9 | | |
| CBE_NNNRRT_21 nt_3-12_015_−_ | UAUUUUAUAAGACUCUAUAUU | 1901 |
| 160664368_saCas9 | | |
| ABE_NGG_20 nt_3-9_002_−_160664446_spCas9 | AGGCAAUGUGGAGCAGCUGA | 1902 |
| CBE_NGG_20 nt_4-9_003_−_160664446_spCas9 | AGGCAAUGUGGAGCAGCUGA | 1903 |
| CBE_NGC_20 nt_4-9_009_+_160664319_spCas9 | AUGAUUCUCUCAGAGACCCA | 1904 |
| ABE_NGC_20 nt_3-9_008_+_160664319_spCas9 | AUGAUUCUCUCAGAGACCCA | 1905 |
| ABE_NGG_20 nt_3-9_002_−_160664444_spCas9 | GCAAUGUGGAGCAGCUGAGG | 1906 |
| CBE_NGG_20 nt_4-9_003_−_160664444_spCas9 | GCAAUGUGGAGCAGCUGAGG | 1907 |
| CBE_NGA_20 nt_4-9_006_−_160664512_spCas9 | GGAGGAAACAAGACUAAUCA | 1908 |
| ABE_NGA_20 nt_3-9_005_−_160664512_spCas9 | GGAGGAAACAAGACUAAUCA | 1909 |
| ABE_NGG_20 nt_3-9_002_−_160664496_spCas9 | AUCAGGAAAGAUGAAGGUCU | 1910 |
| CBE_NGG_20 nt_4-9_003_−_160664496_spCas9 | AUCAGGAAAGAUGAAGGUCU | 1911 |
| ABE_NGG_20 nt_3-9_002_−_160664290_spCas9 | AAUUUGACUAUCUGGUUUGU | 1912 |
| CBE_NGG_20 nt_4-9_003_−_160664290_spCas9 | AAUUUGACUAUCUGGUUUGU | 1913 |
| ABE_NGG_20 nt_3-9_002_−_160664495_spCas9 | UCAGGAAAGAUGAAGGUCUA | 1914 |
| CBE_NGG_20 nt_4-9_003_−_160664495_spCas9 | UCAGGAAAGAUGAAGGUCUA | 1915 |
| CBE_NGC_20 nt_4-9_009_+_160664434_spCas9 | AAGCCAUUUCCCCCCUCAGC | 1916 |
| ABE_NGC_20 nt_3-9_008_+_160664434_spCas9 | AAGCCAUUUCCCCCCUCAGC | 1917 |
| ABE_NNNRRT_21 nt_5-14_014_−_ | GAAGGAGUAAGGAGACAUAAA | 1918 |
| 160664462_saCas9 | | |
| CBE_NNNRRT_21 nt_3-12_015_−_ | GAAGGAGUAAGGAGACAUAAA | 1919 |
| 160664462_saCas9 | | |
| ABE_NGG_20 nt_3-9_002_−_160664494_spCas9 | CAGGAAAGAUGAAGGUCUAG | 1920 |
| CBE_NGG_20 nt_4-9_003_−_160664494_spCas9 | CAGGAAAGAUGAAGGUCUAG | 1921 |
| ABE_NGG_20 nt_3-9_002_−_160664291_spCas9 | UAAUUUGACUAUCUGGUUUG | 1922 |
| CBE_NGG_20 nt_4-9_003_−_160664291_spCas9 | UAAUUUGACUAUCUGGUUUG | 1923 |
| ABE_NNGRRT_21 nt_5-14_011_−_ | UUUUAUUUUAUCCAAAAGAAA | 1924 |
| 160664546_saCas9 | | |
| CBE_NNGRRT_21 nt_3-12_012_−_ | UUUUAUUUUAUCCAAAAGAAA | 1925 |
| 160664546_saCas9 | | |
| CBE_NGC_20 nt_4-9_009_−_160664287_spCas9 | UUGACUAUCUGGUUUGUGGG | 1926 |
| ABE_NGC_20 nt_3-9_008_−_160664287_spCas9 | UUGACUAUCUGGUUUGUGGG | 1927 |
| CBE_NGA_20 nt_4-9_006_+_160664312_spCas9 | UAAGUUAAUGAUUCUCUCAG | 1928 |
| ABE_NGA_20 nt_3-9_005_+_160664312_spCas9 | UAAGUUAAUGAUUCUCUCAG | 1929 |
| CBE_NGC_20 nt_4-9_009_+_160664398_spCas9 | GAGAGUGCAAUGUCAAUAGA | 1930 |
| ABE_NGC_20 nt_3-9_008_+_160664398_spCas9 | GAGAGUGCAAUGUCAAUAGA | 1931 |
| ABE_NGG_20 nt_3-9_002_+_160664408_spCas9 | UGUCAAUAGAUGCUGGGAAG | 1932 |
| CBE_NGG_20 nt_4-9_003_+_160664408_spCas9 | UGUCAAUAGAUGCUGGGAAG | 1933 |
| ABE_NGG_20 nt_3-9_002_−_160664336_spCas9 | ACCCUGCUGAGCCAGUGGCA | 1934 |
| CBE_NGG_20 nt_4-9_003_−_160664336_spCas9 | ACCCUGCUGAGCCAGUGGCA | 1935 |
| CBE_NGC_20 nt_4-9_009_−_160664352_spCas9 | UCAAGGUAAUGUUUGAACCC | 1936 |
| ABE_NGC_20 nt_3-9_008_−_160664352_spCas9 | UCAAGGUAAUGUUUGAACCC | 1937 |
| CBE_NGA_20 nt_4-9_006_−_160664491_spCas9 | GAAAGAUGAAGGUCUAGGGG | 1938 |
| ABE_NGA_20 nt_3-9_005_−_160664491_spCas9 | GAAAGAUGAAGGUCUAGGGG | 1939 |
| ABE_NGG_20 nt_3-9_002_−_160664489_spCas9 | AAGAUGAAGGUCUAGGGGUG | 1940 |
| CBE_NGG_20 nt_4-9_003_−_160664489_spCas9 | AAGAUGAAGGUCUAGGGGUG | 1941 |
| ABE_NNNRRT_21 nt_5- | UAAAAUAUUUGAGAGUGCAAU | 1942 |
| 14_014_+_160664388_saCas9 | | |
| CBE_NNNRRT_21 nt_3- | UAAAAUAUUUGAGAGUGCAAU | 1943 |
| 12_015_+_160664388_saCas9 | | |
| ABE_NGG_20 nt_3-9_002_−_160664513_spCas9 | GGGAGGAAACAAGACUAAUC | 1944 |
| CBE_NGG_20 nt_4-9_003_−_160664513_spCas9 | GGGAGGAAACAAGACUAAUC | 1945 |
| ABE_NGG_20 nt_3-9_002_+_160664335_spCas9 | CCCAUGCCACUGGCUCAGCA | 1946 |
| CBE_NGG_20 nt_4-9_003_+_160664335_spCas9 | CCCAUGCCACUGGCUCAGCA | 1947 |
| CBE_NGA_20 nt_4-9_006_+_160664403_spCas9 | UGCAAUGUCAAUAGAUGCUG | 1948 |
| ABE_NGA_20 nt_3-9_005_+_160664403_spCas9 | UGCAAUGUCAAUAGAUGCUG | 1949 |
| CBE_NGA_20 nt_4-9_006_−_160664449_spCas9 | UAAAGGCAAUGUGGAGCAGC | 1950 |
| ABE_NGA_20 nt_3-9_005_−_160664449_spCas9 | UAAAGGCAAUGUGGAGCAGC | 1951 |
| ABE_NNNRRT_21 nt_5- | GUCUUAUAAAAUAUUUGAGAG | 1952 |
| 14_014_+_160664382_saCas9 | | |
| CBE_NNNRRT_21 nt_3- | GUCUUAUAAAAUAUUUGAGAG | 1953 |
| 12_015_+_160664382_saCas9 | | |
| ABE_NGG_20 nt_3-9_002_−_160664341_spCas9 | UUUGAACCCUGCUGAGCCAG | 1954 |
| CBE_NGG_20 nt_4-9_003_−_160664341_spCas9 | UUUGAACCCUGCUGAGCCAG | 1955 |
| CBE_NGC_20 nt_4-9_009_−_160664347_spCas9 | GUAAUGUUUGAACCCUGCUG | 1956 |
| ABE_NGC_20 nt_3-9_008_−_160664347_spCas9 | GUAAUGUUUGAACCCUGCUG | 1957 |
| CBE_NGC_20 nt_4-9_009_−_160664340_spCas9 | UUGAACCCUGCUGAGCCAGU | 1958 |
| ABE_NGC_20 nt_3-9_008_−_160664340_spCas9 | UUGAACCCUGCUGAGCCAGU | 1959 |
| CBE_NGA_20 nt_4-9_006_−_160664326_spCas9 | GCCAGUGGCAUGGGUCUCUG | 1960 |
| ABE_NGA_20 nt_3-9_005_−_160664326_spCas9 | GCCAGUGGCAUGGGUCUCUG | 1961 |
| ABE_NNNRRT_21 nt_5-14_014_−_ | UUUAUAAGACUCUAUAUUCAA | 1962 |
| 160664365_saCas9 | | |
| CBE_NNNRRT_21 nt_3-12_015_−_ | UUUAUAAGACUCUAUAUUCAA | 1963 |
| 160664365_saCas9 | | |
| CBE_NGA_20 nt_4-9_006_−_160664328_spCas9 | GAGCCAGUGGCAUGGGUCUC | 1964 |
| ABE_NGA_20 nt_3-9_005_−_160664328_spCas9 | GAGCCAGUGGCAUGGGUCUC | 1965 |
| ABE_NNNRRT_21 nt_5-14_014_−_ | AUCAUUAACUUAAUUUGACUA | 1966 |
| 160664297_saCas9 | | |
| CBE_NNNRRT_21 nt_3-12_015_−_ | AUCAUUAACUUAAUUUGACUA | 1967 |
| 160664297_saCas9 | | |
| CBE_NGA_20 nt_4-9_006_+_160664495_spCas9 | CCCUAGACCUUCAUCUUUCC | 1968 |
| ABE_NGA_20 nt_3-9_005_+_160664495_spCas9 | CCCUAGACCUUCAUCUUUCC | 1969 |
| ABE_NNNRRT_21 nt_5- | CAAACCAGAUAGUCAAAUUAA | 1970 |
| 14_014_+_160664294_saCas9 | | |
| CBE_NNNRRT_21 nt_3- | CAAACCAGAUAGUCAAAUUAA | 1971 |
| 12_015_+_160664294_saCas9 | | |
| CBE_NGA_20 nt_4-9_006_+_160664395_spCas9 | UUUGAGAGUGCAAUGUCAAU | 1972 |
| ABE_NGA_20 nt_3-9_005_+_160664395_spCas9 | UUUGAGAGUGCAAUGUCAAU | 1973 |
| CBE_NGA_20 nt_4-9_006_−_160664349_spCas9 | AGGUAAUGUUUGAACCCUGC | 1974 |
| ABE_NGA_20 nt_3-9_005_−_160664349_spCas9 | AGGUAAUGUUUGAACCCUGC | 1975 |
| ABE_NGG_20 nt_3-9_002_+_160664401_spCas9 | AGUGCAAUGUCAAUAGAUGC | 1976 |
| CBE_NGG_20 nt_4-9_003_+_160664401_spCas9 | AGUGCAAUGUCAAUAGAUGC | 1977 |
| CBE_NGA_20 nt_4-9_006_+_160664352_spCas9 | GCAGGGUUCAAACAUUACCU | 1978 |
| ABE_NGA_20 nt_3-9_005_+_160664352_spCas9 | GCAGGGUUCAAACAUUACCU | 1979 |
| ABE_NNNRRT_21 nt_5- | ACCAGAUAGUCAAAUUAAGUU | 1980 |
| 14_014_+_160664297_saCas9 | | |
| CBE_NNNRRT_21 nt_3- | ACCAGAUAGUCAAAUUAAGUU | 1981 |
| 12_015_+_160664297_saCas9 | | |
| ABE_NNNRRT_21 nt_5-14_014_−_ | GAGGAAACAAGACUAAUCAGG | 1982 |
| 160664507_saCas9 | | |
| CBE_NNNRRT_21 nt_3-12_015_−_ | GAGGAAACAAGACUAAUCAGG | 1983 |
| 160664507_saCas9 | | |
| ABE_NNNRRT_21 nt_5-14_014_−_ | GGUCUCUGAGAGAAUCAUUAA | 1984 |
| 160664310_saCas9 | | |
| CBE_NNNRRT_21 nt_3-12_015_−_ | GGUCUCUGAGAGAAUCAUUAA | 1985 |
| 160664310_saCas9 | | |
| ABE_NNNRRT_21 nt_5- | CCCACAAACCAGAUAGUCAAA | 1986 |
| 14_014_+_160664290_saCas9 | | |
| CBE_NNNRRT_21 nt_3- | CCCACAAACCAGAUAGUCAAA | 1987 |
| 12_015_+_160664290_saCas9 | | |
| ABE_NNGRRT_21 nt_5- | AUAGAGUCUUAUAAAAUAUUU | 1988 |
| 14_011_+_160664377_saCas9 | | |
| CBE_NNGRRT_21 nt_3- | AUAGAGUCUUAUAAAAUAUUU | 1989 |
| 12_012_+_160664377_saCas9 | | |
| ABE_NNNRRT_21 nt_5- | AUAUUUGAGAGUGCAAUGUCA | 1990 |
| 14_014_+_160664392_saCas9 | | |
| CBE_NNNRRT_21 nt_3- | AUAUUUGAGAGUGCAAUGUCA | 1991 |
| 12_015_+_160664392_saCas9 | | |
| ABE_NNNRRT_21 nt_5- | CAAUGUCAAUAGAUGCUGGGA | 1992 |
| 14_014_+_160664405_saCas9 | | |
| CBE_NNNRRT_21 nt_3- | CAAUGUCAAUAGAUGCUGGGA | 1993 |
| 12_015_+_160664405_saCas9 | | |
| ABE_NNNRRT_21 nt_5- | CACCCCUAGACCUUCAUCUUU | 1994 |
| 14_014_+_160664492_saCas9 | | |
| CBE_NNNRRT_21 nt_3- | CACCCCUAGACCUUCAUCUUU | 1995 |
| 12_015_+_160664492_saCas9 | | |
| ABE_NNNRRT_21 nt_5- | CCUAGACCUUCAUCUUUCCUG | 1996 |
| 14_014_+_160664496_saCas9 | | |
| CBE_NNNRRT_21 nt_3- | CCUAGACCUUCAUCUUUCCUG | 1997 |
| 12_015_+_160664496_saCas9 | | |
| ABE_NNNRRT_21 nt_5- | UACCUUGAAUAUAGAGUCUUA | 1998 |
| 14_014_+_160664367_saCas9 | | |
| CBE_NNNRRT_21 nt_3- | UACCUUGAAUAUAGAGUCUUA | 1999 |
| 12_015_+_160664367_saCas9 | | |
| ABE_NNGRRT_21 nt_5-14_011_−_ | GAACCCUGCUGAGCCAGUGGC | 2000 |
| 160664334_saCas9 | | |
| CBE_NNGRRT_21 nt_3-12_012_−_ | GAACCCUGCUGAGCCAGUGGC | 2001 |
| 160664334_saCas9 | | |
| ABE_NNNRRT_21 nt_5- | GUGCAAUGUCAAUAGAUGCUG | 2002 |
| 14_014_+_160664402_saCas9 | | |
| CBE_NNNRRT_21 nt_3- | GUGCAAUGUCAAUAGAUGCUG | 2003 |
| 12_015_+_160664402_saCas9 | | |
| ABE_NNGRRT_21 nt_5-14_011_−_ | AAUCAGGAAAGAUGAAGGUCU | 2004 |
| 160664493_saCas9 | | |
| CBE_NNGRRT_21 nt_3-12_012_−_ | AAUCAGGAAAGAUGAAGGUCU | 2005 |
| 160664493_saCas9 | | |
| ABE_NNGRRT_21 nt_5-14_011_−_ | CUUAAUUUGACUAUCUGGUUU | 2006 |
| 160664289_saCas9 | | |
| CBE_NNGRRT_21 nt_3-12_012_−_ | CUUAAUUUGACUAUCUGGUUU | 2007 |
| 160664289_saCas9 | | |
| ABE_NNGRRT_21 nt_5- | GUUCAAACAUUACCUUGAAUA | 2008 |
| 14_011_+_160664357_saCas9 | | |
| CBE_NNGRRT_21 nt_3- | GUUCAAACAUUACCUUGAAUA | 2009 |
| 12_012_+_160664357_saCas9 | | |
| ABE_NNGRRT_21 nt_5-14_011_−_ | GCCAGUGGCAUGGGUCUCUGA | 2010 |
| 160664322_saCas9 | | |
| CBE_NNGRRT_21 nt_3-12_012_−_ | GCCAGUGGCAUGGGUCUCUGA | 2011 |
| 160664322_saCas9 | | |
| ABE_NNGRRT_21 nt_5- | AGACCCAUGCCACUGGCUCAG | 2012 |
| 14_011_+_160664332_saCas9 | | |
| CBE_NNGRRT_21 nt_3- | AGACCCAUGCCACUGGCUCAG | 2013 |
| 12_012_+_160664332_saCas9 | | |
| ABE_NNGRRT_21 nt_5- | CAGCAGGGUUCAAACAUUACC | 2014 |
| 14_011_+_160664350_saCas9 | | |
| CBE_NNGRRT_21 nt_3- | CAGCAGGGUUCAAACAUUACC | 2015 |
| 12_012_+_160664350_saCas9 | | |
| ABE_NNNRRT_21 nt_5-14_014_−_ | GCAUCUAUUGACAUUGCACUC | 2016 |
| 160664394_saCas9 | | |
| CBE_NNNRRT_21 nt_3-12_015_−_ | GCAUCUAUUGACAUUGCACUC | 2017 |
| 160664394_saCas9 | | |

TABLE 2
Exemplary base editor amino acid sequences.

| | | Base | | | Editing |
|---|---|---|---|---|---|
| Plasmid Name | mRNA entity | Editor | Deaminase | PAM | window |
| pHRB-424 | 2928, 4071 | ABE | TadA*8.20 | NGA | 3-9 |

MSEVEFSHEYWMRHALTLAKRARDEREVPVGAVLVLNNRVIGEGWNRAIGLHDPTAHAEIMAL
RQGGLVMQNYRLYDATLYSTFEPCVMCAGAMIHSRIGRVVFGVRNAKTGAAGSLMDVLHHPGM
NHRVEITEGILADECAALLCRFFRMPRRVFNAQKKAQSSTDSGGSSGGSSGSETPGTSESATP
ESSGGSSGGSDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVPSE
EVVKKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIARKKD
WDPKKYGGFVSPTVAYSVLVVAKVEKGKSKKLKSVKELLGITIMERSSFEKNPIDFLEAKGYK
EVKKDLIIKLPKYSLFELENGRKRMLASARELQKGNELALPSKYVNFLYLASHYEKLKGSPED
NEQKQLFVEQHKHYLDEIIEQISEFSKRVILADANLDKVLSAYNKHRDKPIREQAENIIHLFT
LTNLGAPAAFKYFDTTIDRKQYRSTKEVLDATLIHQSITGLYETRIDLSQLGGDEGADKRTAD
GSEFESPKKKRKV (SEQ ID NO: 430)

| | | Base | | | Editing |
|---|---|---|---|---|---|
| Plasmid Name | mRNA entity | Editor | Deaminase | PAM | window |
| PCR-001 | 3165 | ABE | TadA*8.20 | NGA | 3-12 |

MKRTADGSEFESPKKKRKVDKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNS
DKLIARKKDWDPKKYGGFVSPTVAYSVLVVAKVEKGKSKKLKSVKELLGITIMERSSFEKNPI
DFLEAKGYKEVKKDLIIKLPKYSLFELENGRKRMLASARELQKGNELALPSKYVNFLYLASHY
EKLKGGSSGSETPGTSESATPESSGSEVEFSHEYWMRHALTLAKRARDEREVPVGAVLVLNNR
VIGEGWNRAIGLHDPTAHAEIMALRQGGLVMQNYRLYDATLYSTFEPCVMCAGAMIHSRIGRV
VFGVRNAKTGAAGSLMDVLHHPGMNHRVEITEGILADECAALLCRFFRMPRRVFNAQKKAQSS
TDSPEDNEQKQLFVEQHKHYLDEIIEQISEFSKRVILADANLDKVLSAYNKHRDKPIREQAEN
IIHLFTLTNLGAPAAFKYFDTTIDRKQYRSTKEVLDATLIHQSITGLYETRIDLSQLGGDEGA
DKRTADGSEFESPKKKRKV (SEQ ID NO: 429)

| | | Base | | | Editing |
|---|---|---|---|---|---|
| Plasmid Name | mRNA entity | Editor | Deaminase | PAM | window |
| PST-109 | 2626 | ABE | TadA*8.20 | NG | 3-9 |

MKRTADGSEFESPKKKRKVSEVEFSHEYWMRHALTLAKRARDEREVPVGAVLVLNNRVIGEGW
NRAIGLHDPTAHAEIMALRQGGLVMQNYRLYDATLYSTFEPCVMCAGAMIHSRIGRVVFGVRN
AKTGAAGSLMDVLHHPGMNHRVEITEGILADECAALLCRFFRMPRRVFNAQKKAQSSTDSGGS
SGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESIRPKRNSDKLIARKKDWDPKKYGGFVSPTVAYSVLVVAKVEKGKSKKLKSVKELLGITIME
RSSFEKNPIDFLEAKGYKEVKKDLIIKLPKYSLFELENGRKRMLASARFLQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPRAFKYFDTTIDRKAYRSTKEVLDATLIHQSITGLYETR
IDLSQLGGDEGADKRTADGSEFESPKKKRKV (SEQ ID NO: 428)

| | | Base | | | Editing |
|---|---|---|---|---|---|
| Plasmid Name | mRNA entity | Editor | Deaminase | PAM | window |
| PCR-004 | 3167 | ABE | TadA*8.20 | NGC | 3-12 |

MKRTADGSEFESPKKKRKVDKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLS
DAILLSDILRVNTEITKAPLSASMVKRYDEHHQDLTLLKALVRQQLPEKYKEIFFDQSKNGYA
GYIDGGASQEEFYKFIKPILEKMDGTEELLVKLNREDLLRKQRTFDNGIIPHQIHLGELHAIL
RRQGDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRLRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGGHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKGNS
DKLIARKKDWDPKKYGGFMQPTVAYSVLVVAKVEKGKSKKLKSVKELLGITIMERSSFEKNPI
DFLEAKGYKEVKKDLIIKLPKYSLFELENGRKRMLASAKFLQKGNELALPSKYVNFLYLASHY
EKLKGGSSGSETPGTSESATPESSGSEVEFSHEYWMRHALTLAKRARDEREVPVGAVLVLNNR
VIGEGWNRAIGLHDPTAHAEIMALRQGGLVMQNYRLYDATLYSTFEPCVMCAGAMIHSRIGRV
VFGVRNAKTGAAGSLMDVLHHPGMNHRVEITEGILADECAALLCRFFRMPRRVFNAQKKAQSS
TDSPEDNEQKQLFVEQHKHYLDEIIEQISEFSKRVILADANLDKVLSAYNKHRDKPIREQAEN
IIHLFTLTNLGAPRAFKYFDTTIARKEYRSTKEVLDATLIHQSITGLYETRIDLSQLGGDEGA
DKRTADGSEFESPKKKRKV (SEQ ID NO: 431)

| | | Base | | | Editing |
|---|---|---|---|---|---|
| Plasmid Name | mRNA entity | Editor | Deaminase | PAM | window |
| PST-107 | 2743, 3626 | ABE | TadA*8.20 | NGC | 3-9 |

MKRTADGSEFESPKKKRKVSEVEFSHEYWMRHALTLAKRARDEREVPVGAVLVLNNRVIGEGW
NRAIGLHDPTAHAEIMALRQGGLVMQNYRLYDATLYSTFEPCVMCAGAMIHSRIGRVVFGVRN
AKTGAAGSLMDVLHHPGMNHRVEITEGILADECAALLCRFFRMPRRVFNAQKKAQSSTDSGGS
SGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMVKRYDEHHQDLTLLKALVRQQLPEKYKEIF
FDQSKNGYAGYIDGGASQEEFYKFIKPILEKMDGTEELLVKLNREDLLRKQRTFDNGIIPHQI
HLGELHAILRRQGDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRLRYTGWGRLSRKL
INGIRDKQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESILPKGNSDKLIARKKDWDPKKYGGFMQPTVAYSVLVVAKVEKGKSKKLKSVKELLGITIME
RSSFEKNPIDFLEAKGYKEVKKDLIIKLPKYSLFELENGRKRMLASAKFLQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPRAFKYFDTTIARKEYRSTKEVLDATLIHQSITGLYETR
IDLSQLGGDEGADKRTADGSEFESPKKKRKV (SEQ ID NO: 432)

| | | Base | | | Editing |
|---|---|---|---|---|---|
| Plasmid Name | mRNA entity | Editor | Deaminase | PAM | window |
| PCR-007-002 | 3169 | ABE | TadA*8.20 | NGG | 3-12 |

MKRTADGSEFESPKKKRKVDKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASHY
EKLKGGSSGSETPGTSESATPESSGSEVEFSHEYWMRHALTLAKRARDEREVPVGAVLVLNNR
VIGEGWNRAIGLHDPTAHAEIMALRQGGLVMQNYRLYDATLYSTFEPCVMCAGAMIHSRIGRV
VFGVRNAKTGAAGSLMDVLHHPGMNHRVEITEGILADECAALLCRFFRMPRRVFNAQKKAQSS
TDSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDEGA
DKRTADGSEFESPKKKRKV (SEQ ID NO: 433)

| | | Base | | | Editing |
|---|---|---|---|---|---|
| Plasmid Name | mRNA entity | Editor | Deaminase | PAM | window |
| pHRB-419 | 2926, 3991, | ABE | TadA*8.20 | NGG | 3-9 |
| | 4247 | | | | |

MSEVEFSHEYWMRHALTLAKRARDEREVPVGAVLVLNNRVIGEGWNRAIGLHDPTAHAEIMAL
RQGGLVMQNYRLYDATLYSTFEPCVMCAGAMIHSRIGRVVFGVRNAKTGAAGSLMDVLHHPGM
NHRVEITEGILADECAALLCRFFRMPRRVFNAQKKAQSSTDSGGSSGGSSGSETPGTSESATP
ESSGGSSGGSDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVPSE
EVVKKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDEGADKRTAD
GSEFESPKKKRKV (SEQ ID NO: 434)

| | | Base | | | Editing |
|---|---|---|---|---|---|
| Plasmid Name | mRNA entity | Editor | Deaminase | PAM | window |
| pHRB-429 | 2889 | ABE | TadA*8.20 | NNGR | 5-14 |
| | | | | RT | |

MSEVEFSHEYWMRHALTLAKRARDEREVPVGAVLVLNNRVIGEGWNRAIGLHDPTAHAEIMAL
RQGGLVMQNYRLYDATLYSTFEPCVMCAGAMIHSRIGRVVFGVRNAKTGAAGSLMDVLHHPGM
NHRVEITEGILADECAALLCRFFRMPRRVFNAQKKAQSSTDSGGSSGGSSGSETPGTSESATP
ESSGGSSGGSKRNYILGLAIGITSVGYGIIDYETRDVIDAGVRLFKEANVENNEGRRSKRGAR
RLKRRRRHRIQRVKKLLFDYNLLTDHSELSGINPYEARVKGLSQKLSEEEFSAALLHLAKRRG
VHNVNEVEEDTGNELSTKEQISRNSKALEEKYVAELQLERLKKDGEVRGSINRFKTSDYVKEA
KQLLKVQKAYHQLDQSFIDTYIDLLETRRTYYEGPGEGSPFGWKDIKEWYEMLMGHCTYFPEE
LRSVKYAYNADLYNALNDLNNLVITRDENEKLEYYEKFQIIENVFKQKKKPTLKQIAKEILVN
EEDIKGYRVTSTGKPEFTNLKVYHDIKDITARKEIIENAELLDQIAKILTIYQSSEDIQEELT
NLNSELTQEEIEQISNLKGYTGTHNLSLKAINLILDELWHTNDNQIAIFNRLKLVPKKVDLSQ
QKEIPTTLVDDFILSPVVKRSFIQSIKVINAIIKKYGLPNDIIIELAREKNSKDAQKMINEMQ
KRNRQTNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISKT
KKEYLLEERDINRFSVQKDFINRNLVDTRYATRGLMNLLRSYFRVNNLDVKVKSINGGFTSFL
RRKWKFKKERNKGYKHHAEDALIIANADFIFKEWKKLDKAKKVMENQMFEEKQAESMPEIETE
QEYKEIFITPHQIKHIKDFKDYKYSHRVDKKPNRELINDTLYSTRKDDKGNTLIVNNLNGLYD
KDNDKLKKLINKSPEKLLMYHHDPQTYQKLKLIMEQYGDEKNPLYKYYEETGNYLTKYSKKDN
GPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDI
TYREYLENMNDKRPPRIIKTIASKTQSIKKYSTDILGNLYEVKSKKHPQIIKKGEGADKRTAD
GSEFESPKKKRKV (SEQ ID NO: 435)

| | | Base | | | Editing |
|---|---|---|---|---|---|
| Plasmid Name | mRNA entity | Editor | Deaminase | PAM | window |
| pHRB-434 | 2892 | ABE | TadA*8.20 | NNNR | 5-14 |
| | | | | RT | |

MSEVEFSHEYWMRHALTLAKRARDEREVPVGAVLVLNNRVIGEGWNRAIGLHDPTAHAEIMAL
RQGGLVMQNYRLYDATLYSTFEPCVMCAGAMIHSRIGRVVFGVRNAKTGAAGSLMDVLHHPGM
NHRVEITEGILADECAALLCRFFRMPRRVFNAQKKAQSSTDSGGSSGGSSGSETPGTSESATP
ESSGGSSGGSKRNYILGLAIGITSVGYGIIDYETRDVIDAGVRLFKEANVENNEGRRSKRGAR
RLKRRRRHRIQRVKKLLFDYNLLTDHSELSGINPYEARVKGLSQKLSEEEFSAALLHLAKRRG
VHNVNEVEEDTGNELSTKEQISRNSKALEEKYVAELQLERLKKDGEVRGSINRFKTSDYVKEA
KQLLKVQKAYHQLDQSFIDTYIDLLETRRTYYEGPGEGSPFGWKDIKEWYEMLMGHCTYFPEE
LRSVKYAYNADLYNALNDLNNLVITRDENEKLEYYEKFQIIENVFKQKKKPTLKQIAKEILVN
EEDIKGYRVTSTGKPEFTNLKVYHDIKDITARKEIIENAELLDQIAKILTIYQSSEDIQEELT
NLNSELTQEEIEQISNLKGYTGTHNLSLKAINLILDELWHTNDNQIAIFNRLKLVPKKVDLSQ
QKEIPTTLVDDFILSPVVKRSFIQSIKVINAIIKKYGLPNDIIIELAREKNSKDAQKMINEMQ
KRNRQTNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISKT
KKEYLLEERDINRFSVQKDFINRNLVDTRYATRGLMNLLRSYFRVNNLDVKVKSINGGFTSFL
RRKWKFKKERNKGYKHHAEDALIIANADFIFKEWKKLDKAKKVMENQMFEEKQAESMPEIETE
QEYKEIFITPHQIKHIKDFKDYKYSHRVDKKPNRKLINDTLYSTRKDDKGNTLIVNNLNGLYD
KDNDKLKKLINKSPEKLLMYHHDPQTYQKLKLIMEQYGDEKNPLYKYYEETGNYLTKYSKKDN
GPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYKNDLIKINGELYRVIGVNNDLLNRIEVNMIDI
TYREYLENMNDKRPPHIIKTIASKTQSIKKYSTDILGNLYEVKSKKHPQIIKKGGSPKKKRKV
SSDYKDHDGDYKDHDIDYKDDDDKEGADKRTADGSEFESPKKKRKV (SEQ ID NO: 436)

| | | Base | | | Editing |
|---|---|---|---|---|---|
| Plasmid Name | mRNA entity | Editor | Deaminase | PAM | window |
| pRW-006 | 4018 | ABE | TadA*8.20 | NRCH | 3-9 |

MSEVEFSHEYWMRHALTLAKRARDEREVPVGAVLVLNNRVIGEGWNRAIGLHDPTAHAEIMAL
RQGGLVMQNYRLYDATLYSTFEPCVMCAGAMIHSRIGRVVFGVRNAKTGAAGSLMDVLHHPGM
NHRVEITEGILADECAALLCRFFRMPRRVFNAQKKAQSSTDSGGSSGGSSGSETPGTSESATP
ESSGGSSGGSDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDIL
RVNTEITKAPLSASMVKRYDEHHQDLTLLKALVRQQLPEKYKEIFFDQSKNGYAGYIDGGASQ
EEFYKFIKPILEKMDGTEELLVKLNREDLLRKQRTFDNGIIPHQIHLGELHAILRRQGDFYPF
LKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVL
TLTLFEDREMIEERLKTYAHLFDDKVMKQLKRLRYTGWGRLSRKLINGIRDKQSGKTILDFLK
SDGFANRNFMQLIHDDSLTFKEDIQKAQVSGQGDSLHEHIANLAGSPAIKKGILQTVKVVDEL
VKVMGGHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVPSE
EVVKKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKGNSDKLIARKKD
WDPKKYGGFNSPTVAYSVLVVAKVEKGKSKKLKSVKELLGITIMERSSFEKNPIDFLEAKGYK
EVKKDLIIKLPKYSLFELENGRKRMLASAGVLQKGNELALPSKYVNFLYLASHYEKLKGSPED
NEQKQLFVEQHKHYLDEIIEQISEFSKRVILADANLDKVLSAYNKHRDKPIREQAENIIHLFT
LTNLGAPAAFKYFDTTINRKQYNTTKEVLDATLIRQSITGLYETRIDLSQLGGDEGADKRTAD
GSEFESPKKKRKV (SEQ ID NO: 437)

| | | Base | | | Editing |
|---|---|---|---|---|---|
| Plasmid Name | mRNA entity | Editor | Deaminase | PAM | window |
| PST-112 | 2869 | CBE | ppAPOBEC1 | NG | 4-9 |

MKRTADGSEFESPKKKRKVTSEKGPSTGDPTLRRRIESWEFDVFYDPRELRKETCLLYEIKWG
MSRKIWRSSGKNTTNHVEVNFIKKFTSERRFHSSISCSITWFLSWSPCWECSQAIREFLSQHP
GVTLVIYVARLFWHMDQRNRQGLRDLVNSGVTIQIMRASEYYHCWRNFVNYPPGDEAHWPQYP
PLWMMLYALELHCIILSLPPCLKISRRWQNHLAFFRLHLQNCHYQTIPPHILLATGLIHPSVT
WRSGGSSGGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQ
TGGFSKESIRPKRNSDKLIARKKDWDPKKYGGFVSPTVAYSVLVVAKVEKGKSKKLKSVKELL
GITIMERSSFEKNPIDFLEAKGYKEVKKDLIIKLPKYSLFELENGRKRMLASARFLQKGNELA
LPSKYVNFLYLASHYEKLKGSPEDNEQKQLFVEQHKHYLDEIIEQISEFSKRVILADANLDKV
LSAYNKHRDKPIREQAENIIHLFTLTNLGAPRAFKYFDTTIDRKAYRSTKEVLDATLIHQSIT
GLYETRIDLSQLGGDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSGGSGGSTNLSDIIE
KETGKQLVIQESILMLPEEVEEVIGNKPESDILVHTAYDESTDENVMLLTSDAPEYKPWALVI
QDSNGENKIKMLSGGSKRTADGSEFESPKKKRKV (SEQ ID NO: 438)

| | | Base | | | Editing |
|---|---|---|---|---|---|
| Plasmid Name | mRNA entity | Editor | Deaminase | PAM | window |
| pHRB-402 | 2744, 4476 | CBE | ppAPOBEC1 | NGA | 4-9 |

MTSEKGPSTGDPTLRRRIESWEFDVFYDPRELRKETCLLYEIKWGMSRKIWRSSGKNTTNHVE
VNFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILSL
PPCLKISRRWQNHLAFFRLHLQNCHYQTIPPHILLATGLIHPSVTWRSGGSSGGSSGSETPGT
SESATPESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHAILRRQ
EDFYPFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKT
ILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPKRNSDKL
IARKKDWDPKKYGGFVSPTVAYSVLVVAKVEKGKSKKLKSVKELLGITIMERSSFEKNPIDFL
EAKGYKEVKKDLIIKLPKYSLFELENGRKRMLASARELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKQYRSTKEVLDATLIHQSITGLYETRIDLSQLGGDSGG
SGGSGGSTNLSDIIEKETGKQLVIQESILMLPEEVEEVIGNKPESDILVHTAYDESTDENVML
LTSDAPEYKPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIKMLSGGSKR
TADGSEFES PKKKRKV (SEQ ID NO: 439)

| | | Base | | | Editing |
|---|---|---|---|---|---|
| Plasmid Name | mRNA entity | Editor | Deaminase | PAM | window |
| pST-110 | 2867, 3628 | CBE | ppAPOBEC1 | NGC | 4-9 |

MKRTADGSEFESPKKKRKVTSEKGPSTGDPTLRRRIESWEFDVFYDPRELRKETCLLYEIKWG
MSRKIWRSSGKNTTNHVEVNFIKKFTSERRFHSSISCSITWFLSWSPCWECSQAIREFLSQHP
GVTLVIYVARLFWHMDQRNRQGLRDLVNSGVTIQIMRASEYYHCWRNFVNYPPGDEAHWPQYP
PLWMMLYALELHCIILSLPPCLKISRRWQNHLAFFRLHLQNCHYQTIPPHILLATGLIHPSVT
WRSGGSSGGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIY ALAHMIKFRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMVKRYDEHHQDLTLLKALVRQQLPE
KYKEIFFDQSKNGYAGYIDGGASQEEFYKFIKPILEKMDGTEELLVKLNREDLLRKQRTFDNG
IIPHQIHLGELHAILRRQGDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRLRYTGWG
RLSRKLINGIRDKQSGKTILDFLKSDGFANRNFMQLIHDDSLTFKEDIQKAQVSGQGDSLHEH
IANLAGSPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQ
TGGFSKESILPKGNSDKLIARKKDWDPKKYGGFMQPTVAYSVLVVAKVEKGKSKKLKSVKELL
GITIMERSSFEKNPIDFLEAKGYKEVKKDLIIKLPKYSLFELENGRKRMLASAKFLQKGNELA
LPSKYVNFLYLASHYEKLKGSPEDNEQKQLFVEQHKHYLDEIIEQISEFSKRVILADANLDKV
LSAYNKHRDKPIREQAENIIHLFTLTNLGAPRAFKYFDTTIARKEYRSTKEVLDATLIHQSIT
GLYETRIDLSQLGGDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSGGSGGSTNLSDIIE
KETGKQLVIQESILMLPEEVEEVIGNKPESDILVHTAYDESTDENVMLLTSDAPEYKPWALVI
QDSNGENKIKMLSGGSKRTADGSEFESPKKKRKV (SEQ ID NO: 440)

| | | Base | | | Editing |
|---|---|---|---|---|---|
| Plasmid Name | mRNA entity | Editor | Deaminase | PAM | window |
| pHRB-397 | 3922, 3984, | CBE | ppAPOBEC1 | NGG | 4-9 |
| | 4246 | | | | |

MTSEKGPSTGDPTLRRRIESWEFDVFYDPRELRKETCLLYEIKWGMSRKIWRSSGKNTTNHVE
VNFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILSL
PPCLKISRRWQNHLAFFRLHLQNCHYQTIPPHILLATGLIHPSVTWRSGGSSGGSSGSETPGT
SESATPESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHAILRRQ
EDFYPFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKT
ILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDSGG
SGGSGGSTNLSDIIEKETGKQLVIQESILMLPEEVEEVIGNKPESDILVHTAYDESTDENVML
LTSDAPEYKPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIKMLSGGSKR
TADGSEFESPKKKRKV (SEQ ID NO: 441)

| | | Base | | | Editing |
|---|---|---|---|---|---|
| Plasmid Name | mRNA entity | Editor | Deaminase | PAM | window |
| pHRB-407 | 2745 | CBE | ppAPOBEC1 | NNGR | 3-12 |
| | | | | RT | |

MTSEKGPSTGDPTLRRRIESWEFDVFYDPRELRKETCLLYEIKWGMSRKIWRSSGKNTTNHVE
VNFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILSL
PPCLKISRRWQNHLAFFRLHLQNCHYQTIPPHILLATGLIHPSVTWRSGGSSGGSSGSETPGT
SESATPESSGGSSGGSGKRNYILGLAIGITSVGYGIIDYETRDVIDAGVRLFKEANVENNEGR
RSKRGARRLKRRRRHRIQRVKKLLFDYNLLTDHSELSGINPYEARVKGLSQKLSEEEFSAALL
HLAKRRGVHNVNEVEEDTGNELSTKEQISRNSKALEEKYVAELQLERLKKDGEVRGSINRFKT
SDYVKEAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPTLKQI
AKEILVNEEDIKGYRVTSTGKPEFTNLKVYHDIKDITARKEIIENAELLDQIAKILTIYQSSE
DIQEELTNLNSELTQEEIEQISNLKGYTGTHNLSLKAINLILDELWHTNDNQIAIFNRLKLVP
KKVDLSQQKEIPTTLVDDFILSPVVKRSFIQSIKVINAIIKKYGLPNDIIIELAREKNSKDAQ
KMINEMQKRNRQTNERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISYETFKKHILNLAKG
KGRISKTKKEYLLEERDINRFSVQKDFINRNLVDTRYATRGLMNLLRSYFRVNNLDVKVKSIN
GGFTSFLRRKWKFKKERNKGYKHHAEDALIIANADFIFKEWKKLDKAKKVMENQMFEEKQAES
MPEIETEQEYKEIFITPHQIKHIKDFKDYKYSHRVDKKPNRELINDTLYSTRKDDKGNTLIVN
NLNGLYDKDNDKLKKLINKSPEKLLMYHHDPQTYQKLKLIMEQYGDEKNPLYKYYEETGNYLT
KYSKKDNGPVIKKIKYYGNKLNAHLDITDDYPNSRNKVVKLSLKPYRFDVYLDNGVYKFVTVK
NLDVIKKENYYEVNSKCYEEAKKLKKISNQAEFIASFYNNDLIKINGELYRVIGVNNDLLNRI
EVNMIDITYREYLENMNDKRPPRIIKTIASKTQSIKKYSTDILGNLYEVKSKKHPQIIKKGGS
PKKKRKVSSDYKDHDGDYKDHDIDYKDDDDKSGGSGGSGGSTNLSDIIEKETGKQLVIQESIL
MLPEEVEEVIGNKPESDILVHTAYDESTDENVMLLTSDAPEYKPWALVIQDSNGENKIKMLSG
GSGGSGGSTNLSDIIEKETGKQLVIQESILMLPEEVEEVIGNKPESDILVHTAYDESTDENVM
LLTSDAPEYKPWALVIQDSNGENKIKMLSGGSKRTADGSEFESPKKKRKV (SEQ ID NO: 442)

| | | Base | | | Editing |
|---|---|---|---|---|---|
| Plasmid Name | mRNA entity | Editor | Deaminase | PAM | window |
| pHRB-412 | 2225 | CBE | ppAPOBEC1 | NNNR | 3-12 |
| | | | | RT | |

MTSEKGPSTGDPTLRRRIESWEFDVFYDPRELRKETCLLYEIKWGMSRKIWRSSGKNTTNHVE
VNFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILSL
PPCLKISRRWQNHLAFFRLHLQNCHYQTIPPHILLATGLIHPSVTWRSGGSSGGSSGSETPGT
SESATPESSGGSSGGSGKRNYILGLAIGITSVGYGIIDYETRDVIDAGVRLFKEANVENNEGR
RSKRGARRLKRRRRHRIQRVKKLLFDYNLLTDHSELSGINPYEARVKGLSQKLSEEEFSAALL
HLAKRRGVHNVNEVEEDTGNELSTKEQISRNSKALEEKYVAELQLERLKKDGEVRGSINRFKT
SDYVKEAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPTLKQI
AKEILVNEEDIKGYRVTSTGKPEFTNLKVYHDIKDITARKEIIENAELLDQIAKILTIYQSSE
DIQEELTNLNSELTQEEIEQISNLKGYTGTHNLSLKAINLILDELWHTNDNQIAIFNRLKLVP
KKVDLSQQKEIPTTLVDDFILSPVVKRSFIQSIKVINAIIKKYGLPNDIIIELAREKNSKDAQ
KMINEMQKRNRQTNERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISYETFKKHILNLAKG
KGRISKTKKEYLLEERDINRFSVQKDFINRNLVDTRYATRGLMNLLRSYFRVNNLDVKVKSIN
GGFTSFLRRKWKFKKERNKGYKHHAEDALIIANADFIFKEWKKLDKAKKVMENQMFEEKQAES
MPEIETEQEYKEIFITPHQIKHIKDFKDYKYSHRVDKKPNRKLINDTLYSTRKDDKGNTLIVN
NLNGLYDKDNDKLKKLINKSPEKLLMYHHDPQTYQKLKLIMEQYGDEKNPLYKYYEETGNYLT
KYSKKDNGPVIKKIKYYGNKLNAHLDITDDYPNSRNKVVKLSLKPYRFDVYLDNGVYKFVTVK
NLDVIKKENYYEVNSKCYEEAKKLKKISNQAEFIASFYKNDLIKINGELYRVIGVNNDLLNRI
EVNMIDITYREYLENMNDKRPPHIIKTIASKTQSIKKYSTDILGNLYEVKSKKHPQIIKKGGS
PKKKRKVSSDYKDHDGDYKDHDIDYKDDDDKSGGSGGSGGSTNLSDIIEKETGKQLVIQESIL
MLPEEVEEVIGNKPESDILVHTAYDESTDENVMLLTSDAPEYKPWALVIQDSNGENKIKMLSG
GSGGSGGSTNLSDIIEKETGKQLVIQESILMLPEEVEEVIGNKPESDILVHTAYDESTDENVM
LLTSDAPEYKPWALVIQDSNGENKIKMLSGGSKRTADGSEFESPKKKRKV (SEQ ID NO: 443)

| | | Base | | | Editing |
|---|---|---|---|---|---|
| Plasmid Name | mRNA entity | Editor | Deaminase | PAM | window |
| pRW-002 | 4014 | CBE | ppAPOBEC1 | NRCH | 3-9 |

MTSEKGPSTGDPTLRRRIESWEFDVFYDPRELRKETCLLYEIKWGMSRKIWRSSGKNTTNHVE
VNFIKKFTSERRFHSSISCSITWFLSWSPCWECSQAIREFLSQHPGVTLVIYVARLFWHMDQR
NRQGLRDLVNSGVTIQIMRASEYYHCWRNFVNYPPGDEAHWPQYPPLWMMLYALELHCIILSL
PPCLKISRRWQNHLAFFRLHLQNCHYQTIPPHILLATGLIHPSVTWRSGGSSGGSSGSETPGT
SESATPESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAI
LLSDILRVNTEITKAPLSASMVKRYDEHHQDLTLLKALVRQQLPEKYKEIFFDQSKNGYAGYI
DGGASQEEFYKFIKPILEKMDGTEELLVKLNREDLLRKQRTFDNGIIPHQIHLGELHAILRRQ
GDFYPFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRLRYTGWGRLSRKLINGIRDKQSGKT
ILDFLKSDGFANRNFMQLIHDDSLTFKEDIQKAQVSGQGDSLHEHIANLAGSPAIKKGILQTV
KVVDELVKVMGGHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPKGNSDKL
IARKKDWDPKKYGGFNSPTVAYSVLVVAKVEKGKSKKLKSVKELLGITIMERSSFEKNPIDFL
EAKGYKEVKKDLIIKLPKYSLFELENGRKRMLASAGVLQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTINRKQYNTTKEVLDATLIRQSITGLYETRIDLSQLGGDSGG
SGGSGGSTNLSDIIEKETGKQLVIQESILMLPEEVEEVIGNKPESDILVHTAYDESTDENVML
LTSDAPEYKPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIKMLSGGSKR
TADGSEFESPKKKRKV (SEQ ID NO: 444)

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2cl (e.g., SEQ ID NO: 232), Cas12c/C2c3, Cas12d/CasY, Cas12e/CasX, Cas12g, Cas12h, Cas12i, and Cas12j/Cas(D, CARF, DinG, Turbo Cas9 (i.e., an SpCas9 with the amino acid alterations Q844R, V842L, F846Y, L847M, and 1852F), homologues thereof, or modified versions thereof A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref NC_018010.1); Psychroflexus torquis (NCBI Ref NC_018721.1); Streptococcus thermophilus (NCBI Ref: YP_820832.1); Listeria innocua (NCBI Ref: NP_472073.1); Campylobacter jejuni (NCBI Ref YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid..

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition “Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H₈₄₀A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H₈₄₀A mutation. In further embodiments, a catalytically dead polynucleotide programmable nucleotide binding domain comprises a point mutation (e.g., D10A or H₈₄₀A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R.T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1 135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some cases, a Cas9 variant has specificity for the PAM 5′-NGC-3′. In some embodiments, a Cas9 variant includes one or more amino acid substitutions selected from D1135M, S1136Q, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1 135M, S1136Y, G1218K, E1219F, A1322R, D1332A, R1335E, and T1337K of spCas9 (SEQ ID No: 197), or a corresponding mutation in another Cas9. In some embodiments, the a Cas9 variant includes one or more amino acid substitutions selected from D1135L, S1136Y, G1218K, E1219F, A1322R, D1332A, R1335E, and T1337R of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Y, G1218K, E1219F, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1 135L, SI 136Q, G1218K, E1219F, E1250K, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1 135M, 51136Y, G1218K, E1219F, E1250K, A1283D, A1322R, D1332A, R1335E, and T1337R of spCas9 (SEQ ID No: 197), or a corresponding mutation in another Cas9. In some embodiments, a Cas9 variant includes one or more amino acid substitutions selected from R765A, Q768A, D1 135L, S1136Y, G1218K, A1283D, E1219F, A1322R, D1332A, R1335E, and T1337K of spCas9 (SEQ ID No: 197), or a corresponding mutation in another Cas9. In some embodiments, any of the Cas9 proteins provided herein, including an SpCas9 comprises any one, two, three, four, five, six, seven, eight, nine, or ten of the following amino acid substitutions in a corresponding residue: R765A, Q768A, W1126R, R1359W, E1250K, A1239T, A1239V, A1283D, R1335D, D1135L, D1135M, D1135R, D1135W, S1136H, S1136Q, S1136Y, G1218D, G1218K, G1218R, G1218E, G1218L, E1219F, E1219K, E1219N, A1322A, A1322R, A1322K, D1332A, R1335V, T1337K, T1337T, D1332A, D1135V and T1337R.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April;38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof For example, a fusion protein can comprise a deaminase flanked by an N-terminal fragment and a C-terminal fragment of a Cas9 or Cas12 (e.g., Cas12b/C₂c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C₂c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in SEQ ID NO: 197. Cas9 polypeptide regions comprising a higher than average B-factor can include, for example, residues 792-872, 792-906, and 2-791 as numbered in SEQ ID NO: 197.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in SEQ ID NO: 197, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in SEQ ID NO: 197, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in SEQ ID NO: 197, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)ₙ (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2cl, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or 5 GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence: ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, 1156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues.

The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant
(TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | Z | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | T | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA
variant (TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T1661, D167N and S82T (TadA*9v2).

In some embodiments, the adenosine deaminase comprises one or more of M1I, MIS, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V401, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, I49G, I49H, I49V, I49F, I49H, 1491, 149M, I49N, I49K, I49Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, 176I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, I95S, I95G, I95L, I95H, I95V, H96A, H96L, H96R, H96S, H96H, H96N, H96E, S97C, S97G, S97I, S97M, S97R, S97S, S97P, R98K, R98I, R98N, R98Q, R98G, R98H, R98C, R98L, R98R, G100R, G100V, G100K, G100A, G100S, G100M, G100I, R101V, R101R, R101S, R101C, V102A, V102F, V1021, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K1101, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P1241, P124L, P124W, G125H, G1251, G125A, G125M, G125K, G125G, G125P, M126D, M126H, M126K, M1261, M126N, M1260, M126S, M126Y, M126M, M126G, N127H, N127S, N127D, N127K, N127R, N127N, N1271, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I132I, I132F, I132T, I132L, I132V, I132E, T133V, T133E, T133G, T133K, T133T, T133A, T133H, T133F, T133I, E134A, E134E, E134G, E134I, E134H, E134K, E134T, G135G, G135V, G135I, G135P, G135E, I136G, I136L, I136T, I136I, I137A, I137D, I137E, L137M, I137S, L137L, L137I, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145S, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D1471, D147C, F148L, F148F, F148R, F148Y, F148A, F148T, F149C, F149M, F149R, F149Y, F149N, F149F, F149A, F149T, F149V, R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K1571, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S1641, S164R, S164Y, S165S, S165P, S165Q, S165A, S165D, S1651, S165T, S165Y, T166T, T166Q, T166E, T166S, T166D, T166K, T166I, T166N, T166P, T166R, D167S D167D, D167I, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In various embodiments, an adenosine deaminase of the disclosure lacks an N-terminal methionine.

In some embodiments, the disclosure provides TadA variants comprising an alteration at an amino acid selected from one or more of L36, 176, V82, Y147, Q154, and N157 comapred to TadA*7.10. In some embodiments, the disclosure provides TadA variants comprising one or more of the following alterations relative to TadA*7.10: L36H, I76Y, V82T, Y147T, Q154S, and N157K. In some embodiments, the disclosure provides TadA variants comprising the following alterations relative to TadA*7.10: L36H, I76Y, V82T, Y147T, Q154S, and N157K. In some embodiments, the disclosure provides TadA variants comprising the following alterations relative to TadA*7.10: F84Y, A109L, A109V, A109I, A109F, A109S, A109T, A109N, V155S, V155T, V155N, F156Y, F156W, F156R, F156N, and F156Q. In some embodiments, the disclosure provides TadA variants comprising the following alterations relative to TadA*7.10: E3N, E3K, E3G, F6A, H14D, L18A, W23I, W23R, P29T, P29Y, P29Q, V35Q, L36S, N38D, G42M, N46Y, P48A, G50A, H52L, A62V, L63R, L63F, Q65R, G67N, L68V, M70I, N72Y, T79H, Y81V, V82S, M94R, G100V, V102E, V102S, R107A, A114C, GI15E, M118L, D119L, H122T, P124H, P124K, P124Q, H128R, V130F, I132K, I132T, E140L, A142N, A142S, L144Q, L145R, L145N, Y147A, F149A, R152P, F156N, and K160E.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, aY147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table SE. Table SE shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

TABLE 5F
TadA Variants

| Description | | | | | | | | | | |
|---|---|---|---|---|---|---|---|---|---|---|
| Amino Acid No. | 23 | 36 | 48 | 51 | 76 | 82 | 84 | 106 | 108 | 123 |
| TadA (wt) | W | H | P | R | I | V | L | A | D | H |
| TadA*7.10 | R | L | A | L | I | V | F | V | N | Y |
| TadA*8.8 | | | | | | | | | | H |
| TadA*8.13 | | | | | Y | | | | | H |
| TadA*8.17 | | | | | | S | | | | |
| TadA*8.20 | | | | | Y | S | | | | H |
| TadA*8.8 + V82T | | | | | | T | | | | |
| TadA*8.8 + | | | | | | T | | | | H |
| V82T + Y147T + Q154S | | | | | | | | | | |
| TadA*8.17 + V82T | | | | | | T | | | | |
| TadA*8.17 + | | | | | | T | | | | |
| V82T + Y147T + Q154S | | | | | | | | | | |
| TadA*8.20 + V82T | | | | | Y | T | | | | H |
| TadA*8.20 + | | | | | Y | T | | | | H |
| V82T + Y147T + Q154S | | | | | | | | | | |

| Official Name | | | | | | | | |
|---|---|---|---|---|---|---|---|---|
| Amino Acid No. | 146 | 147 | 152 | 154 | 155 | 156 | 157 | 166 |
| TadA (wt) | S | D | R | Q | E | I | K | T |
| TadA*7.10 | C | Y | P | Q | V | F | N | T |
| TadA (wt) | S | D | R | Q | E | I | K | T |
| TadA*8.8 | | R | | R | | | | |
| TadA*8.13 | | R | | R | | | | |
| TadA*8.17 | | | | R | | | | |
| TadA*8.20 | | R | | R | | | | |
| TadA*8.8 + V82T | | R | | R | | | | |
| TadA*8.8 + | | T | | S | | | | |
| V82T + Y147T + Q154S | | | | | | | | |
| TadA*8.17 + V82T | | | | R | | | | |
| TadA*8.17 + | | T | | S | | | | |
| V82T + Y147T + Q154S | | | | | | | | |
| TadA*8.20 + V82T | | R | | R | | | | |
| TadA*8.20 + | | T | | S | | | | |
| V82T + Y147T + Q154S | | | | | | | | |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

C to T Editing In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of R33A, K34A, E63A, H102P, D104N, H121R, H122R, H122L, D124N; R126A, R126E, RT18A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase. In some embodiments, an APOBEC deaminase incorporated into a base editor can comprise one or more combinations of mutations selected from K34A, H122L, and D124N (AALN); H102P and D104N (evoFERNY derived from FERNY); W90Y and R126E (YE1); W90Y and R132E (YE2); R126E and R132E (EE); W90Y, RI26E, and RI32E (YEE), or rAPOBEC1; and any alternative mutation at the corresponding positions, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference. Further non-limiting examples of C to T nucleobase editing proteins are described in PCT Applications No. PCT/US2020/062428 and PCT/US2019/033848, the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains or TadA-derived cytidine deaminases (TadA-CD).

Base editors containing adenosine deaminase variants having both cytidine deaminase and adenosine deaminase activity (i.e., TadA-Dual deaminases) may be referred to as TadA-based dual editors (TadDE). In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell. Examples of adenosine deaminase variants having increased cytidine deaminase activity include those described in International Patent Application Publications No. WO 2024/040083 and WO 2022/204574, the disclosures of which are hereby incorporated by reference in their entireties for all purposes.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V301, R47G, R47S, A48G, 149K, 149M, 149N, 149Q, 149T, G67W, 176H, 176R, 176W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+149K; 1.17+E27G; 1.17+149N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20.

| | location in structure | | | | | | | | | | | |
|---|---|---|---|---|---|---|---|---|---|---|---|---|
| | N/A | Sh1 | Sh1 | Sh1 | NAS | NAS | NAS | NAS | | S | | |
| | Amino Acid No. (*START Met is AA#1) | | | | | | | | | | | |
| | 2 | 8 | 13 | 17 | 27 | 47 | 48 | 49 | 67 | 76 | 77 | |
| TadA*8.20 | S | | R | T | E | R | A | I | G | Y | D | |
| TadA*8.19 | | | | | | | | | | I | | |
| 1.1 | | | | | H | | | | | I | | |
| 1.2 | | | | | H | | | K | | I | | |
| 1.3 | | | | | S | | | K | | I | | |
| 1.4 | | | | | S | | | K | | I | | |
| 1.5 | | | | | K | | | | | | | |
| 1.6 | | | | | K | | | | | | | |
| 1.7 | | | | | H | | | | | I | | |
| 1.8 | | | | | S | | | K | M | | | |
| 1.9 | | | | | | | | T | W | | | |
| 1.10 | | | | | C | | | | | I | | |
| 1.11 | | | G | | Q | | | | | | | |
| 1.12 | | | | A | H | | | M | | I | | |
| 1.13 | | | | | | | | Q | | I | | |
| 1.14 | H | | | | | | | K | | I | | |
| 1.15 | | | | | | S | | | | | | |
| 1.16 | | Q | | | | | | Q | | I | | |
| 1.17 | | | | A | | | G | | | | | |
| 1.18 | | | | | G | | | | | | | |
| 1.19 | | | | | G | | | N | | | | |
| 1.20 | | | | | G | | | | | | G | |
| | location in structure | | | | | | | | | | | |
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

“I” indicates “Internal,”
“S” indicates “Surface,” and
“NAS” indicates “Near Active Site.”

TABLE 6B
Adenosine deaminase variants. Mutations are
indicated with reference to TadA*8.20.

| Position No. | 27 | 29 | 30 | 49 | 82 | 84 | 107 | 112 | 115 | 142 |
|---|---|---|---|---|---|---|---|---|---|---|
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated
with reference to variant 1.2 (Table 6A) .

| | Alternative | Residue identity (START Met is amino | | | | | | | | |
|---|---|---|---|---|---|---|---|---|---|---|
| Variant | Variant | acid #1) | | | | | | | | |
| Name | Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | Alternative | Residue identity (START Met is | | | | | | | | |
| Variant | Variant | amino acid #1) | | | | | | | | |
| Name | Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to
TadA*8.20.

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | M | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | M | G | T | C | H | C | M | N | | P | E | |
| S2.52 | Y | H | K | W | G | T | C | H | C | M | | G | P | E | |
| S2.53 | Y | H | K | M | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

A TadA-derived cytidine deaminase (e.g., TadA-CD), according to certain embodiments, comprises an amino acid sequence that is at least 80% identical, at. least 85% identical, at least 90% identical, at least 95% identical, at least 98% identical, at least 99% identical, and at least 99.5% identical to the amino acid sequence of SEQ ID NO: 489, wherein residue 27 of SEQ ID NO: 489 is any amino acid expect for E (glutamic acid). TadA-CDs with other sequence homologies are also possible. For example, in certain embodiments, the TadA-derived cytidine deaminase (e.g., TadA-CD) comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 489, wherein residue 28 of SEQ ID NO: 489 is any amino acid expect for V (valine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 489, wherein residue 96 of SEQ ID NO: 489 is any amino acid expect for H (histidine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 489, wherein residue 26 of SEQ ID NO: 489 is any amino acid expect for R (arginine). In various embodiments, the TadA-derived cytidine deaminase comprises an alteration at one or more of positions 26, 27, 28, 48, 73, or 96 compared to SEQ ID NO: 489.

As will be appreciated by those of skill in the art, TadA-derived cytidine deaminases (e.g., TadA-CD) may comprise a plurality of mutations relative to the parent adenosine deaminase (e.g., TadA-8e). In some embodiments, the deaminase of the instant application (e.g., TadA-CD) comprises mutations at residues E27, V28, and H96. In some embodiments, the disclosed deaminase further comprises at least one mutation at a residue selected from R26, M61, Y73, I76, M151, Q154, and A158, in the amino acid sequence of SEQ ID NO: 489, or corresponding mutations in a homologous adenosine deaminase.

In some embodiments, the deaminase comprises at least one mutation selected from E27A, E27K, V28G, V28A, and 1-196N, and further comprises at least one mutation at a residue selected from R26G, M611, Y731H, Y73S, Y73C, 176F, M151I, Q154R., Q154H, and A158S, in the amino acid sequence of SEQ ID NO: 489, or a corresponding mutation in a homologous adenosine deaminase. Other mutations are also possible. For example, in certain embodiments, the TadA-CD enzyme comprises mutations selected from E27A, V28C, and H96N, and further comprises at least one mutation selected from R26G, M61I, Y73H, Y73S, Y73C, 176F, M151I, Q154R, Q1541-1, and Al 58S, in the amino acid sequence of SEQ ID NO: 489, or corresponding mutations in a homologous adenosine deaminase.

Other exemplary embodiments may include (1) deaminases comprising mutations E27K, V28G, and H96N, and further comprising at least one mutation selected from R26G, M61I, Y73H, Y73S, Y73C, 176F, M151I, Q154R, Q1 54H, and A158S, in the amino acid sequence of SEQ ID NO: 489 or corresponding mutations in a homologous adenosine deaminase; (2) deaminases comprising mutations E27A, V28A, and H96N, and further comprising at least one mutation selected from R26G3, M611, Y73H, Y73S, Y73C, I76F, M151I, Q154R, Q154H, and A158S, in the amino acid sequence of SEQ ID NO: 489, or corresponding mutations in a homologous adenosine deaminase; (3) deaminases comprising mutations E27K, V28A., and H96N, and further comprising at least one mutation selected from R26G, M61I, Y73H, Y73S, Y73C, 176F, M1511, Q154R, Q1541-1, and A158S, in the amino acid sequence of SEQ ID NO: 489, or corresponding mutations in a homologous adenosine deaminase.

In some embodiments, the TadA-derived cytidine deaminases (TadA-CD) comprise at least two mutations at residues selected from R26, M61, Y73, 176, M151, Q154, and A158 (relative to a reference adenosine deaminase). In other embodiments, the TadA-CD comprises at least two mutations at residues selected from R26G, M61I, Y73H, 176F, M151I, Q154H, Q154R, and A158S.

In some embodiments, the addition of a V106W mutation improves the selectivity by suppressing A deamination to a greater extent than C deamination.

In some embodiments, a TadA-based dual editor comprises an adenosine deaminase variant comprising one, two, three, four, or five mutations selected from R26G, V28A, A48R, Y73S, and H96N (e.g., SEQ ID NO: 495).

As such, in some embodiments, provided herein are deaminases that comprise mutations at residues R26, V28, A48, and. Y73 in the amino acid sequence of SEQ ID NO: 489, or corresponding mutations in a homologous adenosine deaminase. Further provided herein are deaminases that comprise mutations at residues R26, E27, V28, A48, and Y73 (e.g., further comprise a mutation at E27) in the amino acid sequence of SEQ ID NO: 489. In particular embodiments, these deaminases comprise the mutations R26G, V28A, A48R, Y73S, and H96N. In some embodiments, these deaminases comprise the mutations R26G, V28GE, A48R, and Y73C.

TadA-CD variants may comprise at least one mutation selected from R26G, E27A, V28G, 176F, H96N, and M1511 (e.g., TadA-CDa, SEQ ID NO: 490); R26G, E27A, V28G, 176F, H96N, and A158S (e.g., TadA-CDb, SEQ ID NO: 491); R26G, E27A, V28G, I76F, H96N, Q154R, and A158S (e.g., TadA-CDc, SEQ ID NO: 492); E27A, V28G, Y731-H, -196N, Q154H, and A158S (e.g., TadA-CDd, SEQ ID NO: 493); R26G, V28A, A48R, Y73S, and 1H96N (e.g., TadA-CDe, SEQ ID NO. 494); V28A, A48R, and Y73S (e.g., TadA-CDf, SEQ ID NO: 495), and R26G, V28G, A48R, and Y73C (e.g., TadA-CDg, SEQ ID NO: 496).

In some preferred embodiments, the deaminase comprises the mutations R26G, E27A, V28G, 176F, H96N, and A158S (e.g., TadA-CDa., SEQ ID NO: 490), R26G, E27A, V28G, 176F, H96N, Q154R, and A158S (e.g., TadA-CDb, SEQ ID NO: 491), R26G, E27A, V28G, 176F, I-196N, and M151I (e.g.. TadA-CDc. SEQ ID NO: 492), E27K, V28A, M611, and I96N (e.g., TadA-CDd, SEQ ID NO: 493), E27A. V28G, Y73H, H96N, Q154H, and A158S (e.g., TadA-CDe, SEQ ID NO 494), R26G, V28A, A48R., Y73S, and H96N (e.g., TadA-CDf, SEQ ID NO: 495), and R26G, V28G, A48R, and Y73C (e.g., TadA-CDg, SEQ ID NO: 496).

In some embodiments, the TadA-CD variants described above and herein may also comprises a V106W mutation.

In some embodiments, the TadA-CD variants comprise at least 80%, at least 85%, at least 90%, at least 95%, at least 98%, at least 99%, or at least 99.5% to any of the amino acid sequences of SEQ ID NOs: 489-496.

In some embodiments, the evolved. TadA-Dual deaminase comprises the mutations R26G, V28A, N46I, A48R, Y73P, and H96N (TadA-CD-1, SEQ ID NO: 497) relative to the amino acid sequence of SEQ ID NO: 489. In some embodiments, the evolved TadA-Dual deaminase comprises the mutations R26G, V28A. N46T, A48R, Y73P, and H96N (TadA-CD-2, SEQ ID NO: 498) relative to the amino acid sequence of SEQ ID NO: 489. In some embodiments, the evolved TadA-Dual deaminase comprises the mutations R-26G, V28A, N46T, A48R, Y73S, and H96N (TadA-CD-3, SEQ ID NO: 499) relative to the amino acid sequence of SEQ ID NO. 189. In some embodiments, the evolved TadA-Dual deaminase comprises the mutations R26G, V28A, N46V, A48R, Y73S, and H96N (TadA-CD-4, SEQ ID NO:500) relative to the amino acid sequence of SEQ ID NO: 489. In some embodiments, the evolved TadA-Dual deaminase comprises the mutations R26G, V28A, N46V, A48R, Y73P, and H96N (TadA-CD-5, SEQ ID NO: 501) relative to the amino acid sequence of SEQ ID NO: 489. In some embodiments, the evolved TadA-Dual deaminase comprises the mutations R26G, V28A. N46L. A48R Y73P, and H96N (TadA-CD-6, SEQ ID NO: 502) relative to the amino acid sequence of SEQ ID NO: 489. In some embodiments, the evolved TadA-Dual deaminase comprises the mutations V28A, N46L, A48P, and Y73P (TadA-CD-7, SEQ ID NO: 503) relative to the amino acid sequence of SEQ ID NO: 489. In some embodiments, the evolved TadA-Dual deaminase comprises the mutations V28A, N46C, A48P, and Y73P (TadA-CD-8, SEQ ID NO: 504) relative to the amino acid sequence of SEQ ID NO: 489. In some embodiments, the evolved TadA-Dual deaminase comprises the mutations R26G, V28A, N46V, A48R, Y73P, and H96N (TadA-CD-9, SEQ ID NO: 505) relative to the amino acid sequence of SEQ ID NO: 489. In some embodiments, the evolved TadA-Dual deaminase comprises the mutations R26G, V28A, N46V. A48R. Q71H, Y73P, and H96N (TadA-CD-10, SEQ ID NO: 506) relative to the amino acid sequence of SEQ ID NO: 489. In some embodiments, the evolved TadA-Dual deaminase comprises the mutations R26G, V28A, N46L, A48R, Y73P, and H96N (TadA-CD-11, SEQ ID NO: 507) relative to the amino acid sequence of SEQ ID NO: 489. In some embodiments, the evolved TadA-Dual deaminase comprises the mutations R26G, V28A. N46C, A48R, Y73P, and H96N (TadA-CD-12, SEQ ID NO: 508) relative to the amino acid sequence of SEQ ID NO: 489. In some embodiments, the evolved TadA-Dual deaminase comprises the mutations R26G, V28A, N46C, A48R, Y73P. H96N, and A162V (TadA-CD-13, SEQ ID NO: 509) relative to the amino acid sequence of SEQ ID NO: 489.

In some embodiments, the evolved TadA-Dual deaminase comprises the mutations R26G, V28A, N46I, A48R, Y73S, and H96N (TadA-CD-14, SEQ ID NO: 510) relative to the amino acid sequence of SEQ ID NO: 489. In some embodiments, the evolved TadA-Dual deaminase comprises the mutations R26G, V28A, A48R, Q71S, Y73S, and H96N (TadA-CD-15, SEQ ID NO: 511) relative to the amino acid sequence of SEQ ID NO: 489. In some embodiments, the evolved TadA-Dual deaminase comprises the mutations R26G, V28A, N46L, A48R, and Y73P (TadA-CD-16, SEQ ID NO: 512) relative to the amino acid sequence of SEQ ID NO: 489. In some embodiments, the evolved TadA-Dual deaminase comprises the mutations R26G, V28A, N46L, A48R, Y73P, and H96N (TadA-CD-17, SEQ ID NO: 513) relative to the amino acid sequence of SEQ ID NO: 489. In some embodiments, the evolved TadA-Dual deaminase comprises the mutations R26G, V28A, Y73P, and H96N (TadA-CD-18, SEQ ID NO: 514) relative to the amino acid sequence of SEQ ID NO: 489. In some embodiments, the evolved TadA-Dual deaminase comprises the mutations R26G. V28A, N46V, A48R, Y73S, and H96N (TadA-CD-19, SEQ ID NO: 515) relative to the amino acid sequence of SEQ ID NO: 489. In some embodiments, the evolved TadA-Dual deaminase comprises the mutations R26G, V28A, N46V, A48R, Y73P. and H96N (TadA-CD-20. SEQ ID NO: 516) relative to the amino acid sequence of SEQ ID NO: 489. In some embodiments, the evolved TadA-Dual deaminase comprises the mutations R26G and N46L (TadA-CD-21. SEQ ID NO: 517) relative to the amino acid sequence of SEQ ID NO: 489. In some embodiments, the evolved TadA-Dual deaminase comprises the mutations R26G, V28A, N46I, A48R, Y73P, and H96N (TadA-CD-22, SEQ ID NO: 518) relative to the amino acid sequence of SEQ ID NO: 489. In some embodiments, the 10 evolved TadA-Dual deaminase comprises the mutations R26G, V28A, N46V. A48R, Y73P, and H96N (TadA-CD-23, SEQ ID NO: 519) relative to the amino acid sequence of SEQ ID NO: 489. In some embodiments, the evolved TadA-Dual deaminase comprises the mutations R26G, V28A, A48P, Y73H, T79P, and H96N (TadA-CD-24, SEQ ID NO: 520) relative to the amino acid sequence of SEQ ID NO: 489. In some embodiments, the evolved TadA-Dual deaminase comprises the mutations R26G, N46I, and H96N (TadA-CD-25, SEQ ID NO: 521) relative to the amino acid sequence of SEQ ID NO: 489.

In some embodiments, the evolved TadA-Dual deaminase comprises the mutations R26G, V28A, N46V. A48R. Y73P, and H % N (TadA-CD-26, SEQ ID NO: 522) relative to the amino acid sequence of SEQ ID NO: 489. In some embodiments, the evolved TadA-Dual deaminase comprises the mutations R26G, V28A, N46L, A48R, Y73S, and H96N (TadA-CD-27, SEQ ID NO: 523) relative to the amino acid sequence of SEQ ID NO: 489. In some embodiments, the evolved TadA-Dual deaminase comprises the mutations R26G, V28A, N46C, A48R, H96N, and A162V (TadA-CD-28, SEQ ID NO: 524) relative to the amino acid sequence of SEQ ID NO: 489. In some embodiments, the evolved TadA-Dual deaminase comprises the mutations R26G. V28A, N46V, A48R, Q71H, Y73P, and H96N (TadA-CD-29, SEQ ID NO: 525) relative to the amino acid sequence of SEQ ID NO: 489. In some embodiments, the evolved TadA-Dual deaminase comprises the mutations R26G, V28A, N46C, A48R, Y73P, and H96N (TadA-CD-30. SEQ ID NO: 526) relative to the amino acid sequence of SEQ ID NO: 489. In some embodiments, the evolved TadA-Dual deaminase comprises the mutations R26G, V28A, N46C, A48R, Y73P, H96N, and A162V (TadA-CD-31, SEQ ID NO: 527) relative to the amino acid sequence of SEQ ID NO: 489.

In some embodiments, the evolved TadA-Dual deaminase comprises the mutations R26G, V28A, N46V. A48R. Y73P, and H % N (TadA-CD-32, SEQ ID NO: 528) relative to the amino acid sequence of SEQ ID NO: 489. In some embodiments, the evolved TadA-Dual deaminase comprises the mutations R26G, V28A. N46V, A48R, Y73S, and H96N (TadA-CD-33, SEQ ID NO: 529) relative to the amino acid sequence of SEQ ID NO: 489. In some embodiments, the evolved TadA-Dual deaminase comprises the mutations R26G, V28A. N46V, A48P, Y73S. and H96N (TadA-CD-34. SEQ ID NO: 530) relative to the amino acid sequence of SEQ ID NO: 489. In some embodiments, the evolved TadA-Dual deaminase comprises the mutations R26G, V28A, N46C, A48R, Y73P, and H96N (TadA-CD-35, SEQ ID NO: 531) relative to the amino acid sequence of SEQ ID NO: 489. In some embodiments, the evolved TadA-Dual deaminase comprises the mutations R26G, V28A, L34M, N46L, A48R, Y73P, and H96N (TadA-CD-36, SEQ ID NO: 532) relative to the amino acid sequence of SEQ ID NO: 489. In some embodiments, the evolved TadA-Dual deaminase comprises the mutations R26G. V28A, N46L, A48R, Y73P, and H96N (TadA-CD-37, SEQ ID NO: 533) relative to the amino acid sequence of SEQ ID NO: 489. In some embodiments, the evolved TadA-Dual deaminase comprises the mutations R26G, V28A, N46L, A48P, R64K, Y73P, and H96N (TadA-CD-38, SEQ ID NO: 534) relative to the amino acid sequence of SEQ ID NO; 489.

In some embodiments, the evolved TadA-Dual deaminase comprises the mutations N46I, S73P, and H154Q (TadA-CD-1, SEQ ID NO: 497) relative to the amino acid sequence of SEQ ID NO: 495. In some embodiments, the evolved TadA-Dual deaminase comprises the mutations N46T (TadA-CD-2, SEQ ID NO: 498) relative to the amino acid sequence of SEQ ID NO: 495. In some embodiments, the evolved TadA-Dual deaminase comprises the mutations N46T and H154Q (TadA-CD-3, SEQ ID NO: 499) relative to the amino acid sequence of SEQ ID NO: 495. In some embodiments, the evolved TadA-Dual deaminase comprises the mutations N46V and H154Q (TadA-CD-4, SEQ ID NO: 500) relative to the amino acid sequence of SEQ ID NO: 495. In some embodiments, the evolved TadA-Dual deaminase comprises the mutations N46V, S73P, G105S, and H154Q (TadA-CD-5, SEQ ID NO: 501) relative to the amino acid sequence of SEQ ID NO: 495. In some embodiments, the evolved TadA-Dual deaminase comprises the mutations N46L, S73P, and H154Q (TadA-CD-6, SEQ ID NO: 502) relative to the amino acid sequence of SEQ ID NO: 495. In some embodiments, the evolved TadA-Dual deaminase comprises the mutations G26R N46L, R48P, S73P, N96H, and H154Q (TadA-CD-7, SEQ ID NO: 503) relative to the amino acid sequence of SEQ ID NO: 495. In some embodiments, the evolved TadA-Dual deaminase comprises the mutations N46C, N % H, and H154Q (TadA-CD-8, SEQ ID NO: 504) relative to the amino acid sequence of SEQ ID NO: 495. In some embodiments, the evolved TadA-Dual deaminase comprises the mutations N46V, S73P, and H154Q (TadA-CD-9, SEQ ID NO: 505) relative to the amino acid sequence of SEQ ID NO: 495. In some embodiments, the evolved TadA-Dual deaminase comprises the mutations N46V, Q71H, S73P, and H154Q (TadA-CD-10, SEQ ID NO: 506) relative to the amino acid sequence of SEQ ID NO: 495. In some embodiments, the evolved TadA-Dual deaminase comprises the mutations N46L and H154Q (TadA-CD-11. SEQ ID NO: 507) relative to the amino acid sequence of SEQ ID NO: 495. In some embodiments, the evolved TadA-Dual deaminase comprises the mutations N46C, S73P, and H154Q (TadA-CD-12, SEQ ID NO: 508) relative to the amino acid sequence of SEQ ID NO: 495. In some embodiments, the evolved TadA-Dual deaminase comprises the mutations N46C, S73P. H154Q, and A162V (TadA-CD-13, SEQ ID NO: 509) relative to the amino acid sequence of SEQ ID NO: 495.

In some embodiments, the evolved TadA-Dual deaminase comprises the mutations N46I and H154Q (TadA-CD-14, SEQ ID NO: 510) relative to the amino acid sequence of SEQ ID NO: 495. In some embodiments, the evolved TadA-Dual deaminase comprises the mutations Q7IS and H154Q (TadA-CD-15, SEQ ID NO: 511) relative to the amino acid sequence of SEQ ID NO: 489. In some embodiments, the evolved TadA-Dual deaminase comprises the mutations N46L, S73P, N79T, and N96H (TadA-CD-16, SEQ ID NO; 512) relative to the amino acid sequence of SEQ ID NO: 495. In some embodiments, the evolved TadA-Dual deaminase comprises the mutations N46L, S73P, N79T (TadA-CD-17. SEQ ID NO: 513) relative to the amino acid sequence of SEQ ID NO: 495. In some embodiments, the evolved TadA-Dual deaminase comprises the mutations R48A, S73P, and N79T (TadA-CD-18. SEQ ID NO: 514) relative to the amino acid sequence of SEQ ID NO: 495. In some embodiments, the evolved TadA-Dual deaminase comprises the mutations N46V and N79T (TadA-CD-19. SEQ ID NO: 515) relative to the amino acid sequence of SEQ ID NO: 495. In some embodiments, the evolved TadA-Dual deaminase comprises the mutations N46V, S73P, and N79T (TadA-CD-20, SEQ ID NO: 516) relative to the amino acid sequence of SEQ ID NO: 495. In some embodiments, the evolved TadA-Dual deaminase comprises the mutations A28V, N46L, R48A, S73Y, N79T, and N96H (TadA-CD-21, SEQ ID NO: 517) relative to the amino acid sequence of SEQ ID NO: 495. In some embodiments, the evolved TadA-Dual deaminase comprises the mutations N46I, S73P, and N79T (TadA-CD-22, SEQ ID NO: 518) relative to the amino acid sequence of SEQ ID NO: 495. In some embodiments, the evolved TadA-Dual deaminase comprises the mutations N46V, S73P, N79T, and G106S (TadA-CD-23, SEQ ID NO: 519) relative to the amino acid sequence of SEQ ID NO: 495. In some embodiments, the evolved TadA-Dual deaminase comprises the mutations R48P, S73H, and N79P (TadA-CD-24, SEQ ID NO: 520) relative to the amino acid sequence of SEQ ID NO: 495. In some embodiments, the evolved TadA-Dual deaminase comprises the mutations A28V, N46I, R48A, S73Y, and N79T (TadA-CD-25. SEQ ID NO: 521) relative to the amino acid sequence of SEQ ID NO: 495.

In some embodiments, the evolved TadA-Dual deaminase comprises the mutations N46V and S73P (TadA-CD-26, SEQ ID NO: 522) relative to the amino acid sequence of SEQ ID NO: 495. In some embodiments, the evolved TadA-Dual deaminase comprises the mutation N46L (TadA-CD-27, SEQ ID NO: 523) relative to the amino acid sequence of SEQ ID NO: 495. In some embodiments, the evolved TadA-Dual deaminase comprises the mutations N46C, S73Y, and A162V (TadA-CD-28, SEQ ID NO: 524) relative to the amino acid sequence of SEQ ID NO: 495. In some embodiments, the evolved TadA-Dual deaminase comprises the mutations N46V, Q71H, and S73P (TadA-CD-29, SEQ ID NO; 525) relative to the amino acid sequence of SEQ ID NO: 495. In some embodiments, the evolved TadA-Dual deaminase comprises the mutations N46C and S73P (TadA-CD-30, SEQ ID NO: 526) relative to the amino acid sequence of SEQ ID NO: 495. In some embodiments, the evolved TadA-Dual deaminase comprises the mutations N46C, S73P, and A162V (TadA-CD-31, SEQ ID NO: 527) relative to the amino acid sequence of SEQ ID NO: 495. In some embodiments, the evolved TadA-Dual deaminase comprises the mutations N46V and S73P (TadA-CD-32, SEQ ID NO: 528) relative to the amino acid sequence of SEQ ID NO: 495. In some embodiments, the evolved TadA-Dual deaminase comprises the mutation N46V (TadA-CD-33, SEQ ID NO: 529) relative to the amino acid sequence of SEQ ID NO: 495. In some embodiments, the evolved TadA-Dual deaminase comprises the mutations N46V and R48P(TadA-CD-34, SEQ ID NO: 530) relative to the amino acid sequence of SEQ ID NO: 495. In some embodiments, the evolved TadA-Dual deaminase comprises the mutations N46CV and S73P (TadA-CD-35, SEQ ID NO: 531) relative to the amino acid sequence of SEQ ID NO: 495. In some embodiments, the evolved TadA-Dual deaminase comprises the mutations L34M, N46L and S73P (TadA-CD-36, SEQ ID NO: 532) relative to the amino acid sequence of SEQ ID NO: 495. In some embodiments, the evolved TadA-Dual deaminase comprises the mutations N46L and S73P (TadA-CD-37, SEQ ID NO: 533) relative to the amino acid sequence of SEQ ID NO: 495. In some embodiments, the evolved TadA-Dual deaminase comprises the mutations N46L, r48P, R64K and S73P (TadA-CD-38, SEQ ID NO: 534) relative to the amino acid sequence of SEQ ID NO: 495.

In some embodiments, the TadA-CDs evolved from TadA-dual comprise at least 80%, 85%, 90%, 95%. 98%, 99%, or 99.5% identical to any of the amino acid sequences of SEQ ID NOs: 39, 41-54, and 359-383.

Exemplary TadA-derived cytosine base editor amino acid sequences include: TadA-CDa base editor (SpCas9n napDNAbp domain) (TadCBEa) (SEQ ID NO: 535), TadA-CDb base editor (SpCas9n napDNAbp domain) (TadCBEb) (SEQ ID NO: 536), TadA-CDc base editor (SpCas9n napDNAbp domain) (TadCBEc) (SEQ ID NO: 537), TadA-CDd base editor (SpCas9n napDNAbp domain) (TadCBEd) (SEQ ID NO: 538), TadA-CDe base editor (SpCas9n napDNAbp domain) (TadCBEe) (SEQ ID NO: 539), TadA-CDa(V106W) base editor (SpCas9n napDNAbp domain) (TadCBEa(V106W)) (SEQ ID NO: 540), TadA-CDd(V106W) base editor (SpCas9n napDNAbp domain) (TadCBEd(V106W)) (SEQ ID NO: 541), TadA-CDf base editor (SpCas9n napDNAbp domain) (TadCBEf) (SEQ ID NO: 542), TadA-CDg base editor (SpCas9n napDNAbp domain) (TadCBEg) (SEQ ID NO: 543), TadA-CDa:eNme2Cas9 base editor (SEQ ID NO: 544), TadA-CDa:SaCas9 base editor (SEQ ID NO: 545), TadA-CDa:SpCas9-NG base editor (SEQ ID NO: 546), TadA-CDa:enCjCas9 base editor (SEQ ID NO: 547).

Exemplary polynucleotides encoding TadA-derived cytosine base editors of the disclosure include: TadCBEa-eNme2-C-BE4max vector (SEQ ID NO: 548), TadCBEa-enCjCas9-BE4max vector (SEQ ID NO: 549), TadCBEa-SpCas9-BE4max vector (SEQ ID NO: 550). TadCBEa-SaCas9-BE4max vector (SEQ ID NO: 551), TadCBEa-SpCas9-NG-BE4max vector (SEQ ID NO: 552).

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., Ni-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag.

A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C₁₂ spacer, C₃ spacer, C₆ spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV (SEQ ID NO: 328).

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U: G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and VoB, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. co/i TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H+ V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)ₙ motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
(SEQ ID NO: 358)
GGSGGSPGSPAGSPTSTEEGTSESATPESGPGTSTEPSEGSAPGSPAG
SPTSTEEGTSTEPSEGSAPGTSTEPSEGSAPGTSESATPESGPGSEPA
TSGGSGGS,
(SEQ ID NO: 468)
EGGSEEEEESGS,
or
(SEQ ID NO: 469)
KGPKPKKEESEK.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTE
EGTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than T %, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising an adenosine deaminase variant may be expressed in virtually any host cell of interest, including but not limited to animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism.

As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector.

As the expression vector, animal cell expression plasmids (e.g., pA1-11, pXT1, pRc/CMV, pRc/RSV, pcDNAI/Neo) are known in the art; animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like are preferable.

Expression vectors for use in the present disclosure, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or protein complexes disclosed herein.

A fusion protein or complex of the disclosure can be intracellularly expressed by introducing into the cell an expression vector comprising a nucleic acid sequence encoding the fusion protein or complex.

Mammalian cells contemplated in the present disclosure include, but are not limited to, cell lines such as Human Embryonic Kidney (HEK) cells, monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)]and the like are used. The pH of the medium is preferably about 6 to about 8. The culture is performed at generally about 30° C. to about 40° C. Where necessary, aeration and stirring may be performed.

When a higher eukaryotic cell, such as animal cell, is used as a host cell, a DNA encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such as animal cell are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Reducing Expression of Target Genes in Cells

In some embodiments, provided herein is an immune cell with at least one modification in an endogenous gene or one or more regulatory elements thereof. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof In some embodiments, the at least one modification is a single nucleobase modification. In some embodiments, the at least one modification is generated by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the cell is a hepatocyte.

In some embodiments, the gene is an LPA polynucleotide.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE) are used to edit exons by creating STOP codons.

In some embodiments, provided herein is a liver cell with at least one modification in one or more endogenous genes. In some embodiments, the liver cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes.

In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application Publication No. US 2022/0290164 A1, the disclosure of which is incorporated herein by reference in its entirety for all purposes), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6, AAV9, PHP.EB, PHP.B, AAV.CAP-B10, AAV, CAP-B22, AAV-rh10, a PAL family AAV, or any combination thereof. In embodiments, the AAV is capable of crossing the blood-brain barrier (see, e.g., those AAV vectors disclosed in Liu, et al. “Crossing the blood-brain barrier with AAV vectors,” Metabolic Brain Disease, 36:45-52 (2021), the disclosure of which is incorporated herein by reference in its entirety for all purposes). One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, the AAV vector contains a PAL family AAV capsid (see, Stanton, A., et al. Med 4:31-50 (2023) (doi: doi.org/10.1016/j.medj.2022.11.002), the disclosure of which is incorporated herein by reference in its entirety for all purposes).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N(e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632, WO 2024/073385, and WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminal fragment is fused at the C-terminus to an intein-N and the C-terminal fragment is fused to an intein-C at an N-terminal amino acid selected from the group consisting of S303, T310, T313, S355, A456, S460, A463, T466, S469, T472, T474, C574, S577, A589, and S590, referenced to SEQ ID NO: 197. In various embodiments, the SpCas9 is split between amino acid positions 302 and 303, 309 and 310, 312 and 313, 354 and 355, 455 and 456, 459 and 460, 462 and 463, 465 and 466, 468 and 469, 471 and 472, 473 and 474, 573 and 574, 576 and 577, 588 and 589, or 589 and 590, referenced to SEQ ID NO: 197 to yield an N-terminal fragment and a C-terminal fragment, where the N-terminal fragment is fused at the C-terminus to a an intein-N and where the C-terminal fragment is fused at the N-terminus to an intein-C.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a liver). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein.

In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. In particular embodiments, the methods of treatment include administering to a subject in need thereof a lipid nanoparticle containing a base editor system of the disclosure (e.g., a guide RNA and a polynucleotide encoding a base editor) to a subject. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of a cardiovascular disease in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Base Editing of Lipoprotein(a) (LPA) Polynucleotides to Introduce a Single Nucleotidepolymorphism (SNP) Associate with Reducedserum Levels of LPA and/or Reduced Incidence of Coronary Heart Disease (CHD)

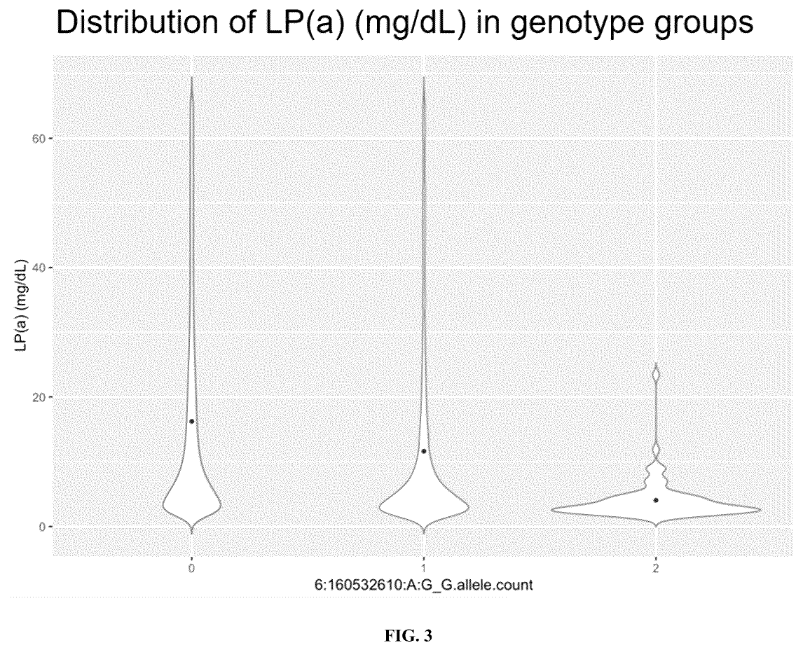

A number of LPA single nucleotide polymorphisms (SNPs) are known to be associated with reduced incidence of CHD and/or reduced serum levels of LPA in a subject (see FIGS. 2 and 3 and Tables 8 and 9). Such SNPs can be referred to as “protective SNPs.” For example, subjects containing two LPA genes having the SNP chr6:160532610:A>G had reduced serum levels of LPA (−4.46 mg/dL LPA on average) and a reduced risk of CHD diagnosis compared to subjects containing only 1 or 0 genes having the SNP. Accordingly, experiments were undertaken to demonstrate that base editing can be used to introduce these SNPs to an LPA polynucleotide as part of a method to prevent or treat coronary heart disease in a subject.

TABLE 8
LPA single nucleotide polymorphisms (SNPs) associated with reduced serum
levels of LPA and/or reduced coronary heart disease (CHD) risk in a subject.¹

| | | | | | | Association with | |
|---|---|---|---|---|---|---|---|
| | | | | | | CHD risk in | |
| | | | | | | UKBB | |
| | | | Association with | | | Odds | |
| | | | LP(a) level in UKBB | | | Ratio | |
| | | | Beta * | | ALT | CHD | P- |
| | | Protein | (mg/ | P- | allele | (95% | value |
| Target SNP | NGS summary | change | dL | value | count | CI) | CHD |
| chr6:160531784: | 3 guides >35% | missense | −4.58 | 7.59E−116 | 16743 | 1.00 | 0.96 |
| T > C | installation | (Y->C) | | | | (0.94-1.06) | |
| chr6:160532531: | 1 guide >40% | Splice site | −6.13 | 1.35E−03 | 183 | 1.06 | 0.82 |
| C > T | installation with silent | causing | | | | (0.63-1.80) | |
| | bystander | null alleles | | | | | |
| chr6:160548552: | 1 guide >30% | missense | −2.22 | 2.59E−01 | 178 | 1.42 | 0.14 |
| G > A | installation with non- | (S->L) | | | | (0.89-2.29) | |
| | silent bystander | | | | | | |
| chr6:160532610: | 1 guide >65% | missense | −4.46 | 9.33E−122 | 19833 | 0.88 | 4.25E−06 |
| A > G | installation with non- | (L->P) | | | | (0.83-0.93) | |
| | silent bystander | | | | | | |
| chr6:160591049: | 1 guide >40% | missense | −4.52 | 1.09E−01 | 93 | 1.06 | 0.87 |
| A > G | installation with non- | (C->S) | | | | (0.52-2.19) | |
| | silent bystander | | | | | | |
| chr6:160635134: | 1 guide >30% | missense | −4.29 | 2.66E−06 | 643 | 1.10 | 0.53 |
| G > A | installation with non- | (P->L) | | | | (0.82-1.48) | |
| | silent bystander | | | | | | |
| chr6:160547886: | 1 guide >30% | missense | −5.5 | 1.00E−02 | 129 | 0.57 | 0.14 |
| A > G | installation with non- | (V->A) | | | | (0.27-1.20) | |
| | silent bystander | | | | | | |

¹An Odds Ratio for CHD <1 indicates that this SNV is associated with a lower risk of coronary heart disease. A p-value <0.05 indicates that is a significant change.
“UKBB” indicates “United Kingdom Biobank,”
“CHD” indicates “coronary heart disease,” and
“NGS” indicates “next generation sequencing.”

TABLE 9
Effect on serum LP(a) concentrations and coronary heart
disease (CHD) risk in subjects having 0, 1, or 2 LPA
genes containing a chr6: 160532610: A > G SNP.

| Sample in LP(a) test | 0 (n = 219383) | 1 (n = 7851) | 2 (n = 31) |
|---|---|---|---|
| LP(a) (mg/dL) | 15.9 ± 16.7 | 11.5 ± 14.8 | 4.19 ± 3.18 |
| mean ± SD | | | |
| Sample in CHD test | 0 (n = 311634) | 1 (n = 13086) | 2 (n = 152) |
| CHD cases | 37573 (12.1%*) | 1411 (10.8%*) | 15 (9.9%²) |

²Number of CHD cases/total number of participants in the genotype group; Participants with 2 genes containing 2 LPA genes containing a chr6:160532610:A>G SNP genotype are less likely diagnosed with CHD compared to subjects with 1 or 0 genes containing the SNP.

Base editor systems (see Tables 1A-1 and 1A-2) for introducing protective SNPs to a LPA polynucleotide were evaluated in HEK293T cells. The cells were transfected with a guide RNA molecule having a sequence selected from those listed in Table 1A-1 and an mRNA molecule encoding a base editor listed in Table 1A-2 as being used in combination with the guide RNA molecule. Sequences for the base editors used are provided in Table 2. Target sites and target LPA polynucleotide modifications corresponding to each base editor system are listed in Table 1A-2. Base editing was measured using next generation sequencing. The base editor systems were effective in introducing a protective SNP to an LPA polynucleotide in the HEK293T cells (see, e.g., Table 8).

### Example 2: Base Editing of a Lipoprotein(a) (LPA) Polynucleotide to Disrupt a Splice Site or Introduce a Stop Codon

One approach for reducing serum concentrations of LPA in a subject to treat or reduce the incidence of coronary heart disease (CHD) in the subject is to reduce expression of LPA in the subject. Accordingly, experiments were undertaken to demonstrate that base editing can be used to introduce a stop codon or disrupt a splice site in an LPA polynucleotide as part of a method to prevent or treat coronary heart disease in a subject.

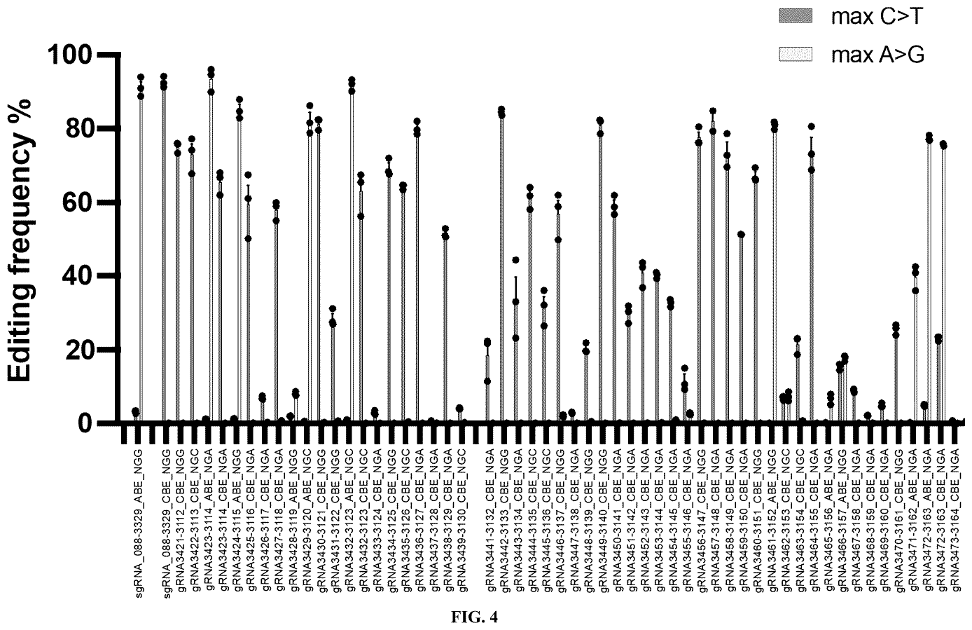

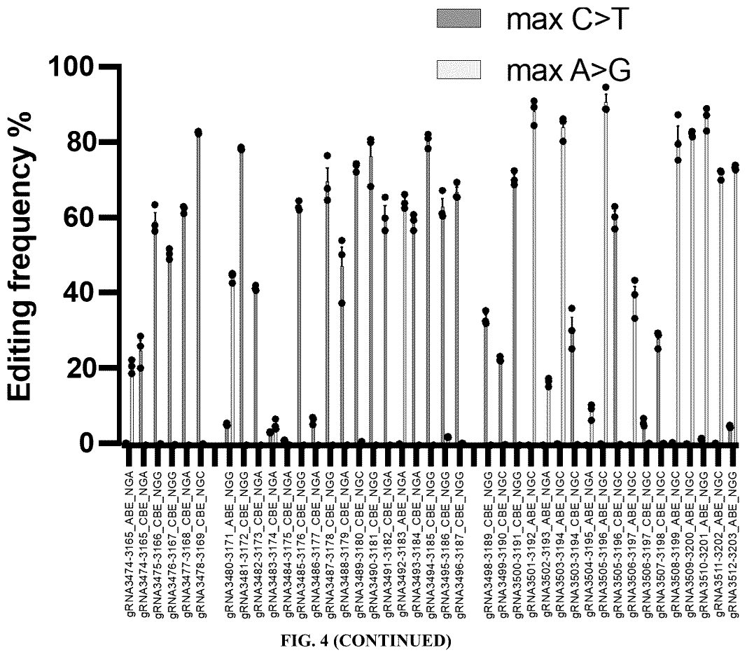

Base editor systems (see Tables 1B-1 and 1B-2) for introducing a stop codon or disrupting a splice site in an LPA polynucleotide were evaluated in HEK293T cells. The cells were transfected with a guide RNA molecule having a sequence selected from those listed in Table 1B-1 and an mRNA molecule encoding a base editor listed in Table 1B-2 as being used in combination with the guide RNA molecule (FIG. 4). Sequences for the base editors used are provided in Table 2. Target sites and target LPA polynucleotide modifications corresponding to each base editor system are listed in Table 1B-2. Base editing was measured using next generation sequencing. Of the base editor systems evaluated, 44 showed base editing frequencies in the HEK293T cells of greater than 60%.

### Example 3: Base Editing of a Lipoprotein(a) (LPA) Polynucleotide to Disrupt a Promoter Region

As noted above, one approach for reducing serum concentrations of LPA in a subject to treat or reduce the incidence of coronary heart disease (CHD) in the subject is to reduce expression of LPA in the subject. Accordingly, experiments were undertaken to demonstrate that base editing can be used to disrupt a promoter region in an LPA polynucleotide as part of a method to prevent or treat coronary heart disease in a subject.

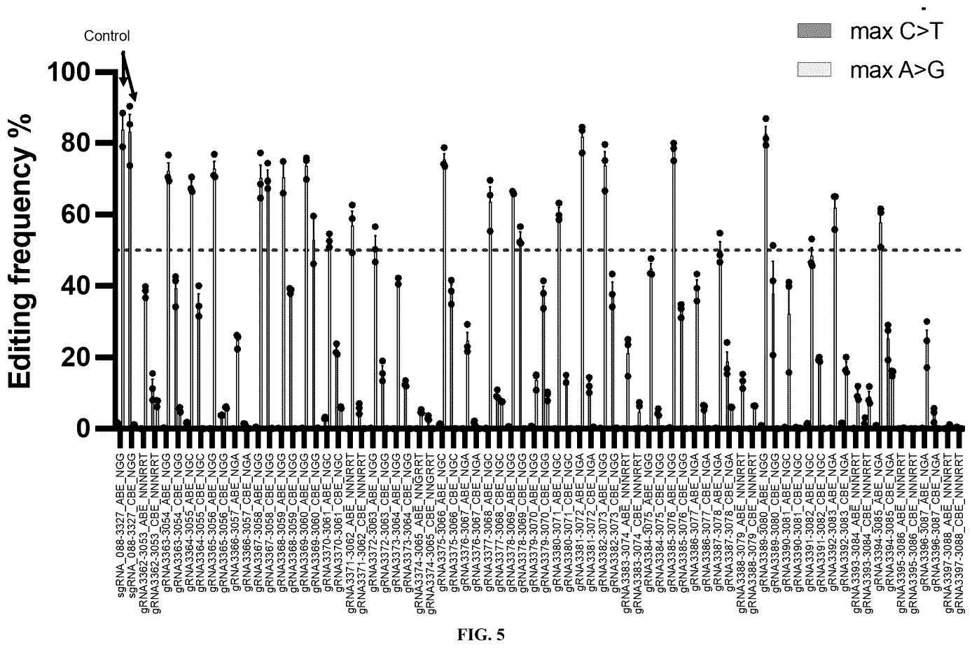

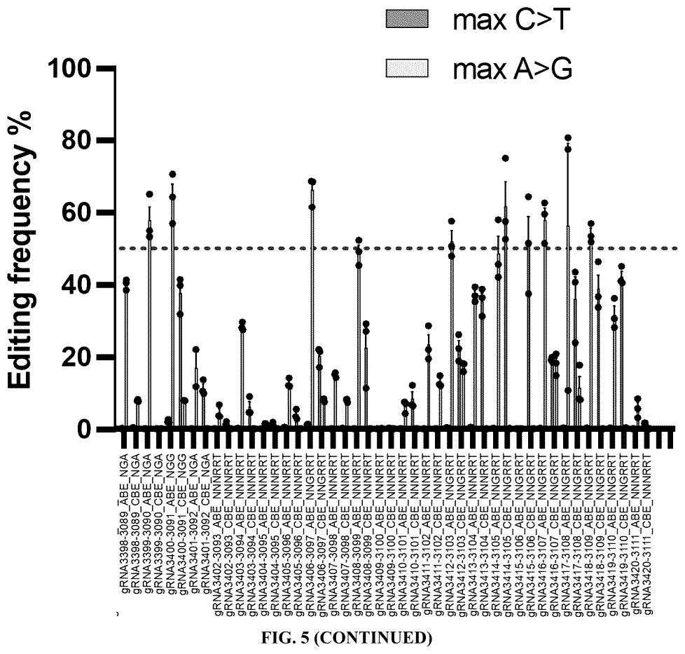

Base editor systems (see Tables 1C-1 and 1C-2) for disrupting a promoter region in an LPA polynucleotide were evaluated in HEK293T cells. The cells were transfected with a guide RNA molecule having a sequence selected from those listed in Table 1C-1 and an mRNA molecule encoding a base editor listed in Table 1C-2 as being used in combination with the guide RNA molecule (FIG. 5). Sequences for the base editors used are provided in Table 2. Target sites corresponding to each base editor system are listed in Table 1C-2. Base editing was measured using next generation sequencing. Of the base editor systems evaluated, 25 showed base editing frequencies in the HEK293T cells of greater than 50%.

### OTHER EMBODIMENTS

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed:

1. A method of editing a nucleobase of a lipoprotein A (LPA) polynucleotide in a cell, the method comprising contacting the LPA polynucleotide with a base editor system comprising a guide RNA, or a polynucleotide encoding said guide RNA, and a base editor comprising a fusion protein or a protein complex comprising a nucleic acid programmable DNA binding protein (napDNAbp) domain and a deaminase domain, or one or more polynucleotides encoding the base editor, wherein said guide RNA targets said base editor to effect an alteration of the nucleobase of the LPA polynucleotide.

2. A method of treating atherosclerosis and/or cardiovascular disease in a subject in need thereof, the method comprising contacting a cell of the subject with a base editor system comprising a base editor comprising a fusion protein or protein complex comprising a nucleic acid programmable DNA binding protein (napDNAbp) domain and a deaminase domain, or one or more polynucleotide encoding the base editor, and a guide RNA, or a polynucleotide encoding said guide RNA, wherein the guide RNA targets the base editor to effect an alteration of a nucleobase of an LPA polynucleotide.

3. The method of claim 1, wherein the guide RNA is selected from the guides listed in Tables 1B-1, 1A-1, and 1C-1.

4. The method of claim 1, wherein the deaminase domain is an adenosine deaminase comprising an amino acid sequence with at least about 90% identity to the following amino acid sequence or a fragment thereof lacking the N-terminal methionine and comprises one or more amino acid alterations selected from the group consisting of I76Y, V82S, Y123H, Y147R, and Q154R compared to the following amino acid sequence:
TadA*7.10
(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD.

5. The method of claim 4, wherein the adenosine deaminase comprises the amino acid alterations I76Y, V82S, Y123H, Y147R, and Q154R.

6. The method of claim 1, wherein the deaminase domain is a cytidine deaminase comprising an amino acid sequence with at least about 90% identity to the following amino acid sequence or a fragment thereof lacking the N-terminal methionine:
ppAPOBEC1
(SEQ ID NO: 23)
MTSEKGPSTGDPTLRRRIESWEFDVFYDPRELRKETCLLYEIKWGMSRKI
WRSSGKNTTNHVEVNFIKKFTSERRFHSSISCSITWFLSWSPCWECSQAI
REFLSQHPGVTLVIYVARLFWHMDQRNRQGLRDLVNSGVTIQIMRASEYY
HCWRNFVNYPPGDEAHWPQYPPLWMMLYALELHCIILSLPPCLKISRRWQ
NHLAFFRLHLQNCHYQTIPPHILLATGLIHPSVTWR.

7. The method of claim 1, wherein alteration of the nucleobase is associated with a reduction in transcription of a polynucleotide sequence encoding the LPA protein; and/or wherein alteration of the nucleobase in the LPA polynucleotide disrupts a splice site.

8. The method of claim 1, wherein the alteration introduces a single nucleotide polymorphism (SNP) into the LPA polynucleotide, wherein the SNP is associated with reduced serum concentrations of LPA in a subject and/or reduced incidence of atherosclerosis and/or cardiovascular disease in a subject, and wherein the SNP is selected from the group consisting of, chr6:160531784:T>C, chr6:160532531:C>T, chr6:160548552:G>A, chr6:160532610:A>G, chr6:160591049:A>G, chr6:160635134:G>A, and chr6:160547886:A>G.

9. The method of claim 2, wherein the method is associated with at least a 10% reduction of incidence of coronary heart disease in the subject.

10. The method of claim 2, wherein the base editor system is administered to the subject using a lipid nanoparticle comprising the guide RNA and an mRNA molecule encoding the base editor.

11. A modified cell comprising an alteration in a nucleobase of a LPA polynucleotide, wherein the alteration is prepared by the method of claim 1.

12. A base editor system comprising a fusion protein or one or more polynucleotides encoding said fusion protein, wherein said fusion protein comprises a nucleic acid programmable DNA binding protein domain (napDNAbp) and a deaminase domain, and a guide RNA, or a guide polynucleotide encoding said guide RNA, wherein said guide RNA targets said base editor to effect an alteration of a nucleobase of an LPA polynucleotide.

13. The base editor system of claim 12, wherein the guide RNA is selected from the guides 5 listed in Tables 1B-1, 1A-1, and 1C-1.

14. The base editor system of claim 12, wherein the deaminase is an adenosine deaminase comprising an amino acid sequence with at least about 90% identity to the following amino acid sequence or a fragment thereof lacking the N-terminal methionine and comprises one or more amino acid alterations selected from the group consisting of I76Y, V82S, Y123H, Y147R, and Q154R compared to the following amino acid sequence:
TadA*7.10
(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD.

15. The base editor system of claim 12, wherein the SNP is selected from the group consisting of, chr6:160531784:T>C, chr6:160532531:C>T, chr6:160548552:G>A, chr6:160532610:A>G, chr6:160591049:A>G, chr6:160635134:G>A, and chr6:160547886:A>G.

16. A polynucleotide or set of polynucleotides encoding the base editor system of claim 12 or a component thereof.

17. A lipid nanoparticle comprising the base editor system of claim 12.

18. A pharmaceutical composition comprising the modified cell of claim 11, and a pharmaceutically acceptable excipient.

19. A kit comprising the modified cell of claim 11, and directions for the use of same.

20. A guide RNA comprising a sequence listed in Table 1B-1, 1B-2, 1A-1, 1A-2, 1C-1, or 1C-2.
